SEQUENCE LISTING (781 sequences)

SEQ ID NO: 1 - protein, 9 aa, synthetic construct
  REGION 1..9: note = Description of Artificial Sequence: Synthetic peptide
GSIFSINAM

SEQ ID NO: 2 - protein, 9 aa, synthetic construct
  REGION 1..9: note = Description of Artificial Sequence: Synthetic peptide
GSIFSINAM

SEQ ID NO: 3 - protein, 9 aa, synthetic construct
  REGION 1..9: note = Description of Artificial Sequence: Synthetic peptide
GIIFSPNAM

SEQ ID NO: 4 - protein, 9 aa, synthetic construct
  REGION 1..9: note = Description of Artificial Sequence: Synthetic peptide
GNIFSINAM

SEQ ID NO: 5 - protein, 9 aa, synthetic construct
  REGION 1..9: note = Description of Artificial Sequence: Synthetic peptide
GRSVSINPM

SEQ ID NO: 6 - protein, 9 aa, synthetic construct
  REGION 1..9: note = Description of Artificial Sequence: Synthetic peptide
GISVNINPM

SEQ ID NO: 7 - protein, 9 aa, synthetic construct
  REGION 1..9: note = Description of Artificial Sequence: Synthetic peptide
GSTFSINAM

SEQ ID NO: 8 - protein, 6 aa, synthetic construct
  REGION 1..6: note = Description of Artificial Sequence: Synthetic peptide
VSINPM

SEQ ID NO: 9 - protein, 9 aa, synthetic construct
  REGION 1..9: note = Description of Artificial Sequence: Synthetic peptide
GRIFSSYDM

SEQ ID NO: 10 - protein, 9 aa, synthetic construct
  REGION 1..9: note = Description of Artificial Sequence: Synthetic peptide
GSIFSIPSM

SEQ ID NO: 11 - protein, 9 aa, synthetic construct
  REGION 1..9: note = Description of Artificial Sequence: Synthetic peptide
GRIFSSYDM

SEQ ID NO: 12 - protein, 9 aa, synthetic construct
  REGION 1..9: note = Description of Artificial Sequence: Synthetic peptide
GRTFSNNAM

SEQ ID NO: 13 - protein, 9 aa, synthetic construct
  REGION 1..9: note = Description of Artificial Sequence: Synthetic peptide
GRTFSRLAM

SEQ ID NO: 14 - protein, 9 aa, synthetic construct
  REGION 1..9: note = Description of Artificial Sequence: Synthetic peptide
GRMFSSYDM

SEQ ID NO: 15 - protein, 9 aa, synthetic construct
  REGION 1..9: note = Description of Artificial Sequence: Synthetic peptide
GSIFSINAV

SEQ ID NO: 16 - protein, 9 aa, synthetic construct
  REGION 1..9: note = Description of Artificial Sequence: Synthetic peptide
GRIFSSYDM

SEQ ID NO: 17 - protein, 9 aa, synthetic construct
  REGION 1..9: note = Description of Artificial Sequence: Synthetic peptide
GRIFSINPM

SEQ ID NO: 18 - protein, 9 aa, synthetic construct
  REGION 1..9: note = Description of Artificial Sequence: Synthetic peptide
GRIFSIYDM

SEQ ID NO: 19 - protein, 9 aa, synthetic construct
  REGION 1..9: note = Description of Artificial Sequence: Synthetic peptide
GFTFSSAWM

SEQ ID NO: 20 - protein, 9 aa, synthetic construct
  REGION 1..9: note = Description of Artificial Sequence: Synthetic peptide
GIIFSPNAM

SEQ ID NO: 21 - protein, 9 aa, synthetic construct
  REGION 1..9: note = Description of Artificial Sequence: Synthetic peptide
GIIFSPNAM

SEQ ID NO: 22 - protein, 9 aa, synthetic construct
  REGION 1..9: note = Description of Artificial Sequence: Synthetic peptide
GIIFSPNAL

SEQ ID NO: 23 - protein, 9 aa, synthetic construct
  REGION 1..9: note = Description of Artificial Sequence: Synthetic peptide
GIIFSPNAM

SEQ ID NO: 24 - protein, 9 aa, synthetic construct
  REGION 1..9: note = Description of Artificial Sequence: Synthetic peptide
GRSVSINPM

SEQ ID NO: 25 - protein, 7 aa, synthetic construct
  REGION 1..7: note = Description of Artificial Sequence: Synthetic peptide
VFILNAM

SEQ ID NO: 26 - protein, 9 aa, synthetic construct
  REGION 1..9: note = Description of Artificial Sequence: Synthetic peptide
RGTFTTDAM

SEQ ID NO: 27 - protein, 9 aa, synthetic construct
  REGION 1..9: note = Description of Artificial Sequence: Synthetic peptide
VRAFSSRAM

SEQ ID NO: 28 - protein, 9 aa, synthetic construct
  REGION 1..9: note = Description of Artificial Sequence: Synthetic peptide
GGTFSDYAW

SEQ ID NO: 29 - protein, 9 aa, synthetic construct
  REGION 1..9: note = Description of Artificial Sequence: Synthetic peptide
GIIFSPNAM

SEQ ID NO: 30 - protein, 9 aa, synthetic construct
  REGION 1..9: note = Description of Artificial Sequence: Synthetic peptide
GIIFSPNAM

SEQ ID NO: 31 - protein, 9 aa, synthetic construct
  REGION 1..9: note = Description of Artificial Sequence: Synthetic peptide
GITNRITTM

SEQ ID NO: 32 - protein, 9 aa, synthetic construct
  REGION 1..9: note = Description of Artificial Sequence: Synthetic peptide
GSVRTINDM

SEQ ID NO: 33 - protein, 9 aa, synthetic construct
  REGION 1..9: note = Description of Artificial Sequence: Synthetic peptide
GIIFSPNAM

SEQ ID NO: 34 - protein, 9 aa, synthetic construct
  REGION 1..9: note = Description of Artificial Sequence: Synthetic peptide
GRTFRSYTM

SEQ ID NO: 35 - protein, 9 aa, synthetic construct
  REGION 1..9: note = Description of Artificial Sequence: Synthetic peptide
GIIFSPNAM

SEQ ID NO: 36 - protein, 9 aa, synthetic construct
  REGION 1..9: note = Description of Artificial Sequence: Synthetic peptide
GRTFSSYVM

SEQ ID NO: 37 - protein, 9 aa, synthetic construct
  REGION 1..9: note = Description of Artificial Sequence: Synthetic peptide
FLENPPFAI

SEQ ID NO: 38 - protein, 9 aa, synthetic construct
  REGION 1..9: note = Description of Artificial Sequence: Synthetic peptide
GSTVTISTV

SEQ ID NO: 39 - protein, 9 aa, synthetic construct
  REGION 1..9: note = Description of Artificial Sequence: Synthetic peptide
GIIFNPNAM

SEQ ID NO: 40 - protein, 9 aa, synthetic construct
  REGION 1..9: note = Description of Artificial Sequence: Synthetic peptide
GIIFSPNAL

SEQ ID NO: 41 - protein, 9 aa, synthetic construct
  REGION 1..9: note = Description of Artificial Sequence: Synthetic peptide
GIIFSPNAM

SEQ ID NO: 42 - protein, 9 aa, synthetic construct
  REGION 1..9: note = Description of Artificial Sequence: Synthetic peptide
GIIFSPNAL

SEQ ID NO: 43 - protein, 12 aa, synthetic construct
  REGION 1..12: note = Description of Artificial Sequence: Synthetic peptide
GIFESTFDATAM

SEQ ID NO: 44 - protein, 9 aa, synthetic construct
  REGION 1..9: note = Description of Artificial Sequence: Synthetic peptide
GSIFSTNVM

SEQ ID NO: 45 - protein, 9 aa, synthetic construct
  REGION 1..9: note = Description of Artificial Sequence: Synthetic peptide
GRTFDKYRI

SEQ ID NO: 46 - protein, 9 aa, synthetic construct
  REGION 1..9: note = Description of Artificial Sequence: Synthetic peptide
GRTFINRSM

SEQ ID NO: 47 - protein, 9 aa, synthetic construct
  REGION 1..9: note = Description of Artificial Sequence: Synthetic peptide
GFTFSMYGM

SEQ ID NO: 48 - protein, 9 aa, synthetic construct
  REGION 1..9: note = Description of Artificial Sequence: Synthetic peptide
GLTFSSYGM

SEQ ID NO: 49 - protein, 9 aa, synthetic construct
  REGION 1..9: note = Description of Artificial Sequence: Synthetic peptide
GRTFSSYAM

SEQ ID NO: 50 - protein, 14 aa, synthetic construct
  REGION 1..14: note = Description of Artificial Sequence: Synthetic peptide
GRDASDGTFSRYVM

SEQ ID NO: 51 - protein, 9 aa, synthetic construct
  REGION 1..9: note = Description of Artificial Sequence: Synthetic peptide
GRTFGSLHM

SEQ ID NO: 52 - protein, 9 aa, synthetic construct
  REGION 1..9: note = Description of Artificial Sequence: Synthetic peptide
GFTFDDYVI

SEQ ID NO: 53 - protein, 9 aa, synthetic construct
  REGION 1..9: note = Description of Artificial Sequence: Synthetic peptide
GFRLNDYYV

SEQ ID NO: 54 - protein, 9 aa, synthetic construct
  REGION 1..9: note = Description of Artificial Sequence: Synthetic peptide
GIIFRINTM

SEQ ID NO: 55 - protein, 9 aa, synthetic construct
  REGION 1..9: note = Description of Artificial Sequence: Synthetic peptide
GFTLGYFAI

SEQ ID NO: 56 - protein, 9 aa, synthetic construct
  REGION 1..9: note = Description of Artificial Sequence: Synthetic peptide
GLAFNTKTM

SEQ ID NO: 57 - protein, 9 aa, synthetic construct
  REGION 1..9: note = Description of Artificial Sequence: Synthetic peptide
GFTFSRYLM

SEQ ID NO: 58 - protein, 9 aa, synthetic construct
  REGION 1..9: note = Description of Artificial Sequence: Synthetic peptide
GRIFGSLHM

SEQ ID NO: 59 - protein, 9 aa, synthetic construct
  REGION 1..9: note = Description of Artificial Sequence: Synthetic peptide
GSISSIKAM

SEQ ID NO: 60 - protein, 9 aa, synthetic construct
  REGION 1..9: note = Description of Artificial Sequence: Synthetic peptide
GRTFSRDAM

SEQ ID NO: 61 - protein, 9 aa, synthetic construct
  REGION 1..9: note = Description of Artificial Sequence: Synthetic peptide
GRTFSSYAM

SEQ ID NO: 62 - protein, 9 aa, synthetic construct
  REGION 1..9: note = Description of Artificial Sequence: Synthetic peptide
GRTFSIYAM

SEQ ID NO: 63 - protein, 9 aa, synthetic construct
  REGION 1..9: note = Description of Artificial Sequence: Synthetic peptide
GLAFSTKTM

SEQ ID NO: 64 - protein, 9 aa, synthetic construct
  REGION 1..9: note = Description of Artificial Sequence: Synthetic peptide
GRTFSSNTM

SEQ ID NO: 65 - protein, 9 aa, synthetic construct
  REGION 1..9: note = Description of Artificial Sequence: Synthetic peptide
GRTFSSYGM

SEQ ID NO: 66 - protein, 9 aa, synthetic construct
  REGION 1..9: note = Description of Artificial Sequence: Synthetic peptide
GRALSAYIM

SEQ ID NO: 67 - protein, 9 aa, synthetic construct
  REGION 1..9: note = Description of Artificial Sequence: Synthetic peptide
VRTFNTYNI

SEQ ID NO: 68 - protein, 9 aa, synthetic construct
  REGION 1..9: note = Description of Artificial Sequence: Synthetic peptide
GRSFSSYNM

SEQ ID NO: 69 - protein, 9 aa, synthetic construct
  REGION 1..9: note = Description of Artificial Sequence: Synthetic peptide
ERTFSSYTM

SEQ ID NO: 70 - protein, 9 aa, synthetic construct
  REGION 1..9: note = Description of Artificial Sequence: Synthetic peptide
GTFFRINYM

SEQ ID NO: 71 - protein, 9 aa, synthetic construct
  REGION 1..9: note = Description of Artificial Sequence: Synthetic peptide
GRTFSSYAM

SEQ ID NO: 72 - protein, 9 aa, synthetic construct
  REGION 1..9: note = Description of Artificial Sequence: Synthetic peptide
GFNFSLYSM

SEQ ID NO: 73 - protein, 9 aa, synthetic construct
  REGION 1..9: note = Description of Artificial Sequence: Synthetic peptide
ARILSSHRM

SEQ ID NO: 74 - protein, 9 aa, synthetic construct
  REGION 1..9: note = Description of Artificial Sequence: Synthetic peptide
GFTLDNYAI

SEQ ID NO: 75 - protein, 9 aa, synthetic construct
  REGION 1..9: note = Description of Artificial Sequence: Synthetic peptide
GFMPDYSAL

SEQ ID NO: 76 - protein, 11 aa, synthetic construct
  REGION 1..11: note = Description of Artificial Sequence: Synthetic peptide
LYSLRTRLQYL

SEQ ID NO: 77 - protein, 9 aa, synthetic construct
  REGION 1..9: note = Description of Artificial Sequence: Synthetic peptide
GFTFSSYWM

SEQ ID NO: 78 - protein, 9 aa, synthetic construct
  REGION 1..9: note = Description of Artificial Sequence: Synthetic peptide
GFTFSNFWM

SEQ ID NO: 79 - protein, 9 aa, synthetic construct
  REGION 1..9: note = Description of Artificial Sequence: Synthetic peptide
ARTFSSYAM

SEQ ID NO: 80 - protein, 9 aa, synthetic construct
  REGION 1..9: note = Description of Artificial Sequence: Synthetic peptide
GRTFINRSM

SEQ ID NO: 81 - protein, 9 aa, synthetic construct
  REGION 1..9: note = Description of Artificial Sequence: Synthetic peptide
GFTLDYFAI

SEQ ID NO: 82 - protein, 9 aa, synthetic construct
  REGION 1..9: note = Description of Artificial Sequence: Synthetic peptide
GRTFSMYAM

SEQ ID NO: 83 - protein, 9 aa, synthetic construct
  REGION 1..9: note = Description of Artificial Sequence: Synthetic peptide
VHSFSNYAL

SEQ ID NO: 84 - protein, 9 aa, synthetic construct
  REGION 1..9: note = Description of Artificial Sequence: Synthetic peptide
GRPFINYNM

SEQ ID NO: 85 - protein, 9 aa, synthetic construct
  REGION 1..9: note = Description of Artificial Sequence: Synthetic peptide
AFTFDDFAV

SEQ ID NO: 86 - protein, 9 aa, synthetic construct
  REGION 1..9: note = Description of Artificial Sequence: Synthetic peptide
GFSLDHSAI

SEQ ID NO: 87 - protein, 9 aa, synthetic construct
  REGION 1..9: note = Description of Artificial Sequence: Synthetic peptide
GFDFNIYWM

SEQ ID NO: 88 - protein, 9 aa, synthetic construct
  REGION 1..9: note = Description of Artificial Sequence: Synthetic peptide
GRTLRSYVM

SEQ ID NO: 89 - protein, 9 aa, synthetic construct
  REGION 1..9: note = Description of Artificial Sequence: Synthetic peptide
GLIFGDYVM

SEQ ID NO: 90 - protein, 9 aa, synthetic construct
  REGION 1..9: note = Description of Artificial Sequence: Synthetic peptide
GRTFSNLAM

SEQ ID NO: 91 - protein, 9 aa, synthetic construct
  REGION 1..9: note = Description of Artificial Sequence: Synthetic peptide
GRTFSSYAM

SEQ ID NO: 92 - protein, 10 aa, synthetic construct
  REGION 1..10: note = Description of Artificial Sequence: Synthetic peptide
ITTGGNTANT

SEQ ID NO: 93 - protein, 11 aa, synthetic construct
  REGION 1..11: note = Description of Artificial Sequence: Synthetic peptide
ITITSGRGGNT

SEQ ID NO: 94 - protein, 7 aa, synthetic construct
  REGION 1..7: note = Description of Artificial Sequence: Synthetic peptide
ITSFGII

SEQ ID NO: 95 - protein, 10 aa, synthetic construct
  REGION 1..10: note = Description of Artificial Sequence: Synthetic peptide
ITTGGSYGNT

SEQ ID NO: 96 - protein, 7 aa, synthetic construct
  REGION 1..7: note = Description of Artificial Sequence: Synthetic peptide
LLPSGRT

SEQ ID NO: 97 - protein, 7 aa, synthetic construct
  REGION 1..7: note = Description of Artificial Sequence: Synthetic peptide
LLPTGTT

SEQ ID NO: 98 - protein, 7 aa, synthetic construct
  REGION 1..7: note = Description of Artificial Sequence: Synthetic peptide
ISRAGST

SEQ ID NO: 99 - protein, 7 aa, synthetic construct
  REGION 1..7: note = Description of Artificial Sequence: Synthetic peptide
LLSMARA

SEQ ID NO: 100 - protein, 8 aa, synthetic construct
  REGION 1..8: note = Description of Artificial Sequence: Synthetic peptide
IRWGNGNT

SEQ ID NO: 101 - protein, 7 aa, synthetic construct
  REGION 1..7: note = Description of Artificial Sequence: Synthetic peptide
ITRSGST

SEQ ID NO: 102 - protein, 8 aa, synthetic construct
  REGION 1..8: note = Description of Artificial Sequence: Synthetic peptide
IRWGNGNT

SEQ ID NO: 103 - protein, 7 aa, synthetic construct
  REGION 1..7: note = Description of Artificial Sequence: Synthetic peptide
ISRAGNT

SEQ ID NO: 104 - protein, 8 aa, synthetic construct
  REGION 1..8: note = Description of Artificial Sequence: Synthetic peptide
ISWSGGNT

SEQ ID NO: 105 - protein, 8 aa, synthetic construct
  REGION 1..8: note = Description of Artificial Sequence: Synthetic peptide
ITKNGRTT

SEQ ID NO: 106 - protein, 11 aa, synthetic construct
  REGION 1..11: note = Description of Artificial Sequence: Synthetic peptide
IGTGGSSSGNT

SEQ ID NO: 107 - protein, 8 aa, synthetic construct
  REGION 1..8: note = Description of Artificial Sequence: Synthetic peptide
IRWGNGNT

SEQ ID NO: 108 - protein, 10 aa, synthetic construct
  REGION 1..10: note = Description of Artificial Sequence: Synthetic peptide
LMTGGKTPDA

SEQ ID NO: 109 - protein, 8 aa, synthetic construct
  REGION 1..8: note = Description of Artificial Sequence: Synthetic peptide
ITWGNGNT

SEQ ID NO: 110 - protein, 8 aa, synthetic construct
  REGION 1..8: note = Description of Artificial Sequence: Synthetic peptide
IYPSGSST

SEQ ID NO: 111 - protein, 7 aa, synthetic construct
  REGION 1..7: note = Description of Artificial Sequence: Synthetic peptide
ITSFGII

SEQ ID NO: 112 - protein, 7 aa, synthetic construct
  REGION 1..7: note = Description of Artificial Sequence: Synthetic peptide
ITSFGII

SEQ ID NO: 113 - protein, 7 aa, synthetic construct
  REGION 1..7: note = Description of Artificial Sequence: Synthetic peptide
IISGGRS

SEQ ID NO: 114 - protein, 7 aa, synthetic construct
  REGION 1..7: note = Description of Artificial Sequence: Synthetic peptide
ITSFGII

SEQ ID NO: 115 - protein, 7 aa, synthetic construct
  REGION 1..7: note = Description of Artificial Sequence: Synthetic peptide
LLPSGRT

SEQ ID NO: 116 - protein, 7 aa, synthetic construct
  REGION 1..7: note = Description of Artificial Sequence: Synthetic peptide
IISFGIK

SEQ ID NO: 117 - protein, 7 aa, synthetic construct
  REGION 1..7: note = Description of Artificial Sequence: Synthetic peptide
KSSGADP

SEQ ID NO: 118 - protein, 8 aa, synthetic construct
  REGION 1..8: note = Description of Artificial Sequence: Synthetic peptide
ISSSGSST

SEQ ID NO: 119 - protein, 8 aa, synthetic construct
  REGION 1..8: note = Description of Artificial Sequence: Synthetic peptide
ISWTGGII

SEQ ID NO: 120 - protein, 7 aa, synthetic construct
  REGION 1..7: note = Description of Artificial Sequence: Synthetic peptide
ITSFGII

SEQ ID NO: 121 - protein, 7 aa, synthetic construct
  REGION 1..7: note = Description of Artificial Sequence: Synthetic peptide
ITSFGII

SEQ ID NO: 122 - protein, 8 aa, synthetic construct
  REGION 1..8: note = Description of Artificial Sequence: Synthetic peptide
IRDDRDAN

SEQ ID NO: 123 - protein, 7 aa, synthetic construct
  REGION 1..7: note = Description of Artificial Sequence: Synthetic peptide
ISSGGNT

SEQ ID NO: 124 - protein, 7 aa, synthetic construct
  REGION 1..7: note = Description of Artificial Sequence: Synthetic peptide
ITSFGII

SEQ ID NO: 125 - protein, 8 aa, synthetic construct
  REGION 1..8: note = Description of Artificial Sequence: Synthetic peptide
ISWSAGST

SEQ ID NO: 126 - protein, 7 aa, synthetic construct
  REGION 1..7: note = Description of Artificial Sequence: Synthetic peptide
ITSSGII

SEQ ID NO: 127 - protein, 8 aa, synthetic construct
  REGION 1..8: note = Description of Artificial Sequence: Synthetic peptide
ISWSGGSS

SEQ ID NO: 128 - protein, 8 aa, synthetic construct
  REGION 1..8: note = Description of Artificial Sequence: Synthetic peptide
ITYCVMEI

SEQ ID NO: 129 - protein, 7 aa, synthetic construct
  REGION 1..7: note = Description of Artificial Sequence: Synthetic peptide
ISSDSTT

SEQ ID NO: 130 - protein, 7 aa, synthetic construct
  REGION 1..7: note = Description of Artificial Sequence: Synthetic peptide
ITSFGII

SEQ ID NO: 131 - protein, 7 aa, synthetic construct
  REGION 1..7: note = Description of Artificial Sequence: Synthetic peptide
IISGGRS

SEQ ID NO: 132 - protein, 7 aa, synthetic construct
  REGION 1..7: note = Description of Artificial Sequence: Synthetic peptide
ITSFGII

SEQ ID NO: 133 - protein, 7 aa, synthetic construct
  REGION 1..7: note = Description of Artificial Sequence: Synthetic peptide
IISGGRS

SEQ ID NO: 134 - protein, 7 aa, synthetic construct
  REGION 1..7: note = Description of Artificial Sequence: Synthetic peptide
IGSRGSI

SEQ ID NO: 135 - protein, 7 aa, synthetic construct
  REGION 1..7: note = Description of Artificial Sequence: Synthetic peptide
ITSGGNT

SEQ ID NO: 136 - protein, 8 aa, synthetic construct
  REGION 1..8: note = Description of Artificial Sequence: Synthetic peptide
ISWNGAYT

SEQ ID NO: 137 - protein, 8 aa, synthetic construct
  REGION 1..8: note = Description of Artificial Sequence: Synthetic peptide
ISSSGSNT

SEQ ID NO: 138 - protein, 8 aa, synthetic construct
  REGION 1..8: note = Description of Artificial Sequence: Synthetic peptide
INSGGART

SEQ ID NO: 139 - protein, 7 aa, synthetic construct
  REGION 1..7: note = Description of Artificial Sequence: Synthetic peptide
IKMSGDT

SEQ ID NO: 140 - protein, 8 aa, synthetic construct
  REGION 1..8: note = Description of Artificial Sequence: Synthetic peptide
INWSGGRI

SEQ ID NO: 141 - protein, 8 aa, synthetic construct
  REGION 1..8: note = Description of Artificial Sequence: Synthetic peptide
MRWNTGSE

SEQ ID NO: 142 - protein, 8 aa, synthetic construct
  REGION 1..8: note = Description of Artificial Sequence: Synthetic peptide
ISAAGGVT

SEQ ID NO: 143 - protein, 8 aa, synthetic construct
  REGION 1..8: note = Description of Artificial Sequence: Synthetic peptide
TSSSDGDT

SEQ ID NO: 144 - protein, 8 aa, synthetic construct
  REGION 1..8: note = Description of Artificial Sequence: Synthetic peptide
TGSRSGRL

SEQ ID NO: 145 - protein, 7 aa, synthetic construct
  REGION 1..7: note = Description of Artificial Sequence: Synthetic peptide
ITRAGST

SEQ ID NO: 146 - protein, 8 aa, synthetic construct
  REGION 1..8: note = Description of Artificial Sequence: Synthetic peptide
ISNSDGST

SEQ ID NO: 147 - protein, 8 aa, synthetic construct
  REGION 1..8: note = Description of Artificial Sequence: Synthetic peptide
ITWGTINT

SEQ ID NO: 148 - protein, 8 aa, synthetic construct
  REGION 1..8: note = Description of Artificial Sequence: Synthetic peptide
VNSGGAMT

SEQ ID NO: 149 - protein, 8 aa, synthetic construct
  REGION 1..8: note = Description of Artificial Sequence: Synthetic peptide
ITAAGGVT

SEQ ID NO: 150 - protein, 7 aa, synthetic construct
  REGION 1..7: note = Description of Artificial Sequence: Synthetic peptide
WRMYSGT

SEQ ID NO: 151 - protein, 8 aa, synthetic construct
  REGION 1..8: note = Description of Artificial Sequence: Synthetic peptide
INWNGRST

SEQ ID NO: 152 - protein, 8 aa, synthetic construct
  REGION 1..8: note = Description of Artificial Sequence: Synthetic peptide
INWSGGRI

SEQ ID NO: 153 - protein, 8 aa, synthetic construct
  REGION 1..8: note = Description of Artificial Sequence: Synthetic peptide
INWSGGRI

SEQ ID NO: 154 - protein, 8 aa, synthetic construct
  REGION 1..8: note = Description of Artificial Sequence: Synthetic peptide
ITWGTSST

SEQ ID NO: 155 - protein, 8 aa, synthetic construct
  REGION 1..8: note = Description of Artificial Sequence: Synthetic peptide
IASSDGAT

SEQ ID NO: 156 - protein, 7 aa, synthetic construct
  REGION 1..7: note = Description of Artificial Sequence: Synthetic peptide
IKVSGDT

SEQ ID NO: 157 - protein, 8 aa, synthetic construct
  REGION 1..8: note = Description of Artificial Sequence: Synthetic peptide
ISSSGSNT

SEQ ID NO: 158 - protein, 8 aa, synthetic construct
  REGION 1..8: note = Description of Artificial Sequence: Synthetic peptide
ISWGRGNT

SEQ ID NO: 159 - protein, 7 aa, synthetic construct
  REGION 1..7: note = Description of Artificial Sequence: Synthetic peptide
ITWSGNT

SEQ ID NO: 160 - protein, 8 aa, synthetic construct
  REGION 1..8: note = Description of Artificial Sequence: Synthetic peptide
ISWSGGNT

SEQ ID NO: 161 - protein, 7 aa, synthetic construct
  REGION 1..7: note = Description of Artificial Sequence: Synthetic peptide
ISSGGST

SEQ ID NO: 162 - protein, 8 aa, synthetic construct
  REGION 1..8: note = Description of Artificial Sequence: Synthetic peptide
IRWTRSST

SEQ ID NO: 163 - protein, 8 aa, synthetic construct
  REGION 1..8: note = Description of Artificial Sequence: Synthetic peptide
ISNLSVRT

SEQ ID NO: 164 - protein, 8 aa, synthetic construct
  REGION 1..8: note = Description of Artificial Sequence: Synthetic peptide
IRWGSGST

SEQ ID NO: 165 - protein, 8 aa, synthetic construct
  REGION 1..8: note = Description of Artificial Sequence: Synthetic peptide
ISRSDGDT

SEQ ID NO: 166 - protein, 8 aa, synthetic construct
  REGION 1..8: note = Description of Artificial Sequence: Synthetic peptide
ISRDGHTY

SEQ ID NO: 167 - protein, 8 aa, synthetic construct
  REGION 1..8: note = Description of Artificial Sequence: Synthetic peptide
TYWPIFCH

SEQ ID NO: 168 - protein, 8 aa, synthetic construct
  REGION 1..8: note = Description of Artificial Sequence: Synthetic peptide
IDTGGGST

SEQ ID NO: 169 - protein, 8 aa, synthetic construct
  REGION 1..8: note = Description of Artificial Sequence: Synthetic peptide
LNTGGGAT

SEQ ID NO: 170 - protein, 8 aa, synthetic construct
  REGION 1..8: note = Description of Artificial Sequence: Synthetic peptide
ISWDGATT

SEQ ID NO: 171 - protein, 7 aa, synthetic construct
  REGION 1..7: note = Description of Artificial Sequence: Synthetic peptide
GSSGSYS

SEQ ID NO: 172 - protein, 8 aa, synthetic construct
  REGION 1..8: note = Description of Artificial Sequence: Synthetic peptide
ISNIDGIT

SEQ ID NO: 173 - protein, 8 aa, synthetic construct
  REGION 1..8: note = Description of Artificial Sequence: Synthetic peptide
INWSGAST

SEQ ID NO: 174 - protein, 7 aa, synthetic construct
  REGION 1..7: note = Description of Artificial Sequence: Synthetic peptide
ITWNAES

SEQ ID NO: 175 - protein, 8 aa, synthetic construct
  REGION 1..8: note = Description of Artificial Sequence: Synthetic peptide
ISWSGDST

SEQ ID NO: 176 - protein, 8 aa, synthetic construct
  REGION 1..8: note = Description of Artificial Sequence: Synthetic peptide
LSSSDGST

SEQ ID NO: 177 - protein, 7 aa, synthetic construct
  REGION 1..7: note = Description of Artificial Sequence: Synthetic peptide
VHHDGTA

SEQ ID NO: 178 - protein, 8 aa, synthetic construct
  REGION 1..8: note = Description of Artificial Sequence: Synthetic peptide
IRSTGDTI

SEQ ID NO: 179 - protein, 8 aa, synthetic construct
  REGION 1..8: note = Description of Artificial Sequence: Synthetic peptide
LSWSGIST

SEQ ID NO: 180 - protein, 7 aa, synthetic construct
  REGION 1..7: note = Description of Artificial Sequence: Synthetic peptide
ISSDSTT

SEQ ID NO: 181 - protein, 7 aa, synthetic construct
  REGION 1..7: note = Description of Artificial Sequence: Synthetic peptide
INWSDNT

SEQ ID NO: 182 - protein, 8 aa, synthetic construct
  REGION 1..8: note = Description of Artificial Sequence: Synthetic peptide
IRWTRSST

SEQ ID NO: 183 - protein, 11 aa, synthetic construct
  REGION 1..11: note = Description of Artificial Sequence: Synthetic peptide
AARGLSYEYDY

SEQ ID NO: 184 - protein, 12 aa, synthetic construct
  REGION 1..12: note = Description of Artificial Sequence: Synthetic peptide
AARGAMTYEYDY

SEQ ID NO: 185 - protein, 13 aa, synthetic construct
  REGION 1..13: note = Description of Artificial Sequence: Synthetic peptide
NAKTFDGTRWRDY

SEQ ID NO: 186 - protein, 12 aa, synthetic construct
  REGION 1..12: note = Description of Artificial Sequence: Synthetic peptide
AARGSQTYEYDY

SEQ ID NO: 187 - protein, 5 aa, synthetic construct
  REGION 1..5: note = Description of Artificial Sequence: Synthetic peptide
NTADF

SEQ ID NO: 188 - protein, 7 aa, synthetic construct
  REGION 1..7: note = Description of Artificial Sequence: Synthetic peptide
YCNTADF

SEQ ID NO: 189 - protein, 14 aa, synthetic construct
  REGION 1..14: note = Description of Artificial Sequence: Synthetic peptide
KASSGSSVYIGFGS

SEQ ID NO: 190 - protein, 5 aa, synthetic construct
  REGION 1..5: note = Description of Artificial Sequence: Synthetic peptide
NTTDF

SEQ ID NO: 191 - protein, 12 aa, synthetic construct
  REGION 1..12: note = Description of Artificial Sequence: Synthetic peptide
AARIVNGGSWDY

SEQ ID NO: 192 - protein, 12 aa, synthetic construct
  REGION 1..12: note = Description of Artificial Sequence: Synthetic peptide
NADFYGLYPRQY

SEQ ID NO: 193 - protein, 11 aa, synthetic construct
  REGION 1..11: note = Description of Artificial Sequence: Synthetic peptide
AARGLAYEYEY

SEQ ID NO: 194 - protein, 14 aa, synthetic construct
  REGION 1..14: note = Description of Artificial Sequence: Synthetic peptide
KASSGSSVYIGVGS

SEQ ID NO: 195 - protein, 21 aa, synthetic construct
  REGION 1..21: note = Description of Artificial Sequence: Synthetic peptide
AAPERSGSYAYTPSRLNEYAY

SEQ ID NO: 196 - protein, 12 aa, synthetic construct
  REGION 1..12: note = Description of Artificial Sequence: Synthetic peptide
AGRRSNADNWDY

SEQ ID NO: 197 - protein, 12 aa, synthetic construct
  REGION 1..12: note = Description of Artificial Sequence: Synthetic peptide
AARGTISYEYDY

SEQ ID NO: 198 - protein, 12 aa, synthetic construct
  REGION 1..12: note = Description of Artificial Sequence: Synthetic peptide
ATRIVNGGSWDY

SEQ ID NO: 199 - protein, 14 aa, synthetic construct
  REGION 1..14: note = Description of Artificial Sequence: Synthetic peptide
YNCDFWGLAYEYDY

SEQ ID NO: 200 - protein, 12 aa, synthetic construct
  REGION 1..12: note = Description of Artificial Sequence: Synthetic peptide
PARIVNGGSWDY

SEQ ID NO: 201 - protein, 24 aa, synthetic construct
  REGION 1..24: note = Description of Artificial Sequence: Synthetic peptide
ATASRRGVVSLTSNPSTSRNDFSS

SEQ ID NO: 202 - protein, 13 aa, synthetic construct
  REGION 1..13: note = Description of Artificial Sequence: Synthetic peptide
NAKTFDGTRWHDY

SEQ ID NO: 203 - protein, 13 aa, synthetic construct
  REGION 1..13: note = Description of Artificial Sequence: Synthetic peptide
NAKAFDGTRWHDY

SEQ ID NO: 204 - protein, 13 aa, synthetic construct
  REGION 1..13: note = Description of Artificial Sequence: Synthetic peptide
NANVYDGNRWRTY

SEQ ID NO: 205 - protein, 13 aa, synthetic construct
  REGION 1..13: note = Description of Artificial Sequence: Synthetic peptide
NAKSFDGSRWNDY

SEQ ID NO: 206 - protein, 5 aa, synthetic construct
  REGION 1..5: note = Description of Artificial Sequence: Synthetic peptide
NTADF

SEQ ID NO: 207 - protein, 13 aa, synthetic construct
  REGION 1..13: note = Description of Artificial Sequence: Synthetic peptide
NGKAFDFNRWHDY

SEQ ID NO: 208 - protein, 17 aa, synthetic construct
  REGION 1..17: note = Description of Artificial Sequence: Synthetic peptide
YRKGQYYRGTYWDNFES

SEQ ID NO: 209 - protein, 19 aa, synthetic construct
  REGION 1..19: note = Description of Artificial Sequence: Synthetic peptide
AAVRPYGSGTYSRTEAYNF

SEQ ID NO: 210 - protein, 22 aa, synthetic construct
  REGION 1..22: note = Description of Artificial Sequence: Synthetic peptide
AAVGRILGWIPTMYRQAASYDY

SEQ ID NO: 211 - protein, 13 aa, synthetic construct
  REGION 1..13: note = Description of Artificial Sequence: Synthetic peptide
NAKSFDGTRWVEH

SEQ ID NO: 212 - protein, 13 aa, synthetic construct
  REGION 1..13: note = Description of Artificial Sequence: Synthetic peptide
NAKAFDGTRWRDY

SEQ ID NO: 213 - protein, 9 aa, synthetic construct
  REGION 1..9: note = Description of Artificial Sequence: Synthetic peptide
NVQTIIRNY

SEQ ID NO: 214 - protein, 17 aa, synthetic construct
  REGION 1..17: note = Description of Artificial Sequence: Synthetic peptide
SQRGQYFTEGYWKEYDN

SEQ ID NO: 215 - protein, 13 aa, synthetic construct
  REGION 1..13: note = Description of Artificial Sequence: Synthetic peptide
NANVYDGNRWRTY

SEQ ID NO: 216 - protein, 15 aa, synthetic construct
  REGION 1..15: note = Description of Artificial Sequence: Synthetic peptide
AAGTKYSDTIITWGS

SEQ ID NO: 217 - protein, 13 aa, synthetic construct
  REGION 1..13: note = Description of Artificial Sequence: Synthetic peptide
NAKAFDGTRWYDY

SEQ ID NO: 218 - protein, 12 aa, synthetic construct
  REGION 1..12: note = Description of Artificial Sequence: Synthetic peptide
AARTALGGTYDY

SEQ ID NO: 219 - protein, 5 aa, synthetic construct
  REGION 1..5: note = Description of Artificial Sequence: Synthetic peptide
HTPHF

SEQ ID NO: 220 - protein, 17 aa, synthetic construct
  REGION 1..17: note = Description of Artificial Sequence: Synthetic peptide
NVVGTYWTGADWRPFDT

SEQ ID NO: 221 - protein, 13 aa, synthetic construct
  REGION 1..13: note = Description of Artificial Sequence: Synthetic peptide
NAITFYGTRWLDY

SEQ ID NO: 222 - protein, 13 aa, synthetic construct
  REGION 1..13: note = Description of Artificial Sequence: Synthetic peptide
NADVYDGNRWRTY

SEQ ID NO: 223 - protein, 13 aa, synthetic construct
  REGION 1..13: note = Description of Artificial Sequence: Synthetic peptide
FAKTFDGTRWCDY

SEQ ID NO: 224 - protein, 13 aa, synthetic construct
  REGION 1..13: note = Description of Artificial Sequence: Synthetic peptide
NAIVYDGNRWRTY

SEQ ID NO: 225 - protein, 5 aa, synthetic construct
  REGION 1..5: note = Description of Artificial Sequence: Synthetic peptide
NSVGH

SEQ ID NO: 226 - protein, 12 aa, synthetic construct
  REGION 1..12: note = Description of Artificial Sequence: Synthetic peptide
AAQTLGSSYYDA

SEQ ID NO: 227 - protein, 17 aa, synthetic construct
  REGION 1..17: note = Description of Artificial Sequence: Synthetic peptide
AAVQSTVIQTSPNRYNY

SEQ ID NO: 228 - protein, 18 aa, synthetic construct
  REGION 1..18: note = Description of Artificial Sequence: Synthetic peptide
AAARLGWGLTISDRIYEY

SEQ ID NO: 229 - protein, 15 aa, synthetic construct
  REGION 1..15: note = Description of Artificial Sequence: Synthetic peptide
AKASLPWFDGSSPDY

SEQ ID NO: 230 - protein, 18 aa, synthetic construct
  REGION 1..18: note = Description of Artificial Sequence: Synthetic peptide
AAARVRTPGWGPQKSYDY

SEQ ID NO: 231 - protein, 21 aa, synthetic construct
  REGION 1..21: note = Description of Artificial Sequence: Synthetic peptide
NADYDNSGSYYYQKGNYEYDY

SEQ ID NO: 232 - protein, 13 aa, synthetic construct
  REGION 1..13: note = Description of Artificial Sequence: Synthetic peptide
TADGPPDYGKYDY

SEQ ID NO: 233 - protein, 17 aa, synthetic construct
  REGION 1..17: note = Description of Artificial Sequence: Synthetic peptide
AAVKYWGRRQRADEYDY

SEQ ID NO: 234 - protein, 21 aa, synthetic construct
  REGION 1..21: note = Description of Artificial Sequence: Synthetic peptide
AAELSLNPGKRLTLEILKYDY

SEQ ID NO: 235 - protein, 22 aa, synthetic construct
  REGION 1..22: note = Description of Artificial Sequence: Synthetic peptide
AAGYGAGDVKRALSSCRGSYVY

SEQ ID NO: 236 - protein, 16 aa, synthetic construct
  REGION 1..16: note = Description of Artificial Sequence: Synthetic peptide
KMNHQLYSDSSYENVY

SEQ ID NO: 237 - protein, 16 aa, synthetic construct
  REGION 1..16: note = Description of Artificial Sequence: Synthetic peptide
ATDTWGNSRCDHDMRY

SEQ ID NO: 238 - protein, 19 aa, synthetic construct
  REGION 1..19: note = Description of Artificial Sequence: Synthetic peptide
ESEALLETTPSRRPYEYNY

SEQ ID NO: 239 - protein, 15 aa, synthetic construct
  REGION 1..15: note = Description of Artificial Sequence: Synthetic peptide
AKGQREYYNDFEFDY

SEQ ID NO: 240 - protein, 16 aa, synthetic construct
  REGION 1..16: note = Description of Artificial Sequence: Synthetic peptide
RTLGCSYYERADEYNY

SEQ ID NO: 241 - protein, 11 aa, synthetic construct
  REGION 1..11: note = Description of Artificial Sequence: Synthetic peptide
YLEIPESRGAF

SEQ ID NO: 242 - protein, 14 aa, synthetic construct
  REGION 1..14: note = Description of Artificial Sequence: Synthetic peptide
AAGEWGIRPYNYDY

SEQ ID NO: 243 - protein, 21 aa, synthetic construct
  REGION 1..21: note = Description of Artificial Sequence: Synthetic peptide
NTDYDNSGSYYYQKGNYEYDY

SEQ ID NO: 244 - protein, 21 aa, synthetic construct
  REGION 1..21: note = Description of Artificial Sequence: Synthetic peptide
NANYDNNGSYYYQKGNYEYDY

SEQ ID NO: 245 - protein, 19 aa, synthetic construct
  REGION 1..19: note = Description of Artificial Sequence: Synthetic peptide
AAAALLETTPSRRPSAYNY

SEQ ID NO: 246 - protein, 17 aa, synthetic construct
  REGION 1..17: note = Description of Artificial Sequence: Synthetic peptide
AGAWGYAGIIPRGAYDD

SEQ ID NO: 247 - protein, 18 aa, synthetic construct
  REGION 1..18: note = Description of Artificial Sequence: Synthetic peptide
AAARIRTPGWGPQKSYDY

SEQ ID NO: 248 - protein, 18 aa, synthetic construct
  REGION 1..18: note = Description of Artificial Sequence: Synthetic peptide
AAGVVTAQAIMAARDFDY

SEQ ID NO: 249 - protein, 17 aa, synthetic construct
  REGION 1..17: note = Description of Artificial Sequence: Synthetic peptide
AADRSREGRTRPNEYDY

SEQ ID NO: 250 - protein, 9 aa, synthetic construct
  REGION 1..9: note = Description of Artificial Sequence: Synthetic peptide
KVRAEDTDY

SEQ ID NO: 251 - protein, 21 aa, synthetic construct
  REGION 1..21: note = Description of Artificial Sequence: Synthetic peptide
AAPERSGSYAYTPSRLNEYAY

SEQ ID NO: 252 - protein, 12 aa, synthetic construct
  REGION 1..12: note = Description of Artificial Sequence: Synthetic peptide
NADFYGLYPRQY

SEQ ID NO: 253 - protein, 18 aa, synthetic construct
  REGION 1..18: note = Description of Artificial Sequence: Synthetic peptide
AADRYYRTDIYRASSYEY

SEQ ID NO: 254 - protein, 13 aa, synthetic construct
  REGION 1..13: note = Description of Artificial Sequence: Synthetic peptide
AKGWTVDVNHIED

SEQ ID NO: 255 - protein, 15 aa, synthetic construct
  REGION 1..15: note = Description of Artificial Sequence: Synthetic peptide
AAKYGGTDLLSRYEY

SEQ ID NO: 256 - protein, 21 aa, synthetic construct
  REGION 1..21: note = Description of Artificial Sequence: Synthetic peptide
ASVYSFDPGRCGPIATMVGHY

SEQ ID NO: 257 - protein, 26 aa, synthetic construct
  REGION 1..26: note = Description of Artificial Sequence: Synthetic peptide
ATDAAGGRGSFFIDHKRTCPSEEYDS

SEQ ID NO: 258 - protein, 13 aa, synthetic construct
  REGION 1..13: note = Description of Artificial Sequence: Synthetic peptide
TADGPPDYGKYDY

SEQ ID NO: 259 - protein, 18 aa, synthetic construct
  REGION 1..18: note = Description of Artificial Sequence: Synthetic peptide
ARVSVIRPPYGVYSDFGS

SEQ ID NO: 260 - protein, 13 aa, synthetic construct
  REGION 1..13: note = Description of Artificial Sequence: Synthetic peptide
TLYGSGAAEKFHS

SEQ ID NO: 261 - protein, 18 aa, synthetic construct
  REGION 1..18: note = Description of Artificial Sequence: Synthetic peptide
AANWGRRRVPTTVHEYDY

SEQ ID NO: 262 - protein, 18 aa, synthetic construct
  REGION 1..18: note = Description of Artificial Sequence: Synthetic peptide
AAARLGWGLTISDRIYEY

SEQ ID NO: 263 - protein, 16 aa, synthetic construct
  REGION 1..16: note = Description of Artificial Sequence: Synthetic peptide
ATDTWGNSRCDHDMRY

SEQ ID NO: 264 - protein, 19 aa, synthetic construct
  REGION 1..19: note = Description of Artificial Sequence: Synthetic peptide
AAGSFSDNKYYTRSQDYEH

SEQ ID NO: 265 - protein, 15 aa, synthetic construct
  REGION 1..15: note = Description of Artificial Sequence: Synthetic peptide
AASSWCQTFDAKYGY

SEQ ID NO: 266 - protein, 13 aa, synthetic construct
  REGION 1..13: note = Description of Artificial Sequence: Synthetic peptide
AADNQHDIPLRPG

SEQ ID NO: 267 - protein, 14 aa, synthetic construct
  REGION 1..14: note = Description of Artificial Sequence: Synthetic peptide
HPSDTTGWTRGRAY

SEQ ID NO: 268 - protein, 14 aa, synthetic construct
  REGION 1..14: note = Description of Artificial Sequence: Synthetic peptide
ATACTRLWKPGRDY

SEQ ID NO: 269 - protein, 6 aa, synthetic construct
  REGION 1..6: note = Description of Artificial Sequence: Synthetic peptide
MRDFYT

SEQ ID NO: 270 - protein, 24 aa, synthetic construct
  REGION 1..24: note = Description of Artificial Sequence: Synthetic peptide
AAASTIKHCYTAVSYYTKDAQYDY

SEQ ID NO: 271 - protein, 17 aa, synthetic construct
  REGION 1..17: note = Description of Artificial Sequence: Synthetic peptide
NVVGTYWTGADWRPFDT

SEQ ID NO: 272 - protein, 22 aa, synthetic construct
  REGION 1..22: note = Description of Artificial Sequence: Synthetic peptide
GVARDSRSYYNFRLNQEDEYDY

SEQ ID NO: 273 - protein, 17 aa, synthetic construct
  REGION 1..17: note = Description of Artificial Sequence: Synthetic peptide
AASHGIGRVVAESLYDY

SEQ ID NO: 274 - protein, 185 aa, Salmonella typhimurium
MKHKLMTSTIASLMFVAGAAVAADPTPVSVSGGTIHFEGKLVNAACAVSTKSADQTVTLGQYRTASFTAIGNTTAQVPFSIVLNDCDPKVAANAAVAFSGQADNTNPNLLAVSSADNSTTATGVGIEILDNTSSPLKPDGATFSAKQSLVEGTNTLRFTARYKATAAATTPGQANADATFIMKYE

SEQ ID NO: 275 - protein, 495 aa, Salmonella typhimurium [sequence omitted]

SEQ ID NO: 276 - protein, 80 aa, Salmonella typhimurium
MATPWSGYLDDVSAKFDTGVDNLQTQVTEALDKLAAKPSDPALLAAYQSKLSEYNLYRNAQSNTVKVFKDIDAAIIQNFR

SEQ ID NO: 277 - protein, 185 aa, Salmonella enteritidis
MKHKLMTSTIASLMFVAGAAVAADPTPVSVSGGTIHFEGKLVNAACAVSTKSADQTVTLGQYRTASFTAIGNTTAQVPFSIVLNDCDPKVAATAAVAFSGQADNTNPNLLAVSSADNSTTATGVGIEILDNTSSPLKPDGATFSAKQALVEGTNTLRFTARYKATATATTPGQANADATFIMKYE

SEQ ID NO: 278 - protein, 505 aa, Salmonella enteritidis
MAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAAAPDKVYVNAANGQLTTDDAENNTAVDLFKTTKSTAGTAEAKAIAGAIKGGKEGDTFDYKGVTFTIDTKTGDDGNGKVSTTINGEKVTLTVADIATGATDVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 279 - protein, 80 aa, Salmonella enteritidis; identical to SEQ ID NO: 276

SEQ ID NO: 280 - protein, 9 aa, synthetic construct
  REGION 1..9: note = Description of Artificial Sequence: Synthetic peptide
GRTSSSAYT

SEQ ID NO: 281 - protein, 9 aa, synthetic construct
  REGION 1..9: note = Description of Artificial Sequence: Synthetic peptide
TRTSSSSYT

SEQ ID NO: 282 - protein, 9 aa, synthetic construct
  REGION 1..9: note = Description of Artificial Sequence: Synthetic peptide
GRTSSSAYT

SEQ ID NO: 283 - protein, 9 aa, synthetic construct
  REGION 1..9: note = Description of Artificial Sequence: Synthetic peptide
GRTSPSSYT

SEQ ID NO: 284 - protein, 9 aa, synthetic construct
  REGION 1..9: note = Description of Artificial Sequence: Synthetic peptide
GSTLSNYAV

SEQ ID NO: 285 - protein, 9 aa, synthetic construct
  REGION 1..9: note = Description of Artificial Sequence: Synthetic peptide
GRTFSSLAM

SEQ ID NO: 286 - protein, 9 aa, synthetic construct
  REGION 1..9: note = Description of Artificial Sequence: Synthetic peptide
GRTFSSLAM

SEQ ID NO: 287 - protein, 9 aa, synthetic construct
  REGION 1..9: note = Description of Artificial Sequence: Synthetic peptide
TAILSIDSM

SEQ ID NO: 288 - protein, 9 aa, synthetic construct
  REGION 1..9: note = Description of Artificial Sequence: Synthetic peptide
GDISTIDVM

SEQ ID NO: 289 - protein, 9 aa, synthetic construct
  REGION 1..9: note = Description of Artificial Sequence: Synthetic peptide
GFTFSSSIM

SEQ ID NO: 290 - protein, 9 aa, synthetic construct
  REGION 1..9: note = Description of Artificial Sequence: Synthetic peptide
GDISSISVM

SEQ ID NO: 291 - protein, 9 aa, synthetic construct
  REGION 1..9: note = Description of Artificial Sequence: Synthetic peptide
GFTFSTNIL

SEQ ID NO: 292 - protein, 9 aa, synthetic construct
  REGION 1..9: note = Description of Artificial Sequence: Synthetic peptide
TSILSINAM

SEQ ID NO: 293 - protein, 9 aa, synthetic construct
  REGION 1..9: note = Description of Artificial Sequence: Synthetic peptide
GSISSITAM

SEQ ID NO: 294 - protein, 9 aa, synthetic construct
  REGION 1..9: note = Description of Artificial Sequence: Synthetic peptide
GSISSITAM

SEQ ID NO: 295 - protein, 9 aa, synthetic construct
  REGION 1..9: note = Description of Artificial Sequence: Synthetic peptide
GFTFSSAIM

SEQ ID NO: 296 - protein, 9 aa, synthetic construct
  REGION 1..9: note = Description of Artificial Sequence: Synthetic peptide
TSILSIDAM

SEQ ID NO: 297 - protein, 9 aa, synthetic construct
  REGION 1..9: note = Description of Artificial Sequence: Synthetic peptide
GSISSITAM

SEQ ID NO: 298 - protein, 9 aa, synthetic construct
  REGION 1..9: note = Description of Artificial Sequence: Synthetic peptide
GSAFSGDAM

SEQ ID NO: 299 - protein, 10 aa, synthetic construct
  REGION 1..10: note = Description of Artificial Sequence: Synthetic peptide
GSAFSGGDAM

SEQ ID NO: 300 - protein, 9 aa, synthetic construct
  REGION 1..9: note = Description of Artificial Sequence: Synthetic peptide
GSAFSGDAM

SEQ ID NO: 301 - protein, 9 aa, synthetic construct
  REGION 1..9: note = Description of Artificial Sequence: Synthetic peptide
GLTFNNYAM

SEQ ID NO: 302 - protein, 9 aa, synthetic construct
  REGION 1..9: note = Description of Artificial Sequence: Synthetic peptide
GIDSSFYVM

SEQ ID NO: 303 - protein, 9 aa, synthetic construct
  REGION 1..9: note = Description of Artificial Sequence: Synthetic peptide
GIDSSFYVM

SEQ ID NO: 304 - protein, 9 aa, synthetic construct
  REGION 1..9: note = Description of Artificial Sequence: Synthetic peptide
GLTFSSYAM

SEQ ID NO: 305 - protein, 8 aa, synthetic construct
  REGION 1..8: note = Description of Artificial Sequence: Synthetic peptide
ESIFSRNA

SEQ ID NO: 306 - protein, 9 aa, synthetic construct
  REGION 1..9: note = Description of Artificial Sequence: Synthetic peptide
GITSSLYVM

SEQ ID NO: 307 - protein, 9 aa, synthetic construct
  REGION 1..9: note = Description of Artificial Sequence: Synthetic peptide
GLTFNNYAM

SEQ ID NO: 308 - protein, 9 aa, synthetic construct
  REGION 1..9: note = Description of Artificial Sequence: Synthetic peptide
GIDSSFYVM

SEQ ID NO: 309 - protein, 10 aa, synthetic construct
  REGION 1..10: note = Description of Artificial Sequence: Synthetic peptide
GSGILFRISA

SEQ ID NO: 310 - protein, 9 aa, synthetic construct
  REGION 1..9: note = Description of Artificial Sequence: Synthetic peptide
ARTLSNYAM

SEQ ID NO: 311 - protein, 9 aa, synthetic construct
  REGION 1..9: note = Description of Artificial Sequence: Synthetic peptide
GSAFSGDAM

SEQ ID NO: 312 - protein, 9 aa, synthetic construct
  REGION 1..9: note = Description of Artificial Sequence: Synthetic peptide
GLTFHNYAM

SEQ ID NO: 313 - protein, 9 aa, synthetic construct
  REGION 1..9: note = Description of Artificial Sequence: Synthetic peptide
GSAFSGDAM

SEQ ID NO: 314 - protein, 8 aa, synthetic construct
  REGION 1..8: note = Description of Artificial Sequence: Synthetic peptide
GRTFSTYA

SEQ ID NO: 315 - protein, 9 aa, synthetic construct
  REGION 1..9: note = Description of Artificial Sequence: Synthetic peptide
GSAFSGDAM

SEQ ID NO: 316 - protein, 9 aa, synthetic construct
  REGION 1..9: note = Description of Artificial Sequence: Synthetic peptide
GLTFNNYAM

SEQ ID NO: 317 - protein, 9 aa, synthetic construct
  REGION 1..9: note = Description of Artificial Sequence: Synthetic peptide
GSAFSGDAM

SEQ ID NO: 318 - protein, 9 aa, synthetic construct
  REGION 1..9: note = Description of Artificial Sequence: Synthetic peptide
GSAFSGDAM

SEQ ID NO: 319 - protein, 9 aa, synthetic construct
  REGION 1..9: note = Description of Artificial Sequence: Synthetic peptide
GSAFSGDAM

SEQ ID NO: 320 - protein, 9 aa, synthetic construct
  REGION 1..9: note = Description of Artificial Sequence: Synthetic peptide
GSAFSGDAM

SEQ ID NO: 321 - protein, 9 aa, synthetic construct
  REGION 1..9: note = Description of Artificial Sequence: Synthetic peptide
ISGFSGDAM

SEQ ID NO: 322 - protein, 9 aa, synthetic construct
  REGION 1..9: note = Description of Artificial Sequence: Synthetic peptide
GIISSAYVM

SEQ ID NO: 323 - protein, 9 aa, synthetic construct
  REGION 1..9: note = Description of Artificial Sequence: Synthetic peptide
GIAFSTYGM

SEQ ID NO: 324 - protein, 9 aa, synthetic construct
  REGION 1..9: note = Description of Artificial Sequence: Synthetic peptide
GSAFSGDAM

SEQ ID NO: 325 - protein, 9 aa, synthetic construct
  REGION 1..9: note = Description of Artificial Sequence: Synthetic peptide
GSAFSGDAM

SEQ ID NO: 326 - protein, 9 aa, synthetic construct
  REGION 1..9: note = Description of Artificial Sequence: Synthetic peptide
GSAFSGDAM

SEQ ID NO: 327 - protein, 9 aa, synthetic construct
  REGION 1..9: note = Description of Artificial Sequence: Synthetic peptide
GSAFSGDAM

SEQ ID NO: 328 - protein, 10 aa, synthetic construct
  REGION 1..10: note = Description of Artificial Sequence: Synthetic peptide
GSAFSGGDAM

SEQ ID NO: 329 - protein, 9 aa, synthetic construct
  REGION 1..9: note = Description of Artificial Sequence: Synthetic peptide
GITFSSDAM

SEQ ID NO: 330 - protein, 8 aa, synthetic construct
  REGION 1..8: note = Description of Artificial Sequence: Synthetic peptide
ISWSGTTT

SEQ ID NO: 331 - protein, 8 aa, synthetic construct
  REGION 1..8: note = Description of Artificial Sequence: Synthetic peptide
ISYSGTTT

SEQ ID NO: 332 - protein, 8 aa, synthetic construct
  REGION 1..8: note = Description of Artificial Sequence: Synthetic peptide
ISWSGTTT

SEQ ID NO: 333 - protein, 8 aa, synthetic construct
  REGION 1..8: note = Description of Artificial Sequence: Synthetic peptide
ISWSGTTT

SEQ ID NO: 334 - protein, 7 aa, synthetic construct
  REGION 1..7: note = Description of Artificial Sequence: Synthetic peptide
ISSGGST

SEQ ID NO: 335 - protein, 8 aa, synthetic construct
  REGION 1..8: note = Description of Artificial Sequence: Synthetic peptide
ISRSGDYT

SEQ ID NO: 336 - protein, 8 aa, synthetic construct
  REGION 1..8: note = Description of Artificial Sequence: Synthetic peptide
ITRSGDYT

SEQ ID NO: 337 - protein, 7 aa, synthetic construct
  REGION 1..7: note = Description of Artificial Sequence: Synthetic peptide
IARGGST

SEQ ID NO: 338 - protein, 7 aa, synthetic construct
  REGION 1..7: note = Description of Artificial Sequence: Synthetic peptide
IARGGTI

SEQ ID NO: 339 - protein, 7 aa, synthetic construct
  REGION 1..7: note = Description of Artificial Sequence: Synthetic peptide
IPSFGSA

SEQ ID NO: 340 - protein, 7 aa, synthetic construct
  REGION 1..7: note = Description of Artificial Sequence: Synthetic peptide
IASGGSV

SEQ ID NO: 341 - protein, 7 aa, synthetic construct
  REGION 1..7: note = Description of Artificial Sequence: Synthetic peptide
ITPFGSA

SEQ ID NO: 342 - protein, 7 aa, synthetic construct
  REGION 1..7: note = Description of Artificial Sequence: Synthetic peptide
IAPGGTT

SEQ ID NO: 343 - protein, 7 aa, synthetic construct
  REGION 1..7: note = Description of Artificial Sequence: Synthetic peptide
IARGGMI

SEQ ID NO: 344 - protein, 7 aa, synthetic construct
  REGION 1..7: note = Description of Artificial Sequence: Synthetic peptide
IARGGMT

SEQ ID NO: 345 - protein, 7 aa, synthetic construct
  REGION 1..7: note = Description of Artificial Sequence: Synthetic peptide
IPSFGSA

SEQ ID NO: 346 - protein, 7 aa, synthetic construct
  REGION 1..7: note = Description of Artificial Sequence: Synthetic peptide
IARGGST

SEQ ID NO: 347 - protein, 7 aa, synthetic construct
  REGION 1..7: note = Description of Artificial Sequence: Synthetic peptide
IARGGMT

SEQ ID NO: 348 - protein, 7 aa, synthetic construct
  REGION 1..7: note = Description of Artificial Sequence: Synthetic peptide
ISSGAIT

SEQ ID NO: 349 - protein, 7 aa, synthetic construct
  REGION 1..7: note = Description of Artificial Sequence: Synthetic peptide
ISSGGIA

SEQ ID NO: 350 - protein, 7 aa, synthetic construct
  REGION 1..7: note = Description of Artificial Sequence: Synthetic peptide
ISSGGIP

SEQ ID NO: 351 - protein, 8 aa, synthetic construct
  REGION 1..8: note = Description of Artificial Sequence: Synthetic peptide
ISRDGTNT

SEQ ID NO: 352 - protein, 7 aa, synthetic construct
  REGION 1..7: note = Description of Artificial Sequence: Synthetic peptide
LGTPDSA

SEQ ID NO: 353 - protein, 7 aa, synthetic construct
  REGION 1..7: note = Description of Artificial Sequence: Synthetic peptide
ISSADSP

SEQ ID NO: 354 - protein, 8 aa, synthetic construct
  REGION 1..8: note = Description of Artificial Sequence: Synthetic peptide
IGWSGGST

SEQ ID NO: 355 - protein, 7 aa, synthetic construct
  REGION 1..7: note = Description of Artificial Sequence: Synthetic peptide
IGSDGST

SEQ ID NO: 356 - protein, 7 aa, synthetic construct
  REGION 1..7: note = Description of Artificial Sequence: Synthetic peptide
INSGDSP

SEQ ID NO: 357 - protein, 8 aa, synthetic construct
  REGION 1..8: note = Description of Artificial Sequence: Synthetic peptide
ISRDGTST

SEQ ID NO: 358 - protein, 9 aa, synthetic construct
  REGION 1..9: note = Description of Artificial Sequence: Synthetic peptide
ISMTSADSP

SEQ ID NO: 359 - protein, 7 aa, synthetic construct
  REGION 1..7: note = Description of Artificial Sequence: Synthetic peptide
ISSGGST

SEQ ID NO: 360 - protein, 8 aa, synthetic construct
  REGION 1..8: note = Description of Artificial Sequence: Synthetic peptide
ISRSGGSI

SEQ ID NO: 361 - protein, 7 aa, synthetic construct
  REGION 1..7: note = Description of Artificial Sequence: Synthetic peptide
ISSGGIT

SEQ ID NO: 362 - protein, 8 aa, synthetic construct
  REGION 1..8: note = Description of Artificial Sequence: Synthetic peptide
ISRDGTNT

SEQ ID NO: 363 - protein, 7 aa, synthetic construct
  REGION 1..7: note = Description of Artificial Sequence: Synthetic peptide
ISSGHIT

SEQ ID NO: 364 - protein, 8 aa, synthetic construct
  REGION 1..8: note = Description of Artificial Sequence: Synthetic peptide
ISRSGDNI

SEQ ID NO: 365 - protein, 7 aa, synthetic construct
  REGION 1..7: note = Description of Artificial Sequence: Synthetic peptide
ISSGGIE

SEQ ID NO: 366 - protein, 8 aa, synthetic construct
  REGION 1..8: note = Description of Artificial Sequence: Synthetic peptide
ISRDGTNT

SEQ ID NO: 367 - protein, 7 aa, synthetic construct
  REGION 1..7: note = Description of Artificial Sequence: Synthetic peptide
ISSGGIT

SEQ ID NO: 368 - protein, 7 aa, synthetic construct
  REGION 1..7: note = Description of Artificial Sequence: Synthetic peptide
ISSGGIA

SEQ ID NO: 369 - protein, 7 aa, synthetic construct
  REGION 1..7: note = Description of Artificial Sequence: Synthetic peptide
ISSGAIT

SEQ ID NO: 370 - protein, 7 aa, synthetic construct
  REGION 1..7: note = Description of Artificial Sequence: Synthetic peptide
ISSGGIT

SEQ ID NO: 371 - protein, 7 aa, synthetic construct
  REGION 1..7: note = Description of Artificial Sequence: Synthetic peptide
ISSGGIT

SEQ ID NO: 372 - protein, 7 aa, synthetic construct
  REGION 1..7: note = Description of Artificial Sequence: Synthetic peptide
ITSGDSP

SEQ ID NO: 373 - protein, 7 aa, synthetic construct
  REGION 1..7: note = Description of Artificial Sequence: Synthetic peptide
ITGNGDD

SEQ ID NO: 374 - protein, 7 aa, synthetic construct
  REGION 1..7: note = Description of Artificial Sequence: Synthetic peptide
ISSGGIT

SEQ ID NO: 375 - protein, 7 aa, synthetic construct
  REGION 1..7: note = Description of Artificial Sequence: Synthetic peptide
ISSGGIT

SEQ ID NO: 376 - protein, 7 aa, synthetic construct
  REGION 1..7: note = Description of Artificial Sequence: Synthetic peptide
ISSGGIT

SEQ ID NO: 377 - protein, 7 aa, synthetic construct
  REGION 1..7: note = Description of Artificial Sequence: Synthetic peptide
ISSGGIP

SEQ ID NO: 378 - protein, 7 aa, synthetic construct
  REGION 1..7: note = Description of Artificial Sequence: Synthetic peptide
ISSGGIT

SEQ ID NO: 379 - protein, 7 aa, synthetic construct
  REGION 1..7: note = Description of Artificial Sequence: Synthetic peptide
ISSGDIT

SEQ ID NO: 380 - protein, 17 aa, synthetic construct
  REGION 1..17: note = Description of Artificial Sequence: Synthetic peptide
AADRRSTIGSPRQQYAY

SEQ ID NO: 381 - protein, 17 aa, synthetic construct
  REGION 1..17: note = Description of Artificial Sequence: Synthetic peptide
AADRRSTIGSPRQQYAY

SEQ ID NO: 382 - protein, 17 aa, synthetic construct
  REGION 1..17: note = Description of Artificial Sequence: Synthetic peptide
AADRRSTIGTPREQYAY

SEQ ID NO: 383 - protein, 17 aa, synthetic construct
  REGION 1..17: note = Description of Artificial Sequence: Synthetic peptide
AADRRSTIGSPRQQYAY

SEQ ID NO: 384 - protein, 14 aa, synthetic construct
  REGION 1..14: note = Description of Artificial Sequence: Synthetic peptide
HTYDFQGWGLRSDY

SEQ ID NO: 385 - protein, 23 aa, synthetic construct
  REGION 1..23: note = Description of Artificial Sequence: Synthetic peptide
AATKIVTPWTSTYYYTKAYEWDY

SEQ ID NO: 386 - protein, 23 aa, synthetic construct
  REGION 1..23: note = Description of Artificial Sequence: Synthetic peptide
AATKIVTPWTSTYYYTKAYEWDY

SEQ ID NO: 387 - protein, 11 aa, synthetic construct
  REGION 1..11: note = Description of Artificial Sequence: Synthetic peptide
AADPGGASPLS

SEQ ID NO: 388 - protein, 10 aa, synthetic construct
  REGION 1..10: note = Description of Artificial Sequence: Synthetic peptide
AVDTGSPRLT

SEQ ID NO: 389 - protein, 5 aa, synthetic construct
  REGION 1..5: note = Description of Artificial Sequence: Synthetic peptide
NTRLY

SEQ ID NO: 390 - protein, 10 aa, synthetic construct
  REGION 1..10: note = Description of Artificial Sequence: Synthetic peptide
AVDTGSPRLT

SEQ ID NO: 391 - protein, 5 aa, synthetic construct
  REGION 1..5: note = Description of Artificial Sequence: Synthetic peptide
NTQLY

SEQ ID NO: 392 - protein, 11 aa, synthetic construct
  REGION 1..11: note = Description of Artificial Sequence: Synthetic peptide
AADPGGQSPLS

SEQ ID NO: 393 - protein, 10 aa, synthetic construct
  REGION 1..10: note = Description of Artificial Sequence: Synthetic peptide
AVDNGDPRLH

SEQ ID NO: 394 - protein, 10 aa, synthetic construct
  REGION 1..10: note = Description of Artificial Sequence: Synthetic peptide
ALDNGDPRLH

SEQ ID NO: 395 - protein, 5 aa, synthetic construct
  REGION 1..5: note = Description of Artificial Sequence: Synthetic peptide
NTRLY

SEQ ID NO: 396 - protein, 11 aa, synthetic construct
  REGION 1..11: note = Description of Artificial Sequence: Synthetic peptide
AADPGGASGLS

SEQ ID NO: 397 - protein, 10 aa, synthetic construct
  REGION 1..10: note = Description of Artificial Sequence: Synthetic peptide
ALYNGDPRLH

SEQ ID NO: 398 - protein, 12 aa, synthetic construct
  REGION 1..12: note = Description of Artificial Sequence: Synthetic peptide
NRIQAVLRGNSG

SEQ ID NO: 399 - protein, 12 aa, synthetic construct
  REGION 1..12: note = Description of Artificial Sequence: Synthetic peptide
NSITAVLRGNSG

SEQ ID NO: 400 - protein, 12 aa, synthetic construct
  REGION 1..12: note = Description of Artificial Sequence: Synthetic peptide
NSISAVLRGNGV

SEQ ID NO: 401 - protein, 20 aa, synthetic construct
  REGION 1..20: note = Description of Artificial Sequence: Synthetic peptide
GVGRGTGYAYTAINEYDYSK

SEQ ID NO: 402 - protein, 8 aa, synthetic construct
  REGION 1..8: note = Description of Artificial Sequence: Synthetic peptide
YGLYRQVY

SEQ ID NO: 403 - protein, 8 aa, synthetic construct
  REGION 1..8: note = Description of Artificial Sequence: Synthetic peptide
YGLYRQVH

SEQ ID NO: 404 - protein, 14 aa, synthetic construct
  REGION 1..14: note = Description of Artificial Sequence: Synthetic peptide
AARRTTAWGKGTDY

SEQ ID NO: 405 - protein, 11 aa, synthetic construct
  REGION 1..11: note = Description of Artificial Sequence: Synthetic peptide
RVVLATSPYNY

SEQ ID NO: 406 - protein, 8 aa, synthetic construct
  REGION 1..8: note = Description of Artificial Sequence: Synthetic peptide
YGLYRQVH

SEQ ID NO: 407 - protein, 20 aa, synthetic construct
  REGION 1..20: note = Description of Artificial Sequence: Synthetic peptide
GVGRGSGYAYSAINEYDYSS

SEQ ID NO: 408 - protein, 8 aa, synthetic construct
  REGION 1..8: note = Description of Artificial Sequence: Synthetic peptide
YGLYRQVH

SEQ ID NO: 409 - protein, 8 aa, synthetic construct
  REGION 1..8: note = Description of Artificial Sequence: Synthetic peptide
NIVGRTDS

SEQ ID NO: 410 - protein, 20 aa, synthetic construct
  REGION 1..20: note = Description of Artificial Sequence: Synthetic peptide
GRARGTGYAYTALNQYDYDY

SEQ ID NO: 411 - protein, 12 aa, synthetic construct
  REGION 1..12: note = Description of Artificial Sequence: Synthetic peptide
NSIKAVLRGNSG

SEQ ID NO: 412 - protein, 20 aa, synthetic construct
  REGION 1..20: note = Description of Artificial Sequence: Synthetic peptide
GVGRGSGYAYTAINEYDYSK

SEQ ID NO: 413 - protein, 12 aa, synthetic construct
  REGION 1..12: note = Description of Artificial Sequence: Synthetic peptide
NSITAVLRGNSG

SEQ ID NO: 414 - protein, 20 aa, synthetic construct
  REGION 1..20: note = Description of Artificial Sequence: Synthetic peptide
GRARGTGYAHTALNQYDYDY

SEQ ID NO: 415 - protein, 12 aa, synthetic construct
  REGION 1..12: note = Description of Artificial Sequence: Synthetic peptide
NLIKAVLRGNSG

SEQ ID NO: 416 - protein, 20 aa, synthetic construct
  REGION 1..20: note = Description of Artificial Sequence: Synthetic peptide
GVGRGTGYAYTAIREHDYSS

SEQ ID NO: 417 - protein, 12 aa, synthetic construct
  REGION 1..12: note = Description of Artificial Sequence: Synthetic peptide
NSITAVLRGNSG

SEQ ID NO: 418 - protein, 12 aa, synthetic construct
  REGION 1..12: note = Description of Artificial Sequence: Synthetic peptide
NTIKAVLRGNAG

SEQ ID NO: 419 - protein, 11 aa, synthetic construct
  REGION 1..11: note = Description of Artificial Sequence: Synthetic peptide
NSITAVLRGNS

SEQ ID NO: 420 - protein, 12 aa, synthetic construct
  REGION 1..12: note = Description of Artificial Sequence: Synthetic peptide
NIISAVLRGNGG

SEQ ID NO: 421 - protein, 12 aa, synthetic construct
  REGION 1..12: note = Description of Artificial Sequence: Synthetic peptide
NTITGVLRGNSG

SEQ ID NO: 422 - protein, 8 aa, synthetic construct
  REGION 1..8: note = Description of Artificial Sequence: Synthetic peptide
YGLYRQVY

SEQ ID NO: 423 - protein, 5 aa, synthetic construct
  REGION 1..5: note = Description of Artificial Sequence: Synthetic peptide
NIGMY

SEQ ID NO: 424 - protein, 12 aa, synthetic construct
  REGION 1..12: note = Description of Artificial Sequence: Synthetic peptide
NSISAVLRGNSG

SEQ ID NO: 425 - protein, 12 aa, synthetic construct
  REGION 1..12: note = Description of Artificial Sequence: Synthetic peptide
NSISAVLRGNGG

SEQ ID NO: 426 - protein, 12 aa, synthetic construct
  REGION 1..12: note = Description of Artificial Sequence: Synthetic peptide
NSITAVLRGNSD

SEQ ID NO: 427 - protein, 12 aa, synthetic construct
  REGION 1..12: note = Description of Artificial Sequence: Synthetic peptide
NIIKTVLRGNAV

SEQ ID NO: 428 - protein, 12 aa, synthetic construct
  REGION 1..12: note = Description of Artificial Sequence: Synthetic peptide
NSITAVLRGNSG

SEQ ID NO: 429 - protein, 12 aa, synthetic construct
  REGION 1..12: note = Description of Artificial Sequence: Synthetic peptide
NTITRLLYGMDY

SEQ ID NO: 430-444 - skipped (15 entries)

SEQ ID NO: 445 - protein, 5 aa, synthetic construct
  REGION 1..5: note = Description of Artificial Sequence: Synthetic peptide
GGGGS

SEQ ID NO: 446 - protein, 15 aa, synthetic construct
  REGION 1..15: note = Description of Artificial Sequence: Synthetic peptide
GGGGSGGGGSGGGGS

SEQ ID NO: 447 - protein, 25 aa, synthetic construct
  REGION 1..25: note = Description of Artificial Sequence: Synthetic peptide
GGGGSGGGGSGGGGSGGGGSGGGGS

SEQ ID NO: 448 - protein, 8 aa, synthetic construct
  REGION 1..8: note = Description of Artificial Sequence: Synthetic peptide
GGGSGGGS

SEQ ID NO: 449 - protein, 10 aa, synthetic construct
  REGION 1..10: note = Description of Artificial Sequence: Synthetic peptide
GGGGGGGGGG

SEQ ID NO: 450 - protein, 30 aa, synthetic construct
  REGION 1..30: note = Description of Artificial Sequence: Synthetic polypeptide
GGAEAAAKEAAAKEAAAKEAAAKEAAAKGG

SEQ ID NO: 451 - protein, 36 aa, synthetic construct
  REGION 1..36: note = Description of Artificial Sequence: Synthetic polypeptide
GGGAAPAAAPAKQEAAAPAPAAKAEAPAAAPAATGG

SEQ ID NO: 452 - protein, 16 aa, synthetic construct
  REGION 1..16: note = Description of Artificial Sequence: Synthetic peptide
GGGGSGGLGGSGGGGS

SEQ ID NO: 453 - protein, 86 aa, synthetic construct [sequence omitted]

SEQ ID NO: 454 - protein, 134 aa, synthetic construct
  REGION 1..134: note = Description of Artificial Sequence: Synthetic polypeptide
MESKRNKPGKATGKGKPVGDKWLDDAGKDSGAPIPDRIADKLRDKEFKSFDDFRKAVWEEVSKDPELSKNLNPCNKSSVSKGYSPFTPKNQQVGGRKVYELHHDKPISQGGEVYDMDNIRVTTPKRHIDIHRGK

SEQ ID NO: 455 - protein, 39 aa, synthetic construct
  REGION 1..39: note = Description of Artificial Sequence: Synthetic polypeptide
LTDTLQAETDQLEDEKSALQTEIANLLKEKEKLEFILAA

SEQ ID NO: 456 - protein, 39 aa, synthetic construct
  REGION 1..39: note = Description of Artificial Sequence: Synthetic polypeptide
RIARLEEKVKTLKAQNSELASTANMLREQVAQLKQKVMN

SEQ ID NO: 457 - protein, 33 aa, synthetic construct
  REGION 1..33: note = Description of Artificial Sequence: Synthetic polypeptide
RMKQLEDKIEELLSKIYHLENEIARLKKLIGER

SEQ ID NO: 458 - protein, 33 aa, synthetic construct
  REGION 1..33: note = Description of Artificial Sequence: Synthetic polypeptide
RMKQIEDKIEEILSKIYHIENEIARIKKLIGER

SEQ ID NO: 459 - protein, 31 aa, synthetic construct
  REGION 1..31: note = Description of Artificial Sequence: Synthetic polypeptide
RMKQIEDKLEEILSKLYHIENELARIKKLLG

SEQ ID NO: 460 - protein, 87 aa, synthetic construct
  REGION 1..87: note = Description of Artificial Sequence: Synthetic polypeptide
ERVVINISGLRFETQLKTLAQFPETLLGDPKKRMRYFDPLRNEYFFDRNRPSFDAILYYYQSGGRLRRPVNVPLDIFSEEIRFYELG

SEQ ID NO: 461 - protein, 301 aa, synthetic construct
  REGION 1..301: note = Description of Artificial Sequence: Synthetic polypeptide
ATPWSGYLDDVSAKFDTGVDNLQTQVTEALDKLAAKPSDPALLAAYQSKLSEYNLYRNAQSNTVKVFKDIDAAIIQNFRGGSGGTTISDAEIWDMVSQNISAIGDSYLGVYENVVAVYTDFYQAFSDILSKMGGWLLPGKDGNTVKLDVTSLKNDLNSLVNKYNQINSNTVLFPAQSGSGVKVATEAEARQWLSELNLPNSCLKSYGSGYVVTVDLTPLQKMVQDIDGLGAPGKDSKLEMDNAKYQAWQSGFKAQEENMKTTLQTLTQKYSNANSLYDNLVKVLSSTISSSLETAKSFLQG

SEQ ID NO: 462 - protein, 253 aa, synthetic construct
  REGION 1..253: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGGSGGGGSGGGGSQVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSS

SEQ ID NO: 463 - protein, 387 aa, synthetic construct
  REGION 1..387: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGGSGGGGSGGGGSQVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGGSGGGGSGGGGSQVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSS

SEQ ID NO: 464 - protein, 237 aa, synthetic construct
  REGION 1..237: note = Description of Artificial Sequence: Synthetic polypeptide
QVQLVESGGGLVQPGGSLTLSCIVSGRSVSINPMYWYRQGPGKQRELVVSLLPSGRTHDAHFAKGRFIISRDNAKNTVYLQMNSLKPEDTAVYYCNTADFWGQGTQVTVSSGGGGSGGGGSGGGGSQVQLVESGGGLVQPGGSLTLSCIVSGRSVSINPMYWYRQGPGKQRELVVSLLPSGRTHDAHFAKGRFIISRDNAKNTVYLQMNSLKPEDTAVYYCNTADFWGQGTQVTVSS

SEQ ID NO: 465 - protein, 255 aa, synthetic construct
  REGION 1..255: note = Description of Artificial Sequence: Synthetic polypeptide
QVQLVESGGGLVQAGDSLRLSCTASGRTFSNNAMGWFRQAPGKQRELVAAISRAGNTNYADSMKGRVTISGDNAKNTVYLQMNSLKPEDTAVYYCKASSGSSVYIGVGSWGQGTQVTVSSGGGGSGGGGSGGGGSQVQLVESGGGLVQAGDSLRLSCTASGRTFSNNAMGWFRQAPGKQRELVAAISRAGNTNYADSMKGRVTISGDNAKNTVYLQMNSLKPEDTAVYYCKASSGSSVYIGVGSWGQGTQVTVSS

SEQ ID NO: 466 - protein, 205 aa, synthetic construct
  REGION 1..205: note = Description of Artificial Sequence: Synthetic polypeptide
MQVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSELKHSISDYTEAEFLQLVTTICNADTSSEEELVKLVTHFEEMTEHPSGSDLIYYPKEGDDDSPSGIVNTVKQWRAANGKSGFKQG

SEQ ID NO: 467 - protein, 253 aa, synthetic construct
  REGION 1..253: note = Description of Artificial Sequence: Synthetic polypeptide
MQVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSESKRNKPGKATGKGKPVGDKWLDDAGKDSGAPIPDRIADKLRDKEFKSFDDFRKAVWEEVSKDPELSKNLNPCNKSSVSKGYSPFTPKNQQVGGRKVYELHADKPISQGGEVYDMDNIRVTTPKRHIDIHRGK

SEQ ID NO: 468 - protein, 160 aa, synthetic construct
  REGION 1..160: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGSGGGSRMKQLEDKIEELLSKIYHLENEIARLKKLIGER

SEQ ID NO: 469 - protein, 160 aa, synthetic construct
  REGION 1..160: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGSGGGSRMKQIEDKIEEILSKIYHIENEIARIKKLIGER

SEQ ID NO: 470 - protein, 158 aa, synthetic construct
  REGION 1..158: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGSGGGSRMKQIEDKLEEILSKLYHIENELARIKKLLG

SEQ ID NO: 471 - protein, 168 aa, synthetic construct
  REGION 1..168: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGGGGGGGGLTDTLQAETDQLEDEKSALQTEIANLLKEKEKLEFILAA

SEQ ID NO: 472 - protein, 168 aa, synthetic construct
  REGION 1..168: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGGGGGGGGRIARLEEKVKTLKAQNSELASTANMLREQVAQLKQKVMN

SEQ ID NO: 473 - protein, 211 aa, synthetic construct
  REGION 1..211: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGGSERVVINISGLRFETQLKTLAQFPETLLGDPKKRMRYFDPLRNEYFFDRNRPSFDAILYYYQSGGRLRRPVNVPLDIFSEEIRFYELG

SEQ ID NO: 474 - protein, 244 aa, synthetic construct
  REGION 1..244: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSSGGGGSGGGGSGGGGSGGGGSGGGGSALSNQITFTTTQQGDIYTVIPQVTLNEPCVCQVQILSVRDGVGGQSHTQQKQTLSLPANQPIELSRLSVNISSEDSVKIIVTVSDGQSLHLSQQWPPSAQ

SEQ ID NO: 475 - protein, 117 aa, synthetic construct
  REGION 1..117: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTV

SEQ ID NO: 476 - protein, 109 aa, synthetic construct
  REGION 1..109: note = Description of Artificial Sequence: Synthetic polypeptide
QVQLVESGGGLVQPGGSLTLSCIVSGRSVSINPMYWYRQGPGKQRELVVSLLPSGRTHDAHFAKGRFIISRDNAKNTVYLQMNSLKPEDTAVYYCNTADFWGQGTQVTV

SEQ ID NO: 477 - protein, 118 aa, synthetic construct
  REGION 1..118: note = Description of Artificial Sequence: Synthetic polypeptide
QVQLVESGGGLVQAGDSLRLSCTASGRTFSNNAMGWFRQAPGKQRELVAAISRAGNTNYADSMKGRVTISGDNAKNTVYLQMNSLKPEDTAVYYCKASSGSSVYIGVGSWGQGTQVTV

SEQ ID NO: 478 - protein, 111 aa, synthetic construct
  REGION 1..111: note = Description of Artificial Sequence: Synthetic polypeptide
QVQLVESGGGLVQPGGSLTLSCIVSGRSVSINPMYWYRQGPGKQRELVVSLLPSGRTHDAHFAKGRFIISRDNAKNTVYLQMNSLKPEDTAVYYCNTADFWGQGTQVTVSS

SEQ ID NO: 479 - protein, 120 aa, synthetic construct
  REGION 1..120: note = Description of Artificial Sequence: Synthetic polypeptide
QVQLVESGGGLVQAGDSLRLSCTASGRTFSNNAMGWFRQAPGKQRELVAAISRAGNTNYADSMKGRVTISGDNAKNTVYLQMNSLKPEDTAVYYCKASSGSSVYIGVGSWGQGTQVTVSS

SEQ ID NO: 480 - protein, 119 aa, synthetic construct
  REGION 1..119: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKTFDGTRWHDYWGQGTQVTVSS

SEQ ID NO: 481 - protein, 119 aa, synthetic construct
  REGION 1..119: note = Description of Artificial Sequence: Synthetic polypeptide
QVKLEESGGGLVQPGGSLEVSCAASGIIFSPNAMGWYRQAPGEQRELVATITSFGIINYADSVKDRFTISRDNAKNTVYLQMTSLKPEDTAVYYCNAKAFDGTRWHDYWGQGTQVTVSS

SEQ ID NO: 482 - protein, 118 aa, synthetic construct
  REGION 1..118: note = Description of Artificial Sequence: Synthetic polypeptide
QVKLEESGGGLVQAGGSLRLSCAVSGSIFSTNVMGWFRQAPGKQRGFVAHITSGGNTDYADSVNGRFTMSRDNAKNTVYLQMNSLKPEDTAVYYCAAQTLGSSYYDAWGQGTQVTVSS

SEQ ID NO: 483 - protein, 128 aa, synthetic construct
  REGION 1..128: note = Description of Artificial Sequence: Synthetic polypeptide
QVQLVESGGGLVQAGGSLRLSCAASGRTFSSYAMGWFRQAPGKEREFVASINWSGGRIYYADSVKGRFTISRDNAKNTVYLQMNSLKPEDTAVYYCNADYDNSGSYYYQKGNYEYDYWGQGTQVTVSS

SEQ ID NO: 484 - protein, 124 aa, synthetic construct
  REGION 1..124: note = Description of Artificial Sequence: Synthetic polypeptide
QVQLQESGGGLVQAGGSLRLSCAASGRTSSSAYTAWFRQAPGNEREFVASISWSGTTTYYADPVKGRFTISRDNAKNTVYLQMNSLKPDDTAVYYCAADRRSTIGSPRQQYAYWGQGTQVTVSS

SEQ ID NO: 485 - protein, 124 aa, synthetic construct
  REGION 1..124: note = Description of Artificial Sequence: Synthetic polypeptide
QVQLQESGGGLVQAGGSLRLSCAASTRTSSSSYTAWFRQAPGNEREFVASISYSGTTTYYADPVKGRFTISRDSAKNTVYLQMNSLKPDDTAVYYCAADRRSTIGSPRQQYAYWGQGTQVTVSS

SEQ ID NO: 486 - protein, 124 aa, synthetic construct
  REGION 1..124: note = Description of Artificial Sequence: Synthetic polypeptide
QVQLQESGGGLVQAGGSLRLSCAASGRTSSSAYTAWFRQAPGNEREFVASISWSGTTTYHAHPVKGRFTIFRDNAKNTVYLQMNSLKPDDTGVYYCAADRRSTIGTPREQYAYWGQGTQVTVSS

SEQ ID NO: 487 - protein, 120 aa, synthetic construct
  REGION 1..120: note = Description of Artificial Sequence: Synthetic polypeptide
QVQLQESGGGLVQAGGSLRLSCAASGSTLSNYAVEWYRQAPGNQREYVARISSGGSTNYADSVKGRFTISRDNAKNTVYLQMNSLKPEDTAVYYCHTYDFQGWGLRSDYWGQGTQVTVSS

SEQ ID NO: 488 - protein, 130 aa, synthetic construct
  REGION 1..130: note = Description of Artificial Sequence: Synthetic polypeptide
QVQLQESGGRLVQAGGSLRLSCAASGRTFSSLAMGWFRQAPGKEREFVAAISRSGDYTYFSDSVKGRFAISRDNAKDTVSLQMNNLKPDDTAVYTCAATKIVTPWTSTYYYTKAYEWDYWGQGTQVTVSS

SEQ ID NO: 489 - protein, 111 aa, synthetic construct
  REGION 1..111: note = Description of Artificial Sequence: Synthetic polypeptide
QVQLVESGGGLVQPGGSLRLSCAASGVAFNSRIMGWYRQAPGKQRELVALITSGGSTNYADSVKGRFTISRNNAKKTVYLQMNSLKPEDTAVYYCNIRNYWGQGTQVTVSS

SEQ ID NO: 490 - protein, 120 aa, synthetic construct
  REGION 1..120: note = Description of Artificial Sequence: Synthetic polypeptide
QVQLVESGGGLVQAGGSLRLSCAASGRTFNTYYMGWFRQAPGKEREFVSAIRWSDGGTWYADSMKGRFTISRDNAKNTGYLQMNSLKPEDTAIYYCNANVYDGNRWRTYWGQGTQVTVSS

SEQ ID NO: 491 - protein, 128 aa, synthetic construct
  REGION 1..128: note = Description of Artificial Sequence: Synthetic polypeptide
QVKLEESGGGLVRAGGSLTLSCGASRGTFRTYSMGWFRQAPGKEREFVAAITWNGKYTYYGDSVQGRFTISKDNAKNTVSLQMNRLNPEDTAVYYCAANPIPTAQPPGIMAARSYVHWGQGTQVTVSS

SEQ ID NO: 492 - protein, 124 aa, synthetic construct
  REGION 1..124: note = Description of Artificial Sequence: Synthetic polypeptide
QVQLQQSGGGLVQAGGSLRLSCAASGRTSPSSYTAWFRQAPGNEREFVASISWSGTTTYYADPVKGRFTISRDNAKNTVYLQMNSLKPDDTAVYYCAADRRSTIGSPRQQYAYWGQGTQVTVSS

SEQ ID NO: 493 - protein, 130 aa, synthetic construct
  REGION 1..130: note = Description of Artificial Sequence: Synthetic polypeptide
QVQLQESGGRLVQAGGSLRLSCAASGRTFSSLAMGWFRQAPGKEREFVAAITRSGDYTYFSDSVKGRFAISRDNAKDTVSLQMNNLKPDDTAVYTCAATKIVTPWTSTYYYTKAYEWDYWGQGTQVTVSS

SEQ ID NO: 494 - protein, 117 aa, synthetic construct
  REGION 1..117: note = Description of Artificial Sequence: Synthetic polypeptide
QVQLQESGGGLVQAGKSLRLSCAASTAILSIDSMGWNRQAVGNQRELVAVIARGGSTKYADSVKGRFTITRDISKNTIYLQMNSLKPEDTGVYYCAADPGGASPLSWGQGTQVTVSL

SEQ ID NO: 495 - protein, 118 aa, synthetic construct
  REGION 1..118: note = Description of Artificial Sequence: Synthetic polypeptide
QVQLQESGGGMVQAGGSLRLACTASGDISTIDVMGWNRQAPGKHRELVAIIARGGTIKYADSVKGRFTISRDNTKNTVTVYLQMNNVNAEDTAVYYCAVDTGSPRLTWGQGTQVTVSS

SEQ ID NO: 496 - protein, 111 aa, synthetic construct
  REGION 1..111: note = Description of Artificial Sequence: Synthetic polypeptide
QVQLQESGGGLVQAGGSLRLSCAASGFTFSSSIMAWYRQAPGKQREAIASIPSFGSAVYADSVKDRFTISRDNNKNMVYLQMNSLKPEDTAVYYCNTRLYWGQGTQVTVSS

SEQ ID NO: 497 - protein, 117 aa, synthetic construct
  REGION 1..117: note = Description of Artificial Sequence: Synthetic polypeptide
QVQLQESGGGLVQAGGSLRLACTASGDISSISVMGWNRQAPGKQQRELVAAIASGGSVKYADSVKGRFTISRDNTKNTVYLQMNSVNAEDTAVYYCAVDTGSPRLTWGQGTQVTVSS

SEQ ID NO: 498 - protein, 111 aa, synthetic construct
  REGION 1..111: note = Description of Artificial Sequence: Synthetic polypeptide
QVQLQESGGGLVQAGGSLRLSCAASGFTFSTNILAWYRQAPGKQREAIASITPFGSAVYANSVKDRFTISRDNNKNMVYLQMNSLKPEDTAVYSCNTQLYWGQGTQVTVST

SEQ ID NO: 499 - protein, 117 aa, synthetic construct
  REGION 1..117: note = Description of Artificial Sequence: Synthetic polypeptide
QVQLQESGGGLVQAGGSLRLSCAASTSILSINAMGWNREAPGNRREMVAIIAPGGTTNYADSVKGRFTITRDISKNTIYLQMNNLKPEDTGVYYCAADPGGQSPLSWGQGTQVTVSL

SEQ ID NO: 500 - protein, 116 aa, synthetic construct
  REGION 1..116: note = Description of Artificial Sequence: Synthetic polypeptide
QVQLQESGGGSVQAGGSLRLSCAASGSISSITAMGWNRQAPGNQRELVAVIARGGMIKYDDSVKGRFTISRDIAKNTVFLQMDSLKPEDTGVYYCAVDNGDPRLHWGQGIQVTVSS

SEQ ID NO: 501 - protein, 117 aa, synthetic construct
  REGION 1..117: note = Description of Artificial Sequence: Synthetic polypeptide
QVQLQESGGGLVQAGGSLRLSCAASGSISSITAMGWNRQAPGNQQRDLVAVIARGGMTKYADSVQGRFTISRDIAKNTVYLQMNSLKPEDTGVYYCALDNGDPRLHWGQGIQVTVSS

SEQ ID NO: 502 - protein, 111 aa, synthetic construct
  REGION 1..111: note = Description of Artificial Sequence: Synthetic polypeptide
QVQLQESGGGLVQAGGSLRLSCAASGFTFSSAIMAWYRQAPGKQREAIASIPSFGSAVYADSVKDRFTISRDNNKNMVYLQMDSLKPEDTAVYYCNTRLYWGQGTQVTVST

SEQ ID NO: 503 - protein, 118 aa, synthetic construct
  REGION 1..118: note = Description of Artificial Sequence: Synthetic polypeptide
QVQLQESGGGLVQAGGSLRLSCAASTSILSIDAMGWNRQAPGNQRRDLVAVIARGGSTQYADSVKGRFTITRDISKNTIYLQMNSLKPEYTGVYYCAADPGGASGLSWGQGTQVTVSL

SEQ ID NO: 504 - protein, 117 aa, synthetic construct
  REGION 1..117: note = Description of Artificial Sequence: Synthetic polypeptide
QVQLQESGGGLVQAGGSLRLSCAASGSISSITAMGWNRQAPGNQQRDLVAVIARGGMTKYADSVQGRFTISRDIANNTVYLQMNSLKPEDTGVYYCALYNGDPRLHWGQGIQVTVSS

SEQ ID NO: 505 - protein, 118 aa, synthetic construct
  REGION 1..118: note = Description of Artificial Sequence: Synthetic polypeptide
QVQLQESGGGLVQAGGSLSLSCTASGSAFSGDAMGWYRRAPGKQREFVAGISSGAITNYMDSVKGRFTISRDNAKNTVYLQMTKLKPEDTAVYYCNRIQAVLRGNSGWGQGTQVTVSS

SEQ ID NO: 506 - protein, 119 aa, synthetic construct
  REGION 1..119: note = Description of Artificial Sequence: Synthetic polypeptide
QVQLQESGGGLVQPGGSLSLSCTASGSAFSGGDAMGWYRRAPGKQREFVAGISSGGIANYMDSVKGRFTISRDNAKKAVYLQMTSLKPEDTALYYCNSITAVLRGNSGWGQGTQVTVSS

SEQ ID NO: 507 - protein, 118 aa, synthetic construct
  REGION 1..118: note = Description of Artificial Sequence: Synthetic polypeptide
QVQLQESGGGLVQAGGSLTLSCTASGSAFSGDAMGWYRRAPGKEREFVAGISSGGIPNYMGSVQGRFTISRDNAKNTVYLQMRRLKPEDTAVYYCNSISAVLRGNGVWGQGTQVTVSS

SEQ ID NO: 508 - protein, 127 aa, synthetic construct
  REGION 1..127: note = Description of Artificial Sequence: Synthetic polypeptide
QVQLQESGGGLVQAGGSLRLSCAASGLTFNNYAMGWFRQAPGKEREFVATISRDGTNTRYADSVKGRFTISRDNAKNTVDLQMNSLKPEDTAVYYCGVGRGTGYAYTAINEYDYSKWGQGTQVTVSS

SEQ ID NO: 509 - protein, 114 aa, synthetic construct
  REGION 1..114: note = Description of Artificial Sequence: Synthetic polypeptide
QVQLQESGGGLVQAGGSLRLSCAASGIDSSFYVMAWYRQAPGKQRELVASLGTPDSATYADFVKGRFIISRDNAKSTVYLQMNSLKPEDTAVYYCYGLYRQVYWGQGTQVTVSS

SEQ ID NO: 510 - protein, 114 aa, synthetic construct
  REGION 1..114: note = Description of Artificial Sequence: Synthetic polypeptide
QVQLQESGGGLVQAGGSLRLSCVASGIDSSFYVMAWYRQAPGKQRELVASISSADSPRYEDFVKGRFTISRDNGKNTVYLQMNSLKPEDTAVYYCYGLYRQVHWGQGTQVTVSS

SEQ ID NO: 511 - protein, 121 aa, synthetic construct
  REGION 1..121: note = Description of Artificial Sequence: Synthetic polypeptide
QVQLQQSGGGLVQAGGSLRLSCAASGLTFSSYAMGWFRQAPGKEREFIVAIGWSGGSTYYADSVKGRFTISRDNAKNTVYLHMNSLKPEDTAVYYCAARRTTAWGKGTDYWGQGTQVTVSS

SEQ ID NO: 512 - protein, 126 aa, synthetic construct
  REGION 1..126: note = Description of Artificial Sequence: Synthetic polypeptide
QVQLQESGGGLVQAGGSLRLSCAASESIFSRNAMGWYRQAPGKERDLVAPGKERELVAGIGSDGSTNYAESVKGRFTISRDNAKNTVYLQMNSLKPEDTAVYYCRVVLATSPYNYWGQGTQVTVSS

SEQ ID NO: 513 - protein, 114 aa, synthetic construct
  REGION 1..114: note = Description of Artificial Sequence: Synthetic polypeptide
QVQLQESGGGLVQAGGSLRLSCAASGITSSLYVMAWYRQAPGKQRELVAHINSGDSPRYADFVQGRFTISRDNGKNTVYLQMNSLKPEDTAVYYCYGLYRQVHWGQGTQVTVSS

SEQ ID NO: 514 - protein, 127 aa, synthetic construct
  REGION 1..127: note = Description of Artificial Sequence: Synthetic polypeptide
QVQLQESGGGLVQAGGSLRLACAASGLTFNNYAMGWFRQAPGKEREFVATISRDGTSTRYADSVKGRFTISRDNAKNTVNLQMNRLKPEDTAVYYCGVGRGSGYAYSAINEYDYSSWGQGTQVTVSS

SEQ ID NO: 515 - protein, 116 aa, synthetic construct
  REGION 1..116: note = Description of Artificial Sequence: Synthetic polypeptide
QVQLQESGGGLVQAGGSLRLSCAASGIDSSFYVMAWYRQAPGQQRELVASISMTSADSPRYADFVKGRFTISRDNAKSTVYLQMNSLKPEDTAVYYCYGLYRQVHWGQGTQVTVSS

SEQ ID NO: 516 - protein, 116 aa, synthetic construct
  REGION 1..116: note = Description of Artificial Sequence: Synthetic polypeptide
QVQLQESGGGLVQAGGSLRLSCAASGSGILFRISAMGWYRQAPGKERDLVAGISSGGSTNYADSVKGRFTISRDNAKNTVYLQMNSLKPEDTAVYYCNIVGRTDSWGQGTQVTVSS

SEQ ID NO: 517 - protein, 127 aa, synthetic construct
  REGION 1..127: note = Description of Artificial Sequence: Synthetic polypeptide
QVQLQESGGGSVQAGGSLRLSCAASARTLSNYAMGWFRQAPGKEREFVATISRSGGSIHYADSVKGRFTISRDNAKNTVNLQMNSLKVEDTAVYYCGRARGTGYAYTALNQYDYDYWGQGTQVTVSS

SEQ ID NO: 518 - protein, 118 aa, synthetic construct
  REGION 1..118: note = Description of Artificial Sequence: Synthetic polypeptide
QVQLQESGGGLVQAGGSLTLSCTTSGSAFSGDAMGWYRRAPGQEREFVAGISSGGITNYMNFVKGRFTISRDNAKNTVYLQMTSLKPEDTAVYYCNSIKAVLRGNSGWGQGTQVTVSS

SEQ ID NO: 519 - protein, 127 aa, synthetic construct
  REGION 1..127: note = Description of Artificial Sequence: Synthetic polypeptide
QVQLQESGGGLVQAGGSLRLSCAASGLTFHNYAMGWFRQAPGKEREFVATISRDGTNTHYADSVKGRFTISRDNAKNTVDLQMNSLKPEDTAVYYCGVGRGSGYAYTAINEYDYSKWGQGTQVTVSS

SEQ ID NO: 520 - protein, 118 aa, synthetic construct
  REGION 1..118: note = Description of Artificial Sequence: Synthetic polypeptide
QVQLQESGGGLVQAGGSLSLSCTASGSAFSGDAMGWYRRAPGKQRDFVAGISSGHITNYMDSVKGRFTISRDNAKNTVYLQMTKLKPEDTAVYYCNSITAVLRGNSGWGQGTQVTVSS

SEQ ID NO: 521 - protein, 127 aa, synthetic construct
  REGION 1..127: note = Description of Artificial Sequence: Synthetic polypeptide
QVQLQESGGGLVQAGGSLRLSCAASGRTFSTYAMGWFRQAPGKEREFVATISRSGDNIYYADSVKGRFTISRDNAKNTVSLQMNSLKVEDTAVYYCGRARGTGYAHTALNQYDYDYWGQGTQVTVSS

SEQ ID NO: 522 - protein, 118 aa, synthetic construct
  REGION 1..118: note = Description of Artificial Sequence: Synthetic polypeptide
QVQLQESGGGLVQAGGSLSLSCRVSGSAFSGDAMGWYRRAPGKQREFVAGISSGGIENYMDSVKGRFTISRDNAKNTVYLRMSSLKPEDTAVYYCNLIKAVLRGNSGWGQGTQVTVSS

SEQ ID NO: 523 - protein, 127 aa, synthetic construct
  REGION 1..127: note = Description of Artificial Sequence: Synthetic polypeptide
QVQLQESGGGLVQAGGSLRLSCAASGLTFNNYAMGWFRQAPGKEREFVATISRDGTNTRYADSVKGRFTISRDNAKNTVNLQMNSLKPEDTAVYYCGVGRGTGYAYTAIREHDYSSWGQGTQVTVSS

SEQ ID NO: 524 - protein, 118 aa, synthetic construct
  REGION 1..118: note = Description of Artificial Sequence: Synthetic polypeptide
QVQLQESGGGLVQAGGSLSLSCTASGSAFSGDAMGWYRRAPGKQREFVAGISSGGITNYMNSVKGRFTISRDNAKNTVYLHMTGVKPADTAVYYCNSITAVLRGNSGWGPGTQVTVSS

SEQ ID NO: 525 - protein, 118 aa, synthetic construct
  REGION 1..118: note = Description of Artificial Sequence: Synthetic polypeptide
QVQLQESGGGLVQPGGSLTLSCTASGSAFSGDAMGWYRRAPGKQREFVAGISSGGIANYMDSTEGRFTISRDDAKNTVYLQMTGVKPADTAVYYCNTIKAVLRGNAGWGQGTQVTVSS

SEQ ID NO: 526 - protein, 118 aa, synthetic construct
  REGION 1..118: note = Description of Artificial Sequence: Synthetic polypeptide
QVQLQESGGGLVQAGGSLSLSCTASGSAFSGDAMGWYRRAPGKQREFVAGISSGAITNYMDSVKGRFTISRDNAKNMVYLQMTKLKPEDTAVYYCNSITAVLRGNSGWGQGTQVTVSS

SEQ ID NO: 527 - protein, 118 aa, synthetic construct
  REGION 1..118: note = Description of Artificial Sequence: Synthetic polypeptide
QVQLQESGGGLVQAGGSLSLSCTASGSAFSGDAMGWYRRAPGKQREFVAGISSGGITNYMDSVKGRFTISRDNAKNTVYLQMTSLKPEDTAVYYCNIISAVLRGNGGWGQGTQVTVSS

SEQ ID NO: 528 - protein, 118 aa, synthetic construct
  REGION 1..118: note = Description of Artificial Sequence: Synthetic polypeptide
QVQLQQSGGGLVQAGGSLSLSCTASISGFSGDAMGWYRRAPGKQREFVAGISSGGITNYMDSVKGRFTISRDNAKNTVYLQMTNLKPEDTAVYYCNTITGVLRGNSGWGQGTQVTVSS

SEQ ID NO: 529 - protein, 114 aa, synthetic construct
  REGION 1..114: note = Description of Artificial Sequence: Synthetic polypeptide
QVQLQESGGGLVQAGGSLRLSCAGSGIISSAYVMAWYRQRPGKQRELVASITSGDSPRYEDFVKGRFTISRDNAKSTVYLQMNSLKPEDTAVYYCYGLYRQVYWGQGTQVTVSS

SEQ ID NO: 530 - protein, 111 aa, synthetic construct
  REGION 1..111: note = Description of Artificial Sequence: Synthetic polypeptide
QVQLQQSGGGLVQAGGSLKLSCAASGIAFSTYGMNWFRQTPGKQREYVAYITGNGDDNVAQSMEGRFTISRDNAKNTGYLQMNSLKPEDTGVYYCNIGMYWGQGTQVTVSS

SEQ ID NO: 531 - protein, 118 aa, synthetic construct
  REGION 1..118: note = Description of Artificial Sequence: Synthetic polypeptide
QVQLQESGGGLVQAGGSLSLSCTASGSAFSGDAMGWYRRAPGKQREFVAGISSGGITNYMGFVKGRFTISRDNAKNTVYLQMTSLKPEDTAVYYCNSISAVLRGNSGWGQGTQVTVSA

SEQ ID NO: 532 - protein, 118 aa, synthetic construct
  REGION 1..118: note = Description of Artificial Sequence: Synthetic polypeptide
QVQLQESGGGLVQAGGSLSLSCTASGSAFSGDAMGWYRRAPGKQRDFVAGISSGGITNYMDSVKGRFTISRDNAKNTVYLQMTSLKPEDTAVYYCNSISAVLRGNGGWGQGTQVTVSS

SEQ ID NO: 533 - protein, 118 aa, synthetic construct
  REGION 1..118: note = Description of Artificial Sequence: Synthetic polypeptide
QVQLQESGGGLVQPGGSLSLSCTASGSAFSGDAMGWYRRAPGKQREFVAGISSGGITNYMDSVKDRFTISRDNAKNTLYLQMTNLKPEDTAVYYCNSITAVLRGNSDWGQGTQVTVSS

SEQ ID NO: 534 - protein, 118 aa, synthetic construct
  REGION 1..118: note = Description of Artificial Sequence: Synthetic polypeptide
QVQLQESGGGLVQAGGSLSLSCTTSGSAFSGDAMGWYRRAPGKQREFVAGISSGGIPNYMGFVRGRFTISRDNTKNTVYLQMTSLKPDDTAVYYCNIIKTVLRGNAVWGQGTQVTVSS

SEQ ID NO: 535 - protein, 119 aa, synthetic construct
  REGION 1..119: note = Description of Artificial Sequence: Synthetic polypeptide
QVQLQESGGGLVQAGGSLSLSCTASGSAFSGGDAMGWYRRAPGKQREFVAGISSGGITNYMDFVKGRFTISRDNAKNTVYLQMTSLKPEDTAVYYCNSITAVLRGNSGWGQGTQVTVSS

SEQ ID NO: 536 - protein, 118 aa, synthetic construct
  REGION 1..118: note = Description of Artificial Sequence: Synthetic polypeptide
QVQLQESGGGLVQAGGSLRLSCVASGITFSSDAMGWYRQAPGKQREFVAGISSGDITNYPDSVKGRFAISRDNAKNTVYLQMNSLKPEDTAVYYCNTITRLLYGMDYWGKGTLVTVSS

SEQ ID NO: 537 - protein, 124 aa, synthetic construct
  REGION 1..124: note = Description of Artificial Sequence: Synthetic polypeptide
QVQLQESGGGLVQPGGSLRLSCAASGFTLDGYAIGWFRQAPGKEREWVSCIIYRDGSPAYADSVWGRFTISRDNAKNNVYLEMNSLKPEDTAVYYCAARPGGACSRYPSNYDTWGQGTQVTASS

SEQ ID NO: 538 - protein, 120 aa, synthetic construct
  REGION 1..120: note = Description of Artificial Sequence: Synthetic polypeptide
QVKLEESGGGLVQAGGSLRLSCAASGSIFSINAMGWYRQAPGKQRELVAAITTGGNTANTAYADSVKGRFTISRDKAKNTVYLQMNSLKPEDTAVYYCAARGLSYEYDYWGQGTQVTVSS

SEQ ID NO: 539 - protein, 122 aa, synthetic construct
  REGION 1..122: note = Description of Artificial Sequence: Synthetic polypeptide
QVQLVESGGGLVQAGGSLRLSCAASGSIFSINAMGWYRQAPGKQRELVAAITITSGRGGNTAYADSVKGRFTISRDNAKNTVYLQMNTLKPEDTAVYYCAARGAMTYEYDYWGQGTQVTVSS

SEQ ID NO: 540 - protein, 117 aa, synthetic construct
  REGION 1..117: note = Description of Artificial Sequence: Synthetic polypeptide
QVQLVESGGGLVQAGGSLRLSCAASGIIFSPNAMGWYRQAPGKQRELVSTITSFGIINYADSVKDTISRDNAKNTVYLQMTSLKPEDTAVYYCNAKTFDGTRWRDYWGQGAQVTVSS

SEQ ID NO: 541 - protein, 121 aa, synthetic construct
  REGION 1..121: note = Description of Artificial Sequence: Synthetic polypeptide
QVKLEESGGGLVQAGGSLRLSCAASGNIFSINAMGWYRQAPGKQRELVAAITTGGSYGNTNYADSVKGRFTISRDNAKNTVYLQMNSLKPEDTAVYYCAARGSQTYEYDYWGQGTQVTVSS

SEQ ID NO: 542 - protein, 119 aa, synthetic construct
  REGION 1..119: note = Description of Artificial Sequence: Synthetic polypeptide
QVKLEESGGGLVQPGGSLTLSCAASGRIFSIYDMGWFRQAPGKERELVSAITWGNGNTAYGDSVKGRFSISRDFAKNTVYLQIDSLKAEDTAVYYCPARIVNGGSWDYWGQGTQVTVSS

SEQ ID NO: 543 - protein, 119 aa, synthetic construct; identical to SEQ ID NO: 542
  REGION 1..119: note = Description of Artificial Sequence: Synthetic polypeptide

SEQ ID NO: 544 - protein, 119 aa, synthetic construct
  REGION 1..119: note = Description of Artificial Sequence: Synthetic polypeptide
QVKLEESGGGLVQAGGSLRLSCAASGRMFSSYDMGWFRQAPGKERDIVAAITKNGRTTSYANSVKGRFTISRDNTKSTVYLQIHSLKPEDTAVYYCAGRRSNADNWDYWGQGTQVTVSS

SEQ ID NO: 545 - protein, 122 aa, synthetic construct
  REGION 1..122: note = Description of Artificial Sequence: Synthetic polypeptide
QVKLEESGGGLVQAGGSLRLSCAVSGSIFSINAVGWYRQAPGKQRELVAAIGTGGSSSGNTAYADSVKGRFTISNDAAKNTVYLQMNSLKPDDTAVYYCAARGTISYEYDYWGQGTQVTVSS

SEQ ID NO: 546 - protein, 111 aa, synthetic construct
  REGION 1..111: note = Description of Artificial Sequence: Synthetic polypeptide
QVQLVESGGGLVQAGGSLTLSCIVSGISVNINPMYWYRPGPGNQRELVVSLLPTGTTHDAHFTKGRFIISKDDAKNTVYLLMNSLKPEDTAVYYCNTADFWGQGTQVTVSS

SEQ ID NO: 547 - protein, 120 aa, synthetic construct
  REGION 1..120: note = Description of Artificial Sequence: Synthetic polypeptide
QVKLEESGGGLVQAGGSLRLSCAASGSTFSINAMGWFRQAPGKQRELVAAISRAGSTNTADSVKGRFTISRDNAKNTVYLQMNSLKPEDTAVYYCKASSGSSVYIGFGSWGQGTQVTVSS

SEQ ID NO: 548 - protein, 111 aa, synthetic construct
  REGION 1..111: note = Description of Artificial Sequence: Synthetic polypeptide
QVQLVESESGLVLAGGSLTLTRFCSVSSVSINPMYWYRQGPGKQRELVLILLSMARAQNAHFPNGQFLISIYEDDNTMYLQLSIQKPEDADVYDCNTTDFWGQGTQVTVSS

SEQ ID NO: 549 - protein, 128 aa, synthetic construct
  REGION 1..128: note = Description of Artificial Sequence: Synthetic polypeptide
QVKLEESGGGLVQAGGSLRLSCAASGRTFSRLAMGWFRQAPGKEREFVVAISWSGGNTNYADSVKGRFTISRDNAKNTVYLQMNSLNAEDTAVYYCAAPERSGSYAYTPSRLNEYAYWGQGTQVTVSS

SEQ ID NO: 550 - protein, 118 aa, synthetic construct
  REGION 1..118: note = Description of Artificial Sequence: Synthetic polypeptide
QVQLVESGGGLVQAGGSLRLSCAASGRIFSSYDMGWFRQAPGKERELVAAIRWGNGNTAYGDSVKGRFSISRDFAKNTVYLHIDSLKAEDTAVYYCAARGLAYEYEYWGQGTQVTVSS

SEQ ID NO: 551 - protein, 119 aa, synthetic construct
  REGION 1..119: note = Description of Artificial Sequence: Synthetic polypeptide
QVQLVESGGGLVQAGGSLRLSCAASGRIFSSYDMGWFRQAPGKERELVAAIRWGNGNTAYGDSVKGRFSISRDFAKNTVYLHIDSLKAEDTAVYYCAARIVNGGSWDYWGQGTQVTVSS

SEQ ID NO: 552 - protein, 119 aa, synthetic construct
  REGION 1..119: note = Description of Artificial Sequence: Synthetic polypeptide
QVQLVESGGGSVQPAGSLRLSCAVSGIIFSPNALGWYRPAPGKERELVASIISGGRSDYADSVKDRFTIARDNPKNTVTLQMNSLKPEDTAIYYCNANVYDGNRWRTYWGQGTQVTVSS

SEQ ID NO: 553 - protein, 122 aa, synthetic construct
  REGION 1..122: note = Description of Artificial Sequence: Synthetic polypeptide
QVKLEESGGGLVQAGGSLRLSCAASGRTFRSYTMGWFRQAPGLEREIIAAISWSAGSTRYADSMSDRFTISRDNAKNTVYLGMDSLKPEDTAVYYCAAGTKYSDTIITWGSWGQGTQVTVSS

SEQ ID NO: 554 - protein, 122 aa, synthetic construct
  REGION 1..122: note = Description of Artificial Sequence: Synthetic polypeptide
QVQLVESGGGLVQPGGSLRLSCAVSGSTVTISTVGWYRQAPGNQRVLVASISSDSTTNYAHSVKGTISRHNAENPVSRLQMNSLKPEDTAVYYCNVVGTYWTGADWRPFDTWGRGTQVTVSS

SEQ ID NO: 555 - protein, 113 aa, synthetic construct
  REGION 1..113: note = Description of Artificial Sequence: Synthetic polypeptide
QVKLEESGGGLVQAGGSLRLSCAASGRSFSSYNMGWFRQAPGKEREFVAAITWSGNTYYADSVKGTISRDNAKNTVYLQMNSLKPEDTAVYFCKVRAEDTDYWGRGTQVTVSS

SEQ ID NO: 556 - protein, 122 aa, synthetic construct
  REGION 1..122: note = Description of Artificial Sequence: Synthetic polypeptide
QVKLEESGGGLVQPGGSLRLSCAASGFTFSMYGMTWVRQAPGKGLEWVSAINSGGARTSYADSVKGRFTISRDNAKNTLYLQMNSLKPEDTAVYYCAKASLPWFDGSSPDYWGQGTQVTVSS

SEQ ID NO: 557 - protein, 124 aa, synthetic construct
  REGION 1..124: note = Description of Artificial Sequence: Synthetic polypeptide
QVKLEESGGGLVQAGGSLRLSCAASGLTFSSYGMGWFRQGPGKERESVAAIKMSGDTYYTDSVKGRFTISRDNAKNTVYLQMDSLKPEDTAVYFCAAARVRTPGWGPQKSYDYWGQGTQVTVSS

SEQ ID NO: 558 - protein, 124 aa, synthetic construct
  REGION 1..124: note = Description of Artificial Sequence: Synthetic polypeptide
QVKLEESGGGLVQAGGSLRLSCAASGRTFGSLHMGWFRQAPGKEREFVSAISAAGGVTDYADSAKGRFTISRDNAKNTVYLQMNSLKPEDTAVYYCAAVKYWGRRQRADEYDYWGQGTQVTVSS

SEQ ID NO: 559 - protein, 117 aa, synthetic construct
  REGION 1..117: note = Description of Artificial Sequence: Synthetic polypeptide
QVRLEKSGGGLVQPGGSLTLSCTASGSISSIKAMGWYRQAPGKQRELAALWRMYSGTAYGDSVKGRSNLSVNHTNNTAYLQMNSLRPEDTAVYWCYLEIPESRGAFWGHGTQVTVSS

SEQ ID NO: 560 - protein, 121 aa, synthetic construct
  REGION 1..121: note = Description of Artificial Sequence: Synthetic polypeptide
QVQLVESGGGLVQAGGSLRLSCAASGRTFSRDAMGWFRQAPGKEREFVATINWNGRSTYYTESVKGRFTISRDNAKNTVYLQMNSLKPEDTAVYYCAAGEWGIRPYNYDYWGQGTQVTVSS

SEQ ID NO: 561 - protein, 128 aa, synthetic construct
  REGION 1..128: note = Description of Artificial Sequence: Synthetic polypeptide
QVQLVESGGGLVQAGGSLRLSCAASGRTFSSYAMGWFRQAPGKEREFVASINWSGGRIYYADSVKGRFTISRDNAKNTVYLQMNSLKPEDTAVYYCNTDYDNSGSYYYQKGNYEYDYWGQGTQVTVSS

SEQ ID NO: 562 - protein, 117 aa, synthetic construct
  REGION 1..117: note = Description of Artificial Sequence: Synthetic polypeptide
QVQLVESGGGLVQPGGSLKVSCAASGIIFSPNAMGWYRQAPGKQRDLVATITSSGIINYADSVKGTISRDNAKNTVYLQMTSLKPEDTAVYYCNAKAFDGTRWYDYWGQGTQVTVSS

SEQ ID NO: 563 - protein, 117 aa, synthetic construct
  REGION 1..117: note = Description of Artificial Sequence: Synthetic polypeptide
QVKLEESGGGLVQAGGSLRLSCAASGRTFSSYVMGWFREAPGKERRFVASISWSGGSSSYADSVKGTISRDYAENMVYLQMNSLKPEDTATYYCAARTALGGTYDYWGQGTQVTVSS

SEQ ID NO: 564 - protein, 117 aa, synthetic construct
  REGION 1..117: note = Description of Artificial Sequence: Synthetic polypeptide
QVQLAESGVGLVEPAGSLKFSCAASGIIFNPNAMGWHRQAPENQRELVATITSFGIINYADSVKDSISRDHDTNAVYLQMTNLRPDDPAVYYCNAITFYGTRWLDYWGQGTQVTVSS

SEQ ID NO: 565 - protein, 117 aa, synthetic construct
  REGION 1..117: note = Description of Artificial Sequence: Synthetic polypeptide
QVQLVESGGGLVQPGGSLRLSCAVSGIIFSPNALGWYRQAPGKERELVASIISGGRSDYADSVKDTIARDNPKNTVTLQMNSLKPEDTAIYYCNADVYDGNRWRTYWGQGTQVTVSS

SEQ ID NO: 566 - protein, 117 aa, synthetic construct
  REGION 1..117: note = Description of Artificial Sequence: Synthetic polypeptide
QVKLEESGGGLVQPGGSLKVSCAASGIIFSPNAMGWYRQAPGKQRELVATITSFGIINYADSVKDTISRDNAKNTVYLQMTSLKPEDTAVFYCFAKTFDGTRWCDYWGQGTQVTVSS

SEQ ID NO: 567 - protein, 117 aa, synthetic construct
  REGION 1..117: note = Description of Artificial Sequence: Synthetic polypeptide
QVQLVESGAGLVQPGGSLRLSCAVSGIIFSPNALGWYRQAPGKERELVASIISGGRSDYADSVKDTIARDNPKNAVTLQMNSLKPEDTAIYYCNAIVYDGNRWRTYWGQGTQVTVSS

SEQ ID NO: 568 - protein, 123 aa, synthetic construct
  REGION 1..123: note = Description of Artificial Sequence: Synthetic polypeptide
QVKLEESGEGLVQAGGSLRLSCAASGRTFSSYAMAWFRQAPGKEREFVARIRWTRSSTVYADSVKGTISGDNAKNTMYLQMNSLKPEDTAVYYCAADRYYRTDIYRASSYEYWGQGTQVTVSS

SEQ ID NO: 569 - protein, 118 aa, synthetic construct
  REGION 1..118: note = Description of Artificial Sequence: Synthetic polypeptide
QVKLEESGGGLVQAGGSLRLSCVVSGRPFINYNMGWFRQAPGKEHEFVAAISWSGDSTYYEDSVKGTVSRDNAKNTVYLQMNNLKPEDTAVYYCAADNQHDIPLRPGGWQGTQVTVSS

SEQ ID NO: 570 - protein, 126 aa, synthetic construct
  REGION 1..126: note = Description of Artificial Sequence: Synthetic polypeptide
QVQLVESGGGLVEAGGSLTLSCAASGLAFNTKTMAWFRQAPDKERAVVATITWGTINTSYADSVKGRFTISRDNAKNMVYLRMDSLKPEDTDVYYCESEALLETTPSRRPYEYNYWGPGTQVTVSS

SEQ ID NO: 571 - protein, 124 aa, synthetic construct
  REGION 1..124: note = Description of Artificial Sequence: Synthetic polypeptide
QVKLEESGGGLVQAGGSLRLSCAASGRIFGSLHMGWFRQAPGKEREFVSPITAAGGVTDYDSSNEGIHSVLHKQRQEHVSSPMNSLKPDTHGRLLLCRTLGCSYYERADEYNYWGQGTQVTVSS

SEQ ID NO: 572 - protein, 118 aa, synthetic construct
  REGION 1..118: note = Description of Artificial Sequence: Synthetic polypeptide
QVKLEESGGGLVQAGGSLRLSCAVSGSIFSTNLMGWYRQAPGKQRGFVAHITSGGNTDYLDSVKGRFTISRDNAKNTVYLQMNSLKPEDTAVYYCAAQTLGSSYYDAWGQGTQVTVSS

SEQ ID NO: 573 - protein, 9 aa, synthetic construct
  REGION 1..9: note = Description of Artificial Sequence: Synthetic peptide
GFTLDGYAI

SEQ ID NO: 574 - protein, 8 aa, synthetic construct
  REGION 1..8: note = Description of Artificial Sequence: Synthetic peptide
IIYRDGSP

SEQ ID NO: 575 - protein, 17 aa, synthetic construct
  REGION 1..17: note = Description of Artificial Sequence: Synthetic peptide
AARPGGACSRYPSNYDT

SEQ ID NO: 576 - protein, 25 aa, synthetic construct
  REGION 1..25: note = Description of Artificial Sequence: Synthetic peptide
QVKLEESGGGLVQAGGSLRLSCAVS

SEQ ID NO: 577 - protein, 25 aa, synthetic construct
  REGION 1..25: note = Description of Artificial Sequence: Synthetic peptide
QVQLVESGGGLVQAGGSLRLSCAAS

SEQ ID NO: 578 - protein, 25 aa, synthetic construct
  REGION 1..25: note = Description of Artificial Sequence: Synthetic peptide
QVQLVESGGGLVQPGGSLRLSCAAS

SEQ ID NO: 579 - protein, 25 aa, synthetic construct
  REGION 1..25: note = Description of Artificial Sequence: Synthetic peptide
QVKLEESGGGLVRAGGSLTLSCGAS

SEQ ID NO: 580 - protein, 25 aa, synthetic construct
  REGION 1..25: note = Description of Artificial Sequence: Synthetic peptide
QVQLQQSGGGLVQAGGSLRLSCAAS

SEQ ID NO: 581 - protein, 25 aa, synthetic construct
  REGION 1..25: note = Description of Artificial Sequence: Synthetic peptide
QVQLQESGGRLVQAGGSLRLSCAAS

SEQ ID NO: 582 - protein, 25 aa, synthetic construct
  REGION 1..25: note = Description of Artificial Sequence: Synthetic peptide
QVQLQESGGGLVQAGKSLRLSCAAS

SEQ ID NO: 583 - protein, 25 aa, synthetic construct
  REGION 1..25: note = Description of Artificial Sequence: Synthetic peptide
QVQLQESGGGMVQAGGSLRLACTAS

SEQ ID NO: 584 - protein, 25 aa, synthetic construct
  REGION 1..25: note = Description of Artificial Sequence: Synthetic peptide
QVQLQESGGGLVQAGGSLRLSCAAS

SEQ ID NO: 585 - protein, 25 aa, synthetic construct
  REGION 1..25: note = Description of Artificial Sequence: Synthetic peptide
QVQLQESGGGLVQAGGSLRLACTAS

SEQ ID NO: 586 - protein, 25 aa, synthetic construct
  REGION 1..25: note = Description of Artificial Sequence: Synthetic peptide
QVQLQESGGGSVQAGGSLRLSCAAS

SEQ ID NO: 587 - protein, 25 aa, synthetic construct
  REGION 1..25: note = Description of Artificial Sequence: Synthetic peptide
QVQLQESGGGLVQAGGSLSLSCTAS

SEQ ID NO: 588 - protein, 25 aa, synthetic construct
  REGION 1..25: note = Description of Artificial Sequence: Synthetic peptide
QVQLQESGGGLVQPGGSLSLSCTAS

SEQ ID NO: 589 - protein, 25 aa, synthetic construct
  REGION 1..25: note = Description of Artificial Sequence: Synthetic peptide
QVQLQESGGGLVQAGGSLTLSCTAS

SEQ ID NO: 590 - protein, 25 aa, synthetic construct
  REGION 1..25: note = Description of Artificial Sequence: Synthetic peptide
QVQLQESGGGLVQAGGSLTLSCTTS

SEQ ID NO: 591 - protein, 25 aa, synthetic construct
  REGION 1..25: note = Description of Artificial Sequence: Synthetic peptide
QVQLQESGGGLVQAGGSLSLSCRVS

SEQ ID NO: 592 - protein, 25 aa, synthetic construct
  REGION 1..25: note = Description of Artificial Sequence: Synthetic peptide
QVQLQESGGGLVQPGGSLTLSCTAS

SEQ ID NO: 593 - protein, 25 aa, synthetic construct
  REGION 1..25: note = Description of Artificial Sequence: Synthetic peptide
QVQLQQSGGGLVQAGGSLSLSCTAS

SEQ ID NO: 594 - protein, 25 aa, synthetic construct
  REGION 1..25: note = Description of Artificial Sequence: Synthetic peptide
QVQLQESGGGLVQAGGSLRLSCAGS

SEQ ID NO: 595 - protein, 25 aa, synthetic construct
  REGION 1..25: note = Description of Artificial Sequence: Synthetic peptide
QVQLQQSGGGLVQAGGSLKLSCAAS

SEQ ID NO: 596 - protein, 25 aa, synthetic construct
  REGION 1..25: note = Description of Artificial Sequence: Synthetic peptide
QVQLQESGGGLVQAGGSLSLSCTTS

SEQ ID NO: 597 - protein, 25 aa, synthetic construct
  REGION 1..25: note = Description of Artificial Sequence: Synthetic peptide
QVQLQESGGGLVQAGGSLRLSCVAS

SEQ ID NO: 598 - protein, 25 aa, synthetic construct
  REGION 1..25: note = Description of Artificial Sequence: Synthetic peptide
QVQLQESGGGLVQPGGSLRLSCAAS

SEQ ID NO: 599 - protein, 25 aa, synthetic construct
  REGION 1..25: note = Description of Artificial Sequence: Synthetic peptide
QVKLEESGGGLVQAGGSLRLSCAAS

SEQ ID NO: 600 - protein, 25 aa, synthetic construct
  REGION 1..25: note = Description of Artificial Sequence: Synthetic peptide
QVKLEESGGGLVQPGGSLTLSCAAS

SEQ ID NO: 601 - protein, 25 aa, synthetic construct
  REGION 1..25: note = Description of Artificial Sequence: Synthetic peptide
QVKLEESGEGLVQAGGSLRLSCAAS

SEQ ID NO: 602 - protein, 25 aa, synthetic construct
  REGION 1..25: note = Description of Artificial Sequence: Synthetic peptide
QVQLVESGGGLVQAGGSLTLSCIVS

SEQ ID NO: 603 - protein, 25 aa, synthetic construct
  REGION 1..25: note = Description of Artificial Sequence: Synthetic peptide
QVQLVESESGLVLAGGSLTLTRFCS

SEQ ID NO: 604 - protein, 25 aa, synthetic construct
  REGION 1..25: note = Description of Artificial Sequence: Synthetic peptide
QVQLVESGGGSVQPAGSLRLSCAVS

SEQ ID NO: 605 - protein, 25 aa, synthetic construct
  REGION 1..25: note = Description of Artificial Sequence: Synthetic peptide
QVQLVESGGGLVQPGGSLRLSCAVS

SEQ ID NO: 606 - protein, 25 aa, synthetic construct
  REGION 1..25: note = Description of Artificial Sequence: Synthetic peptide
QVKLEESGGGLVQPGGSLRLSCAAS

SEQ ID NO: 607 - protein, 25 aa, synthetic construct
  REGION 1..25: note = Description of Artificial Sequence: Synthetic peptide
QVRLEKSGGGLVQPGGSLTLSCTAS

SEQ ID NO: 608 - protein, 25 aa, synthetic construct
  REGION 1..25: note = Description of Artificial Sequence: Synthetic peptide
QVQLVESGGGLVQPGGSLKVSCAAS

SEQ ID NO: 609 - protein, 25 aa, synthetic construct
  REGION 1..25: note = Description of Artificial Sequence: Synthetic peptide
QVQLAESGVGLVEPAGSLKFSCAAS

SEQ ID NO: 610 - protein, 25 aa, synthetic construct
  REGION 1..25: note = Description of Artificial Sequence: Synthetic peptide
QVKLEESGGGLVQPGGSLKVSCAAS

SEQ ID NO: 611 - protein, 25 aa, synthetic construct
  REGION 1..25: note = Description of Artificial Sequence: Synthetic peptide
QVQLVESGAGLVQPGGSLRLSCAVS

SEQ ID NO: 612 - protein, 25 aa, synthetic construct
  REGION 1..25: note = Description of Artificial Sequence: Synthetic peptide
QVKLEESGGGLVQAGGSLRLSCVVS

SEQ ID NO: 613 - protein, 25 aa, synthetic construct
  REGION 1..25: note = Description of Artificial Sequence: Synthetic peptide
QVQLVESGGGLVEAGGSLTLSCAAS

SEQ ID NO: 614 - protein, 16 aa, synthetic construct
  REGION 1..16: note = Description of Artificial Sequence: Synthetic peptide
YWYRQGPGKQRELVVS

SEQ ID NO: 615 - protein, 16 aa, synthetic construct
  REGION 1..16: note = Description of Artificial Sequence: Synthetic peptide
GWFRQAPGKQRELVAA

SEQ ID NO: 616 - protein, 16 aa, synthetic construct
  REGION 1..16: note = Description of Artificial Sequence: Synthetic peptide
GWYRQAPGEQRELVAT

SEQ ID NO: 617 - protein, 16 aa, synthetic construct
  REGION 1..16: note = Description of Artificial Sequence: Synthetic peptide
GWFRQAPGKQRGFVAH

SEQ ID NO: 618 - protein, 16 aa, synthetic construct
  REGION 1..16: note = Description of Artificial Sequence: Synthetic peptide
GWFRQAPGKEREFVAS

SEQ ID NO: 619 - protein, 16 aa, synthetic construct
  REGION 1..16: note = Description of Artificial Sequence: Synthetic peptide
AWFRQAPGNEREFVAS

SEQ ID NO: 620 - protein, 16 aa, synthetic construct
  REGION 1..16: note = Description of Artificial Sequence: Synthetic peptide
EWYRQAPGNQREYVAR

SEQ ID NO: 621 - protein, 16 aa, synthetic construct
  REGION 1..16: note = Description of Artificial Sequence: Synthetic peptide
GWFRQAPGKEREFVAA

SEQ ID NO: 622 - protein, 16 aa, synthetic construct
  REGION 1..16: note = Description of Artificial Sequence: Synthetic peptide
GWYRQAPGKQRELVAL

SEQ ID NO: 623 - protein, 16 aa, synthetic construct
  REGION 1..16: note = Description of Artificial Sequence: Synthetic peptide
GWFRQAPGKEREFVSA

SEQ ID NO: 624 - protein, 16 aa, synthetic construct
  REGION 1..16: note = Description of Artificial Sequence: Synthetic peptide
GWNRQAVGNQRELVAV

SEQ ID NO: 625 - protein, 16 aa, synthetic construct
  REGION 1..16: note = Description of Artificial Sequence: Synthetic peptide
GWNRQAPGKHRELVAI

SEQ ID NO: 626 - protein, 16 aa, synthetic construct
  REGION 1..16: note = Description of Artificial Sequence: Synthetic peptide
AWYRQAPGKQREAIAS

SEQ ID NO: 627 - protein, 17 aa, synthetic construct
  REGION 1..17: note = Description of Artificial Sequence: Synthetic peptide
GWNRQAPGKQQRELVAA

SEQ ID NO: 628 - protein, 16 aa, synthetic construct
  REGION 1..16: note = Description of Artificial Sequence: Synthetic peptide
GWNREAPGNRREMVAI

SEQ ID NO: 629 - protein, 16 aa, synthetic construct
  REGION 1..16: note = Description of Artificial Sequence: Synthetic peptide
GWNRQAPGNQRELVAV

SEQ ID NO: 630 - protein, 17 aa, synthetic construct
  REGION 1..17: note = Description of Artificial Sequence: Synthetic peptide
GWNRQAPGNQQRDLVAV

SEQ ID NO: 631 - protein, 17 aa, synthetic construct
  REGION 1..17: note = Description of Artificial Sequence: Synthetic peptide
GWNRQAPGNQRRDLVAV

SEQ ID NO: 632 - protein, 16 aa, synthetic construct
  REGION 1..16: note = Description of Artificial Sequence: Synthetic peptide
GWYRRAPGKQREFVAG

SEQ ID NO: 633 - protein, 16 aa, synthetic construct
  REGION 1..16: note = Description of Artificial Sequence: Synthetic peptide
GWYRRAPGKEREFVAG

SEQ ID NO: 634 - protein, 16 aa, synthetic construct
  REGION 1..16: note = Description of Artificial Sequence: Synthetic peptide
GWFRQAPGKEREFVAT

SEQ ID NO: 635 - protein, 16 aa, synthetic construct
  REGION 1..16: note = Description of Artificial Sequence: Synthetic peptide
AWYRQAPGKQRELVAS

SEQ ID NO: 636 - protein, 17 aa, synthetic construct
  REGION 1..17: note = Description of Artificial Sequence: Synthetic peptide
GWFRQAPGKEREFIVAI

SEQ ID NO: 637 - protein, 16 aa, synthetic construct
  REGION 1..16: note = Description of Artificial Sequence: Synthetic peptide
GWYRQAPGKERDLVAP

SEQ ID NO: 638 - protein, 16 aa, synthetic construct
  REGION 1..16: note = Description of Artificial Sequence: Synthetic peptide
AWYRQAPGKQRELVAH

SEQ ID NO: 639 - protein, 16 aa, synthetic construct
  REGION 1..16: note = Description of Artificial Sequence: Synthetic peptide
AWYRQAPGQQRELVAS

SEQ ID NO: 640 - protein, 16 aa, synthetic construct
  REGION 1..16: note = Description of Artificial Sequence: Synthetic peptide
GWYRQAPGKERDLVAG

SEQ ID NO: 641 - protein, 16 aa, synthetic construct
  REGION 1..16: note = Description of Artificial Sequence: Synthetic peptide
GWYRRAPGQEREFVAG

SEQ ID NO: 642 - protein, 16 aa, synthetic construct
  REGION 1..16: note = Description of Artificial Sequence: Synthetic peptide
GWYRRAPGKQRDFVAG

SEQ ID NO: 643 - protein, 16 aa, synthetic construct
  REGION 1..16: note = Description of Artificial Sequence: Synthetic peptide
AWYRQRPGKQRELVAS

SEQ ID NO: 644 - protein, 16 aa, synthetic construct
  REGION 1..16: note = Description of Artificial Sequence: Synthetic peptide
NWFRQTPGKQREYVAY

SEQ ID NO: 645 - protein, 16 aa, synthetic construct
  REGION 1..16: note = Description of Artificial Sequence: Synthetic peptide
GWFRQAPGKEREWVSC

SEQ ID NO: 646 - protein, 16 aa, synthetic construct
  REGION 1..16: note = Description of Artificial Sequence: Synthetic peptide
GWYRQAPGKQRELVAA

SEQ ID NO: 647 - protein, 16 aa, synthetic construct
  REGION 1..16: note = Description of Artificial Sequence: Synthetic peptide
GWYRQAPGKQRELVST

SEQ ID NO: 648 - protein, 16 aa, synthetic construct
  REGION 1..16: note = Description of Artificial Sequence: Synthetic peptide
GWFRQAPGKERELVSA

SEQ ID NO: 649 - protein, 16 aa, synthetic construct
  REGION 1..16: note = Description of Artificial Sequence: Synthetic peptide
GWFRQAPGKERELVAA

SEQ ID NO: 650 - protein, 16 aa, synthetic construct
  REGION 1..16: note = Description of Artificial Sequence: Synthetic peptide
GWFRQAPGKERDIVAA

SEQ ID NO: 651 - protein, 16 aa, synthetic construct
  REGION 1..16: note = Description of Artificial Sequence: Synthetic peptide
YWYRPGPGNQRELVVS

SEQ ID NO: 652 - protein, 16 aa, synthetic construct
  REGION 1..16: note = Description of Artificial Sequence: Synthetic peptide
YWYRQGPGKQRELVLI

SEQ ID NO: 653 - protein, 16 aa, synthetic construct
  REGION 1..16: note = Description of Artificial Sequence: Synthetic peptide
GWFRQAPGKEREFVVA

SEQ ID NO: 654 - protein, 16 aa, synthetic construct
  REGION 1..16: note = Description of Artificial Sequence: Synthetic peptide
GWYRPAPGKERELVAS

SEQ ID NO: 655 - protein, 16 aa, synthetic construct
  REGION 1..16: note = Description of Artificial Sequence: Synthetic peptide
GWFRQAPGLEREIIAA

SEQ ID NO: 656 - protein, 16 aa, synthetic construct
  REGION 1..16: note = Description of Artificial Sequence: Synthetic peptide
GWYRQAPGNQRVLVAS

SEQ ID NO: 657 - protein, 16 aa, synthetic construct
  REGION 1..16: note = Description of Artificial Sequence: Synthetic peptide
TWVRQAPGKGLEWVSA

SEQ ID NO: 658 - protein, 16 aa, synthetic construct
  REGION 1..16: note = Description of Artificial Sequence: Synthetic peptide
GWFRQGPGKERESVAA

SEQ ID NO: 659 - protein, 16 aa, synthetic construct
  REGION 1..16: note = Description of Artificial Sequence: Synthetic peptide
GWYRQAPGKQRELAAL

SEQ ID NO: 660 - protein, 16 aa, synthetic construct
  REGION 1..16: note = Description of Artificial Sequence: Synthetic peptide
GWYRQAPGKQRDLVAT

SEQ ID NO: 661 - protein, 16 aa, synthetic construct
  REGION 1..16: note = Description of Artificial Sequence: Synthetic peptide
GWFREAPGKERRFVAS

SEQ ID NO: 662 - protein, 16 aa, synthetic construct
  REGION 1..16: note = Description of Artificial Sequence: Synthetic peptide
GWHRQAPENQRELVAT

SEQ ID NO: 663 - protein, 16 aa, synthetic construct
  REGION 1..16: note = Description of Artificial Sequence: Synthetic peptide
GWYRQAPGKERELVAS

SEQ ID NO: 664 - protein, 16 aa, synthetic construct
  REGION 1..16: note = Description of Artificial Sequence: Synthetic peptide
GWYRQAPGKQRELVAT

SEQ ID NO: 665 - protein, 16 aa, synthetic construct
  REGION 1..16: note = Description of Artificial Sequence: Synthetic peptide
AWFRQAPGKEREFVAR

SEQ ID NO: 666 - protein, 16 aa, synthetic construct
  REGION 1..16: note = Description of Artificial Sequence: Synthetic peptide
GWFRQAPGKEHEFVAA

SEQ ID NO: 667 - protein, 16 aa, synthetic construct
  REGION 1..16: note = Description of Artificial Sequence: Synthetic peptide
AWFRQAPDKERAVVAT

SEQ ID NO: 668 - protein, 16 aa, synthetic construct
  REGION 1..16: note = Description of Artificial Sequence: Synthetic peptide
GWFRQAPGKEREFVSP

SEQ ID NO: 669 - protein, 16 aa, synthetic construct
  REGION 1..16: note = Description of Artificial Sequence: Synthetic peptide
GWYRQAPGKQRGFVAH

SEQ ID NO: 670 - protein, 38 aa, synthetic construct
  REGION 1..38: note = Description of Artificial Sequence: Synthetic polypeptide
HDAHFAKGRFIISRDNAKNTVYLQMNSLKPEDTAVYYC

SEQ ID NO: 671 - protein, 38 aa, synthetic construct
  REGION 1..38: note = Description of Artificial Sequence: Synthetic polypeptide
NYADSMKGRVTISGDNAKNTVYLQMNSLKPEDTAVYYC

SEQ ID NO: 672 - protein, 38 aa, synthetic construct
  REGION 1..38: note = Description of Artificial Sequence: Synthetic polypeptide
NYADSVKDRFTISRDNAKNTVYLQMTSLKPEDTAVYYC

SEQ ID NO: 673 - protein, 38 aa, synthetic construct
  REGION 1..38: note = Description of Artificial Sequence: Synthetic polypeptide
DYADSVNGRFTMSRDNAKNTVYLQMNSLKPEDTAVYYC

SEQ ID NO: 674 - protein, 38 aa, synthetic construct
  REGION 1..38: note = Description of Artificial Sequence: Synthetic polypeptide
YYADSVKGRFTISRDNAKNTVYLQMNSLKPEDTAVYYC

SEQ ID NO: 675 - protein, 38 aa, synthetic construct
  REGION 1..38: note = Description of Artificial Sequence: Synthetic polypeptide
YYADPVKGRFTISRDNAKNTVYLQMNSLKPDDTAVYYC

SEQ ID NO: 676 - protein, 38 aa, synthetic construct
  REGION 1..38: note = Description of Artificial Sequence: Synthetic polypeptide
YYADPVKGRFTISRDSAKNTVYLQMNSLKPDDTAVYYC

SEQ ID NO: 677 - protein, 38 aa, synthetic construct
  REGION 1..38: note = Description of Artificial Sequence: Synthetic polypeptide
YHAHPVKGRFTIFRDNAKNTVYLQMNSLKPDDTGVYYC

SEQ ID NO: 678 - protein, 38 aa, synthetic construct
  REGION 1..38: note = Description of Artificial Sequence: Synthetic polypeptide
NYADSVKGRFTISRDNAKNTVYLQMNSLKPEDTAVYYC

SEQ ID NO: 679 - protein, 38 aa, synthetic construct
  REGION 1..38: note = Description of Artificial Sequence: Synthetic polypeptide
YFSDSVKGRFAISRDNAKDTVSLQMNNLKPDDTAVYTC

SEQ ID NO: 680 - protein, 38 aa, synthetic construct
  REGION 1..38: note = Description of Artificial Sequence: Synthetic polypeptide
NYADSVKGRFTISRNNAKKTVYLQMNSLKPEDTAVYYC

SEQ ID NO: 681 - protein, 38 aa, synthetic construct
  REGION 1..38: note = Description of Artificial Sequence: Synthetic polypeptide
WYADSMKGRFTISRDNAKNTGYLQMNSLKPEDTAIYYC

SEQ ID NO: 682 - protein, 38 aa, synthetic construct
  REGION 1..38: note = Description of Artificial Sequence: Synthetic polypeptide
YYGDSVQGRFTISKDNAKNTVSLQMNRLNPEDTAVYYC

SEQ ID NO: 683 - protein, 38 aa, synthetic construct
  REGION 1..38: note = Description of Artificial Sequence: Synthetic polypeptide
KYADSVKGRFTITRDISKNTIYLQMNSLKPEDTGVYYC

SEQ ID NO: 684 - protein, 40 aa, synthetic construct
  REGION 1..40: note = Description of Artificial Sequence: Synthetic polypeptide
KYADSVKGRFTISRDNTKNTVTVYLQMNNVNAEDTAVYYC

SEQ ID NO: 685 - protein, 38 aa, synthetic construct
  REGION 1..38: note = Description of Artificial Sequence: Synthetic polypeptide
KYADSVKGRFTISRDNTKNTVYLQMNSVNAEDTAVYYC

SEQ ID NO: 686 - protein, 38 aa, synthetic construct
  REGION 1..38: note = Description of Artificial Sequence: Synthetic polypeptide
VYANSVKDRFTISRDNNKNMVYLQMNSLKPEDTAVYSC

SEQ ID NO: 687 - protein, 38 aa, synthetic construct
  REGION 1..38: note = Description of Artificial Sequence: Synthetic polypeptide
NYADSVKGRFTITRDISKNTIYLQMNNLKPEDTGVYYC

SEQ ID NO: 688 - protein, 38 aa, synthetic construct
  REGION 1..38: note = Description of Artificial Sequence: Synthetic polypeptide
KYDDSVKGRFTISRDIAKNTVFLQMDSLKPEDTGVYYC

SEQ ID NO: 689 - protein, 38 aa, synthetic construct
  REGION 1..38: note = Description of Artificial Sequence: Synthetic polypeptide
KYADSVQGRFTISRDIAKNTVYLQMNSLKPEDTGVYYC

SEQ ID NO: 690 - protein, 38 aa, synthetic construct
  REGION 1..38: note = Description of Artificial Sequence: Synthetic polypeptide
VYADSVKDRFTISRDNNKNMVYLQMDSLKPEDTAVYYC

SEQ ID NO: 691 - protein, 38 aa, synthetic construct
  REGION 1..38: note = Description of Artificial Sequence: Synthetic polypeptide
QYADSVKGRFTITRDISKNTIYLQMNSLKPEYTGVYYC

SEQ ID NO: 692 - protein, 38 aa, synthetic construct
  REGION 1..38: note = Description of Artificial Sequence: Synthetic polypeptide
KYADSVQGRFTISRDIANNTVYLQMNSLKPEDTGVYYC

SEQ ID NO: 693 - protein, 38 aa, synthetic construct
  REGION 1..38: note = Description of Artificial Sequence: Synthetic polypeptide
NYMDSVKGRFTISRDNAKNTVYLQMTKLKPEDTAVYYC

SEQ ID NO: 694 - protein, 38 aa, synthetic construct
  REGION 1..38: note = Description of Artificial Sequence: Synthetic polypeptide
NYMDSVKGRFTISRDNAKKAVYLQMTSLKPEDTALYYC

SEQ ID NO: 695 - protein, 38 aa, synthetic construct
  REGION 1..38: note = Description of Artificial Sequence: Synthetic polypeptide
NYMGSVQGRFTISRDNAKNTVYLQMRRLKPEDTAVYYC

SEQ ID NO: 696 - protein, 38 aa, synthetic construct
  REGION 1..38: note = Description of Artificial Sequence: Synthetic polypeptide
RYADSVKGRFTISRDNAKNTVDLQMNSLKPEDTAVYYC

SEQ ID NO: 697 - protein, 38 aa, synthetic construct
  REGION 1..38: note = Description of Artificial Sequence: Synthetic polypeptide
TYADFVKGRFIISRDNAKSTVYLQMNSLKPEDTAVYYC

SEQ ID NO: 698 - protein, 38 aa, synthetic construct
  REGION 1..38: note = Description of Artificial Sequence: Synthetic polypeptide
RYEDFVKGRFTISRDNGKNTVYLQMNSLKPEDTAVYYC

SEQ ID NO: 699 - protein, 38 aa, synthetic construct
  REGION 1..38: note = Description of Artificial Sequence: Synthetic polypeptide
YYADSVKGRFTISRDNAKNTVYLHMNSLKPEDTAVYYC

SEQ ID NO: 700 - protein, 38 aa, synthetic construct
  REGION 1..38: note = Description of Artificial Sequence: Synthetic polypeptide
NYAESVKGRFTISRDNAKNTVYLQMNSLKPEDTAVYYC

SEQ ID NO: 701 - protein, 38 aa, synthetic construct
  REGION 1..38: note = Description of Artificial Sequence: Synthetic polypeptide
RYADFVQGRFTISRDNGKNTVYLQMNSLKPEDTAVYYC

SEQ ID NO: 702 - protein, 38 aa, synthetic construct
  REGION 1..38: note = Description of Artificial Sequence: Synthetic polypeptide
RYADSVKGRFTISRDNAKNTVNLQMNRLKPEDTAVYYC

SEQ ID NO: 703 - protein, 38 aa, synthetic construct
  REGION 1..38: note = Description of Artificial Sequence: Synthetic polypeptide
RYADFVKGRFTISRDNAKSTVYLQMNSLKPEDTAVYYC

SEQ ID NO: 704 - protein, 38 aa, synthetic construct
  REGION 1..38: note = Description of Artificial Sequence: Synthetic polypeptide
HYADSVKGRFTISRDNAKNTVNLQMNSLKVEDTAVYYC

SEQ ID NO: 705 - protein, 38 aa, synthetic construct
  REGION 1..38: note = Description of Artificial Sequence: Synthetic polypeptide
NYMNFVKGRFTISRDNAKNTVYLQMTSLKPEDTAVYYC

SEQ ID NO: 706 - protein, 38 aa, synthetic construct
  REGION 1..38: note = Description of Artificial Sequence: Synthetic polypeptide
HYADSVKGRFTISRDNAKNTVDLQMNSLKPEDTAVYYC

SEQ ID NO: 707 - protein, 38 aa, synthetic construct
  REGION 1..38: note = Description of Artificial Sequence: Synthetic polypeptide
YYADSVKGRFTISRDNAKNTVSLQMNSLKVEDTAVYYC

SEQ ID NO: 708 - protein, 38 aa, synthetic construct
  REGION 1..38: note = Description of Artificial Sequence: Synthetic polypeptide
NYMDSVKGRFTISRDNAKNTVYLRMSSLKPEDTAVYYC

SEQ ID NO: 709 - protein, 38 aa, synthetic construct
  REGION 1..38: note = Description of Artificial Sequence: Synthetic polypeptide
RYADSVKGRFTISRDNAKNTVNLQMNSLKPEDTAVYYC

SEQ ID NO: 710 - protein, 38 aa, synthetic construct
  REGION 1..38: note = Description of Artificial Sequence: Synthetic polypeptide
NYMNSVKGRFTISRDNAKNTVYLHMTGVKPADTAVYYC

SEQ ID NO: 711 - protein, 38 aa, synthetic construct
  REGION 1..38: note = Description of Artificial Sequence: Synthetic polypeptide
NYMDSTEGRFTISRDDAKNTVYLQMTGVKPADTAVYYC

SEQ ID NO: 712 - protein, 38 aa, synthetic construct
  REGION 1..38: note = Description of Artificial Sequence: Synthetic polypeptide
NYMDSVKGRFTISRDNAKNMVYLQMTKLKPEDTAVYYC

SEQ ID NO: 713 - protein, 38 aa, synthetic construct
  REGION 1..38: note = Description of Artificial Sequence: Synthetic polypeptide
NYMDSVKGRFTISRDNAKNTVYLQMTSLKPEDTAVYYC

SEQ ID NO: 714 - protein, 38 aa, synthetic construct
  REGION 1..38: note = Description of Artificial Sequence: Synthetic polypeptide
NYMDSVKGRFTISRDNAKNTVYLQMTNLKPEDTAVYYC

SEQ ID NO: 715 - protein, 38 aa, synthetic construct
  REGION 1..38: note = Description of Artificial Sequence: Synthetic polypeptide
RYEDFVKGRFTISRDNAKSTVYLQMNSLKPEDTAVYYC

SEQ ID NO: 716 - protein, 38 aa, synthetic construct
  REGION 1..38: note = Description of Artificial Sequence: Synthetic polypeptide
NVAQSMEGRFTISRDNAKNTGYLQMNSLKPEDTGVYYC

SEQ ID NO: 717 - protein, 38 aa, synthetic construct
  REGION 1..38: note = Description of Artificial Sequence: Synthetic polypeptide
NYMGFVKGRFTISRDNAKNTVYLQMTSLKPEDTAVYYC

SEQ ID NO: 718 - protein, 38 aa, synthetic construct
  REGION 1..38: note = Description of Artificial Sequence: Synthetic polypeptide
NYMDSVKDRFTISRDNAKNTLYLQMTNLKPEDTAVYYC

SEQ ID NO: 719 - protein, 38 aa, synthetic construct
  REGION 1..38: note = Description of Artificial Sequence: Synthetic polypeptide
NYMGFVRGRFTISRDNTKNTVYLQMTSLKPDDTAVYYC

SEQ ID NO: 720 - protein, 38 aa, synthetic construct
  REGION 1..38: note = Description of Artificial Sequence: Synthetic polypeptide
NYMDFVKGRFTISRDNAKNTVYLQMTSLKPEDTAVYYC

SEQ ID NO: 721 - protein, 38 aa, synthetic construct
  REGION 1..38: note = Description of Artificial Sequence: Synthetic polypeptide
NYPDSVKGRFAISRDNAKNTVYLQMNSLKPEDTAVYYC

SEQ ID NO: 722 - protein, 38 aa, synthetic construct
  REGION 1..38: note = Description of Artificial Sequence: Synthetic polypeptide
AYADSVWGRFTISRDNAKNNVYLEMNSLKPEDTAVYYC

SEQ ID NO: 723 - protein, 38 aa, synthetic construct
  REGION 1..38: note = Description of Artificial Sequence: Synthetic polypeptide
AYADSVKGRFTISRDKAKNTVYLQMNSLKPEDTAVYYC

SEQ ID NO: 724 - protein, 38 aa, synthetic construct
  REGION 1..38: note = Description of Artificial Sequence: Synthetic polypeptide
AYADSVKGRFTISRDNAKNTVYLQMNTLKPEDTAVYYC

SEQ ID NO: 725 - protein, 36 aa, synthetic construct
  REGION 1..36: note = Description of Artificial Sequence: Synthetic polypeptide
NYADSVKDTISRDNAKNTVYLQMTSLKPEDTAVYYC

SEQ ID NO: 726 - protein, 38 aa, synthetic construct
  REGION 1..38: note = Description of Artificial Sequence: Synthetic polypeptide
AYGDSVKGRFSISRDFAKNTVYLQIDSLKAEDTAVYYC

SEQ ID NO: 727 - protein, 38 aa, synthetic construct
  REGION 1..38: note = Description of Artificial Sequence: Synthetic polypeptide
SYANSVKGRFTISRDNTKSTVYLQIHSLKPEDTAVYYC

SEQ ID NO: 728 - protein, 38 aa, synthetic construct
  REGION 1..38: note = Description of Artificial Sequence: Synthetic polypeptide
AYADSVKGRFTISNDAAKNTVYLQMNSLKPDDTAVYYC

SEQ ID NO: 729 - protein, 38 aa, synthetic construct
  REGION 1..38: note = Description of Artificial Sequence: Synthetic polypeptide
HDAHFTKGRFIISKDDAKNTVYLLMNSLKPEDTAVYYC

SEQ ID NO: 730 - protein, 38 aa, synthetic construct
  REGION 1..38: note = Description of Artificial Sequence: Synthetic polypeptide
NTADSVKGRFTISRDNAKNTVYLQMNSLKPEDTAVYYC

SEQ ID NO: 731 - protein, 38 aa, synthetic construct
  REGION 1..38: note = Description of Artificial Sequence: Synthetic polypeptide
QNAHFPNGQFLISIYEDDNTMYLQLSIQKPEDADVYDC

SEQ ID NO: 732 - protein, 38 aa, synthetic construct
  REGION 1..38: note = Description of Artificial Sequence: Synthetic polypeptide
NYADSVKGRFTISRDNAKNTVYLQMNSLNAEDTAVYYC

SEQ ID NO: 733 - protein, 38 aa, synthetic construct
  REGION 1..38: note = Description of Artificial Sequence: Synthetic polypeptide
AYGDSVKGRFSISRDFAKNTVYLHIDSLKAEDTAVYYC

SEQ ID NO: 734 - protein, 38 aa, synthetic construct
  REGION 1..38: note = Description of Artificial Sequence: Synthetic polypeptide
DYADSVKDRFTIARDNPKNTVTLQMNSLKPEDTAIYYC

SEQ ID NO: 735 - protein, 38 aa, synthetic construct
  REGION 1..38: note = Description of Artificial Sequence: Synthetic polypeptide
RYADSMSDRFTISRDNAKNTVYLGMDSLKPEDTAVYYC

SEQ ID NO: 736 - protein, 37 aa, synthetic construct
  REGION 1..37: note = Description of Artificial Sequence: Synthetic polypeptide
NYAHSVKGTISRHNAENPVSRLQMNSLKPEDTAVYYC

SEQ ID NO: 737 - protein, 36 aa, synthetic construct
  REGION 1..36: note = Description of Artificial Sequence: Synthetic polypeptide
YYADSVKGTISRDNAKNTVYLQMNSLKPEDTAVYFC

SEQ ID NO: 738 - protein, 38 aa, synthetic construct
  REGION 1..38: note = Description of Artificial Sequence: Synthetic polypeptide
SYADSVKGRFTISRDNAKNTLYLQMNSLKPEDTAVYYC

SEQ ID NO: 739 - protein, 38 aa, synthetic construct
  REGION 1..38: note = Description of Artificial Sequence: Synthetic polypeptide
YYTDSVKGRFTISRDNAKNTVYLQMDSLKPEDTAVYFC

SEQ ID NO: 740 - protein, 38 aa, synthetic construct
  REGION 1..38: note = Description of Artificial Sequence: Synthetic polypeptide
DYADSAKGRFTISRDNAKNTVYLQMNSLKPEDTAVYYC

SEQ ID NO: 741 - protein, 38 aa, synthetic construct
  REGION 1..38: note = Description of Artificial Sequence: Synthetic polypeptide
AYGDSVKGRSNLSVNHTNNTAYLQMNSLRPEDTAVYWC

SEQ ID NO: 742 - protein, 38 aa, synthetic construct
  REGION 1..38: note = Description of Artificial Sequence: Synthetic polypeptide
YYTESVKGRFTISRDNAKNTVYLQMNSLKPEDTAVYYC

SEQ ID NO: 743 - protein, 36 aa, synthetic construct
  REGION 1..36: note = Description of Artificial Sequence: Synthetic polypeptide
NYADSVKGTISRDNAKNTVYLQMTSLKPEDTAVYYC

SEQ ID NO: 744 - protein, 36 aa, synthetic construct
  REGION 1..36: note = Description of Artificial Sequence: Synthetic polypeptide
SYADSVKGTISRDYAENMVYLQMNSLKPEDTATYYC

SEQ ID NO: 745 - protein, 36 aa, synthetic construct
  REGION 1..36: note = Description of Artificial Sequence: Synthetic polypeptide
NYADSVKDSISRDHDTNAVYLQMTNLRPDDPAVYYC

SEQ ID NO: 746 - protein, 36 aa, synthetic construct
  REGION 1..36: note = Description of Artificial Sequence: Synthetic polypeptide
DYADSVKDTIARDNPKNTVTLQMNSLKPEDTAIYYC

SEQ ID NO: 747 - protein, 36 aa, synthetic construct
  REGION 1..36: note = Description of Artificial Sequence: Synthetic polypeptide
NYADSVKDTISRDNAKNTVYLQMTSLKPEDTAVFYC

SEQ ID NO: 748 - protein, 36 aa, synthetic construct
  REGION 1..36: note = Description of Artificial Sequence: Synthetic polypeptide
DYADSVKDTIARDNPKNAVTLQMNSLKPEDTAIYYC

SEQ ID NO: 749 - protein, 36 aa, synthetic construct
  REGION 1..36: note = Description of Artificial Sequence: Synthetic polypeptide
VYADSVKGTISGDNAKNTMYLQMNSLKPEDTAVYYC

SEQ ID NO: 750 - protein, 36 aa, synthetic construct
  REGION 1..36: note = Description of Artificial Sequence: Synthetic polypeptide
YYEDSVKGTVSRDNAKNTVYLQMNNLKPEDTAVYYC

SEQ ID NO: 751 - protein, 38 aa, synthetic construct
  REGION 1..38: note = Description of Artificial Sequence: Synthetic polypeptide
SYADSVKGRFTISRDNAKNMVYLRMDSLKPEDTDVYYC

SEQ ID NO: 752 - protein, 38 aa, synthetic construct
  REGION 1..38: note = Description of Artificial Sequence: Synthetic polypeptide
DYLDSVKGRFTISRDNAKNTVYLQMNSLKPEDTAVYYC

SEQ ID NO: 753 - protein, 11 aa, synthetic construct
  REGION 1..11: note = Description of Artificial Sequence: Synthetic peptide
WGQGTQVTVSS

SEQ ID NO: 754 - protein, 11 aa, synthetic construct
  REGION 1..11: note = Description of Artificial Sequence: Synthetic peptide
WGQGTQVTVSL

SEQ ID NO: 755 - protein, 11 aa, synthetic construct
  REGION 1..11: note = Description of Artificial Sequence: Synthetic peptide
WGQGTQVTVST

SEQ ID NO: 756 - protein, 11 aa, synthetic construct
  REGION 1..11: note = Description of Artificial Sequence: Synthetic peptide
WGQGIQVTVSS

SEQ ID NO: 757 - protein, 11 aa, synthetic construct
  REGION 1..11: note = Description of Artificial Sequence: Synthetic peptide
WGPGTQVTVSS

SEQ ID NO: 758 - protein, 11 aa, synthetic construct
  REGION 1..11: note = Description of Artificial Sequence: Synthetic peptide
WGQGTQVTVSA

SEQ ID NO: 759 - protein, 11 aa, synthetic construct
  REGION 1..11: note = Description of Artificial Sequence: Synthetic peptide
WGKGTLVTVSS

SEQ ID NO: 760 - protein, 11 aa, synthetic construct
  REGION 1..11: note = Description of Artificial Sequence: Synthetic peptide
WGQGTQVTASS

SEQ ID NO: 761 - protein, 11 aa, synthetic construct
  REGION 1..11: note = Description of Artificial Sequence: Synthetic peptide
WGQGAQVTVSS

SEQ ID NO: 762 - protein, 11 aa, synthetic construct
  REGION 1..11: note = Description of Artificial Sequence: Synthetic peptide
WGRGTQVTVSS

SEQ ID NO: 763 - protein, 11 aa, synthetic construct
  REGION 1..11: note = Description of Artificial Sequence: Synthetic peptide
WGHGTQVTVSS

SEQ ID NO: 764 - protein, 10 aa, synthetic construct
  REGION 1..10: note = Description of Artificial Sequence: Synthetic peptide
WQGTQVTVSS

SEQ ID NO: 765 - protein, 9 aa, synthetic construct
  REGION 1..9: note = Description of Artificial Sequence: Synthetic peptide
GSIFSTNLM

SEQ ID NO: 766 - protein, 9 aa, synthetic construct
  REGION 1..9: note = Description of Artificial Sequence: Synthetic peptide
GVAFNSRIM

SEQ ID NO: 767 - protein, 9 aa, synthetic construct
  REGION 1..9: note = Description of Artificial Sequence: Synthetic peptide
GRTFNTYYM

SEQ ID NO: 768 - protein, 9 aa, synthetic construct
  REGION 1..9: note = Description of Artificial Sequence: Synthetic peptide
RGTFRTYSM

SEQ ID NO: 769 - protein, 7 aa, synthetic construct
  REGION 1..7: note = Description of Artificial Sequence: Synthetic peptide
ITSGGNT

SEQ ID NO: 770 - protein, 7 aa, synthetic construct
  REGION 1..7: note = Description of Artificial Sequence: Synthetic peptide
ITSGGST

SEQ ID NO: 771 - protein, 8 aa, synthetic construct
  REGION 1..8: note = Description of Artificial Sequence: Synthetic peptide
IRWSDGGT

SEQ ID NO: 772 - protein, 8 aa, synthetic construct
  REGION 1..8: note = Description of Artificial Sequence: Synthetic peptide
ITWNGKYT

SEQ ID NO: 773 - protein, 12 aa, synthetic construct
  REGION 1..12: note = Description of Artificial Sequence: Synthetic peptide
AAQTLGSSYYDA

SEQ ID NO: 774 - protein, 5 aa, synthetic construct
  REGION 1..5: note = Description of Artificial Sequence: Synthetic peptide
NIRNY

SEQ ID NO: 775 - protein, 13 aa, synthetic construct
  REGION 1..13: note = Description of Artificial Sequence: Synthetic peptide
NANVYDGNRWRTY

SEQ ID NO: 776 - protein, 21 aa, synthetic construct
  REGION 1..21: note = Description of Artificial Sequence: Synthetic peptide
AANPIPTAQPPGIMAARSYVH

SEQ ID NO: 777 - protein, 25 aa, synthetic construct
  REGION 1..25: note = Description of Artificial Sequence: Synthetic peptide
QVQLVESGGGLVQPGGSLTLSCIVS

SEQ ID NO: 778 - protein, 25 aa, synthetic construct
  REGION 1..25: note = Description of Artificial Sequence: Synthetic peptide
QVQLVESGGGLVQAGDSLRLSCTAS

SEQ ID NO: 779 - protein, 25 aa, synthetic construct
  REGION 1..25: note = Description of Artificial Sequence: Synthetic peptide
QVKLEESGGGLVQPGGSLEVSCAAS

SEQ ID NO: 780 - protein, 343 aa, Salmonella enterica
MLNIQNYSASPHPGIVAERPQTPSASEHVETAVVPSTTEHRGTDIISLSQAATKIQQAQQTLQSTPPISEENNDERTLARQQLTSSLNALAKSGVSLSAEQNENLRSAFSAPTSALFSASPMAQPRTTISDAEIWDMVSQNISAIGDSYLGVYENVVAVYTDFYQAFSDILSKMGGWLLPGKDGNTVKLDVTSLKNDLNSLVNKYNQINSNTVLFPAQSGSGVKVATEAEARQWLSELNLPNSCLKSYGSGYVVTVDLTPLQKMVQDIDGLGAPGKDSKLEMDNAKYQAWQSGFKAQEENMKTTLQTLTQKYSNANSLYDNLVKVLSSTISSSLETAKSFLQG

SEQ ID NO: 781 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2024/0400655 A1
(43) Pub. Date: Dec. 5, 2024

(54) ANTIBODIES AGAINST MICROORGANISMS AND USES THEREOF

(71) Applicant: NOVOBIND LIVESTOCK THERAPEUTICS INC., Vancouver (CA)

(72) Inventor: Hamlet ABNOUSI, Vancouver (CA)

(21) Appl. No.: 18/599,359

(22) Filed: Mar. 8, 2024

Related U.S. Application Data
(63) Continuation of application No. 17/409,559, filed on Aug. 23, 2021, now Pat. No. 11,939,371.
Application No. 17/409,559 is a division of application No. 16/300,457, filed on Nov. 9, 2018, now Pat. No. 11,130,800.
(60) Provisional application No. 62/339,732, filed on May 20, 2016.
(60) Provisional application No. 62/339,735, filed on May 20, 2016.

(51) Int. Cl. C07K 16/12; A23K 20/147; A23K 20/195; A23K 50/75; A23L 33/18; A61K 9/00; A61K 39/00; A61P 31/04

(52) U.S. Cl. CPC C07K 16/1235; A23K 20/147; A23K 20/195; A23K 50/75; A23L 33/18; A61K 9/0053; A61P 31/04; A23V 2002/00; A61K 2039/505; A61K 2039/54; A61K 2039/55; A61K 2039/552; C07K 2317/10; C07K 2317/24; C07K 2317/31; C07K 2317/35; C07K 2317/56; C07K 2317/565; C07K 2317/569; C07K 2317/64; C07K 2317/70; C07K 2317/94

(57) ABSTRACT

Described herein are methods and antibodies useful for reducing, eliminating, or preventing infection with a bacterial population in domestic animals or humans. Also described herein are antigens useful for targeting by heavy chain antibodies and V_{H}H fragments for reducing a bacterial population in domestic animals or humans.

### CROSS REFERENCE TO RELATED APPLICATIONS

This application is a Continuation of U.S. patent application Ser. No. 17/409,559, filed on Aug. 23, 2021, which is a Divisional of U.S. patent application Ser. No. 16/300,457, filed on Nov. 9, 2018, which is a U.S. National of International Application No. PCT/IB2017/000684, filed May 19, 2017, which claims priority to U.S. Provisionals with Ser. Nos. 62/339,732 and 62/339,735 both filed on May 20, 2016 both of which are incorporated by reference herein.

### BACKGROUND OF THE INVENTION

Salmonella bacteria are a major cause of foodborne illness, making infections caused by these pathogens a significant public health and economic concern. In terms of healthcare costs, loss of work due to illness and premature deaths, non-typhoidal Salmonella are estimated to be among the top five costliest foodborne pathogens along with Toxoplasma gondii, Listeria monocytogenes, Norovirus and Campylobacter. The most well-known sources of Salmonella infection include contaminated meat and poultry products, as well as eggs and fresh produce. Thus, there are significant economic costs to the food companies and restaurants supplying the contaminated poultry products. Increased foreign trade, distribution and travel have made salmonellae a widespread problem. As such, methods of controlling Salmonella contamination of poultry products are a viable approach to reducing infections in humans.

Current methods of controlling Salmonella in pre-harvest flocks include vaccination and the use of antibiotics, and while advances have been made in reducing the frequency of contamination in poultry products, there is still mounting pressure on commercial growers to eliminate these pathogens from pre-harvest production facilities. Several vaccines against salmonellae are commercially available but the production and use of vaccines can be expensive and labor intensive. Antibiotics are also commonly used to control pathogen colonization and prevent disease in livestock but there are concerns that misuse in livestock may be contributing to antibiotic resistance in both animals and humans. Furthermore, antibiotics have been shown to modify natural microbial communities in the gastrointestinal tracts of chickens, which could compromise the health of the birds. Therefore, a need exists for alternatives to antibiotics as well as methods to prevent and treat Salmonella colonization of domestic animals in order to reduce the incidence of Salmonella-associated health problems due to contaminated food products.

### SUMMARY OF THE INVENTION

In certain embodiments, described herein, is the use of an antibody for preventing infection in a domestic animal by a pathogenic Enterobacteriaceae bacterial population. In certain embodiments, preventing infection in the domestic animal by the pathogenic Enterobacteriaceae bacterial population prevents transmission of disease to a human. In certain embodiments, the antibody is selected from the group comprised of: a heavy chain antibody (hcIgG); a variable region fragment of a heavy chain antibody (V_{H}H); a single chain antibody; a polypeptide; an immunoglobulin new antigen receptor (IgNAR); a variable region fragment of an immunoglobulin new antigen receptor; or any fragment thereof that retains a capacity to specifically bind a target antigen. In certain embodiments, the antibody is a variable region fragment of a heavy chain antibody (V_{H}H). In certain embodiments, the antibody is altered to reduce immunogenicity in the domestic animal. In certain embodiments, the domestic animal is a chicken. In certain embodiments, the antibody originates from a species of the Camelidae family, or from a species of the Chondrichthyes class. In certain embodiments, the Camelidae species is a llama. In certain embodiments, the bacterial population comprises Salmonella bacteria. In certain embodiments, the bacterial population comprises Salmonella enterica. In certain embodiments, the bacterial population comprises Salmonella enterica serotype Typhimurium, Enteritidis, Newport, Heidelberg, Gallinarum, Hadar, Javiana, Infantis, Montevideo, Muenchen, Braenderup, Saintpaul, Thompson, Agona, Litchfield, Anatum, Berta, Mbandaka, Oranienburg, Poona, Uganda, Senftenberg, Weltevreden, I 4,[5],12:i:-, I 13,23:b:-, or any combination thereof. In certain embodiments, the domestic animal is from the superorder Galloanserae. In certain embodiments, the domestic animal is a poultry animal. In certain embodiments, the poultry animal is a chicken, turkey, duck, or goose. In certain embodiments, the poultry animal is a chicken. In certain embodiments, wherein the antibody specifically binds a Salmonella biomolecule. In certain embodiments, the antibody specifically binds a Salmonella biomolecule that functions in bacterial motility. In certain embodiments, the antibody reduces bacterial motility compared to a negative control antibody. In certain embodiments, the antibody reduces bacterial adhesion compared to a negative control antibody. In certain embodiments, the antibody reduces bacterial invasion compared to a negative control antibody. In certain embodiments, the Salmonella biomolecule is a Salmonella derived polypeptide. In certain embodiments, the biomolecule is a component of a flagellum. In certain embodiments, the biomolecule comprises a polypeptide derived from the flagella filament structural protein (FliC). In certain embodiments, the biomolecule comprises a polypeptide derived from the protein PrgI. In certain embodiments, the biomolecule comprises a polypeptide derived from the Fimbrial protein A (FimA). In certain embodiments, the antibody is monoclonal. In certain embodiments, the antibody is polyclonal. In certain embodiments, the antibody is incorporated into a spray for application to the exterior of a domestic animal. In certain embodiments, the antibody is formulated for oral administration. In certain embodiments, the antibody is formulated for injection. In certain embodiments, the antibody is formulated as a liquid. In certain embodiments, the antibody is not a chicken antibody. In certain embodiments, the antibody is not an IgY. In certain embodiments, the antibody does not specifically bind bacterial lipopolysaccharide (LPS).

In certain embodiments, described herein, is the use of an antibody for reducing a pathogenic Enterobacteriaceae bacterial population in a domestic animal. In certain embodiments, reducing the pathogenic Enterobacteriaceae bacterial population in a domestic animal prevents transmission of disease to a human. In certain embodiments, the antibody is selected from the group comprising a heavy chain antibody (hcIgG); a variable region fragment of a heavy chain antibody; a single chain antibody; a nanobody; a polypeptide; an immunoglobulin new antigen receptor (IgNAR); a variable region fragment of an immunoglobulin new antigen receptor; or any fragment thereof that retains a capacity to specifically bind a target antigen. In certain embodiments, the antibody is a variable region fragment of a heavy chain antibody (V_{H}H). In certain embodiments, the antibody is altered to reduce immunogenicity in a domestic animal. In certain embodiments, the domestic animal is a chicken. In certain embodiments, the antibody originates from a species of the Camelidae family, or from a species of the Chondrichthyes class. In certain embodiments, the Camelidae species is a llama. In certain embodiments, the bacterial population comprises Salmonella bacteria. In certain embodiments, the bacterial population comprises Salmonella enterica. In certain embodiments, the bacterial population comprises Salmonella enterica serotype Typhimurium, Enteritidis, Newport, Heidelberg, Gallinarum, Hadar, Javiana, Infantis, Montevideo, Muenchen, Braenderup, Saintpaul, Thompson, Agona, Litchfield, Anatum, Berta, Mbandaka, Oranienburg, Poona, Uganda, Senftenberg, Weltevreden, I 4,[5],12:i:-, I 13,23:b:-, or any combination thereof.

In certain embodiments, the domestic animal is from the superorder Galloanserae. In certain embodiments, the domestic animal is a poultry animal. In certain embodiments, the poultry animal is a chicken, turkey, duck, or goose. In certain embodiments, the poultry animal is a chicken. In certain embodiments, the antibody specifically binds a Salmonella biomolecule. In certain embodiments, the antibody specifically binds a Salmonella biomolecule that functions in bacterial motility. In certain embodiments, the antibody reduces bacterial motility compared to a negative control antibody. In certain embodiments, wherein the antibody reduces bacterial adhesion compared to a negative control antibody. In certain embodiments, the antibody reduces bacterial invasion compared to a negative control antibody. In certain embodiments, the Salmonella biomolecule is a Salmonella derived polypeptide. In certain embodiments, the biomolecule is a component of a flagellum. In certain embodiments, the biomolecule comprises a polypeptide derived from the flagella filament structural protein (FliC). In certain embodiments, the biomolecule comprises a polypeptide derived from the PrgI protein. In certain embodiments, the biomolecule comprises a polypeptide derived from the Fimbrial protein A (FimA). In certain embodiments, the antibody is monoclonal. In certain embodiments, the antibody is polyclonal. In certain embodiments, the antibody is incorporated into a spray for application to the exterior of a domestic animal. In certain embodiments, the antibody is formulated for oral administration. In certain embodiments, the antibody is formulated for injection. In certain embodiments, the antibody is formulated as a liquid. In certain embodiments, the antibody is not a chicken antibody. In certain embodiments, the antibody is not an IgY. In certain embodiments, the antibody does not specifically bind bacterial lipopolysaccharide (LPS).

In certain embodiments, described herein, is the use of a variable region fragment of a heavy chain antibody (V_{H}H) which specifically binds a Salmonella FliC protein, a Salmonella PrgI protein, a Salmonella FimA protein, or any combination thereof, for preventing infection with a pathogenic Salmonella enterica bacterial population in a domestic poultry animal.

In certain embodiments, described herein, is the use of a variable region fragment of a heavy chain antibody (V_{H}H) which specifically binds a Salmonella FliC protein, a Salmonella PrgI protein, a Salmonella FimA protein, or any combination thereof, for reducing a pathogenic Salmonella enterica bacterial population in a domestic poultry animal.

In certain embodiments, described herein, is an animal feed comprising an antibody that specifically binds an Enterobacteriaceae bacterial species. In certain embodiments, the animal feed comprises water. In certain embodiments, the animal feed comprises corn or a corn derivative. In certain embodiments, the animal feed comprises wheat or a wheat derivative. In certain embodiments, the animal feed comprises a poultry feed. In certain embodiments, the animal feed comprises a chicken feed. In certain embodiments, the animal feed comprises a probiotic, a prebiotic, a vitamin supplement, an additive spray, a toxin binder, a short chain fatty acid, a medium chain fatty acid, yeast, a yeast extract, sugar, a digestive enzyme, a digestive compound, an essential mineral, an essential salt, fiber, or any combination thereof. In certain embodiments, the antibody is selected from the group comprising: a heavy chain antibody (hcIgG); a variable region fragment of a heavy chain antibody; a single chain antibody; a nanobody; a polypeptide; an immunoglobulin new antigen receptor (IgNAR); a variable region fragment of an immunoglobulin new antigen receptor; or any fragment thereof that retains a capacity to specifically bind a target antigen. In certain embodiments, the antibody is a variable region fragment of a heavy chain antibody (V_{H}H). In certain embodiments, the antibody has been altered to reduce immunogenicity in a domestic animal. In certain embodiments, the domestic animal is a chicken. In certain embodiments, the antibody originates from a species of the Camelidae family, or from a species of the Chondrichthyes class. In certain embodiments, the Camelidae species is a llama. In certain embodiments, the bacterial population comprises Salmonella bacteria. In certain embodiments, the bacterial population comprises Salmonella enterica. In certain embodiments, the bacterial population comprises Salmonella enterica serotype Typhimurium, Enteritidis, Newport, Heidelberg, Gallinarum, Hadar, Javiana, Infantis, Montevideo, Muenchen, Braenderup, Saintpaul, Thompson, Agona, Litchfield, Anatum, Berta, Mbandaka, Oranienburg, Poona, Uganda, Senftenberg, Weltevreden, I 4,[5],12:i:-, I 13,23:b:-, or any combination thereof. In certain embodiments, the domestic animal is from the superorder Galloanserae. In certain embodiments, the domestic animal is a poultry animal. In certain embodiments, the poultry animal is a chicken, turkey, duck, or goose. In certain embodiments, the poultry animal is a chicken. In certain embodiments, the antibody specifically binds a Salmonella biomolecule. In certain embodiments, the antibody specifically binds a Salmonella biomolecule that functions in bacterial motility. In certain embodiments, the antibody reduces bacterial motility compared to a negative control antibody. In certain embodiments, the antibody reduces bacterial adhesion compared to a negative control antibody. In certain embodiments, the antibody reduces bacterial invasion compared to a negative control antibody. In certain embodiments, the Salmonella biomolecule is a Salmonella derived polypeptide. In certain embodiments, the biomolecule is a component of a flagellum. In certain embodiments, the biomolecule comprises a polypeptide derived from the flagella filament structural protein (FliC). In certain embodiments, the biomolecule comprises a polypeptide derived from the PrgI protein. In certain embodiments, the biomolecule comprises a polypeptide derived from the Fimbrial protein A (FimA). In certain embodiments, the antibody is monoclonal. In certain embodiments, the antibody is polyclonal. In certain embodiments, the antibody is incorporated into a spray for application to the exterior of a domestic animal. In certain embodiments, the antibody is formulated for oral administration. In certain embodiments, the antibody is formulated for injection. In certain embodiments, the antibody is formulated as a liquid. In certain embodiments, the antibody is not a chicken antibody. In certain embodiments, the antibody is not an IgY. In certain embodiments, the antibody does not specifically bind bacterial lipopolysaccharide (LPS).

In certain embodiments, described herein, is a domestic poultry feed comprising a variable region fragment of a heavy chain antibody (V_{H}H) which specifically binds a Salmonella flagella FliC protein, a Salmonella PrgI protein, a Salmonella FimA protein, or any combination thereof.

In certain embodiments, described herein, is a substance for introduction to an alimentary canal of a domestic poultry animal, the substance comprising a liquid, a gel, a spray, a tablet, or a pellet, for administration to the animal, which comprises a variable region fragment of a heavy chain antibody (V_{H}H) which specifically binds a Salmonella FliC protein, a Salmonella PrgI protein, a Salmonella FimA protein, or any combination thereof.

In certain embodiments, described herein, is a method for preventing infection with an Enterobacteriaceae bacterial population in a domestic animal, the method comprising introducing an antibody to an alimentary canal of the domestic animal. In certain embodiments, the antibody is selected from the group comprising: a heavy chain antibody (hcIgG); a variable region fragment of a heavy chain antibody (V_{H}H); a single chain antibody; a nanobody; a polypeptide; an immunoglobulin new antigen receptor (IgNAR); a variable region fragment of an immunoglobulin new antigen receptor; or any fragment thereof that retains a capacity to specifically bind a target antigen. In certain embodiments, the antibody is introduced to the alimentary canal of the domestic animal in an admixture of the antibody and a nutritional source. In certain embodiments, the nutritional source comprises water. In certain embodiments, the nutritional source comprises corn or a corn derivative. In certain embodiments, the nutritional source comprises wheat or a wheat derivative. In certain embodiments, the nutritional source comprises a poultry feed. In certain embodiments, the nutritional source comprises a chicken feed. In certain embodiments, the feed comprises a probiotic, a prebiotic, a vitamin supplement, an additive spray, a toxin binder, a short chain fatty acid, a medium chain fatty acid, yeast, a yeast extract, sugar, a digestive enzyme, a digestive compound, an essential mineral, an essential salt, fiber, or any combination thereof. In certain embodiments, the antibody is selected from the group comprising: a heavy chain antibody (hcIgG); a variable region fragment of a heavy chain antibody; a single chain antibody; a nanobody; a polypeptide; an immunoglobulin new antigen receptor (IgNAR); a variable region fragment of an immunoglobulin new antigen receptor; or any fragment thereof that retains a capacity to specifically bind a target antigen. In certain embodiments, the antibody is a variable region fragment of a heavy chain antibody (V_{H}H). In certain embodiments, wherein the antibody has been altered to reduce immunogenicity in a domestic animal. In certain embodiments, the domestic animal is a chicken. In certain embodiments, the antibody originates from a species of the Camelidae family, or from a species of the Chondrichthyes class. In certain embodiments, the Camelidae species is a llama. In certain embodiments, the bacterial population comprises Salmonella bacteria. In certain embodiments, the bacterial population comprises Salmonella enterica. In certain embodiments, the bacterial population comprises Salmonella enterica serotype Typhimurium, Enteritidis, Newport, Heidelberg, Gallinarum, Hadar, Javiana, Infantis, Montevideo, Muenchen, Braenderup, Saintpaul, Thompson, Agona, Litchfield, Anatum, Berta, Mbandaka, Oranienburg, Poona, Uganda, Senftenberg, Weltevreden, I 4,[5],12:i:-, I 13,23:b:-, or any combination thereof. In certain embodiments, the domestic animal is from the superorder Galloanserae. In certain embodiments, the domestic animal is a poultry animal. In certain embodiments, the poultry animal is a chicken, turkey, duck, or goose. In certain embodiments, the poultry animal is a chicken. In certain embodiments, the antibody specifically binds a Salmonella biomolecule. In certain embodiments, the antibody specifically binds a Salmonella biomolecule that functions in bacterial motility. In certain embodiments, the antibody reduces bacterial adhesion compared to a negative control antibody. In certain embodiments, the antibody reduces bacterial invasion compared to a negative control antibody. In certain embodiments, the antibody reduces bacterial motility compared to a negative control antibody. In certain embodiments, the Salmonella biomolecule is a Salmonella derived polypeptide. In certain embodiments, the biomolecule is a component of a flagellum. In certain embodiments, the biomolecule comprises a polypeptide derived from the flagella filament structural protein (FliC). In certain embodiments, the biomolecule comprises a polypeptide derived from the PrgI protein. In certain embodiments, the biomolecule comprises a polypeptide derived from the Fimbrial protein A (FimA). In certain embodiments, the antibody is monoclonal. In certain embodiments, the antibody is polyclonal. In certain embodiments, the antibody is incorporated into a spray for application to the exterior of a domestic animal. In certain embodiments, the antibody is formulated for oral administration. In certain embodiments, the antibody is formulated for injection. In certain embodiments, the antibody is formulated as a liquid. In certain embodiments, the antibody is not a chicken antibody. In certain embodiments, the antibody is not an IgY. In certain embodiments, the antibody does not specifically bind bacterial lipopolysaccharide (LPS).

In certain embodiments, described herein, is a method for reducing an Enterobacteriaceae bacterial population in a domestic animal, the method comprising introducing an antibody to an alimentary canal of the domestic animal. In certain embodiments, the antibody is introduced to the alimentary canal of the domestic animal in an admixture of the antibody and a nutritional source. In certain embodiments, the nutritional source comprises water. In certain embodiments, the nutritional source comprises corn or a corn derivative. In certain embodiments, the nutritional source comprises wheat or a wheat derivative. In certain embodiments, the nutritional source comprises a poultry feed. In certain embodiments, the nutritional source comprises a chicken feed. In certain embodiments, the nutritional source comprises a probiotic, a prebiotic, a vitamin supplement, an additive spray, a toxin binder, a short chain fatty acid, a medium chain fatty acid, yeast, a yeast extract, sugar, a digestive enzyme, a digestive compound, an essential mineral, an essential salt, fiber, or any combination thereof. In certain embodiments, the antibody is selected from the group comprising: a heavy chain antibody (hcIgG); a variable region fragment of a heavy chain antibody; a single chain antibody; a nanobody; a polypeptide; an immunoglobulin new antigen receptor (IgNAR); a variable region fragment of an immunoglobulin new antigen receptor; or any fragment thereof that retains a capacity to specifically bind a target antigen. In certain embodiments, the antibody is a variable region fragment of a heavy chain antibody (V_{H}H). In certain embodiments, the antibody has been altered to reduce immunogenicity in a domestic animal. In certain embodiments, the domestic animal is a chicken. In certain embodiments, the antibody originates from a species of the Camelidae family, or from a species of the Chondrichthyes class. In certain embodiments, the Camelidae species is a llama. In certain embodiments, wherein the bacterial population comprises Salmonella bacteria. In certain embodiments, the bacterial population comprises Salmonella enterica. In certain embodiments, the bacterial population comprises Salmonella enterica serotype Typhimurium, Enteritidis, Newport, Heidelberg, Gallinarum, Hadar, Javiana, Infantis, Montevideo, Muenchen, Braenderup, Saintpaul, Thompson, Agona, Litchfield, Anatum, Berta, Mbandaka, Oranienburg, Poona, Uganda, Senftenberg, Weltevreden, I 4,[5],12:i:-, I 13,23:b:-, or any combination thereof. In certain embodiments, the domestic animal is from the superorder Galloanserae. In certain embodiments, the domestic animal is a poultry animal. In certain embodiments, the poultry animal is a chicken, turkey, duck, or goose. In certain embodiments, the poultry animal is a chicken. In certain embodiments, the antibody specifically binds a Salmonella biomolecule. In certain embodiments, the antibody specifically binds a Salmonella biomolecule that functions in bacterial motility. In certain embodiments, the antibody reduces bacterial motility compared to a negative control antibody. In certain embodiments, the antibody reduces bacterial adhesion compared to a negative control antibody. In certain embodiments, the antibody reduces bacterial invasion compared to a negative control antibody. In certain embodiments, the Salmonella biomolecule is a Salmonella derived polypeptide. In certain embodiments, the biomolecule is a component of a flagellum. In certain embodiments, the biomolecule comprises a polypeptide derived from the flagella filament structural protein (FliC). In certain embodiments, the biomolecule comprises a polypeptide derived from the PrgI protein. In certain embodiments, the biomolecule comprises a polypeptide derived from the Fimbrial protein A (FimA). In certain embodiments, the antibody is monoclonal. In certain embodiments, the antibody is polyclonal. In certain embodiments, the antibody is incorporated into a spray for application to the exterior of a domestic animal. In certain embodiments, the antibody is formulated for oral administration. In certain embodiments, the antibody is formulated for injection. In certain embodiments, the antibody is formulated as a liquid. In certain embodiments, the antibody is not a chicken antibody. In certain embodiments, the antibody is not an IgY. In certain embodiments, the antibody does not specifically bind bacterial lipopolysaccharide (LPS).

In certain embodiments, described herein, is a method for preventing infection with a Salmonella enterica bacterial population in a domestic poultry animal, the method comprising introducing to an alimentary canal of the animal a monoclonal variable region fragment of a heavy chain antibody (V_{H}H) which specifically binds a Salmonella flagella filament structural protein (FliC), a Salmonella PrgI protein, a Salmonella FimA protein, or any combination thereof.

In certain embodiments, described herein, is a method for reducing a Salmonella enterica bacterial population in a domestic poultry animal, the method comprising introducing to an alimentary canal of the animal a variable region fragment of a heavy chain antibody (V_{H}H) which specifically binds a Salmonella flagella filament structural protein (FliC), a Salmonella PrgI protein, a Salmonella FimA protein, or any combination thereof.

In certain embodiments, described herein, is a heavy chain antibody which specifically binds Enterobacteriaceae. In certain embodiments, the heavy chain antibody is a variable region fragment of a heavy chain antibody; a single chain antibody; a nanobody; a polypeptide; an immunoglobulin new antigen receptor (IgNAR); a variable region fragment of an immunoglobulin new antigen receptor; or any fragment thereof that retains a capacity to specifically bind a target antigen. In certain embodiments, the heavy chain antibody is a variable region fragment of a heavy chain antibody (V_{H}H). In certain embodiments, the heavy chain antibody is altered to reduce immunogenicity in a domestic animal. In certain embodiments, the domestic animal is a chicken. In certain embodiments, the antibody originates from a species of the Camelidae family, or from a species of the Chondrichthyes class. In certain embodiments, the Camelidae species is a llama. In certain embodiments, the heavy chain antibody specifically binds Salmonella bacteria. In certain embodiments, the heavy chain antibody specifically binds Salmonella enterica. In certain embodiments, the heavy chain antibody specifically binds any of the Salmonella enterica serotypes Typhimurium, Enteritidis, Newport, Heidelberg, Gallinarum, Hadar, Javiana, Infantis, Montevideo, Muenchen, Braenderup, Saintpaul, Thompson, Agona, Litchfield, Anatum, Berta, Mbandaka, Oranienburg, Poona, Uganda, Senftenberg, Weltevreden, I 4,[5],12:i:-, I 13,23:b:-, or any combination thereof. In certain embodiments, the heavy chain antibody specifically binds a Salmonella biomolecule. In certain embodiments, the heavy chain antibody specifically binds a Salmonella biomolecule that functions in bacterial motility. In certain embodiments, the antibody reduces bacterial motility compared to a negative control antibody. In certain embodiments, the antibody reduces bacterial adhesion compared to a negative control antibody. In certain embodiments, wherein the antibody reduces bacterial invasion compared to a negative control antibody. In certain embodiments, the Salmonella biomolecule is a Salmonella derived polypeptide. In certain embodiments, the biomolecule is a component of a flagellum. In certain embodiments, the biomolecule comprises a polypeptide derived from the flagella filament structural protein (FliC). In certain embodiments, the biomolecule comprises a polypeptide derived from the PrgI protein. In certain embodiments, the biomolecule comprises a polypeptide derived from the Fimbrial protein A (FimA). In certain embodiments, the heavy chain antibody is monoclonal. In certain embodiments, the heavy chain antibody is polyclonal. In certain embodiments, the heavy chain antibody is incorporated into a spray for application to the exterior of a domestic animal. In certain embodiments, the heavy chain antibody is formulated for injection. In certain embodiments, the heavy chain antibody is formulated as a liquid. In certain embodiments, the heavy chain antibody is not a chicken antibody. In certain embodiments, the heavy chain antibody is not an IgY. In certain embodiments, the heavy chain antibody does not specifically bind bacterial lipopolysaccharide (LPS). In certain embodiments, the peptide sequence of the CDR1 is set forth in any of SEQ ID NOs: 1 to 91. In certain embodiments, the peptide sequence of the CDR2 is set forth in any of SEQ ID NOs: 92 to 182. In certain embodiments, the peptide sequence of the CDR3 is set forth in any of SEQ ID NOs: 183 to 273. In certain embodiments, the peptide sequence of the CDR1 is set forth in any of SEQ ID NOs: 1 to 91, the peptide sequence of the CDR2 is set forth in any of SEQ ID NOs: 92 to 182, and the peptide sequence of the CDR3 is set forth in any of SEQ ID NOs: 183 to 273. In certain embodiments, the peptide sequence of the CDR1 is set forth in SEQ ID NO: 1, SEQ ID NO:4, SEQ ID NO: 12, SEQ ID NO: 20, SEQ ID NO: 21, or SEQ ID NO: 60. In certain embodiments, the peptide sequence of the CDR2 is set forth in SEQ ID NO: 92, SEQ ID NO: 95, SEQ ID NO: 103, SEQ ID NO: 111, SEQ ID NO: 112, or SEQ ID NO: 151. In certain embodiments, the peptide sequence of the CDR3 is set forth in SEQ ID NO: 183, SEQ ID NO: 186, SEQ ID NO: 194, SEQ ID NO: 202, SEQ ID NO: 203, or SEQ ID NO: 242. In certain embodiments, the peptide sequence of the CDR1 is set forth in SEQ ID NO: 1, the peptide sequence of the CDR2 is set forth in SEQ ID NO: 92, and the peptide sequence of the CDR3 is set forth in SEQ ID NO: 183. In certain embodiments, the peptide sequence of the CDR1 is set forth in SEQ ID NO: 4, the peptide sequence of the CDR2 is set forth in SEQ ID NO: 95, and the peptide sequence of the CDR3 is set forth in SEQ ID NO: 186. In certain embodiments, the peptide sequence of the CDR1 is set forth in SEQ ID NO: 12, the peptide sequence of the CDR2 is set forth in SEQ ID NO: 103, and the peptide sequence of the CDR3 is set forth in SEQ ID NO: 194. In certain embodiments, the peptide sequence of the CDR1 is set forth in SEQ ID NO: 20, the peptide sequence of the CDR2 is set forth in SEQ ID NO: 111, and the peptide sequence of the CDR3 is set forth in SEQ ID NO: 202. In certain embodiments, the peptide sequence of the CDR1 is set forth in SEQ ID NO: 21, the peptide sequence of the CDR2 is set forth in SEQ ID NO: 112, and the peptide sequence of the CDR3 is set forth in SEQ ID NO: 203. In certain embodiments, the peptide sequence of the CDR1 is set forth in SEQ ID NO: 60, the peptide sequence of the CDR2 is set forth in SEQ ID NO: 151, and the peptide sequence of the CDR3 is set forth in SEQ ID NO: 242. In certain embodiments, the peptide sequence of the CDR1 is set forth in any of SEQ ID NOs: 280 to 329, 573, or 765-768. In certain embodiments, the peptide sequence of the CDR2 is set forth in any of SEQ ID NOs: 330 to 379, 574, or 769-772. In certain embodiments, the peptide sequence of the CDR3 is set forth in any of SEQ ID NOs: 380-429, 575 to 773-776. In certain embodiments, the heavy chain antibody is for use with any of the uses of this disclosure. In certain embodiments, the heavy chain antibody is for use with an animal feed. In certain embodiments, the heavy chain antibody is for use with any of the methods of this disclosure.

In certain embodiments, described herein, is a polypeptide comprising a plurality of variable region fragments of a heavy chain antibody (V_{H}Hs) which specifically bind Salmonella enterica. In certain embodiments, the plurality of V_{H}Hs comprise at least three V_{H}Hs. In certain embodiments, one or more of the plurality of V_{H}Hs comprise an amino acid sequence with at least 80% identity to the amino acid sequence set forth in SEQ ID NO: 463. In certain embodiments, any one or more of the plurality of V_{H}Hs is identical to another V_{H}H of the plurality of V_{H}Hs. In certain embodiments, a minimum concentration of the polypeptide required for 50% motility inhibition of Salmonella enterica is reduced by at least 20-fold when compared to a minimum concentration of a control polypeptide required for 50% motility inhibition of Salmonella enterica, the control polypeptide comprising a single V_{H}H that is identical to any one of the plurality of V_{H}Hs of the polypeptide. In certain embodiments, the plurality of V_{H}Hs are covalently coupled to one another by a linker, the linker comprising one or more amino acids. Also described is a polypeptide complex, wherein the polypeptide comprises a first component polypeptide and a second component polypeptide, wherein the first component polypeptide and the second component polypeptide are not covalently linked together and are coupled together by a protein-protein interaction, a small molecule-protein interaction, or a small molecule-small molecule interaction, wherein each of the first and the second component polypeptides comprise a V_{H}H which specifically binds Salmonella enterica. In certain embodiments, a nucleic acid encodes the polypeptide or the polypeptide complex. In certain embodiments, a plurality of nucleic acids encodes the polypeptide complex. In certain embodiments, a cell comprises the nucleic acid or the plurality of nucleic acids. In certain embodiments, the cell is a yeast cell. In certain embodiments, the yeast is of the genus Pichia. In certain embodiments, a method of producing the polypeptide or the polypeptide complex, comprises (a) incubating the cell in a medium suitable for secretion of the polypeptide from the cell; and (b) purifying the polypeptide from the medium. In certain embodiments, the polypeptide or the polypeptide complex is for use in reducing or preventing a pathogenic Salmonella enterica infection of a human individual or a domestic animal. In certain embodiments, described herein is a use of the polypeptide or the polypeptide complex for reducing or preventing a pathogenic Salmonella enterica infection of a human individual or a domestic animal. In certain embodiments, the use of the polypeptide or polypeptide complex is for oral administration to the human or animal.

In a certain aspect, described herein, is a polypeptide comprising a variable region fragment of a heavy chain antibody (V_{H}H), wherein the V_{H}H specifically binds a Salmonella enterica virulence factor, wherein the virulence factor is involved in bacterial motility, adhesion, invasion, or biofilm formation. In certain embodiments, the virulence factor comprises a flagellum, FliC, PrgI, FimA, or SipD. In certain embodiments, the virulence factor comprises a flagellum, FliC, PrgI, or FimA. In certain embodiments, the polypeptide that specifically binds a Salmonella enterica virulence factor specifically binds a virulence factor of any of the Salmonella enterica serotypes Typhimurium, Enteritidis, Newport, Heidelberg, Gallinarum, Hadar, Javiana, Infantis, Montevideo, Muenchen, Braenderup, Saintpaul, Thompson, Agona, Litchfield, Anatum, Berta, Mbandaka, Oranienburg, Poona, Uganda, Senftenberg, Weltevreden, I 4,[5],12:i:-, I 13,23:b:-, or any combination thereof. In certain embodiments, the amino acid sequence of the V_{H}H comprises: a CDR1 sequence set forth in SEQ ID NO: 1, a CDR2 sequence set forth in SEQ ID NO: 92, and a CDR3 sequence set forth in SEQ ID NO: 183; a CDR1 sequence set forth in SEQ ID NO: 4, a CDR2 sequence set forth in SEQ ID NO: 95, and a CDR3 sequence set forth in SEQ ID NO: 186; a CDR1 sequence set forth in SEQ ID NO: 12, a CDR2 sequence set forth in SEQ ID NO: 103, and a CDR3 sequence set forth in SEQ ID NO: 194; a CDR1 sequence set forth in SEQ ID NO: 20, a CDR2 sequence set forth in SEQ ID NO: 111, and a CDR3 sequence set forth in SEQ ID NO: 202 a CDR1 sequence set forth in SEQ ID NO: 21, a CDR2 sequence set forth in SEQ ID NO: 112, and a CDR3 sequence set forth in SEQ ID NO: 203; or a CDR1 sequence set forth in SEQ ID NO: 60, a CDR2 sequence set forth in SEQ ID NO: 151, and a CDR3 sequence set forth in SEQ ID NO: 242. In certain embodiments, the amino acid sequence of the V_{H}H comprises: a CDR1 sequence set forth in SEQ ID NO: 5, a CDR2 sequence set forth in SEQ ID NO: 96, and a CDR3 sequence set forth in SEQ ID NO: 187; a CDR1 sequence set forth in SEQ ID NO: 285, a CDR2 sequence set forth in SEQ ID NO: 335, and a CDR3 sequence set forth in SEQ ID NO: 385; or a CDR1 sequence set forth in SEQ ID NO: 284, a CDR2 sequence set forth in SEQ ID NO: 334, and a CDR3 sequence set forth in SEQ ID NO: 384. In certain embodiments, the amino acid sequence of the V_{H}H comprises an amino acid sequence at least 80% identical to that set forth in any one SEQ ID NOs: 478 to 488. In certain embodiments, the amino acid sequence of the V_{H}H comprises an amino acid sequence identical to that set forth in any one SEQ ID NOs: 478 to 488. In certain embodiments, the V_{H}H reduces bacterial motility of Salmonella enterica compared to a negative control antibody by at least 40%. In certain embodiments, the V_{H}H reduces biofilm formation by at least 10%. In certain embodiments, described herein is a nucleic acid encoding the polypeptide. In certain embodiments, described herein is a cell comprising the nucleic acid. In certain embodiments, the cell is a yeast cell. In certain embodiments, the yeast is of the genus Pichia. In certain embodiments, a method of producing the polypeptide comprises (a) incubating a cell in a medium suitable for secretion of the polypeptide from the cell; and (b) purifying the polypeptide from the medium. In certain embodiments, is described herein is a composition comprising a polypeptide and an animal feed. In certain embodiments, the composition is for use in reducing or preventing infection of a domestic animal with a pathogenic Salmonella enterica. In certain embodiments, described herein, is a method of reducing or preventing infection of a domestic animal with a pathogenic Salmonella enterica comprising administering to the domestic animal a polypeptide described herein or the composition described herein. In certain embodiments, described herein, is a composition comprising a polypeptide and a pharmaceutically acceptable stabilizer, excipient or diluent. In certain embodiments, the composition is for use in reducing or preventing infection in a human individual with a pathogenic Salmonella enterica. In certain embodiments, described herein, is a method of reducing or preventing infection of a human individual with a pathogenic Salmonella enterica comprising administering to the human individual a polypeptide, described herein, or the composition described herein. In certain embodiments, is a use of the polypeptide or of the composition for reducing or preventing a pathogenic Salmonella enterica infection in a domestic animal. In certain embodiments, is a use of a polypeptide or the composition for reducing or preventing a pathogenic Salmonella enterica infection of a human individual.

In a certain aspect, described herein, is an animal feed comprising a nutritional source and a polypeptide comprising a variable region fragment of a heavy chain antibody (V_{H}H), wherein the V_{H}H specifically binds an Enterobacteriaceae virulence factor, wherein the virulence factor is involved in bacterial motility, adhesion, invasion, or biofilm formation. In certain embodiments, the Enterobacteriaceae is Salmonella enterica. In certain embodiments, the amino acid sequence of the V_{H}H comprises: a CDR1 sequence set forth in SEQ ID NO: 1, a CDR2 sequence set forth in SEQ ID NO: 92, and a CDR3 sequence set forth in SEQ ID NO: 183; a CDR1 sequence set forth in SEQ ID NO: 4, a CDR2 sequence set forth in SEQ ID NO: 95, and a CDR3 sequence set forth in SEQ ID NO: 186; a CDR1 sequence set forth in SEQ ID NO: 12, a CDR2 sequence set forth in SEQ ID NO: 103, and a CDR3 sequence set forth in SEQ ID NO: 194; a CDR1 sequence set forth in SEQ ID NO: 20, a CDR2 sequence set forth in SEQ ID NO: 111, and a CDR3 sequence set forth in SEQ ID NO: 202 a CDR1 sequence set forth in SEQ ID NO: 21, a CDR2 sequence set forth in SEQ ID NO: 112, and a CDR3 sequence set forth in SEQ ID NO: 203; or a CDR1 sequence set forth in SEQ ID NO: 60, a CDR2 sequence set forth in SEQ ID NO: 151, and a CDR3 sequence set forth in SEQ ID NO: 242. In certain embodiments, the amino acid sequence of the V_{H}H comprises: a CDR1 sequence set forth in SEQ ID NO: 5, a CDR2 sequence set forth in SEQ ID NO: 96, and a CDR3 sequence set forth in SEQ ID NO: 187; a CDR1 sequence set forth in SEQ ID NO: 285, a CDR2 sequence set forth in SEQ ID NO: 335, and a CDR3 sequence set forth in SEQ ID NO: 385; or a CDR1 sequence set forth in SEQ ID NO: 284, a CDR2 sequence set forth in SEQ ID NO: 334, and a CDR3 sequence set forth in SEQ ID NO: 384. In certain embodiments, the amino acid sequence of the V_{H}H comprises an amino acid sequence at least 80% identical to that set forth in any one SEQ ID NOs: 478 to 488. In certain embodiments, the amino acid sequence of the V_{H}H comprises an amino acid sequence identical to that set forth in any one SEQ ID NOs: 478 to 488.

### BRIEF DESCRIPTION OF THE DRAWINGS

FIGS. 1A, 1B, and 1C show pictures of a plate-based motility assay performed using agar plates.

FIGS. 2A, 2B, and 2C show pictures of a plate-based motility assay performed using agar plates.

FIGS. 3A, 3B, and 3C show pictures of a plate-based motility assay performed using agar plates.

FIG. 4 is a picture of a plate-based motility assay performed using agar plates.

FIG. 5 is a graph that shows the velocity of Salmonella enterica serotype Typhimurium strain SL1344 in a live imaging motility assay.

FIG. 6 is a graph that shows the differences in specific binding among 17 different monoclonal V_{H}H antibodies (NBXs) raised against whole Salmonella enterica serotype Typhimurium flagella or recombinant FliC versus a Salmonella enterica serotype Typhimurium flagella capture surface.

FIG. 7 is a graph that shows the differences in specific binding among 17 different monoclonal V_{H}H antibodies (NBXs) raised against whole Salmonella Typhimurium flagella or recombinant FliC versus a Salmonella enterica serotype Enteritidis flagella capture surface.

FIG. 8 is a graph that shows the differences in specific binding among 17 different monoclonal V_{H}H antibodies (NBXs) raised against whole Salmonella enterica serotype Typhimurium flagella or recombinant FliC versus a recombinant FliC capture surface.

FIG. 9 is a graph that shows the velocity of Salmonella enterica serotype Typhimurium strain SL1344 in a live imaging motility assay in the presence of NovoBind NBX0001, NBX0006, NBX0012, NBX0014, NBX0015, NBX0018 and NBX0019.

FIG. 10 is a graph that shows the velocity of Salmonella enterica serotype Typhimurium strain SL1344 in a live imaging motility assay in the presence of NovoBind NBX0002, NBX0010, NBX0015, NBX0016, NBX0017 and NBX0021.

FIG. 11 is a graph that shows the velocity of Salmonella enterica serotype Typhimurium strain SL1344 in a live imaging motility assay in the presence of NovoBind NBX0022, NBX0023, NBX0024, NBX0025, NBX0026 and NBX0027.

FIG. 12 is a graph that shows the velocity of Salmonella enterica serotype Typhimurium strain SL1344 in a live imaging motility assay in the presence of NovoBind NBX0005, NBX0008, NBX0009, NBX0028, NBX0029 and NBX0030.

FIG. 13 shows that NBX can inhibit biofilm formation. In the absence of an NBX (PBS), a robust biofilm is formed after 24 hours. Treatment with four different NBXs (NBX0018, NBX0005, NBX0015, NBX0026) reduced biofilm formation by between 12% and 59%. Data presented in the graph represent the mean of five replicate wells and error bars represent the standard deviations.

FIG. 14A to 14E show that multimeric V_{H}H promote Salmonella aggregation. PBS treatment (A) and NBX0018 (B) fail to aggregate Salmonella enterica serovar Typhimurium strain SL1344 (black dots), while NBX0018-NBX0018 (C) induced small bacterial aggregates. Bacterial aggregates formed by NBX0018-NBX0018-NBX0018 (D) exceeded the size of those formed by the polyclonal antibody positive control (E). Representative aggregates are circled in black for visualization purposes.

FIG. 15 shows that multimeric V_{H}H block HeLa cell invasion by Salmonella enterica serovar Typhimurium strain SL1344 is reduced by more than 50% in the presence of NBX0018-NBX0018-NBX0018 (NBX trimer). Control refers to uninfected HeLa cells treated with PBS throughout the experiment. Shown are the means of duplicate wells and the error bars represent the standard deviations.

FIG. 16A to 16E show the production system dependency of V_{H}H activity. Salmonella enterica serovar Typhimurium strain SL1344 clumps Saccharomyces cerevisiae during PBS treatment (A) or treatment with NBX0018 recombinantly produced in Escherichia coli (D). In the presence of Salmonella enterica serovar Typhimurium strain SL1344 lacking the Type-1 Fimbriae (FimA mutant strain), Saccharomyces cerevisiae cells remain unclumped (B). In the presence of Salmonella enterica serovar Typhimurium strain SL1344 with intact Type-1 Fimbriae and excess soluble mannose (C) or NBX0018 recombinantly produced in Pichia pastoris (E), Saccharomyces cerevisiae cells remain unclumped, indicating that the interaction between Type-1 Fimbriae and mannose sugars on the surface of the Saccharomyces cerevisiae cells has been blocked.

FIG. 17 Shows phage ELISA binding data for NBXs to FimA. Black bars show binding to wells coated with FimA in phosphate-buffered saline (PBS). Grey bars are negative controls that show binding to wells coated with PBS only. In all cases binding to the antigen target is at least four-fold above background.

FIG. 18 Shows phage ELISA binding data for NBXs to FliC. Black bars show binding to wells coated with FliC in phosphate-buffered saline (PBS). Grey bars are negative controls that show binding to wells coated with PBS only. In all cases binding to the antigen target is at least four-fold above background.

FIG. 19 Shows phage ELISA binding data for NBXs to PrgI. Black bars show binding to wells coated with PrgI in phosphate-buffered saline (PBS). Grey bars are negative controls that show binding to wells coated with PBS only. In all cases binding to the antigen target is at least four-fold above background.

FIG. 20 Shows phage ELISA binding data for NBXs to PrgI-SipD. Black bars show binding to wells coated with PrgI-SipD in phosphate-buffered saline (PBS). Grey bars are negative controls that show binding to wells coated with PBS only. In all cases binding to the antigen target is at least four-fold above background.

FIG. 21 Shows phage ELISA binding data for NBXs to SipD. Black bars show binding to wells coated with SipD in phosphate-buffered saline (PBS). Grey bars are negative controls that show binding to wells coated with PBS only. In all cases binding to the antigen target is at least four-fold above background.

FIG. 22 Shows Phage ELISA binding data. Black bars show binding to wells coated with appropriate antigen target (FimA for NBX0100 and PrgI for NBX0104, NBX0105, and NBX0108) dissolved in phosphate-buffered saline (PBS). Grey bars are negative controls that show binding to wells coated with PBS only. In all cases binding to the antigen target is at least four-fold above background.

FIG. 23 shows stability of V_{H}Hs in chicken GI fluids.

FIG. 24 shows presence of V_{H}Hs in GI tract of a chicken after oral administration.

FIG. 25A to 25B show that V_{H}H are non-toxic to Chicken and have no impact on bodyweight (A) or spleen weight (B).

FIG. 26A and 26B show a schematic of camelid heavy chain only antibodies and their relationship to V_{H}H domains and complementarity determining regions (CDRs).

FIG. 27A and 27B show a schematic to describe the principle behind NBX complex formation via protein-protein interactions. (A) NBX1-A and NBX2-B are two constructs expressed and purified individually. Protein domains A and B form a highly stable interaction. When NBX1-A and NBX2-B are mixed together, a complex is formed, driven by the interaction of A and B, that keeps NBX1 and NBX2 together. (B) Multimers of the same NBX can come together immediately upon production if protein domain A naturally self-oligomerizes.

### DETAILED DESCRIPTION OF THE INVENTION

In certain embodiments, described herein, is the use of an antibody for preventing infection in a domestic animal by a pathogenic Enterobacteriaceae bacterial population.

In certain embodiments, described herein, is the use of an antibody for reducing a pathogenic Enterobacteriaceae bacterial population in a domestic animal.

In certain embodiments, described herein, is the use of an antibody for preventing infection in a human individual by a pathogenic Enterobacteriaceae bacterial population.

In certain embodiments, described herein, is the use of an antibody for reducing a pathogenic Enterobacteriaceae bacterial population in a human individual.

In certain embodiments, described herein, is the use of a variable region fragment of a heavy chain antibody (V_{H}H) which specifically binds a Salmonella FliC protein, a Salmonella PrgI protein, a Salmonella FimA protein, or any combination thereof, for preventing infection with a pathogenic Salmonella enterica bacterial population in a domestic poultry animal.

In certain embodiments, described herein, is the use of a variable region fragment of a heavy chain antibody (V_{H}H) which specifically binds a Salmonella FliC protein, a Salmonella PrgI protein, a Salmonella FimA protein, or any combination thereof, for reducing a pathogenic Salmonella enterica bacterial population in a domestic poultry animal.

In certain embodiments, described herein, is the use of a variable region fragment of a heavy chain antibody (V_{H}H) which specifically binds a Salmonella FliC protein, a Salmonella PrgI protein, a Salmonella FimA protein, or any combination thereof, for preventing infection with a pathogenic Salmonella enterica bacterial population in a human individual.

In certain embodiments, described herein, is the use of a variable region fragment of a heavy chain antibody (V_{H}H) which specifically binds a Salmonella FliC protein, a Salmonella PrgI protein, a Salmonella FimA protein, or any combination thereof, for reducing a pathogenic Salmonella enterica bacterial population in a human individual.

In certain embodiments, described herein, is an animal feed comprising an antibody that specifically binds an Enterobacteriaceae bacterial species.

In certain embodiments, described herein, is a substance for introduction to an alimentary canal of a domestic poultry animal, the substance comprising a liquid, a gel, a spray, a tablet, or a pellet, for administration to the animal, which comprises a variable region fragment of a heavy chain antibody (V_{H}H) which specifically binds a Salmonella FliC protein, a Salmonella PrgI protein, a Salmonella FimA protein, or any combination thereof.

In certain embodiments, described herein, is a method for preventing infection with a Salmonella bacterial population in a domestic animal, the method comprising introducing an antibody to an alimentary canal of the domestic animal.

In certain embodiments, described herein, is a method for reducing a Salmonella bacterial population in a domestic animal, the method comprising introducing an antibody to an alimentary canal of the domestic animal.

In certain embodiments, described herein, is a method for preventing infection with a Salmonella enterica bacterial population in a domestic poultry animal, the method comprising introducing to an alimentary canal of the animal a monoclonal variable region fragment of a heavy chain antibody (V_{H}H) which specifically binds a Salmonella flagella filament structural protein (FliC), a Salmonella PrgI protein, a Salmonella FimA protein, or any combination thereof.

In certain embodiments, described herein, is a method for reducing a Salmonella enterica bacterial population in a domestic poultry animal, the method comprising introducing to an alimentary canal of the animal a variable region fragment of a heavy chain antibody (V_{H}H) which specifically binds a Salmonella flagella filament structural protein (FliC), a Salmonella PrgI protein, a Salmonella FimA protein, or any combination thereof.

In certain embodiments, described herein, is a variable region fragment of an antibody which specifically binds Salmonella.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about refers to an amount that is near the stated amount by about 10%, 5%, or 1%.

As referred to herein “antibody fragment” refers to any portion of a conventional or heavy chain antibody that retains a capacity to specifically bind a target antigen and may include a single chain antibody, a variable region fragment of a heavy chain antibody, a nanobody; a polypeptide or an immunoglobulin new antigen receptor (IgNAR).

As referred to herein an “antibody originates from a species” when any of the CDR regions of the antibody were raised in an animal of said species. Antibodies that are raised in a certain species and then optimized by an in vitro method (e.g., phage display) are considered to have originated from that species.

As referred to herein “conventional antibody” refers to any full-sized immunoglobulin that comprises two heavy chain molecules and two light chain molecules joined together by a disulfide bond. In certain embodiments, the antibodies, compositions, animal feeds, and methods described herein do not utilize conventional antibodies.

As referred to herein “heavy chain antibody” refers to an antibody that comprises two heavy chains and lacking the two light chains normally found in a conventional antibody. The heavy chain antibody may originate from a species of the Camelidae family or Chondrichthyes class. Heavy chain antibodies retain specific binding to an antigen in the absence of any light chain.

As referred to herein “poultry” refers to any domesticated or captive raised bird that is kept for its egg, meat, feathers, or combination thereof.

As referred to herein “specific binding” or “specifically binds” refers to binding that occurs between an antibody and its target molecule that is mediated by at least one complementarity determining region (CDR) of the antibody's variable region. Binding that is between the constant region and another molecule, such as Protein A, for example, does not constitute specific binding.

As referred to herein “V_{H}H” refers to an antibody or antibody fragment comprising a single heavy chain variable region which may be derived from natural or synthetic sources. NBXs referred to herein are an example of a V_{H}H.

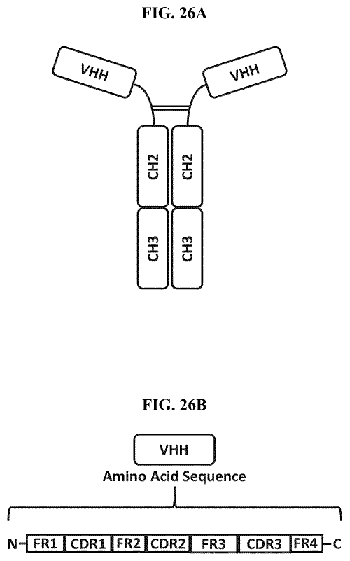

A schematic of camelid heavy chain only antibodies and their relationship to V_{H}H domains and complementarity determining regions (CDRs) is shown in FIG. 26. (Panel A) A camelid heavy chain only antibody consists of two heavy chains linked by a disulphide bridge. Each heavy chain contains two constant immunoglobulin domains (CH2 and CH3) linked through a hinge region to a variable immunoglobulin domain (V_{H}H). (Panel B) are derived from single V_{H}H domains. Each V_{H}H domain contains an amino acid sequence of approximately 110-130 amino acids. The V_{H}H domain consists of the following regions starting at the N-terminus (N): framework region 1 (FR1), complementarity-determining region 1 (CDR1), framework region 2 (FR2), complementarity-determining region 2 (CDR2), framework region 3 (FR3), complementarity-determining region 3 (CDR3), and framework region 4 (FR4). The domain ends at the C-terminus (C). The complementarity-determining regions are highly variable, determine antigen binding by the antibody, and are held together in a scaffold by the framework regions of the V_{H}H domain The framework regions consist of more conserved amino acid sequences; however, some variability exists in these regions.

### Use of Antibodies for Preventing or Reducing a Bacterial Population

In certain embodiments, described herein are compositions, methods, and antibodies for use in preventing or reducing a pathogenic bacterial population in a domestic animal. In certain embodiments, described herein are compositions, methods, and antibodies for use in preventing or reducing a pathogenic bacterial population in the alimentary canal in a domestic animal. In certain embodiments, described herein are compositions, methods, and antibodies for use in preventing or reducing attachment of a pathogenic bacterial population in the alimentary canal in a domestic animal. In certain embodiments, described herein are compositions, methods, and antibodies for use in preventing or reducing transmission of a pathogenic bacterial population from a domestic animal to a human. In certain embodiments, described herein are compositions, methods, and antibodies for use in preventing or reducing transmission of a pathogenic bacterial population from the egg, dairy or meat products of a domestic animal to a human. In certain embodiments, described herein are compositions, methods, and antibodies for use in preventing or reducing transmission of a pathogenic bacterial population from a domestic animal to another domestic animal. In certain embodiments, preventing or reducing transmission of a pathogenic or bacterial population comprises reducing disease or symptoms of a disease caused by the bacteria in a human or domestic animal. In certain embodiments, the disease is a gastrointestinal disease with any one or more of the following symptoms in humans: nausea, vomiting, diarrhea, constipation, abdominal cramps, dehydration, fatigue, chills, fever, malnutrition, wasting, or bloody stool.

In certain embodiments, described herein are compositions, methods, and antibodies for use in preventing or reducing a pathogenic bacterial population in a human subject. In certain embodiments, described herein are compositions, methods, and antibodies for use in preventing or reducing a pathogenic bacterial population in the alimentary canal in a human subject. In certain embodiments, described herein are compositions, methods, and antibodies for use in preventing or reducing attachment of a pathogenic bacterial population in the alimentary canal of a human subject. In certain embodiments, described herein are compositions, methods, and antibodies for use in preventing or reducing transmission of a pathogenic bacterial population from a human subject to another human subject. In certain embodiments, preventing or reducing transmission of a pathogenic or bacterial population comprises reducing disease or symptoms of a disease caused by the bacteria in a human. In certain embodiments, the disease is a gastrointestinal disease with any one or more of the following symptoms in humans: nausea, vomiting, diarrhea, constipation, abdominal cramps, dehydration, fatigue, chills, fever, malnutrition, wasting, or bloody stool.

### Domestic Animals

In certain embodiments, the uses, compositions and methods described herein are for reducing or preventing a bacterial infection in any domestic animal. In certain embodiments, the domestic animal is a cow, pig, sheep, goat, horse, duck, chicken, goose, turkey or Cornish hen. In certain embodiments, the domestic animal is a pig. In certain embodiments, the domestic animal is from the superorder Galloanserae. In certain embodiments, the domestic animal is a poultry animal. In certain embodiments, the domestic animal is a duck, chicken, goose, turkey or Cornish hen. In certain embodiments, the domestic animal is a duck. In certain embodiments, the domestic animal is a turkey. In certain embodiments, the domestic animal is a chicken. In certain embodiments, the domestic animal is not a cow. In certain embodiments, the domestic animal is not a pig.

### Bacteria

In certain embodiments, the bacterial population comprises any member of the Enterobacteriaceae family. In certain embodiments, the bacterial population comprises any member of the genus Salmonella. In certain embodiments, the bacterial population comprises any member of the species Salmonella enterica. In certain embodiments, the bacterial population comprises any member of the Salmonella enterica serotypes Typhimurium, Enteritidis, Newport, Heidelberg, Gallinarum, Hadar, Javiana, Infantis, Montevideo, Muenchen, Braenderup, Saintpaul, Thompson, Agona, Litchfield, Anatum, Berta, Mbandaka, Oranienburg, Poona, Uganda, Senftenberg, Weltevreden, I 4,[5],12:i:- or I 13,23:b:-. In certain embodiments, the bacterial population is not E. coli (Escherichia coli).

### Antibodies

In certain embodiments, the antibody for the uses, compositions and methods described herein is an IgA, IgG, or IgM antibody. In certain embodiments, the antibody for the uses, compositions and methods described herein is a heavy chain antibody. In certain embodiments, the antibody for the uses, compositions and methods described herein is a V_{H}H. In certain embodiments, the antibody for the uses, compositions and methods described herein is a nanobody; a polypeptide. In certain embodiments, the antibody for the uses, compositions and methods described herein is synthetic. In certain embodiments, the antibody originates from a species of the Camildae family. In certain embodiments, the antibody originates from a species of the Chondrichthyes class. In certain embodiments, the antibody originates from a camel. In certain embodiments, the antibody originates from a llama. In certain embodiments, the antibody originates from a cartilaginous fish. In certain embodiments, the antibody originates from a shark. In certain embodiments, the antibody originates from a human, mouse, rat, rabbit, goat, sheep, horse, cow, donkey or Guinea pig. In certain embodiments, the antibody is a monoclonal antibody. In certain embodiments, the antibody is a polyclonal antibody. In certain embodiments, the antibody is a chimeric or CDR grafted antibody. In certain embodiments, the antibody has been altered to reduce immunogenicity in a poultry species. In certain embodiments, the antibody has been altered to reduce immunogenicity in a duck, chicken, goose, turkey or Cornish hen. In certain embodiments, the antibody has been altered to reduce immunogenicity in chickens. In certain embodiments, the antibody is not a chicken antibody. In certain embodiments, the antibody is not an IgY antibody.

### Heavy Chain Antibodies

Heavy chain antibodies are a type of antibody that comprises two heavy chains without associated light chains. Some species such as those from the family Camelidae and Chondrichthyes class raise heavy chain antibodies in response to infection. In some embodiments, the heavy chain antibody is a variable region fragment of a heavy chain antibody (V_{H}H). In some embodiments, the heavy chain antibody is a single chain antibody. In some embodiments, the heavy chain antibody is a nanobody; a polypeptide. In some embodiments, the heavy chain antibody is an immunoglobulin new antigen receptor (IgNAR). In some embodiments, the heavy chain antibody is a variable region fragment of an immunoglobulin new antigen receptor. In some embodiments, the heavy chain antibody is a synthetic or expressed polypeptide. In some embodiments, the variable region fragment of a heavy chain antibody (V_{H}H) is synthetic. In some embodiments, the heavy chain antibody is any fragment thereof that retains a capacity to specifically bind a target antigen. In certain embodiments, the heavy chain antibody comprises a sequence set forth either identically or with at least 80%, 90%, 95%, 98%, or 99% identity to any one of SEQ ID NOs: 478-488. In certain embodiments, the heavy chain antibody comprises a sequence set forth either identically or with at least 80%, 90%, 95%, 98%, or 99% identity to any one of SEQ ID NOs: 478-572. In certain embodiments, the heavy chain antibody comprises a sequence set forth in any one of SEQ ID NOs: 478-488. In certain embodiments, the heavy chain antibody comprises a CDR1 set forth in Table 1 or Table 2. In certain embodiments, the heavy chain antibody comprises a CDR2 set forth in Table 1 or Table 2. In certain embodiments, the heavy chain antibody comprises a CDR3 set forth in Table 1 or Table 2. In a certain embodiment, the heavy chain antibody is any of NBX0001, NBX005, NBX0006, NBX0015, NBX0018, NBX0019, NBX0030, or a combination thereof. In certain embodiments, the heavy chain antibody comprises an amino acid sequence with at least 80%, 90%, 95%, 97%, or 99% identity to SEQ ID NO: 475. In certain embodiments, the heavy chain antibody comprises an amino acid sequence with 100% identity to SEQ ID NO: 475. In certain embodiments, the heavy chain antibody comprises an amino acid sequence with at least 80%, 90%, 95%, 97%, or 99% identity to SEQ ID NO: 476. In certain embodiments, the heavy chain antibody comprises an amino acid sequence with 100% identity to SEQ ID NO: 476. In certain embodiments, the heavy chain antibody comprises an amino acid sequence with at least 80%, 90%, 95%, 97%, or 99% identity to SEQ ID NO: 477. In certain embodiments, the heavy chain antibody comprises an amino acid sequence with 100% identity to SEQ ID NO: 477. In certain embodiments, the heavy chain antibody comprises an amino acid sequence with at least 80%, 90%, 95%, 97%, or 99% identity to SEQ ID NO: 478. In certain embodiments, the heavy chain antibody comprises an amino acid sequence with 100% identity to SEQ ID NO: 478. In certain embodiments, the heavy chain antibody comprises an amino acid sequence with at least 80%, 90%, 95%, 97%, or 99% identity to SEQ ID NO: 479. In certain embodiments, the heavy chain antibody comprises an amino acid sequence with 100% identity to SEQ ID NO: 479. In certain embodiments, the heavy chain antibody comprises an amino acid sequence with at least 80%, 90%, 95%, 97%, or 99% identity to SEQ ID NO: 480. In certain embodiments, the heavy chain antibody comprises an amino acid sequence with 100% identity to SEQ ID NO: 480. In certain embodiments, the heavy chain antibody comprises an amino acid sequence with at least 80%, 90%, 95%, 97%, or 99% identity to SEQ ID NO: 481. In certain embodiments, the heavy chain antibody comprises an amino acid sequence with 100% identity to SEQ ID NO: 481. In certain embodiments, the heavy chain antibody comprises an amino acid sequence with at least 80%, 90%, 95%, 97%, or 99% identity to SEQ ID NO: 482. In certain embodiments, the heavy chain antibody comprises an amino acid sequence with 100% identity to SEQ ID NO: 482. In certain embodiments, the heavy chain antibody comprises an amino acid sequence with at least 80%, 90%, 95%, 97%, or 99% identity to SEQ ID NO: 483. In certain embodiments, the heavy chain antibody comprises an amino acid sequence with 100% identity to SEQ ID NO: 483. In certain embodiments, the heavy chain antibody comprises an amino acid sequence with at least 80%, 90%, 95%, 97%, or 99% identity to SEQ ID NO: 484. In certain embodiments, the heavy chain antibody comprises an amino acid sequence with 100% identity to SEQ ID NO: 484. In certain embodiments, the heavy chain antibody comprises an amino acid sequence with at least 80%, 90%, 95%, 97%, or 99% identity to SEQ ID NO: 485. In certain embodiments, the heavy chain antibody comprises an amino acid sequence with 100% identity to SEQ ID NO: 485. In certain embodiments, the heavy chain antibody comprises an amino acid sequence with at least 80%, 90%, 95%, 97%, or 99% identity to SEQ ID NO: 486. In certain embodiments, the heavy chain antibody comprises an amino acid sequence with 100% identity to SEQ ID NO: 486. In certain embodiments, the heavy chain antibody comprises an amino acid sequence with at least 80%, 90%, 95%, 97%, or 99% identity to SEQ ID NO: 487. In certain embodiments, the heavy chain antibody comprises an amino acid sequence with 100% identity to SEQ ID NO: 487. In certain embodiments, the heavy chain antibody comprises an amino acid sequence with at least 80%, 90%, 95%, 97%, or 99% identity to SEQ ID NO: 488. In certain embodiments, the heavy chain antibody comprises an amino acid sequence with 100% identity to SEQ ID NO: 488.

TABLE 1
unique SEQ IDs for CDRs of the V_{H}H antibodies of this disclosure

| | | | | | | | CDR3 | |
|---|---|---|---|---|---|---|---|---|
| | | CDR1 Amino | CDR1 | CDR2 | CDR2 | CDR3 | SEQ | |
| | | Acid | SEQ ID | Amino Acid | SEQ ID | Amino Acid | ID | |
| Clone | NBX | Sequence | NO: | Sequence | NO: | Sequence | NO: | Antigen |
| 1 | 0001 | GSIFSINAM | 1 | ITTGGNTAN | 92 | AARGLSYEY | 183 | Flagella |
| | | | | T | | DY | | |
| 2 | 0002 | GSIFSINAM | 2 | ITITSGRGGN | 93 | AARGAMTY | 184 | Flagella |
| | | | | T | | EYDY | | |
| 3 | 0004 | GIIFSPNAM | 3 | ITSFGII | 94 | NAKTFDGTR | 185 | Flagella |
| | | | | | | WRDY | | |
| 4 | 0006 | GNIFSINAM | 4 | ITTGGSYGN | 95 | AARGSQTYE | 186 | Flagella |
| | | | | T | | YDY | | |
| 5 | 0005 | GRSVSINPM | 5 | LLPSGRT | 96 | NTADF | 187 | Flagella |
| 6 | 0011 | GISVNINPM | 6 | LLPTGTT | 97 | YCNTADF | 188 | Flagella |
| 7 | 0012 | GSTFSINAM | 7 | ISRAGST | 98 | KASSGSSVYI | 189 | Flagella |
| | | | | | | GFGS | | |
| 8 | 0013 | VSINPM | 8 | LLSMARA | 99 | NTTDF | 190 | Flagella |
| 9 | 0017 | GRIFSSYDM | 9 | IRWGNGNT | 100 | AARIVNGGS | 191 | Flagella |
| | | | | | | WDY | | |
| 10 | 0042 | GSIESIRSM | 10 | 1TRSGST | 101 | NADFYGLYP | 192 | Flagella |
| | | | | | | RQY | | |
| 11 | 0016 | GRIFSSYDM | 11 | IRWGNGNT | 102 | AARGLAYEY | 193 | Flagella |
| | | | | | | EY | | |
| 12 | 0015 | GRTFSNNAM | 12 | ISRAGNT | 103 | KASSGSSVYI | 194 | Flagella |
| | | | | | | GVGS | | |
| 13 | 0014 | GRTFSRLAM | 13 | ISWSGGNT | 104 | AAPERSGSY | 195 | Flagella |
| | | | | | | AYTPSRLNEY | | |
| | | | | | | AY | | |
| 14 | 0009 | GRMFSSYDM | 14 | ITKNGRTT | 105 | AGRRSNAD | 196 | Flagella |
| | | | | | | NWDY | | |
| 15 | 0010 | GSIFSINAV | 15 | IGTGGSSSG | 106 | AARGTISYEY | 197 | Flagella |
| | | | | NT | | DY | | |
| 16 | 0008 | GRIFSSYDM | 16 | IRWGNGNT | 107 | ATRIVNGGS | 198 | Flagella |
| | | | | | | WDY | | |
| 17 | 0043 | GRIFS1NPM | 17 | LMTGGKTPD | 108 | YNCDFWGL | 199 | Flagella |
| | | | | A | | AYEYDY | | |
| 18 | 0007 | GRIFSIYDM | 18 | ITWGNGNT | 109 | PARIVNGGS | 200 | Flagella |
| | | | | | | WDY | | |
| 19 | 0044 | GFTFSSAWM | 19 | IYPSGSST | 110 | ATASRRGVV | 201 | FliC |
| | | | | | | SLTSNPSTSR | | |
| | | | | | | NDFSS | | |
| 20 | 0018 | GIIFSPNAM | 20 | ITSFGII | 111 | NAKTFDGTR | 202 | FliC |
| | | | | | | WHDY | | |
| 21 | 0019 | GIIFSPNAM | 21 | ITSFGII | 112 | NAKAFDGTR | 203 | FliC |
| | | | | | | WHDY | | |
| 22 | 0020 | GIIFSPNAL | 22 | IISGGRS | 113 | NANVYDGN | 204 | FliC |
| | | | | | | RWRTY | | |
| 23 | 0045 | GIIFSPNAM | 23 | ITSFG11 | 114 | NAKSFDGSR | 205 | FliC |
| | | | | | | AINDY | | |
| 24 | 0046 | GRSVSINPM | 24 | LLPSGRT | 115 | NTADF | 206 | FliC |
| 25 | 0047 | VFILNAM | 25 | 11SFGIK | 116 | NGKAFDFNR | 207 | FliC |
| | | | | | | WHDY | | |
| 26 | 0048 | REFFTTDAM | 26 | KSSGADP | 117 | YRKGQYYRG | 208 | FliC |
| | | | | | | TYWDNFES | | |
| 27 | 0049 | VRAFSSRAM | 27 | ISSSGSSI | 118 | AAVRPYGSG | 209 | FliC |
| | | | | | | TYSRTEAYNF | | |
| 28 | 0050 | GGTFSDYAW | 28 | ISWTGGII | 119 | AAVGRILGW | 210 | FliC |
| | | | | | | IPTIVIYRQAA | | |
| | | | | | | SYDY | | |
| 29 | 0051 | GIIFSPNAM | 29 | ITSFGII | 120 | NAKSFDGTR | 211 | FliC |
| | | | | | | WVEH | | |
| 30 | 0052 | GIIFSPNAM | 30 | ITSFGII | 121 | NAKAFDGTR | 212 | FliC |
| | | | | | | WRDY | | |
| 31 | 0053 | GITNRITTM | 31 | IRDDRDAN | 122 | NVQTIIRNY | 213 | FliC |
| 32 | 0054 | GSVRTINDM | 32 | 1SSGGNT | 123 | SQRGQYFTE | 214 | FliC |
| | | | | | | GYWKEYDN | | |
| 33 | 0055 | GIIFSPNAM | 33 | ITSFG11 | 124 | NANVYDGN | 215 | FliC |
| | | | | | | RWRTY | | |
| 34 | 0021 | GRTFRSYTM | 34 | ISWSAGST | 125 | AAGTKYSDTI | 216 | FliC |
| | | | | | | ITWGS | | |
| 35 | 0032 | GIIFSPNAM | 35 | ITSSGII | 126 | NAKAFDGTR | 217 | FliC |
| | | | | | | WYDY | | |
| 36 | 0033 | GRTFSSYVM | 36 | ISWSGGSS | 127 | AARTALGGT | 218 | FliC |
| | | | | | | YDY | | |
| 37 | 0056 | FLENPPFAI | 37 | ITYCVMEI | 128 | HPHF | 219 | FliC |
| 38 | 0022 | GSTVTISTV | 38 | ISSDSTT | 129 | NVVGTYWT | 220 | FliC |
| | | | | | | GADWRPFD | | |
| | | | | | | T | | |
| 39 | 0034 | GIIFNPNAM | 39 | ITSFGII | 130 | NAITFYGTR | 221 | FliC |
| | | | | | | WLDY | | |
| 40 | 0035 | GIIFSPNAL | 40 | IISGGRS | 131 | NADVYDGN | 222 | FliC |
| | | | | | | RWRTY | | |
| 41 | 0036 | GIIFSPNAM | 41 | ITSFGII | 132 | FAKTFDGTR | 223 | FliC |
| | | | | | | WCDY | | |
| 42 | 0037 | GIIFSPNAL | 42 | IISGGRS | 133 | NAIVYDGNR | 224 | FliC |
| | | | | | | WRTY | | |
| 43 | 0057 | GIFESTFDA | 43 | IGSRGSI | 134 | NSVGH | 225 | FimA |
| | | TAM | | | | | | |
| 44 | 0024 | GSIFSTNVM | 44 | ITSGGNT | 135 | AAQTLGSSY | 226 | FimA |
| | | | | | | YDA | | |
| 45 | 0058 | GRTFDKYRI | 45 | ISWNGAYT | 136 | AAVQSTVIQ | 227 | FimA |
| | | | | | | TSPNRYNY | | |
| 46 | 0059 | GRTFINRSM | 46 | 1SSSGSNT | 137 | AAARLGWG | 228 | FimA |
| | | | | | | LTISDRIYEY | | |
| 47 | 0025 | GFTFSMYGM | 47 | INSGGART | 138 | AKASLPWFD | 229 | FimA |
| | | | | | | GSSPDY | | |
| 48 | 0026 | GLTFSSYGM | 48 | IKMSGDT | 139 | AAARVRTPG | 230 | FimA |
| | | | | | | WGPQKSYD | | |
| | | | | | | Y | | |
| 49 | 0027 | GRTFSSYAM | 49 | INWSGGRI | 140 | NADYDNSGS | 231 | FimA |
| | | | | | | YYYQKGNYE | | |
| | | | | | | YDY | | |
| 50 | 0060 | GRDASDGTE | 50 | MRWNTGSE | 141 | TADGPPDYG | 232 | FimA |
| | | SRYVM | | | | KYDY | | |
| 51 | 0028 | GRTFGSLHM | 51 | ISAAGGVT | 142 | AAVKYWGR | 233 | FimA |
| | | | | | | RQRADEYDY | | |
| 52 | 0061 | GFTFDDYVI | 52 | TSSSDGDT | 143 | AAELSLNPG | 234 | FimA |
| | | | | | | KRLTLE1LKY | | |
| | | | | | | DY | | |
| 53 | 0062 | GFRLNDYYV | 53 | TGSRSGRL | 144 | AAGYGAGD | 235 | FimA |
| | | | | | | VKRALSSCR | | |
| | | | | | | GSYVY | | |
| 54 | 0063 | GIIFRINTM | 54 | ITRAGST | 145 | KMNHQLYS | 236 | FimA |
| | | | | | | DSSYENVY | | |
| 55 | 0064 | GFTLGYFAI | 55 | ISNSDGST | 146 | ATDTWGNS | 237 | FimA |
| | | | | | | RCDHDMRY | | |
| 56 | 0040 | GLAFNTKTM | 56 | ITWGTINT | 147 | ESEALLETTP | 238 | FimA |
| | | | | | | SRRPYEYNY | | |
| 57 | 0065 | GFTESRYLM | 57 | VNSGGAMT | 148 | AKGQREYYN | 239 | FimA |
| | | | | | | DFEFDY | | |
| 58 | 0041 | GRIFGSLHM | 58 | ITAAGGVT | 149 | RTLGCSYYER | 240 | FimA |
| | | | | | | ADEYNY | | |
| 59 | 0029 | GSISSIKAM | 59 | WRMYSGT | 150 | YLEIPESRGA | 241 | FimA |
| | | | | | | F | | |
| 60 | 0030 | GRTFSRDAM | 60 | INWNGRST | 151 | AAGEWGIRP | 242 | FimA |
| | | | | | | YNYDY | | |
| 61 | 0031 | GRTFSSYAM | 61 | INWSGGRI | 152 | NTDYDNSGS | 243 | FimA |
| | | | | | | YYYQKGNYE | | |
| | | | | | | YDY | | |
| 62 | 0066 | GRTFS1YAM | 62 | ININSGGRI | 153 | NANYDNNG | 244 | FimA |
| | | | | | | SYYYQKGNY | | |
| | | | | | | EYDY | | |
| 63 | 0067 | GLAFSTKTM | 63 | 1TWGTSST | 154 | AAAALLETTP | 245 | Prgl |
| | | | | | | SRRPSAYNY | | |
| 64 | 0068 | GRTFSSNTM | 64 | 1ASSDGAT | 155 | AGAWGYAG | 246 | Prgl |
| | | | | | | IIPRGAYDD | | |
| 65 | 0069 | GRTESSYGM | 65 | 1KVSGDT | 156 | AAARIRTPG | 247 | Prgl |
| | | | | | | WGPQKSYD | | |
| | | | | | | Y | | |
| 66 | 0070 | GRALSAYIM | 66 | 1SSSGSNT | 157 | AAGVVTAQ | 248 | Prgl |
| | | | | | | AIMAARDFD | | |
| | | | | | | Y | | |
| 67 | 0071 | VRTFNTYNI | 67 | ISWGRGNT | 158 | AADRSREGR | 249 | Prgl |
| | | | | | | TRPNEYDY | | |
| 68 | 0023 | GRSFSSYNM | 68 | ITWSGNT | 159 | KVRAEDTDY | 250 | Prgl |
| | | | | | | AAPERSGSY | | |
| 69 | 0072 | ERTFSSYTM | 69 | ISWSGGNT | 160 | AYTPSRLNEY | 251 | Prgl |
| | | | | | | AY | | |
| 70 | 0073 | GTFFRINYM | 70 | 1SSGGST | 161 | NADFYGLYP | 252 | Prgl |
| | | | | | | RQY | | |
| 71 | 0038 | GRTFSSYAM | 71 | IRWTRSST | 162 | AADRYYRTD | 253 | Prgl |
| | | | | | | IYRASSYEY | | |
| 72 | 0074 | GFNFSLYSM | 72 | ISNLSVRT | 163 | AKGWTVDV | 254 | Prgl |
| | | | | | | NHIED | | |
| 73 | 0075 | ARILSSFIRM | 73 | IRWGSGST | 164 | AAKYGGTDL | 255 | Prgl |
| | | | | | | LSRYEY | | |
| 74 | 0076 | GFILDNYAI | 74 | ISRSDGDT | 165 | ASVYSFDPG | 256 | Prgl |
| | | | | | | RCGPIATMV | | |
| | | | | | | GHY | | |
| 75 | 0077 | GFMPDYSAL | 75 | ISRDGHTY | 166 | ATDAAGGR | 257 | Prgl |
| | | | | | | GSFFIDHKRT | | |
| | | | | | | CPSEEYDS | | |
| 76 | 0078 | LYSLRTRLQ | 76 | TYWPIECH | 167 | TADGPPDYG | 258 | Prgl |
| | | YL | | | | KYDY | | |
| 77 | 0079 | GFTESSYWM | 77 | 1DTGGGST | 168 | ARVSVIRPPY | 259 | Prgl |
| | | | | | | GVYSDFGS | | |
| 78 | 0080 | GFTESNFWM | 78 | LNTGGGAT | 169 | TLYGSGAAE | 260 | Prgl |
| | | | | | | KFHS | | |
| 79 | 0081 | ARTFSSYAM | 79 | 1SWDGATT | 170 | AANWGRRR | 261 | Prgl |
| | | | | | | VPTTVHEYD | | |
| | | | | | | V | | |
| 80 | 0082 | GRTFINRSM | 80 | GSSGSYS | 171 | AAARLGWG | 262 | Prgl |
| | | | | | | LTISDRIYEY | | |
| 81 | 0083 | GFTLDYFA1 | 81 | ISNIDGIT | 172 | ATDTWGNS | 263 | Prgl |
| | | | | | | RCDHDMRY | | |
| 82 | 0084 | GRTFSMYAM | 82 | 1NWSGAST | 173 | AAGSFSDNK | 264 | Prgl |
| | | | | | | YYTRSQDYE | | |
| | | | | | | H | | |
| 83 | 0085 | VHSFSNYAL | 83 | 1TWNAES | 174 | AASSWCQTF | 265 | Prgl |
| | | | | | | DAKYGY | | |
| 84 | 0039 | GRPFINYNM | 84 | ISWSGDST | 175 | AADNQHDIP | 266 | Prgl |
| | | | | | | LRPG | | |
| 85 | 0086 | AFTFDDFAV | 85 | LSSSDGST | 176 | HPSDTTGW | 267 | Prgl |
| | | | | | | TRGRAY | | |
| 86 | 0087 | GESLDHSAI | 86 | VHHDGTA | 177 | ATACTRLWK | 268 | Prgl |
| | | | | | | PGRDY | | |
| 87 | 0088 | GFDFNIYWM | 87 | IRSTGDTI | 178 | MRDFYT | 269 | Prgl |
| 88 | 0089 | GRTLRSYVM | 88 | LSWSGIST | 179 | AAASTIKHCY | 270 | Prgl |
| | | | | | | TAVSYYTKD | | |
| | | | | | | AQYDY | | |
| 89 | 0090 | GL1FGDYVM | 89 | ISSDSTT | 180 | NVVGTYWT | 271 | Prgl |
| | | | | | | GADWRPFD | | |
| | | | | | | T | | |
| 90 | 0091 | GRTFSNLAM | 90 | IWSDNT | 181 | GVARDSRSY | 272 | Prgl |
| | | | | | | YNFRLNQED | | |
| | | | | | | EYDY | | |
| 91 | 0092 | GRTFSSYAM | 91 | IRWTRSST | 182 | AASHGIGRV | 273 | Prgl |
| | | | | | | VAESLYDY | | |

TABLE 2
unique SEQ IDs for CDRs of the V_{H}H antibodies of this disclosure

| | | | | | CDR2 | | | |
|---|---|---|---|---|---|---|---|---|
| | | | | | SEQ ID | | | |
| | | | | | NO: | | CDR3 | |
| | | CDR1 Amino | CDR1 | CDR2 | CDR1 | CDR3 | SEQ | |
| | | Acid | SEQ ID | Amino Acid | SEQ ID | Amino Acid | ID | |
| Clone | NBX | Sequence | NO: | Sequence | NO: | Sequence | NO: | Antigen |
| 92 | 0100 | GSIFSTNLM | 765 | ITSGGNT | 769 | AAQTLGSSY | 773 | FimA |
| | | | | | | YDA | | |
| 93 | 0104 | GVAFNSRIM | 766 | ITSGGST | 770 | NIRNY | 774 | Prgl |
| 94 | 0105 | GRTFNTYYM | 767 | IRWSDGGT | 771 | NANVYDGN | 775 | Prgl |
| | | | | | | RWRTY | | |
| 95 | 0108 | RGTFRTYSM | 768 | ITWNGKYT | 772 | AANPIPTAQ | 776 | Prgl |
| | | | | | | PPGIMAARS | | |
| | | | | | | YVH | | |
| 97 | 0200 | GRTSSSAYT | 280 | ISWSGTTT | 330 | AADRRSTIGS | 380 | FimA |
| | | | | | | PRQQYAY | | |
| 98 | 0201 | TRTSSSSYT | 281 | ISYSGTTT | 331 | AADRRSTIGS | 381 | FimA |
| | | | | | | PRQQYAY | | |
| 99 | 0202 | GRTSSSAYT | 282 | ISWSGTTT | 332 | AADRRSTIG | 382 | FimA |
| | | | | | | TPREQYAY | | |
| 100 | 0203 | GRTSPSSYT | 283 | ISWSGTTT | 333 | AADRRSTIGS | 383 | FimA |
| | | | | | | PRQQYAY | | |
| 101 | 0204 | GSTLSNYAV | 284 | ISSGGST | 334 | HTYDFQGW | 384 | FliC |
| | | | | | | GLRSDY | | |
| 102 | 0205 | GRTFSSLAM | 285 | ISRSGDYT | 335 | AATKIVTPW | 385 | FliC |
| | | | | | | TSTYYYTKAY | | |
| | | | | | | EWDY | | |
| 103 | 0206 | GRTFSSLAM | 286 | ITRSGDYT | 336 | AATKIVTPW | 386 | FliC |
| | | | | | | TSTYYYTKAY | | |
| | | | | | | EWDY | | |
| 104 | 0207 | TAI LSI DSM | 287 | IARGGST | 337 | AADPGGASP | 387 | Prgl |
| | | | | | | LS | | |
| 105 | 0208 | GDISTIDVM | 288 | IARGGTI | 338 | AVDTGSPRL | 388 | Prgl |
| | | | | | | T | | |
| 106 | 0209 | GFTFSSSIM | 289 | IPSFGSA | 339 | NTRLY | 389 | Prgl |
| 107 | 0210 | GDISSISVM | 290 | IASGGSV | 340 | AVDTGSPRL | 390 | Prgl |
| | | | | | | T | | |
| 108 | 0211 | GFTFSTN IL | 291 | ITPFGSA | 341 | NTQLY | 391 | Prgl |
| 109 | 0212 | TSI LSI NAM | 292 | IAPGGTT | 342 | AADPGGQS | 392 | Prgl |
| | | | | | | PLS | | |
| 110 | 0213 | GSISSITAM | 293 | IARGGMI | 343 | AVDNGDPRL | 393 | Prgl |
| | | | | | | H | | |
| 111 | 0214 | GSISSITAM | 294 | IARGGMT | 344 | ALDNGDPRL | 394 | Prgl |
| | | | | | | H | | |
| 112 | 0215 | GFTFSSAIM | 295 | IPSFGSA | 345 | NTRLY | 395 | Prgl |
| 113 | 0216 | TSILSIDAM | 296 | IARGGST | 346 | AADPGGAS | 396 | Prgl |
| | | | | | | GLS | | |
| 114 | 0217 | GSISSITAM | 297 | IARGGMT | 347 | ALYNGDPRL | 397 | Prgl |
| | | | | | | H | | |
| 115 | 0218 | GSAFSGDAM | 298 | ISSGAIT | 348 | NRIQAVLRG | 398 | Prgl-SipD |
| | | | | | | NSG | | |
| 116 | 0219 | GSAFSGGDAM | 299 | ISSGGIA | 349 | NSITAVLRG | 399 | Prgl-SipD |
| | | | | | | NSG | | |
| 117 | 0220 | GSAFSGDAM | 300 | ISSGGIP | 350 | NSISAVLRG | 400 | Prgl-SipD |
| | | | | | | NGV | | |
| 118 | 0221 | GLTFNNYAM | 301 | ISRDGTNT | 351 | GVGRGTGY | 401 | Prgl-SipD |
| | | | | | | AYTAINEYDY | | |
| | | | | | | SK | | |
| 119 | 0222 | GIDSSFYVM | 302 | LGTPDSA | 352 | YGLYRQVY | 402 | Prgl-SipD |
| 120 | 0223 | GIDSSFYVM | 303 | ISSADSP | 353 | YGLYRQVH | 403 | Prgl-SipD |
| 121 | 0224 | GLTFSSYAM | 304 | IGWSGGST | 354 | AARRTTAW | 404 | Prgl-SipD |
| | | | | | | GKGTDY | | |
| 122 | 0225 | ESIFSRNA | 305 | IGSDGST | 355 | RVVLATSPY | 405 | Prgl-SipD |
| | | | | | | NY | | |
| 123 | 0226 | GITSSLYVM | 306 | INSGDSP | 356 | YGLYRQVH | 406 | Prgl-SipD |
| 124 | 0227 | GLTFNNYAM | 307 | ISRDGTST | 357 | GVGRGSGY | 407 | Prgl-SipD |
| | | | | | | AYSAINEYDY | | |
| | | | | | | SS | | |
| 125 | 0228 | GIDSSFYVM | 308 | ISMTSADSP | 358 | YGLYRQVH | 408 | Prgl-SipD |
| 126 | 0229 | GSGILFRISA | 309 | ISSGGST | 359 | NIVGRTDS | 409 | Prgl-SipD |
| 127 | 0230 | ARTLSNYAM | 310 | ISRSGGSI | 360 | GRARGTGYA | 410 | Prgl-SipD |
| | | | | | | YTALNQYDY | | |
| | | | | | | DY | | |
| 128 | 0231 | GSAFSGDAM | 311 | ISSGGIT | 361 | NSIKAVLRG | 411 | Prgl-SipD |
| | | | | | | NSG | | |
| 129 | 0232 | GLTFHNYAM | 312 | ISRDGTNT | 362 | GVGRGSGY | 412 | Prgl-SipD |
| | | | | | | AYTAINEYDY | | |
| | | | | | | SK | | |
| 130 | 0233 | GSAFSGDAM | 313 | ISSGHIT | 363 | NSITAVLRG | 413 | Prgl-SipD |
| | | | | | | NSG | | |
| 131 | 0234 | GRTFSTYA | 314 | ISRSGDNI | 364 | GRARGTGYA | 414 | Prgl-SipD |
| | | | | | | HTALNQYDY | | |
| | | | | | | DY | | |
| 132 | 0235 | GSAFSGDAM | 315 | ISSGGIE | 365 | NLIKAVLRG | 415 | Prgl-SipD |
| | | | | | | NSG | | |
| 133 | 0236 | GLTFNNYAM | 316 | ISRDGTNT | 366 | GVGRGTGY | 416 | Prgl-SipD |
| | | | | | | AYTAIREHDY | | |
| | | | | | | SS | | |
| 134 | 0237 | GSAFSGDAM | 317 | ISSGGIT | 367 | NSITAVLRG | 417 | SipD |
| | | | | | | NSG | | |
| 135 | 0238 | GSAFSGDAM | 318 | ISSGGIA | 368 | NTIKAVLRG | 418 | SipD |
| | | | | | | NAG | | |
| 136 | 0239 | GSAFSGDAM | 319 | ISSGAIT | 369 | NSITAVLRG | 419 | SipD |
| | | | | | | NS | | |
| 137 | 0240 | GSAFSGDAM | 320 | ISSGGIT | 370 | NIISAVLRGN | 420 | SipD |
| | | | | | | GG | | |
| 138 | 0241 | ISGFSGDAM | 321 | ISSGGIT | 371 | NTITGVLRG | 421 | SipD |
| | | | | | | NSG | | |
| 139 | 0242 | GIISSAYVM | 322 | ITSGDSP | 372 | YGLYRQVY | 422 | SipD |
| 140 | 0243 | GIAFSTYGM | 323 | ITGNGDD | 373 | NIGMY | 423 | SipD |
| 141 | 0244 | GSAFSGDAM | 324 | ISSGGIT | 374 | NSISAVLRG | 424 | SipD |
| | | | | | | NSG | | |
| 142 | 0245 | GSAFSGDAM | 325 | ISSGGIT | 375 | NSISAVLRG | 425 | SipD |
| | | | | | | NGG | | |
| 143 | 0246 | GSAFSGDAM | 326 | ISSGGIT | 376 | NSITAVLRG | 426 | SipD |
| | | | | | | NSD | | |
| 144 | 0247 | GSAFSGDAM | 327 | ISSGGIP | 377 | NIIKTVLRGN | 427 | SipD |
| | | | | | | AV | | |
| 145 | 0248 | GSAFSGGDAM | 328 | ISSGGIT | 378 | NSITAVLRG | 428 | SipD |
| | | | | | | NSG | | |
| 146 | 0249 | GITFSSDAM | 329 | ISSGDIT | 379 | NTITRLLYG | 429 | SipD |
| | | | | | | MDY | | |
| 147 | 0250 | GFTLDGYAI | 573 | IIYRDGSP | 574 | AARPGGACS | 575 | FimA |
| | | | | | | RYPSNYDT | | |

### Generic Framework Regions

A full heavy chain variable region will generally comprise the structure according to Framework1-CDR1-Framework2-CDR2-Framework 3-CDR3-Framework 4. The CDRs listed in tables 1 and 2 can be combined with the framework regions listed in SEQ ID NOs: 573-613 (FR1); 614-669 (FR2); 670-752 (FR3); and 753-764 (FR4) for recombinant construction of an anti-Salmonella V_{H}H.

### In Vivo Stability of V_{H}Hs

For passive immunization or oral administration, the V_{H}Hs described herein should be stable in a GI environment. The V_{H}Hs of this disclosure exhibit stability in GI tract fluids from a chicken. In certain instances, the V_{H}Hs exhibit less than 50%, 40%, 30%, 20%, 10%, 5% or less degradation when incubated at 42° for 30 minutes in gizzard extract. In certain embodiments, the V_{H}Hs exhibit less than 50%, 40%, 30%, 20%, 10%, 5% or less degradation when incubated at 37° at a pH of about 3 for 30 minutes to an hour. In certain embodiments, the V_{H}Hs exhibit less than 50%, 40%, 30%, 20%, 10%, 5% or less degradation when incubated with human gastric fluid or an acceptable substitute for 30 minutes to an hour at 37°.

### Bacterial Antigens

Flagella/FliC: Flagella are large, whip-like, multi-component structures that are anchored to the cell envelope of the bacteria and project outside of the cell. Each flagellum consists of tens of thousands of molecules of the FliC subunits plus a number of other accessory proteins. An individual Salmonella cell possesses many flagella found all over the cell body (peritrichous flagella). Bacteria rotate their flagella in an energy-dependent manner, to propel themselves (swimming motility) towards attractants and away from repellents.

FimA: Type-1 fimbriae are thin appendages found on the surfaces of many bacteria and project out from the cell. Type-1 fimbriae consists of thousands of subunits of FimA plus a number of other accessory proteins. Type-1 fimbriae allow Salmonella to bind to eukaryotic cells via a high-affinity interaction between one of the accessory proteins, the lectin domain of FimH, and mannose sugars located on the outside of eukaryotic cells. This interaction allows Salmonella to adhere to eukaryotic tissues.

PrgI and SipD: The Type III Secretion System is a needle-like protein complex that protrudes from the surface of Gram-negative bacteria, interacts with host cell membranes, and injects effector proteins into the host cytoplasm. The Type III Secretion System needle is constructed from hundreds of molecules of PrgI. The tip of the needle is constructed from a ring of SipD proteins, which is necessary for the interaction between the secretion system and the eukaryotic cells.

In certain embodiments, the antibodies for use with the compositions and methods described herein specifically bind to any biomolecule of Enterobacteriaceae. In certain embodiments, the antibodies for use with the compositions and methods described herein specifically bind to any biomolecule of Salmonella. In certain embodiments, the biomolecule of Salmonella may function in bacterial motility. In certain embodiments, the Salmonella biomolecule may be a component of the flagellum. In certain embodiments, the Salmonella biomolecule may be a protein or polypeptide component of the flagellum. In certain embodiments, the antibody may specifically bind the Salmonella flagellin protein (FliC). In certain embodiments, the biomolecule of Salmonella may function in bacterial adhesion. In certain embodiments, the antibody may specifically bind the Salmonella fimbrial protein subunit A protein (FimA). In certain embodiments, the biomolecule of Salmonella may function in bacterial invasion. In certain embodiments, the antibody may specifically bind the Salmonella PrgI protein. In certain embodiments, the antibody may specifically bind the Salmonella SipD protein. In certain embodiments, the antibody does not specifically bind bacterial lipopolysaccharide (LPS). In certain embodiments, the antibody does not specifically bind bacterial O-antigen. In certain embodiments, the antibody does not specifically bind FimH or OmpD. The specific antigens disclosed herein FliC, FimA, and PrgI are highly variable among Salmonella serovars, for example, the sequence identity between Enteritidis and Newport FliC protein is 48%. It will be appreciated by one of skill in the art that antigens derived from the same genes of different serovars will perform similarly. In certain embodiments, the antibody may be raised against a component of Salmonella flagellum. In certain embodiments, the antibody may be raised against a protein or polypeptide with at least 40% sequence identity to SEQ ID NO: 274 or SEQ ID NO: 277. In certain embodiments, the antibody may be raised against a protein or polypeptide with at least 40% sequence identity to SEQ ID NO: 275 or SEQ ID NO: 278. In certain embodiments, the antibody may be raised against a protein or polypeptide with at least 40% sequence identity to SEQ ID NO: 276 or SEQ ID NO: 279. In certain embodiments, the antibody may be raised against a component of Salmonella flagellum. In certain embodiments, the antibody may be raised against a protein or polypeptide with at least 50% sequence identity to SEQ ID NO: 274 or SEQ ID NO: 277. In certain embodiments, the antibody may be raised against a protein or polypeptide with at least 50% sequence identity to SEQ ID NO: 275 or SEQ ID NO: 278. In certain embodiments, the antibody may be raised against a protein or polypeptide with at least 50% sequence identity to SEQ ID NO: 276 or SEQ ID NO: 279. In certain embodiments, the antibody may be raised against a component of Salmonella flagellum. In certain embodiments, the antibody may be raised against a protein or polypeptide with at least 60% sequence identity to SEQ ID NO: 274 or SEQ ID NO: 277. In certain embodiments, the antibody may be raised against a protein or polypeptide with at least 60% sequence identity to SEQ ID NO: 275 or SEQ ID NO: 278. In certain embodiments, the antibody may be raised against a protein or polypeptide with at least 60% sequence identity to SEQ ID NO: 276 or SEQ ID NO: 279. In certain embodiments, the antibody may be raised against a component of Salmonella flagellum. In certain embodiments, the antibody may be raised against a protein or polypeptide with at least 70% sequence identity to SEQ ID NO: 274 or SEQ ID NO: 277. In certain embodiments, the antibody may be raised against a protein or polypeptide with at least 70% sequence identity to SEQ ID NO: 275 or SEQ ID NO: 278. In certain embodiments, the antibody may be raised against a protein or polypeptide with at least 70% sequence identity to SEQ ID NO: 276 or SEQ ID NO: 279. In certain embodiments, the antibody may be raised against a protein or polypeptide with at least 80% sequence identity to SEQ ID NO: 274 or SEQ ID NO: 277. In certain embodiments, the antibody may be raised against a protein or polypeptide with at least 80% sequence identity to SEQ ID NO: 275 or SEQ ID NO: 278. In certain embodiments, the antibody may be raised against a protein or polypeptide with at least 80% sequence identity to SEQ ID NO: 276 or SEQ ID NO: 279. In certain embodiments, the antibody may be raised against a protein or polypeptide with at least 90% sequence identity to SEQ ID NO: 274 or SEQ ID NO: 277. In certain embodiments, the antibody may be raised against a protein or polypeptide with at least 90% sequence identity to SEQ ID NO: 275 or SEQ ID NO: 278. In certain embodiments, the antibody may be raised against a protein or polypeptide with at least 90% sequence identity to SEQ ID NO: 276 or SEQ ID NO: 279. In certain embodiments, the antibody may be raised against a protein or polypeptide with at least 95% sequence identity to SEQ ID NO: 274 or SEQ ID NO: 277. In certain embodiments, the antibody may be raised against a protein or polypeptide with at least 95% sequence identity to SEQ ID NO: 275 or SEQ ID NO: 278. In certain embodiments, the antibody may be raised against a protein or polypeptide with at least 95% sequence identity to SEQ ID NO: 276 or SEQ ID NO: 279. In certain embodiments, the antibody may be raised against a protein or polypeptide with at least 98% sequence identity to SEQ ID NO: 274 or SEQ ID NO: 277. In certain embodiments, the antibody may be raised against a protein or polypeptide with at least 98% sequence identity to SEQ ID NO: 275 or SEQ ID NO: 278. In certain embodiments, the antibody may be raised against a protein or polypeptide with at least 98% sequence identity to SEQ ID NO: 276 or SEQ ID NO: 279. In certain embodiments, the antibody may be raised against a protein or polypeptide with 100% sequence identity to SEQ ID NO: 274 or SEQ ID NO: 277. In certain embodiments, the antibody may be raised against a protein or polypeptide with 100% sequence identity to SEQ ID NO: 275 or SEQ ID NO: 278. In certain embodiments, the antibody may be raised against a protein or polypeptide with 100% sequence identity to SEQ ID NO: 276 or SEQ ID NO: 279. In certain embodiments, the antibody may be raised against a protein or polypeptide with at least 40% sequence identity to SEQ ID NO: 460 or SEQ ID NO: 461. In certain embodiments, the antibody may be raised against a protein or polypeptide with at least 50% sequence identity to SEQ ID NO: 460 or SEQ ID NO: 461. In certain embodiments, the antibody may be raised against a protein or polypeptide with at least 60% sequence identity to SEQ ID NO: 460 or SEQ ID NO: 461. In certain embodiments, the antibody may be raised against a protein or polypeptide with at least 70% sequence identity to SEQ ID NO: 460 or SEQ ID NO: 461. In certain embodiments, the antibody may be raised against a protein or polypeptide with at least 80% sequence identity to SEQ ID NO: 460 or SEQ ID NO: 461. In certain embodiments, the antibody may be raised against a protein or polypeptide with at least 90% sequence identity to SEQ ID NO: 460 or SEQ ID NO: 461. In certain embodiments, the antibody may be raised against a protein or polypeptide with at least 95% sequence identity to SEQ ID NO: 460 or SEQ ID NO: 461. In certain embodiments, the antibody may be raised against a protein or polypeptide with at least 98% sequence identity to SEQ ID NO: 460 or SEQ ID NO: 461. In certain embodiments, the antibody may be raised against a protein or polypeptide with 100% sequence identity to SEQ ID NO: 460 or SEQ ID NO: 461.

### Reduction in Bacterial Motility

Salmonella bacteria possess the ability to move towards attractants and away from repellents by means of flagella-dependent swimming motility. Within the GI tract of an animal Salmonella bacteria encounter numerous stimuli to which the bacteria would move towards or away from. Several studies have suggested that correlation exists between motility and the ability of Salmonella to colonize the GI tract. In certain embodiments, the antibodies for use with the compositions and methods described herein reduce bacterial motility. In certain embodiments, the antibody that reduces motility targets a component of the bacterial flagellum. In certain embodiments, the antibody that reduces motility targets the FliC antigen. In certain embodiments, the antibodies for use with the compositions and methods described herein reduce bacterial motility in an in vitro assay. In certain embodiments, the antibody reduces bacterial motility compared to a negative control antibody. In certain embodiments, the antibody reduces bacterial motility by at least 10% compared to a negative control antibody. In certain embodiments, the antibody reduces bacterial motility by at least 20% compared to a negative control antibody. In certain embodiments, the antibody reduces bacterial motility by at least 30% compared to a negative control antibody. In certain embodiments, the antibody reduces bacterial motility by at least 40% compared to a negative control antibody. In certain embodiments, the antibody reduces bacterial motility by at least 50% compared to a negative control antibody. In certain embodiments, the antibody reduces bacterial motility by at least 60% compared to a negative control antibody. In certain embodiments, the antibody reduces bacterial motility by at least 70% compared to a negative control antibody. In certain embodiments, motility is reduced at a concentration of less than 10 μM, 25 μM, 50 μM, 75 μM, 100 μM, 125 μM, 150 μM, 175 μM, 200 μM, 300 μM, 400 μM, or 500 μM. In certain embodiments, a negative control antibody is an isotype control, a non-targeting antibody, or a pre-immune serum.

### Reduction in Bacterial Adhesion

Salmonella bacteria use adhesins located on their surface to contact and attach to epithelial cells prior to invading the epithelial cells. Salmonella possesses several putative adhesins that are thought to participate in adhesion to animal epithelial cells. Reduction in adhesion may contribute to decreased ability to colonize an animal. In certain embodiments, the antibodies for use with the compositions and methods described herein reduce bacterial adhesion. In certain embodiments, the antibody that reduces adhesion targets a component of the bacterial fimbriae. In certain embodiments, the antibody that reduces adhesion targets the FimA antigen. In certain embodiments, the antibodies for use with the compositions and methods described herein reduce bacterial adhesion in an in vitro assay. In certain embodiments, the antibody reduces bacterial adhesion compared to a negative control antibody. In certain embodiments, the antibody reduces bacterial adhesion by at least 10% compared to a negative control antibody. In certain embodiments, the antibody reduces bacterial adhesion by at least 20% compared to a negative control antibody. In certain embodiments, the antibody reduces bacterial adhesion by at least 30% compared to a negative control antibody. In certain embodiments, the antibody reduces bacterial adhesion by at least 40% compared to a negative control antibody. In certain embodiments, the antibody reduces bacterial adhesion by at least 50% compared to a negative control antibody. In certain embodiments, the antibody reduces bacterial adhesion by at least 60% compared to a negative control antibody. In certain embodiments, the antibody reduces bacterial adhesion by at least 70% compared to a negative control antibody. In certain embodiments, adhesion is reduced at a concentration of less than 10 μM, 25 μM, 50 μM, 75 μM, 100 μM, 125 μM, 150 μM, 175 μM, 200 μM, 300 μM, 400 μM, or 500 μM. In certain embodiments, a negative control antibody is an isotype control, a non-targeting antibody, or a pre-immune serum.

### Reduction in Bacterial Invasion

To successfully colonize the GI tract of an animal, Salmonella bacteria invade epithelial cells lining the GI tract. Within vacuoles of the epithelial cells, Salmonella can manipulate the environment to enable survival and bacterial proliferation. Loss of epithelial cell invasion has been correlated with decreased animal colonization. In certain embodiments, the antibodies for use with the compositions and methods described herein reduce bacterial invasion. In certain embodiments, the antibody that reduces invasion targets a component of the bacterial needle complex. In certain embodiments, the antibody that reduces invasion targets the PrgI or SipD antigen. In certain embodiments, the antibodies for use with the compositions and methods described herein reduce bacterial invasion in an in vitro assay. In certain embodiments, the antibody reduces bacterial invasion compared to a negative control antibody. In certain embodiments, the antibody reduces bacterial invasion by at least 10% compared to a negative control antibody. In certain embodiments, the antibody reduces bacterial invasion by at least 20% compared to a negative control antibody. In certain embodiments, the antibody reduces bacterial invasion by at least 30% compared to a negative control antibody. In certain embodiments, the antibody reduces bacterial invasion by at least 40% compared to a negative control antibody. In certain embodiments, the antibody reduces bacterial invasion by at least 50% compared to a negative control antibody. In certain embodiments, the antibody reduces bacterial invasion by at least 60% compared to a negative control antibody. In certain embodiments, the antibody reduces bacterial invasion by at least 70% compared to a negative control antibody. In certain embodiments, invasion is reduced at a concentration of less than 10 μM, 25 μM, 50 μM, 75 μM, 100 μM, 125 μM, 150 μM, 175 μM, 200 μM, 300 μM, 400 μM, or 500 μM. In certain embodiments, a negative control antibody is an isotype control, a non-targeting antibody, or a pre-immune serum.

### Reduction in Biofilm Formation

Biofilms are multi-cellular bacterial communities that can protect bacteria from host defenses and antibiotics. Salmonella can form biofilms on many types of surfaces including chicken intestinal epithelium and this is thought contribute to colonization and disease in animals. Many putative Salmonella virulence factors have been implicated in biofilm formation. In certain embodiments, the antibodies for use with the compositions and methods described herein reduce biofilm formation. In certain embodiments, the antibodies for use with the compositions and methods described herein reduce biofilm formation in an in vitro assay. In certain embodiments, the antibody reduces biofilm formation by at least 10%. In certain embodiments, the antibody reduces biofilm formation by at least 20%. In certain embodiments, the antibody reduces biofilm formation by at least 30%. In certain embodiments, the antibody reduces biofilm formation by at least 40%. In certain embodiments, the antibody reduces biofilm formation by at least 50%. In certain embodiments, the antibody reduces biofilm formation by at least 60%. In certain embodiments, the antibody reduces biofilm formation by at least 70%. In certain embodiments, the antibody reduces biofilm formation by at least 80%. In certain embodiments, the antibody reduces biofilm formation by at least 90%. In certain embodiments, invasion is reduced at a concentration of less than 10 μM, 25 μM, 50 μM, 75 μM, 100 μM, 125 μM, 150 μM, 175 μM, 200 μM, 300 μM, 400 μM, or 500 μM. In certain embodiments, a negative control antibody is an isotype control, a non-targeting antibody, or a pre-immune serum.

### Multimers of V_{H}H Proteins

The antibodies and V_{H}H fragments of this disclosure are useful as multimers. Also known as protein concatemers, these V_{H}H fragments can be linked together in a single polypeptide comprising 2, 3, 4, 5, 6, 7, 8, 9, 10 or more V_{H}Hs. The V_{H}Hs can be linked using any of the linkers in Table 3. In certain embodiments, a multimer comprises, the same V_{H}H repeated 1, 2, 3, 4, 5, 6, 7, 8, 9, 10 or more times. In certain embodiments, a multimer comprises 1, 2, 3, 4, 5, 6, 7, 8, 9, 10 or more different V_{H}Hs. In a certain embodiment, the multimer comprises an amino acid sequence at least 80%, 85%, 90%, 95%, 97%, 99%, or 100% identical to the sequence set forth in SEQ ID NO 462. In a certain embodiment, the multimer comprises an amino acid sequence at least 80%, 85%, 90%, 95%, 97%, 99%, or 100% identical to the sequence set forth in SEQ ID NO 463. In a certain embodiment, the multimer comprises an amino acid sequence at least 80%, 85%, 90%, 95%, 97%, 99% or 100% identical to the sequence set forth in SEQ ID NO 464. In a certain embodiment, the multimer comprises an amino acid sequence at least 80%, 85%, 90%, 95%, 97%, 99%, or 100% identical to the sequence set forth in SEQ ID NO 465.

TABLE 3
Exemplary linkers for the multimeric NBX of this
disclosure

| | SEQ ID | |
|---|---|---|
| Linker Name | NO: | Linker Amino Acid Sequence |
| 1X G4S Linker | 445 | GGGGS |
| 3X G4S Linker | 446 | GGGGSGGGGSGGGGS |
| 5X G4S Linker | 447 | GGGGSGGGGSGGGGSGGGGSGGGGS |
| 2X G3S Linker | 448 | GGGSGGGS |
| Glycine Only | 449 | GGGGGGGGGG |
| Linker | | |
| Helical Linker | 450 | GGAEAAAKEAAAKEAAAKEAAAKEA |
| | | AAKGG |
| Rigid Proline | 451 | GGGAAPAAAPAKQEAAAPAPAAKAE |
| Linker | | APAAAPAATGG |
| Cleavable Linker | 452 | GGGGSGGLGGSGGGGS |

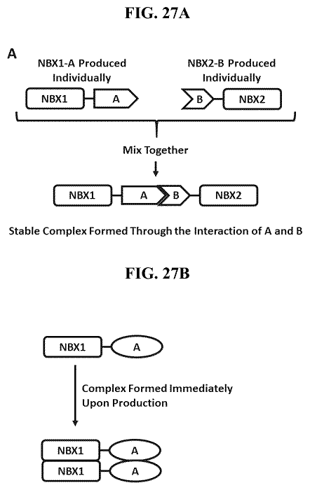

The antibody and V_{H}H fragment multimers of this disclosure can also comprise two separate polypeptides forming a polypeptide complex via protein-protein interactions. FIG. 27A and FIG. 27B show a schematic to describe the principle behind NBX complex formation via protein-protein interactions. (A) NBX1-A and NBX2-B are two constructs expressed and purified individually as component polypeptides. Protein domains A and B form a highly stable interaction. When NBX1-A and NBX2-B are mixed together, a complex is formed, driven by the interaction of A and B, that keeps NBX1 and NBX2 closely associated. (B) Multimers of the same NBX can come together immediately upon production if protein domain A naturally self-oligomerizes. This interaction can also be provided by a protein-small molecule interaction, or a small molecule-small molecule interaction, such as for example biotin-streptavidin. In certain embodiments, the V_{H}Hs can be complexed with the aid of any of the heterocomplex forming sequences in SEQ ID NO 453-456 shown in Table 4. In certain embodiments, the V_{H}Hs can be complexed with the aide of any of the complex forming sequences in SEQ ID NO 457-460 shown in Table 4. In a certain embodiment, the multimer comprises an amino acid sequence at least 80%, 85%, 90%, 95%, 97%, or 99% identical to the sequence set forth in SEQ ID NO 466. In a certain embodiment, the multimer comprises an amino acid sequence at least 80%, 85%, 90%, 95%, 97%, or 99% identical to the sequence set forth in SEQ ID NO 467. In a certain embodiment, the multimer comprises an amino acid sequence at least 80%, 85%, 90%, 95%, 97%, or 99% identical to the sequence set forth in SEQ ID NO 468. In a certain embodiment, the multimer comprises an amino acid sequence at least 80%, 85%, 90%, 95%, 97%, or 99% identical to the sequence set forth in SEQ ID NO 469. In a certain embodiment, the multimer comprises an amino acid sequence at least 80%, 85%, 90%, 95%, 97%, or 99% identical to the sequence set forth in SEQ ID NO 470. In a certain embodiment, the multimer comprises an amino acid sequence at least 80%, 85%, 90%, 95%, 97%, or 99% identical to the sequence set forth in SEQ ID NO 471. In a certain embodiment, the multimer comprises an amino acid sequence at least 80%, 85%, 90%, 95%, 97%, or 99% identical to the sequence set forth in SEQ ID NO 472. In a certain embodiment, the multimer comprises an amino acid sequence at least 80%, 85%, 90%, 95%, 97%, or 99% identical to the sequence set forth in SEQ ID NO 473. In a certain embodiment, the multimer comprises an amino acid sequence at least 80%, 85%, 90%, 95%, 97%, or 99% identical to the sequence set forth in SEQ ID NO 474.

In certain embodiments, the multimers formed herein either as a concatemers or protein complexes show an increase in affinity for target antigen. In certain embodiments, this increase in affinity is at least 2-fold more than any V_{H}H by itself. In certain embodiments, this increase in affinity is at least 3-fold more than any V_{H}H by itself. In certain embodiments, this increase in affinity is at least 5-fold more than any V_{H}H by itself. In certain embodiments, this increase in affinity is at least 10-fold more than any V_{H}H by itself. In certain embodiments, this increase in affinity is at least 20-fold more than any V_{H}H by itself. In certain embodiments, this increase in affinity is at least 50-fold more than any V_{H}H by itself. In certain embodiments, this increase in affinity is at least 100-fold more than any V_{H}H by itself. In certain embodiments, this increase in affinity is at least 200-fold more than any V_{H}H by itself. In certain embodiments, this increase in affinity is at least 300-fold more than any V_{H}H by itself.

TABLE 4
Exemplary protein A and protein B partners
useful in the construction of NBX multimers.

| | SEQ | Protein A | SEQ | | Protein B |
|---|---|---|---|---|---|
| Protein | ID | Amino Acid | ID | Protein | Amino Acid |
| A | NO: | Sequence | O: | B | Sequence |
| E9 | 453 | MELKHSISDY | 454 | Colicin | MESKRNKP |
| Immunity | | TEAEFLQLVT | | E9 | GKATGKGK |
| Protein | | TICNADTSSE | | | PVGDKWLD |
| | | EELVKLVTHF | | | DAGKDSGA |
| | | EEMTEHPSGS | | | PIPDRIADKL |
| | | DLIYYPKEGD | | | RDKEFKSFD |
| | | DDSPSGIVNT | | | DFRKAVWE |
| | | VKQWRAANGK | | | EVSKDPELS |
| | | SGFKQG | | | KNLNPCNKS |
| | | | | | SVSKGYSPF |
| | | | | | TPKNQQVG |
| | | | | | GRKVYELHH |
| | | | | | DKPISQGGE |
| | | | | | VYDMDNIR |
| | | | | | VTTPKRHIDI |
| | | | | | HRGK |
| Fos | 455 | LTDTLQAETD | 456 | Jun | RIARLEEKVK |
| Leucine | | QLEDEKSALQ | | Leucine | TLKAQNSEL |
| Zipper | | TEIANLLKEK | | Zipper | ASTANMLR |
| | | EKLEFILAA | | | EQVAQLKQ |
| | | | | | KVMN |

Protein A self-oligomerizers useful in the
construction of NBX multimers.

| | | Protein | |
|---|---|---|---|
| Protein | | Self- | |
| | | Oligomer- | |
| | | ization | Protein A |
| A | | Status | Amino Acid Sequence |
| GCN4 PIL | 457 | Dimer | RMKQLEDKIEELLSKIYHL |
| | | | ENEIARLKKLIGER |
| GCN4 PII | 457 | Trimer | RMKQIEDKIEEILSKIYHI |
| | | | ENEIARIKKLIGER |
| GCN4 PLI | 458 | Tetramer | RMKQIEDKLEEILSKLYHI |
| | | | ENELARIKKLLG |
| Kv1.2 T1 | 460 | Tetramer | ERVVINISGLRFETQLKTL |
| | | | AQFPETLLGDPKKRMRYFD |
| | | | PLRNEYFFDRNRPSFDAIL |
| | | | YYYQSGGRLRRPVNVPLDI |
| | | | FSEEIRFYELG |

### Routes of Administration

In certain embodiments, the heavy chain antibodies and V_{H}Hs for use with the compositions and methods described herein are administered to an animal in any suitable manner. In certain embodiments, the antibodies for use with the compositions and methods described herein are administered to the alimentary canal of an animal. In certain embodiments, the antibodies for use with the compositions and methods described herein are administered to the alimentary canal of an animal orally. In certain embodiments, the antibodies for use with the compositions and methods described herein are administered to the alimentary canal of an animal in liquid form. In certain embodiments, the antibodies for use with the compositions and methods described herein are administered to the alimentary canal of an animal in solid form. In certain embodiments, the antibodies for use with the compositions and methods described herein are administered to the exterior surface of an animal. In certain embodiments, the antibodies for use with the compositions and methods described herein are administered to the exterior surface of an animal in a liquid formulation. In certain embodiments, the antibodies for use with the compositions and methods described herein are administered to the exterior surface of an animal in a spray formulation. In certain embodiments, the antibodies for use with the compositions and methods described herein are administered to the exterior surface of an animal in a gelatinized spray formulation. In certain embodiments, the antibodies for use with the compositions and methods described herein are administered by an injection.

In certain embodiments, the heavy chain antibodies and V_{H}Hs for use with the compositions and methods described herein are administered to a human subject in any suitable manner. In certain embodiments, the antibodies for use with the compositions and methods described herein are administered to the alimentary canal of a human subject. In certain embodiments, the antibodies for use with the compositions and methods described herein are administered to the alimentary canal of a human subject orally. In certain embodiments, the antibodies for use with the compositions and methods described herein are administered to the alimentary canal of a human subject in liquid form. In certain embodiments, the antibodies for use with the compositions and methods described herein are administered to the alimentary canal of a human subject in solid form. In certain embodiments, the antibodies for use with the compositions and methods described herein are administered by an injection. In certain embodiments, the antibodies for use with the compositions and methods described herein are administered intravenously. In certain embodiments, the antibodies for use with the compositions and methods described herein are administered in food or a nutritional supplement.

### Antibody Mixtures, Concentrations and Dosage Schedules

In certain embodiments, one or a plurality of heavy chain antibodies or V_{H}H comprising polypeptides is administered in a composition to an animal in any suitable manner described herein. In certain embodiments, a mixture of two or more antibodies that target any combination of two virulence factors comprising of bacterial motility, adhesion, invasion, or biofilm formation are administered simultaneously. In certain embodiments, a mixture of three or more antibodies that target any combination of three virulence factors involved in bacterial motility, adhesion, invasion, or biofilm formation are administered simultaneously. In certain embodiments, a mixture of antibodies that target any combination of flagella/FliC, PrgI, or FimA antigens are administered to the animal. In certain embodiments, more than one distinct antibody is administered to the animal. In certain embodiments, more than two distinct antibodies are administered to the animal. In certain embodiments, more than three distinct antibodies are administered to the animal. In certain embodiments, more than four distinct antibodies are administered to the animal. In certain embodiments, more than five distinct antibodies are administered to the animal. In certain embodiments, the antibodies are administered concurrently. In certain embodiments, the antibodies are administered sequentially. In certain embodiments, antibodies are administered to an animal at a concentration in excess of 1 mg/kg of body weight. In certain embodiments, antibodies are administered to an animal at a concentration in excess of 5 mg/kg of body weight. In certain embodiments, antibodies are administered to an animal at a concentration in excess of 10 mg/kg of body weight. In certain embodiments, antibodies are administered to an animal at a concentration in excess of 50 mg/kg of body weight. In certain embodiments, antibodies are administered to an animal at a concentration in excess of 100 mg/kg of body weight. In certain embodiments, antibodies are administered to an animal at a concentration less than 1 mg/kg of body weight. In certain embodiments, antibodies are administered to an animal at a concentration less than 500 mg/kg of body weight. In certain embodiments, antibodies are administered to an animal at a concentration less than 100 mg/kg of body weight. In certain embodiments, antibodies are administered to an animal at a concentration less than 50 mg/kg of body weight. In certain embodiments, antibodies are administered to an animal at a concentration less than 10 mg/kg of body weight. In certain embodiments, antibodies are administered once a day. In certain embodiments, antibodies are administered twice a day. In certain embodiments, antibodies are administered once a week. In certain embodiments, antibodies are administered twice a week. In certain embodiments, antibodies are administered three times a week. In certain embodiments, antibodies are administered four times a week. In certain embodiments, antibodies are administered once a month. In certain embodiments, antibodies are administered twice a month. In certain embodiments, antibodies are administered three times a month. In certain embodiments, antibodies are administered four times a month.

In certain embodiments, one or a plurality of antibodies or V_{H}H comprising polypeptides is administered to a human subject in any suitable manner described herein. In certain embodiments, a mixture of any one or more antibodies that target any combination of virulence factors comprising bacterial motility, adhesion, invasion, or biofilm formation are administered simultaneously. In certain embodiments, a mixture of two or more antibodies that target any combination of two virulence factors comprising bacterial motility, adhesion, invasion, or biofilm formation are administered simultaneously. In certain embodiments, a mixture of three or more antibodies that target any combination of three virulence factors involved in bacterial motility, adhesion, invasion, or biofilm formation are administered simultaneously. In certain embodiments, a mixture of antibodies that target any combination of flagella/FliC, PrgI, or FimA antigens are administered to a human subject. In certain embodiments, more than one distinct antibody is administered to a human subject. In certain embodiments, more than two distinct antibodies are administered to a human subject. In certain embodiments, more than three distinct antibodies administered to a human subject. In certain embodiments, more than four distinct antibodies are administered to a human subject. In certain embodiments, more than five distinct antibodies are administered to a human subject. In certain embodiments, the antibodies are administered concurrently. In certain embodiments, the antibodies are administered sequentially. In certain embodiments, antibodies are administered to a human subject at a concentration in excess of 1 mg/kg of body weight. In certain embodiments, antibodies are administered to a human subject at a concentration in excess of 5 mg/kg of body weight. In certain embodiments, antibodies are administered to a human subject at a concentration in excess of 10 mg/kg of body weight. In certain embodiments, antibodies are administered to a human subject at a concentration in excess of 50 mg/kg of body weight. In certain embodiments, antibodies are administered to a human subject at a concentration in excess of 100 mg/kg of body weight. In certain embodiments, antibodies are administered to a human subject at a concentration less than 1 mg/kg of body weight. In certain embodiments, antibodies are administered to a human subject at a concentration less than 500 mg/kg of body weight. In certain embodiments, antibodies are administered to a human subject at a concentration less than 100 mg/kg of body weight. In certain embodiments, antibodies are administered to a human subject at a concentration less than 50 mg/kg of body weight. In certain embodiments, antibodies are administered to a human subject at a concentration less than 10 mg/kg of body weight. In certain embodiments, antibodies are administered to a human subject at a concentration less than 1 mg/kg of body weight. In certain embodiments, antibodies are administered to a human subject at a concentration less than 1 μg/kg of body weight. In certain embodiments, antibodies are administered to a human subject at a concentration less than 1 ng/kg of body weight. In certain embodiments, antibodies are administered once a day. In certain embodiments, antibodies are administered twice a day. In certain embodiments, antibodies are administered once a week. In certain embodiments, antibodies are administered twice a week. In certain embodiments, antibodies are administered three times a week. In certain embodiments, antibodies are administered four times a week. In certain embodiments, antibodies are administered once a month. In certain embodiments, antibodies are administered twice a month. In certain embodiments, antibodies are administered three times a month. In certain embodiments, antibodies are administered four times a month.

### Animal Feed

In certain embodiments, the antibodies for use with the compositions and methods described herein are administered in an animal feed. In certain embodiments, the antibodies for use with the compositions and methods described herein are administered mixed with feed. In certain embodiments, the antibodies are administered mixed with feed specific for the type of animal that the antibody is administered to. In certain embodiments, the antibodies are administered mixed with a poultry feed. In certain embodiments, the antibodies are administered mixed with a chicken feed. In certain embodiments, the antibodies are administered mixed with a duck feed. In certain embodiments, the antibodies are administered mixed with a turkey feed. In certain embodiments, the antibodies are administered mixed with a goose feed. In certain embodiments, the antibodies are administered mixed with a grain. In certain embodiments, the grain is whole, milled, or ground. In certain embodiments, the grain is corn, rice, barley, wheat, soybean, alfalfa, grass, hay, straw, or a combination thereof. In certain embodiments, the antibodies are administered in water. In certain embodiments, the antibodies are administered in vitamin supplements. In certain embodiments, the antibodies are mixed with an animal feed or supplement at a concentration of between about 1 ng/kg and about 100 g/kg. In certain embodiments, the antibodies are mixed with an animal feed or supplement at a concentration of between about 1 μg/kg and about 100 g/kg. In certain embodiments, the antibodies are mixed with an animal feed or supplement at a concentration of between about 1 mg/kg and about 100 g/kg. In certain embodiments, the antibodies are mixed with an animal feed or supplement at a concentration of between about 1 mg/kg and about 10 g/kg. In certain embodiments, the antibodies are mixed with an animal feed or supplement at a concentration of between about 10 mg/kg and about 10 g/kg. In certain embodiments, the antibodies are mixed with an animal feed or supplement at a concentration of between about 100 mg/kg and about 10 g/kg. In certain embodiments, the antibodies are mixed with an animal feed or supplement at a concentration of between about 1 g/kg and about 10 g/kg. In certain embodiments, the antibodies are mixed with an animal feed or supplement at a concentration of between about 100 mg/kg and about 1 g/kg. In certain embodiments, the antibodies are mixed with an animal feed or supplement at a concentration of between about 1 mg/kg and about 1 g/kg. In certain embodiments, the antibodies are mixed with an animal feed or supplement at a concentration of between about 1 μg/kg and about 1 g/kg. In certain embodiments, the antibodies are mixed with an animal feed or supplement at a concentration of between about 1 ng/kg and about 1 g/kg. In certain embodiments, the antibodies are mixed with an animal feed or supplement at a concentration of no greater than 10 mg/kg. In certain embodiments, the antibodies are mixed with an animal feed or supplement at a concentration of no greater than 5 mg/kg. In certain embodiments, the antibodies are mixed with an animal feed or supplement at a concentration of no greater than 1 mg/kg. In certain embodiments, the antibodies are mixed with an animal feed or supplement at a concentration of no greater than 5 μg/kg. In certain embodiments, the antibodies are mixed with an animal feed or supplement at a concentration of no greater than 1 μg/kg. In certain embodiments, the antibodies are mixed with an animal feed or supplement at a concentration of no greater than 5 ng/kg. In certain embodiments, the antibodies are mixed with an animal feed or supplement at a concentration of no greater than 1 ng/kg.

### Pharmaceutically Acceptable Vehicle, Carrier, Excipient, or Diluent

In certain embodiments, described herein, are compositions of matter that comprise one or more isolated, and purified V_{H}H polypeptides and a pharmaceutically acceptable vehicle, carrier, or excipient. In certain embodiments, the pharmaceutically acceptable vehicle, carrier, or excipient comprises a pH buffer or pH modifier. In certain embodiments, the pH buffer or pH modifier comprises sodium bicarbonate, HEPES, MOPS, MEPES, phosphate buffer, succinate buffer, citric acid, ascorbic acid, or any combination thereof. In certain embodiments, the pharmaceutically acceptable vehicle, carrier or excipient comprises a salt solution. In certain embodiments, the salt solution comprises sodium chloride, potassium chloride, calcium chloride, hemin chloride, benzethonium chloride, or any combination thereof. In certain embodiments, the pharmaceutically acceptable vehicle, carrier or excipient comprises a carbohydrate. In certain embodiments, the carbohydrate comprises sucrose, dextrose, trehalose, lactose, cellulose, sorbitol, galactose, dextran, xanthan, or any combination thereof. In certain embodiments, the pharmaceutically acceptable vehicle, carrier or excipient comprises an amino acid or protein. In certain embodiments, the amino acid or protein comprises gelatin, egg protein, yeast extract, glutamate, albumin, In certain embodiments, the pharmaceutically acceptable vehicle, carrier or excipient comprises an emulsifier. In certain embodiments, the emulsifier comprises octylphenol ethoxylate (Triton X-100), polysorbate 20, polysorbate 80 (Tween 80), sodium deoxy cholate, or any combination thereof. In certain embodiments, the pharmaceutically acceptable vehicle, carrier or excipient comprises a chelating agent. In certain embodiments, the chelating agent comprises ethylene diamine tetra acetic acid sodium (EDTA), EGTA, or any combination thereof. In certain embodiments, the carrier is poly D,L-lactide-co-glycolide (PLGA). In certain embodiments, the V_{H}H is diluted in a liquid suitable for consumption by a human individual or a domestic animal.

### Nucleic Acids Encoding V_{H}H Polypeptides

The isolated, and purified V_{H}H polypeptides of the current disclosure can be produced in cell based protein production systems that have been modified by nucleic acids to express the V_{H}H polypeptides. Therefore, any of the engineered V_{H}H polypeptides described herein can be encoded by a nucleic acid. In certain embodiments, the nucleic acid is a plasmid. In certain embodiments, the plasmid comprises an origin or replication for propagation in E. coli. In certain embodiments, the nucleic acid is encoded on a plasmid suitable for transforming yeast. In certain embodiments, the plasmid is suitable for homologous recombination in yeast. In certain embodiments, the plasmid comprises a gene for a yeast auxotrophy such as histidine, tryptophan, leucine, lysine, methionine, or uracil. In certain embodiments, the plasmid has a gene that confers antibiotic resistance to ampicillin, kanamycin, neomycin, G418, carbenicillin, chloramphenicol, blasticidin, zeocin, or any combination thereof. In a certain embodiment, the plasmid is pPIC9 SHUTTLE. In certain embodiments, the nucleic acid is a linear single or double stranded DNA molecule able to undergo homologous recombination in yeast. In certain embodiments, the nucleic acid is a double stranded linear DNA molecule that comprises any of the V_{H}H polypeptides of the current disclosure. In certain embodiments, the nucleic acid is a PCR product that comprises any of the engineered V_{H}H polypeptides of the current disclosure.

### Cell Based Systems for Production of V_{H}H Polypeptides

The V_{H}H polypeptides described herein can be isolated and purified from a cellular expression system. The isolated, purified V_{H}H polypeptides of the current disclosure are purified from a cell based protein production system that has been transformed, transfected, or infected with a nucleic acid encoding an engineered V_{H}H polypeptide. In certain embodiments, the cell based protein production system is stably transformed with the nucleic acid, such that the nucleic acid integrates into at least one chromosome of the cell based protein production system. In certain embodiments, the eukaryotic system is yeast. In certain embodiments, the yeast is a Pichia pastoris strain. In certain embodiments, the yeast is a Saccharomyces cerevisiae strain. In certain embodiments, the strain of Pichia pastoris is modified to produce a human glycosylation pattern in polypeptides produced using the system. In certain embodiments, the cell based protein purification system is not a mammalian cell line.

### Master Cell Bank and Transgenic Yeast

In a certain embodiment, described herein is a master cell bank comprising a yeast or bacteria that comprises a nucleic acid encoding one or more polypeptides integrated into its genome creating a transgenic bacteria or yeast strain. In certain embodiments, the nucleic acid is maintained extrachromosomal, on a plasmid, bacterial artificial chromosome, or yeast artificial chromosome. In certain embodiments, the nucleic acid is integrated into a chromosomal location. In certain embodiments, the yeast is Pichia pastoris . In certain embodiments, the Pichia pastoris is a GS115 strain. In certain embodiments, the transgenic yeast is created by transformation with linearized plasmid, a PCR product, or a synthesized double stranded DNA molecule. In certain embodiments, the transgenic yeast is created by homologous recombination. In certain embodiments, the master cell bank comprises a cryopreservative suitable for freezing to at least about −80° or below. In certain embodiments, the master cell bank comprises glycerol at between 10 and 30%, and is suitable for long term storage at about −80° or below. In certain embodiments, the master cell bank can preserve a transgenic yeast strain for at least 1, 2, 3, 4, 5, 6, 7, 8, 9, 10 or more years.

### Additional Active Ingredients

In certain embodiments, the antibodies for use with the compositions and methods described herein are administered with an additional active ingredient that is effective in controlling bacteria. In certain embodiments, the antibodies are administered with an antibiotic. In certain embodiments, the antibodies are administered with a probiotic. In certain embodiments, the antibodies are administered with a hormone.

### Diagnostic and Research Uses

In certain embodiments, any of the antibodies described herein are useful for use in diagnostic assays or for research purposes. Any of the antibodies described herein can be used in an in vitro assay. In certain embodiments, the antibodies described herein are useful in a companion diagnostic.

### EXAMPLES

The following illustrative examples are representative of embodiments of the software applications, systems, and methods described herein and are not meant to be limiting in any way

### Example 1—Purification of Flagella and Recombinant Target Antigens

Flagella were isolated from whole-bacterial cells. Bacterial cells were grown overnight at 37° C. with shaking. Overnight cultures were centrifuged and the cell pellets resuspended in 10 mM Tris pH 8.0, 0.9% NaCl. Resuspended cells were homogenized and centrifuged for 20 minutes at 10,000 rpm. Supernatants were subjected to an additional round of centrifugation at 10,000 rpm followed by ultracentrifugation at 45,000 rpm for 1 hour. Pellets were resuspended in 1 ml 10 mM Tris pH 8.0, 0.9% NaCl and checked for purity using SDS-PAGE.

FliC protein was purified from an E. coli expression system. FliC was expressed at 30° C. in E. coli Rosetta (DE3) pLacI cells (Novagen), induced at an absorbance of ˜0.6 at 600 nm by adding 0.4 mM isopropyl-β-D-thiogalactoside (IPTG) and grown for 3.5 more hours before collection. Cells were lysed by sonication in buffer A (250 mM KCl and 10 mM HEPES, pH 7.4) with 25 μg/ml DNase I, 25 μg/ml lysozyme, 14 mM β-mercaptoethanol and 1 mM phenylmethylsulphonyl fluoride. The lysate was applied to a 25 ml Poros MC column (Tosoh Bioscience), washed with five column volumes of buffer A and eluted with 30% (vol/vol) buffer B (250 mM KCl and 500 mM imidazole, pH 7.4). The protein was dialyzed overnight against buffer C (20 mM KCl, 10 mM Tris, pH 8.0 and 14 mM (3-mercaptoethanol) at 4° C. The sample was then applied to a 20 ml HiLoad Q column (GE Healthcare). The protein was eluted with a gradient of 0% to 35% buffer D (1.0 M KCl, 10 mM Tris pH 8.0 and 14 mM β-mercaptoethanol). Lastly, the elution was loaded onto a HiLoad 16/60 Superdex 200 prep grade (GE Healthcare) gel filtration column using buffer A plus 14 mM β-mercaptoethanol. The protein sample was then concentrated to 2.3 mg/mL using Amicon concentrators (30 kDa molecular weight cutoff (MWCO); Millipore). The purified protein was stored at −80° C.

PrgI protein was purified form an E. coli expression system. PrgI was expressed at 30° C. in E. coli Rosetta (DE3) pLacI cells (Novagen), induced at an absorbance of ˜0.6 at 600 nm by adding 0.4 mM isopropyl-β-D-thiogalactoside (IPTG) and grown for 3.5 more hours before collection. Cells were lysed by sonication in buffer A (250 mM KCl and 10 mM HEPES, pH 7.4) with 25 μg/ml DNase I, 25 μg/ml lysozyme and 1 mM phenylmethylsulphonyl fluoride. The lysate was applied to a 25 ml Poros MC column (Tosoh Bioscience), washed with five column volumes of buffer A and eluted with 30% (vol/vol) buffer B (250 mM KCl and 500 mM imidazole, pH 7.4). The elution was then loaded onto a HiLoad 16/60 Superdex 200 prep grade (GE Healthcare) gel filtration column using buffer A. The protein sample was then concentrated to 950 uM using Amicon concentrators (3 kDa molecular weight cutoff (MWCO); Millipore). The purified protein was stored at −80° C.

FimA protein was purified form an E. coli expression system. FimA was expressed at 18° C. in E. coli Rosetta (DE3) pLacI cells (Novagen), induced at an absorbance of ˜0.6 at 600 nm by adding 0.4 mM isopropyl-β-D-thiogalactoside (IPTG) and grown for 20 more hours before collection. Cells were lysed by sonication in buffer A (250 mM KCl and 10 mM HEPES, pH 7.4) with 25 μg/ml DNase I, 25 μg/ml lysozyme, 14 mM β-mercaptoethanol and 1 mM phenylmethylsulphonyl fluoride. The lysate was applied to a 25 ml Poros MC column (Tosoh Bioscience), washed with five column volumes of buffer A and eluted with 30% (vol/vol) buffer B (250 mM KCl and 500 mM imidazole, pH 7.4). The protein was dialyzed overnight against buffer C (10 mM KCl, 10 mM Tris, pH 8.8, and 14 mM β-mercaptoethanol) at 4° C. The sample was then applied to a 20 ml HiLoad Q column (GE Healthcare). The protein was in the flow-through of the column. Last, the sample was run on a HiLoad 10/300GL Superdex 75 (GE Healthcare) gel filtration column using buffer A plus 14 mM β-mercaptoethanol. The protein sample was then concentrated to 350 uM using Amicon concentrators (10 kDa molecular mass cutoff; Millipore). The purified protein was stored at −80° C.

### Example 2—Generation of Antibodies in Llamas

A single llama was immunized with a mixture of purified flagella and recombinant target antigens, FliC, PrgI and FimA. The llama immunization was performed by Cedarlane where 250 μg of each antigen were pooled and injected for a total of four injections. Antigens were mixed at NRC and supplied frozen on dry ice to Cedarlane. At the time of injection, the antigens were thawed and the volume increased to 1 ml with PBS. The 1 ml antigen-PBS mixture was then mixed with 1 ml of CFA or IFA for a total of 2 ml. A total of 2 ml was immunized per injection. Whole llama blood and sera were collected from the immunized animal on days 0, 35, 42, 75 and 87. Sera from days 35, 42 and 75 were then fractionated to separate V_{H}H from conventional antibodies. Fractionation was done according to standard protocols by the National Research Council (NRC). ELISA was used to measure reactivity against target antigens in polyclonal and V_{H}H-enriched fractions.

### Example 3—In Vitro Generation of Monoclonal Antibodies

RNA isolated from purified llama lymphocytes was used to generate cDNA for cloning into phagemids. The resulting phagemids were used to transform E. coli to generate a library of expressed V_{H}H genes. The phagemid library size was ˜2.5×10⁷ total transformants and the estimated number of phagemid containing V_{H}H inserts was estimated to be ˜100%. High affinity antibodies were then selected by panning against Salmonella antigens used for llama immunization. A total of two rounds of panning were performed and clones arising from rounds 1 and 2 were sequenced according to their CDR regions. Phage ELISA was then performed on phage bearing unique single domain antibody clones identified from panning/DNA sequencing to prioritize and select single domain antibodies for soluble expression and purification.

### Example 4—Motility Assays

Inhibition of Salmonella motility by polyclonal antibody serum and V_{H}H antibodies was tested using either a standard plate motility assay or live-imaging microscopy.

### Plate-Based Motility Assay Protocol

Plate-based motility assays were used to determine whether llama-derived polyclonal antibody serum was capable of inhibiting the motility of five poultry-contaminating Salmonella strains. The strains used were Salmonella enterica serotype Typhimurium strain SL1344, Salmonella enterica serotype Enteritidis strain PT4, Salmonella enterica serotype Enteritidis strain LK5, Salmonella enterica serotype Newport and Salmonella enterica serotype Heidelberg. Overnight cultures of Salmonella were mixed in 1:1 volumes with polyclonal antibody serum, pre-immune serum or PBS. The mixtures were incubated for 30 minutes at room temperature and following incubation, 10 μl from each mixture was spotted in the center of a petri dish containing 0.25% agar and incubated at 37° C. Bacterial motility was determined by measuring the diameter of growth six hours after plating.

### Plate-Based Motility Assay Results

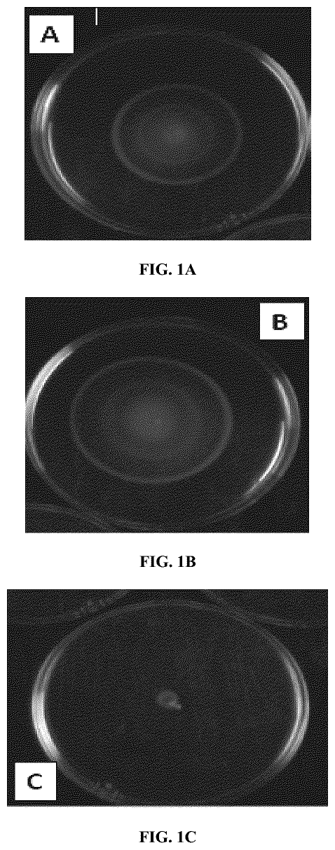

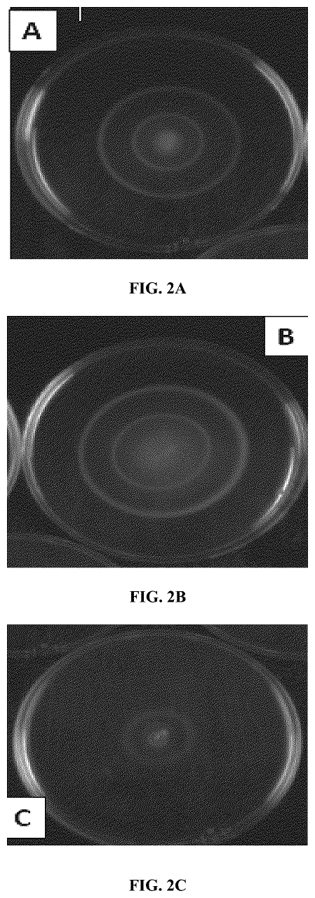

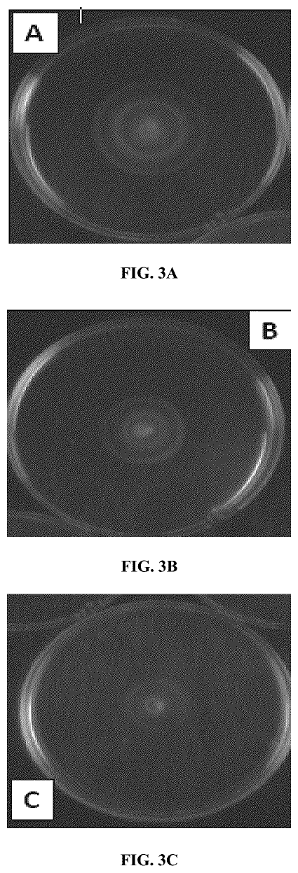

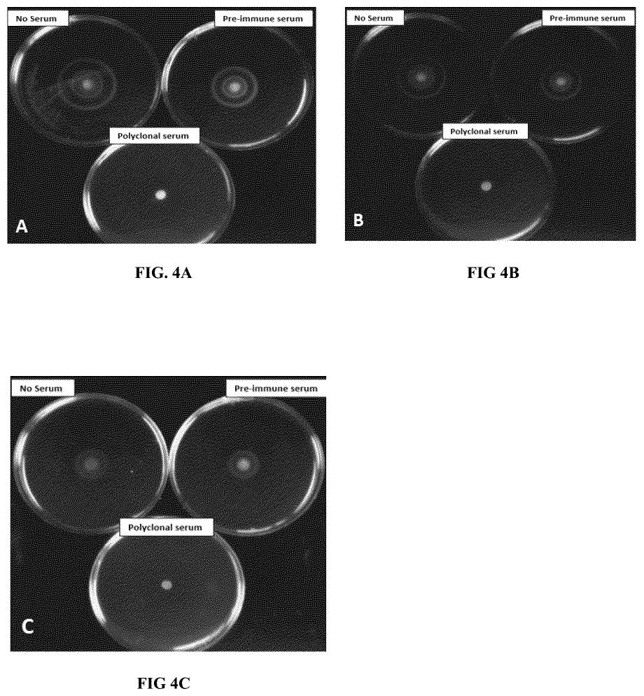

FIGS. 1A to 4C show that the motility of each Salmonella strain is inhibited in the presence of polyclonal antibody serum in comparison to pre-immune serum or no serum controls. Referring to FIGS. 1A, 1B, and 1C the motility of Salmonella enterica serotype Typhimurium strain SL1344 is inhibited in the presence of NovoBind polyclonal antibody serum. (A) Salmonella enterica serotype Typhimurium strain SL1344 incubated with phosphate buffered saline as a control; (B) Salmonella enterica serotype Typhimurium strain SL1344 incubated with pre-immune serum; (C) Salmonella enterica serotype Typhimurium strain SL1344 incubated with polyclonal antibody serum. Referring to FIGS. 2A, 2B, and 2C motility of Salmonella enterica serotype Enteritidis strain PT4 is inhibited in the presence of NovoBind polyclonal antibody serum. (A) Salmonella enterica serotype Enteritidis strain PT4 incubated with phosphate buffered saline as a control; (B) Salmonella enterica serotype Enteritidis strain PT4 incubated with pre-immune serum; (C) Salmonella enterica serotype Enteritidis strain PT4 incubated with polyclonal antibody serum. Referring to FIGS. 3A, 3B, and 3C motility of Salmonella enterica serotype Enteritidis strain LK5 is inhibited in the presence of NovoBind polyclonal antibody serum. (A) Salmonella enterica serotype Enteritidis strain LK5 incubated with phosphate buffered saline as a control; (B) Salmonella enterica serotype Enteritidis strain LK5 incubated with pre-immune serum; (C) Salmonella enterica serotype Enteritidis strain LK5 incubated with polyclonal antibody serum. Referring to FIGS. 4A, 4B, and 4C motility of Salmonella enterica serotype Enteritidis strain PT4, Salmonella enterica serotype Newport and Salmonella enterica serotype Heidelberg is inhibited in the presence of NovoBind polyclonal antibody serum. (A) Salmonella enterica serotype Enteritidis strain PT4 incubated with phosphate buffered saline, pre-immune serum, or NovoBind polyclonal antibody serum; (B) Salmonella enterica serotype Newport phosphate buffered saline, pre-immune serum, or NovoBind polyclonal antibody serum; (C) Salmonella enterica serotype Heidelberg incubated with phosphate buffered saline, pre-immune serum, or NovoBind polyclonal antibody serum.

### Live Imaging Motility Assay Protocol

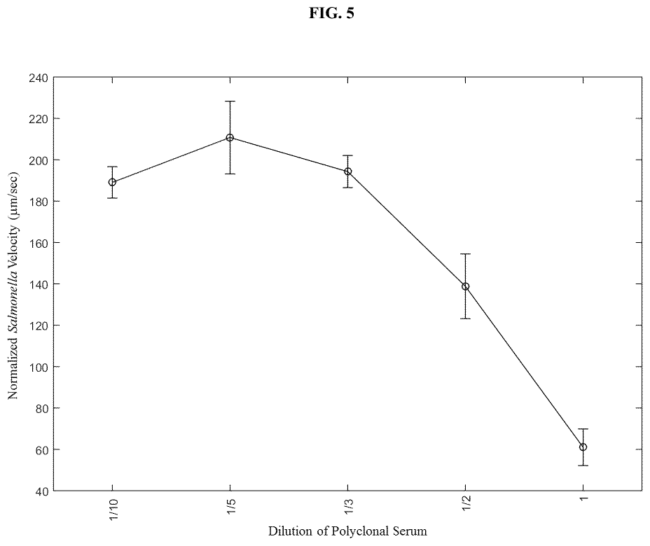

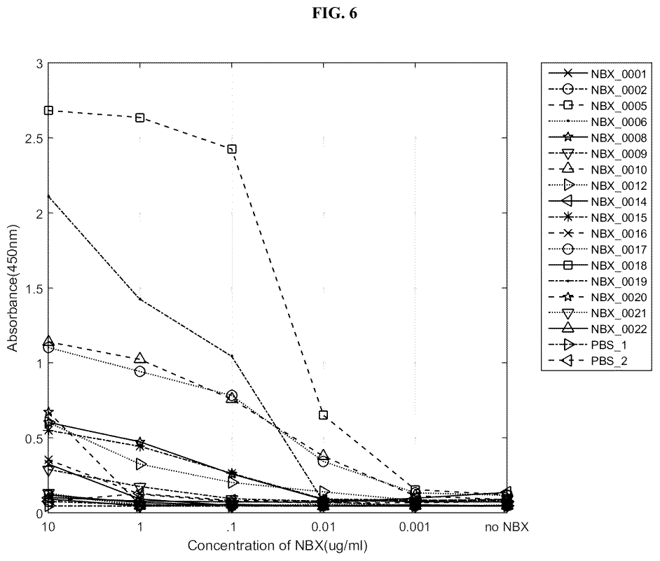

Live-imaging microscopy was used to quantify the inhibitory effect of polyclonal antibody serum and monoclonal V_{H}H antibodies (NBXs) on motility of Salmonella. Briefly, an overnight culture of Salmonella enterica serotype Typhimurium strain SL1344 was used to inoculate a subculture which was grown at 37° C. in a shaking incubator until logarithmic growth was reached. Five μl of log phase Salmonella enterica serotype Typhimurium strain SL1344 was mixed with 10 μl of polyclonal antibody serum or monoclonal V_{H}H antibodies diluted in PBS as indicated (FIGS. 5, 9-12). Mixtures were incubated at room temperature for 1 hour and observed at 400× magnification with an Olympus IX70 inverted microscope with an Olympus DP80 camera. Salmonella motility was tracked and analyzed using Velocity image analysis software and bacterial movement was normalized and expressed as a percentage of a control that was considered to be 100% motile.

### Live Imaging Motility Assay Results

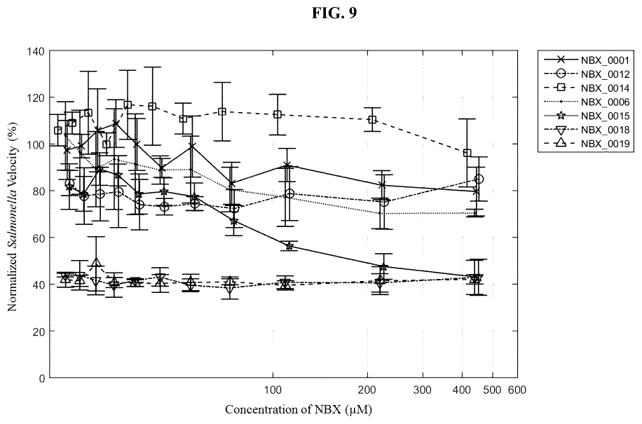

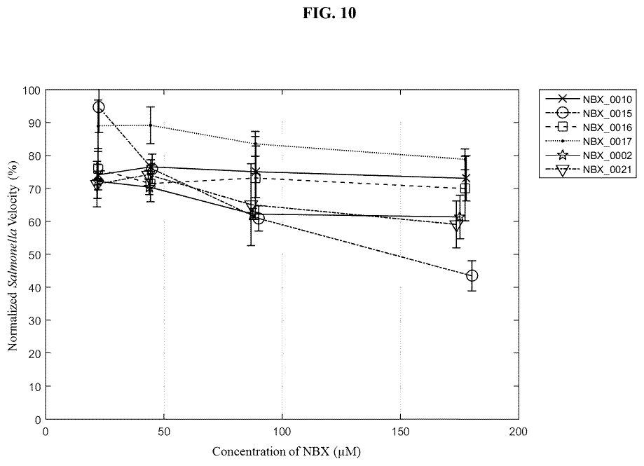

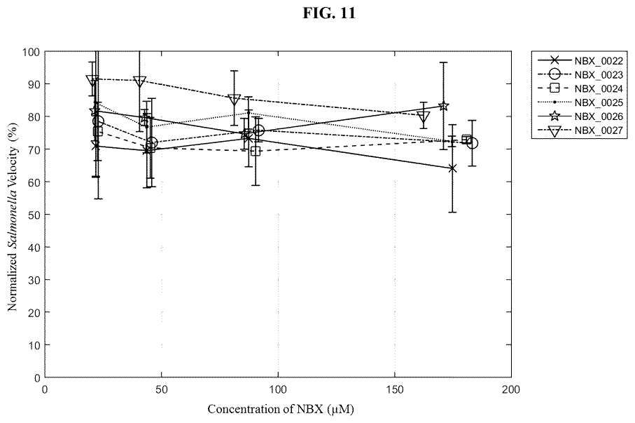

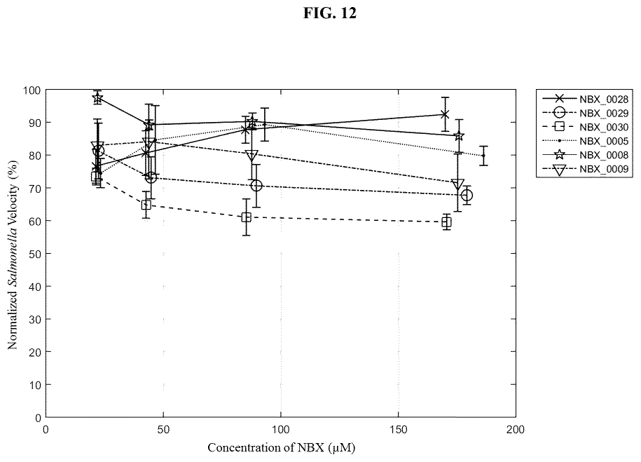

FIG. 5 and FIGS. 9-12 show results from live imaging motility assays. Referring to FIG. 5 Salmonella enterica serotype Typhimurium strain SL1344 motility is inhibited in the presence of NovoBind polyclonal antibody serum using a live imaging motility assay. The inhibitory effect of NovoBind polyclonal antibody serum decreases with increased dilution of serum. Referring to FIG. 9 the motility of Salmonella enterica serotype Typhimurium strain SL1344 is inhibited in the presence of NovoBind NBX0018 and NBX0019. Referring to FIG. 10 the motility of Salmonella enterica serotype Typhimurium strain SL1344 is inhibited in the presence of NovoBind NBX0015. Referring to FIG. 11 the motility of Salmonella enterica serotype Typhimurium strain SL1344 is inhibited in the presence of NovoBind NBX0022-NBX0027. Referring to FIG. 12 the motility of Salmonella enterica serotype Typhimurium strain SL1344 is inhibited in the presence of NovoBind NBX0005, NBX0008, NBX0009, NBX0028, NBX0029 and NBX0030.

### Example 5—In Vitro Analysis of Antibody Binding

### In Vitro Analysis of Antibody Binding Protocol

Antibody binding was assessed using ELISA. Each well of a microtitre plate was coated with 100 μl containing 0.2 μg of appropriate antigen and plates were incubated overnight at 4° C. Wells were emptied and blocked with 5% skim milk in PBST (PBS 0.1% tween-20), covered with parafilm and incubated at 37° C. for 1 hour. Wells were emptied and 100 μl of the appropriate NBX was added at the appropriate concentration to each well; for wells containing no NBX, 100 μl of PBS was added. Plates were wrapped with parafilm and incubated at room temperature for 1 hour. Following incubation, well contents were removed and wells were washed 3× with PBST. Next, 100 μl of a 1:5000 dilution of anti-6-His-HRP secondary antibody was added to each well, plates were sealed with parafilm and incubated at room temperature for 1 hour. Well contents were removed and wells were again washed 3× with PBST. Signal was detected by adding 100 μl of TMB substrate mixed in equal volume with TMB developing solution to each well for 5 min at room temperature. Reactions were stopped by adding 100 μl of 1 M phosphoric acid and absorbance was read at 450 nm.

### In Vitro Analysis of Antibody Binding Results

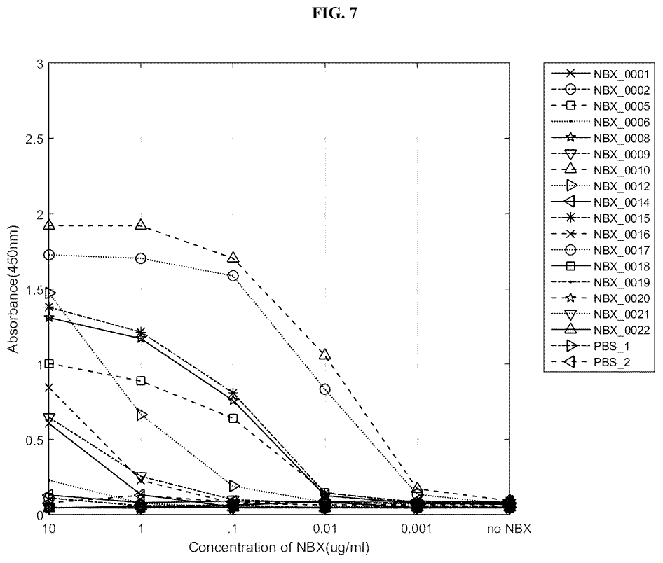

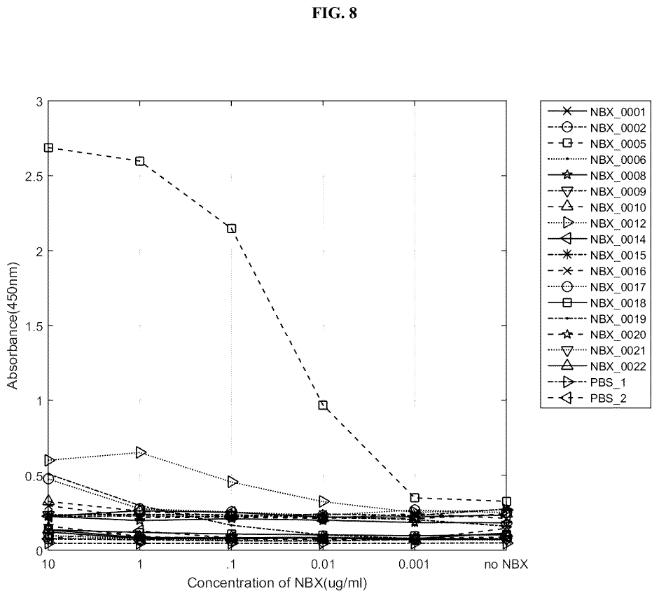

FIGS. 6-8 show results of ELISA based antibody binding assays. FIG. 6 shows the differences in specific binding among 17 different monoclonal NBXs raised against whole Salmonella enterica serotype Typhimurium flagella or recombinant FliC versus a Salmonella enterica serotype Typhimurium flagella capture surface. FIG. 7 shows the differences in specific binding among 17 different monoclonal NBXs raised against whole Salmonella enterica serotype Typhimurium flagella or recombinant FliC versus a Salmonella enterica serotype Enteritidis flagella capture surface. FIG. 8 shows the differences in specific binding among 17 different monoclonal NBXs raised against whole Salmonella enterica serotype Typhimurium flagella or recombinant FliC versus a recombinant FliC capture surface.

### Example 6—Efficacy of V_{H}H Antibodies (NBXs) in Inhibiting Bacterial Motility In Vitro

Shown in Table 5 are the effective in vitro concentrations of different NBX antibodies obtained in a motility assay as performed in Example 4.

TABLE 5
Effect of individual V_{H}H antibodies (NBXs) on Salmonella
motility and their effective concentrations

| V_{H}H | Percent Motility^{a} | Effective Concentration^{b} (μM) |
|---|---|---|
| NBX0001 | 50% | 90 |
| NBX0002 | 62% | 85 |
| NBX0005 | 80% | 190 |
| NBX0006 | 58% | 85 |
| NBX0008 | 85% | 175 |
| NBX0009 | 72% | 175 |
| NBX0010 | 75% | 90 |
| NBX0012 | 70% | 180 |
| NBX0014 | 70% | 75 |
| NBX0015 | 42% | 450 |
| NBX0016 | 72% | 40 |
| NBX0017 | 78% | 175 |
| NBX0018 | 40% | 30 |
| NBX0019 | 40% | 30 |
| NBX0021 | 60% | 170 |
| NBX0022 | 65% | 170 |
| NBX0023 | 72% | 45 |
| NBX0024 | 70% | 45 |
| NBX0025 | 72% | 170 |
| NBX0026 | 75% | 80 |
| NBX0027 | 80% | 165 |
| NBX0028 | 77% | 25 |
| NBX0029 | 67% | 180 |
| NBX0030 | 60% | 170 |

^{a}Percent motility of Salmonella enterica serotype Typhimurium strain SL1344 normalized to a 100% motile control.
^{b}Lowest molar concentration required to achieve lowest percent motility.

### Example 7—Efficacy of V_{H}H Antibodies (NBXs) on Biofilm Formation

Biofilms are multi-cellular bacterial communities. Salmonella can form biofilms on many types of surfaces including chicken intestinal epithelium and this contributes to colonization and disease in animals. Therefore, the ability to inhibit biofilm formation may decrease the burden of Salmonella in the GI tract of animals. This assay was completed with monomeric NBX.

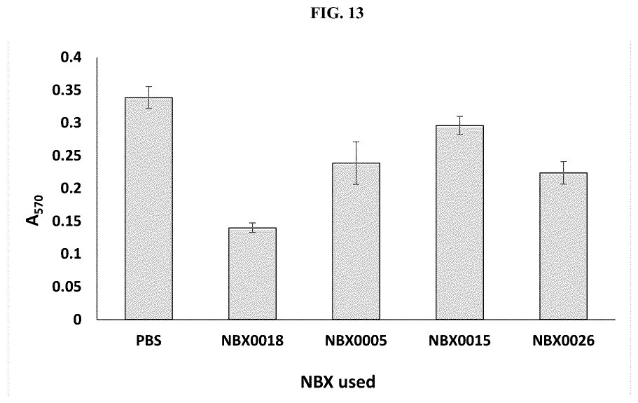

Salmonella enterica serovar Typhimurium strain SL1344 was grown overnight in 5 mL of LB media at 37° C. with shaking at 240 RPM. Bacteria were diluted to an OD₆₀₀=0.1 in LB media and grown for 4 hours at 37° C. with shaking at 240 RPM. Four mL of bacterial culture was pelleted by centrifugation and the supernatant was removed. The bacteria were resuspended in 1 mL of LB with no salt. The bacterial culture was pelleted again by centrifugation and the supernatant was removed. The bacteria were resuspended in 1 mL of LB with no salt and the optical density at 600 nm (OD₆₀₀) was measured. The bacteria were diluted to an OD₆₀₀ of 1.25 in LB with no salt. For each condition tested, quintuplicate 96-well plate wells were used. Each well received 80 μl of LB with no salt, 10 μl of bacterial culture in LB with no salt, and 10 μl of NBX stock solution dissolved in PBS or PBS. The final concentration of NBX was 1.0 mg/ml. The 96-well plate was incubated at room temperature for 24 hours. Non-adherent bacteria were removed and adherent biofilms were washed three times with 200 μl of water. Biofilms were fixed to 96-well plates by heat treatment (60° C. for 1 hour). Biofilms were stained with 150 μl of 0.1% (weight/volume) crystal violet for 30 minutes at room temperature. Excess stain was removed and wells were washed three times with 200 μl of water. Plates were inverted and left to dry for 24 hours. Cells were de-stained by addition of 150 μl of 95% (volume/volume) ethanol to wells and incubate 1 hour at room temperature to de-stain crystal violet from the cells. To quantify biofilm formation, absorbance was measured at 570 nm (A₅₇₀) of the solution to determine the amount of crystal violet that had stained the biofilms. FIG. 13 shows that all NBXs tested reduced biofilm formation between 12% and 59% NBX0018 had the highest reduction in biofilm formation followed by NBX0026, NBX0005, and NBX0026.

### Example 8—Multimeric V_{H}H Antibodies Display Enhanced Efficacy

To increase the effectiveness of V_{H}Hs (NBXs), we produced concatemer proteins consisting of up to three NBX subunits linked by 15 amino acid (3xG4S) linker sequences. These protein sequences were constructed and proteins were expressed and produced by means similar to monomeric (V_{H}H) NBX molecules. Multimeric V_{H}H were assessed in a Live imaging motility assay protocol as per example 4. Results are shown in Table 6. A trimer of NBX0018 inhibits motility at a concentration 300-fold less than a monomer, and the dual NBX0018 inhibits motility at a concentration 20-fold less than the monomer.

TABLE 6
Inhibition of Salmonella motility by multimeric V_{H}H

| | Minimum Concentration Required | |
|---|---|---|
| NBX Construct | for 50% Motility Inhibition | |
| NBX0018 | 510 | nM |
| NBX0018-NBX0018 | 24.4 | nM |
| NBX0018-NBX0018- | 1.7 | nM |
| NBX0018 | | |
| NBX0005 | >372 | μM |
| NBX0005-NBX0005 | 2.6 | μM |
| NBX0015 | 52 | μM |
| NBX0015-NBX0015 | 25 | μM |

Minimum protein concentrations required by NBX constructs to inhibit Salmonella enterica serovar Typhimurium strain SL1344 motility by >50%.

### Example 9—Multimeric V_{H}H Antibodies Aggregate Salmonella Bacteria

Although bacteria, including Salmonella can aggregate into ordered structures known as biofilms, the uncontrolled aggregation of bacteria into clumps has been shown to be detrimental to the ability of Salmonella to cause infections in animal models, including chickens. Therefore, we tested the ability of multimeric V_{H}H to induce aggregation of Salmonella.

Salmonella enterica serovar Typhimurium strain SL1344 was grown overnight in 5 mL of LB media at 37° C. with shaking at 200 RPM. One mL of bacterial culture was pelleted by centrifugation and the supernatant was removed. The bacteria were resuspended in 1 mL of PBS to remove trace amounts of media. The bacterial culture was pelleted again by centrifugation and the supernatant was removed. The bacteria were resuspended in 1 mL of PBS and the optical density at 600 nm (OD₆₀₀) was measured. The bacteria were diluted to an OD₆₀₀ of 1.0 in PBS. 15 μl volumes of bacteria were placed in 1.5 mL centrifuge tubes and 15 μl of PBS (negative control), NBX (test molecules), or 3% anti-Salmonella enterica serovar Typhimurium strain SL1344 polyclonal antibody (positive control) were added to appropriate tubes. Mixtures were incubated at 4° C. for 20 hours. 10 μl of each mixture were placed in chamber microscope slides and visualized at 400× magnification. For each microscope slide, 20-30 random fields of view are observed and 3-5 representative images are photographed. If no bacterial aggregations are observed to this point, an additional 20-30 fields of view are observed to confirm lack of aggregates.

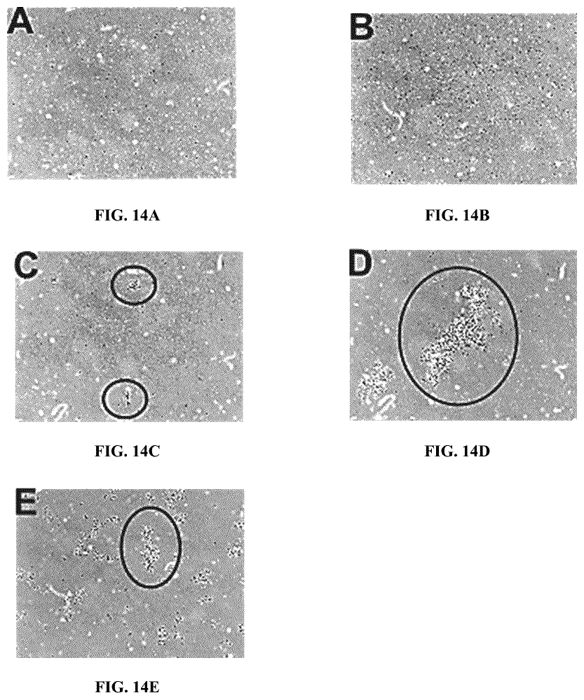

FIG. 14 shows that while single monomers of V_{H}H (NBX0018) were ineffective at aggregating Salmonella (14B), compared to vehicle control (14A), dimers of V_{H}H (NBX0018-NBX0018) induced aggregation (14C, circled), and trimers (NBX0018-NBX0018-NBX0018) induced a large amount of aggregation (14D, circled), even greater than a positive control antibody (a polyclonal anti-sera) (14E, circled).

### Example 10—Multimeric V_{H}H Antibodies Block Cellular Invasion by Salmonella Bacteria

A key process that contributes to the virulence of Salmonella is its ability to invade host intestinal epithelial cells. Invasion of host cells can allow Salmonella to multiple intracellularly, spread to the lymph nodes and systemic circulation, and potentially infect other organ systems. A method to assess invasion in vitro is the HeLa cell invasion assay. This assay involves incubation of the bacteria and HeLa cells, incubation with an antibiotic to remove external Salmonella, then lysis of HeLa cells followed by enumeration of invaded Salmonella. The number of Salmonella that have invaded HeLa cells can be used to determine if a V_{H}H can block this process.

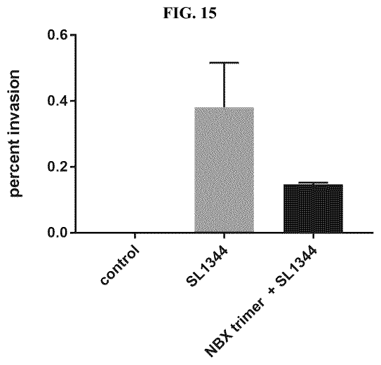

HeLa cells (5×10⁴ cells/well) were seeded in tissue culture media and maintained at 37° C. in 5% CO₂. An overnight culture of Salmonella enterica serovar Typhimurium strain SL1344, was prepared with shaking at 37° C. After 16 hours of incubation, a subculture was prepared (1:33 in LB Broth with streptomycin) for 3 hours. Salmonella were quantified by measuring OD₆₀₀. HeLa cells were incubated with 5×10⁵ cells/well for 1 hour with or without 1 mg/ml NBX0018-NBX0018-NBX0018. Input Salmonella were then plated in serial dilutions. Cells were then washed 3 times with PBS and incubated with gentamycin (50 μg/mL) for a period of 1 hour. Following incubation with antibiotic, cells were washed 3 times with PBS and lysed. Serial dilutions were made and plated for Salmonella enumeration. % invasion was calculated as the number of Salmonella from HeLa cells/input Salmonella. Results in FIG. 15 show that HeLa cell invasion by Salmonella enterica serovar Typhimurium strain SL1344 is reduced by more than 50% in the presence of NBX0018-NBX0018-NBX0018 (NBX trimer). Control refers to uninfected HeLa cells treated with PBS throughout the experiment.

### Example 11—V_{H}H Block Salmonella Activity Dependent on Production System

In order to colonize and establish successful infections, Salmonella must adhere to eukaryotic cells lining the GI tract. One mechanism by which Salmonella does this is to use its Type-1 Fimbriae to bind to mannose sugars on the surface of the eukaryotic cells. Disruption of the interaction between Type-1 Fimbriae and surface-expressed mannose sugars will lead to decreases in Salmonella colonization. Yeast cells also make these sugars on their surface and if exposed to Salmonella with function Type-1 Fimbriae, the yeast cells will clump together. The presence or absence of yeast clumping can be used as an in vitro proxy to determine if a V_{H}H is blocking Type-1 Fimbriae function. V_{H}Hs that are recombinantly produced in yeast (Pichia pastoris) rather than the bacterium Escherichia coli will be glycosylated with many sugars including mannose. This mannose glycosylation on the V_{H}H can add a function to the V_{H}H and block Type-1 Fimbriae function.

Salmonella enterica serovar Typhimurium strain SL1344 was grown for 96 hours in 5 mL of LB media at 37° C. without shaking. 0.45 mL of bacterial culture was pelleted by centrifugation and the supernatant was removed. The bacteria were resuspended in 0.45 mL of PBS to remove trace amounts of media. The bacterial culture was pelleted again by centrifugation and the supernatant was removed. The bacteria were resuspended in 0.045 mL of PBS, PBS containing 1 mg/ml NBX, or PBS containing 100 mM mannose (positive control for Type-1 Fimbriae inhibition). Bacteria were incubated for 1 hour at room temperature. Saccharomyces cerevisiae yeast cells were resuspended in PBS at an OD₆₀₀=10 and 5 μl was added to the bacteria. Mixtures were incubated at 4° C. for 20 hours. 10 μl of each mixture were placed in chamber microscope slides and visualized at 400× magnification. For each microscope slide, 20-30 random fields of view are observed and 3-5 representative images are photographed. If no yeast clumps are observed to this point, an additional 20-30 fields of view are observed to confirm lack of clumps.

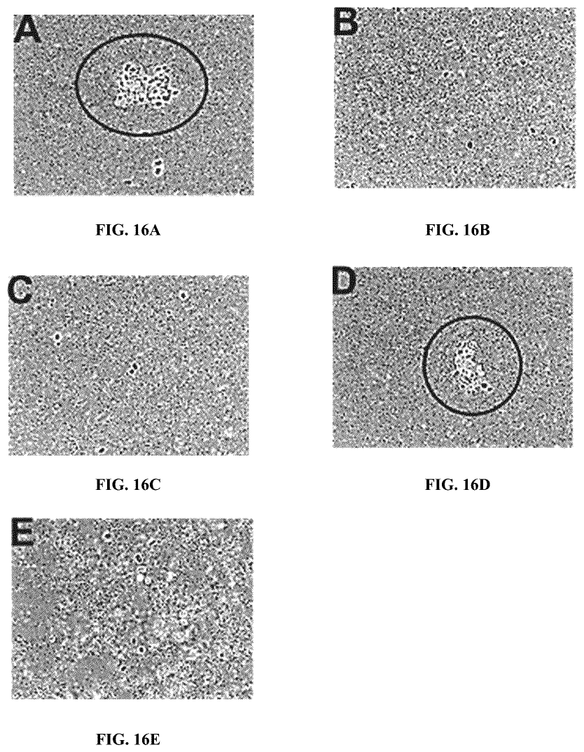

Results in FIG. 16 show Salmonella enterica serovar Typhimurium strain SL1344 clumps Saccharomyces cerevisiae during PBS treatment (16A) or treatment with NBX0018 recombinantly produced in Escherichia coli (16D). In the presence of Salmonella enterica serovar Typhimurium strain SL1344 lacking the Type-1 Fimbriae (FimA mutant strain), Saccharomyces cerevisiae cells remain unclumped (16B). In the presence of Salmonella enterica serovar Typhimurium strain SL1344 with intact Type-1 Fimbriae and excess soluble mannose (16C) or NBX0018 recombinantly produced in Pichia pastoris (16E), Saccharomyces cerevisiae cells remain unclumped, indicating that the interaction between Type-1 Fimbriae and mannose sugars on the surface of the Saccharomyces cerevisiae cells has been blocked.

### Example 12—Additional V_{H}H from Phage Display

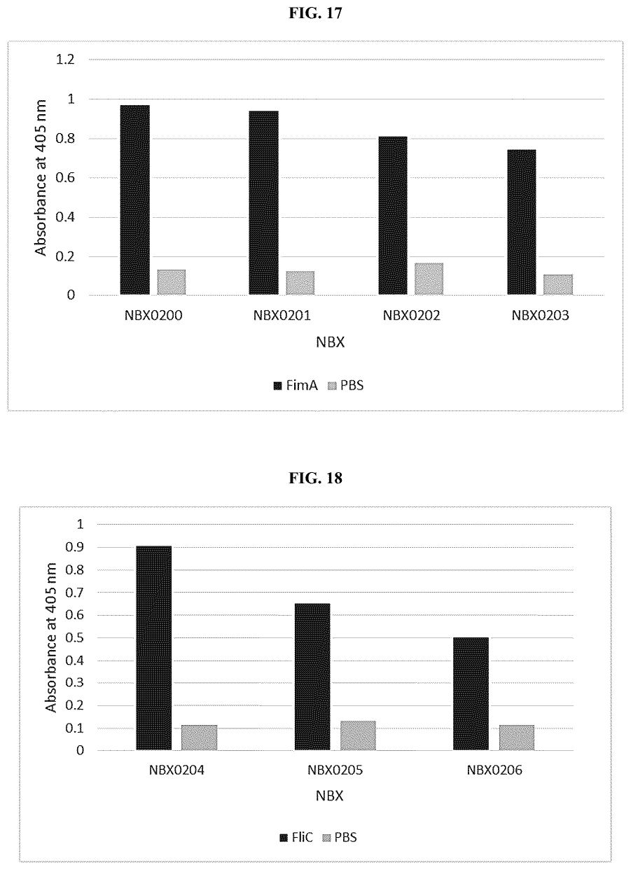

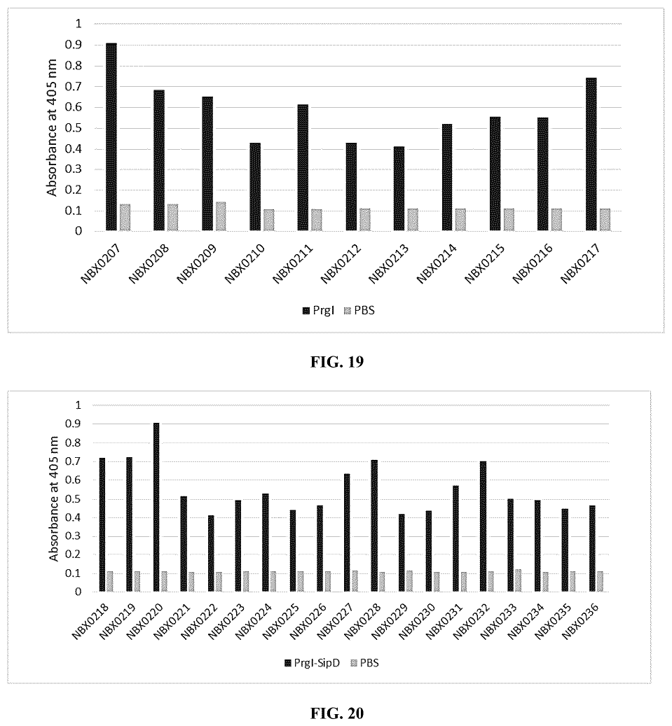

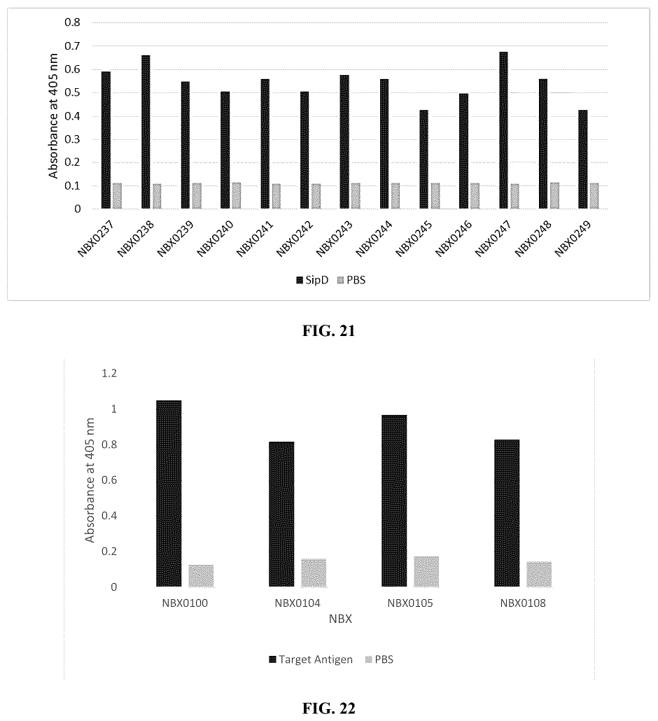

M13KO7 phage carrying individual NBX genes were rescued from 1 mL cultures of Escherichia coli strain TG-1 upon superinfection with 10⁹ plaque forming units of M13KO7 helper phage. Phage were separated from bacteria by centrifugation and stored at −20oC until needed. ELISA plate wells were coated overnight at 4oC with either 100 μl of PBS (negative control) or 100 μl of 5 μg/ml antigen. Wells were blocked for 2 hours at 37oC with 200 μl of PBS+0.05% (volume/volume) Tween-20+5% (weight/volume) Skim Milk Powder. 100 μl of phage preparations were added to wells for 1 hour at 37oC. Wells were washed four times with 300 μl of PBS+0.05% (volume/volume) Tween-20. 100 μl of anti-M13 IgG-horse radish peroxidase diluted 1 in 5000 in PBS was added for 1 hour at room temperature. Wells were washed four times with 300 μl of PBS+0.05% (volume/volume) Tween-20. 100 μl of 1 mg/ml 2,2′-azino-bis(3-ethylbenzothiazoline-6-sulphonic acid)+0.03% (volume/volume) hydrogen peroxide in sodium citrate-phosphate buffer pH 5.0 was added to wells for 15 minutes at room temperature. 100 μl of sodium dodecyl sulfate was added to wells. Absorbance at 405 nm was measured for each well. Data for additional V_{H}Hs specific for these targets is shown in FIG. 17-FIG. 22. CDR sequences are shown in Table. 2.

### Example 13—Stability of V_{H}Hs in GI Tract Fluid

Background: The GI tract contains proteases (proteins that degrade other proteins) and, in some compartments, is acidic. NBXs identified as useful in in vitro studies should be able to survive the GI tract conditions in order to be useful in vivo. We have developed an ex vivo model using the contents of chicken GI tract organs to mimic in vivo conditions and determine survival of V_{H}H under these conditions.

Detailed Protocol: The GI tract was removed from a chicken and segmented by organ. The contents are collected from the organs, diluted 1:2 in water, centrifuged to pellet solids, and the supernatant is collected. NBXs are added to gut fluid samples or normal saline (untreated) at a final concentration of 0.625 μg/μL. The samples are incubated at 42° C. for a time equivalent to the natural transit time of material through the organ. In the case of the gizzard, provided as an example below, the incubation lasted for 30 minutes. Reactions are stopped on ice with the addition of sodium dodecyl sulfate loading dye, immediately vortexed, and boiled at 95° C. Samples are separated by size using sodium dodecyl sulfate-polyacrylamide gel electrophoresis (SDS-PAGE) and gels are stained with Coomassie R-250.

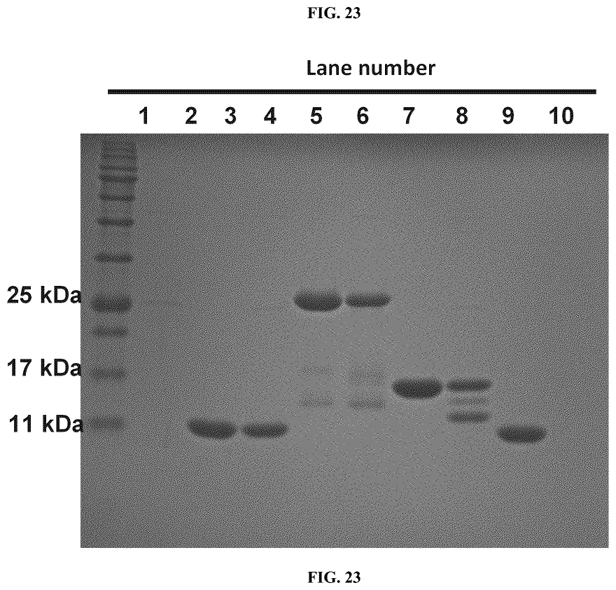

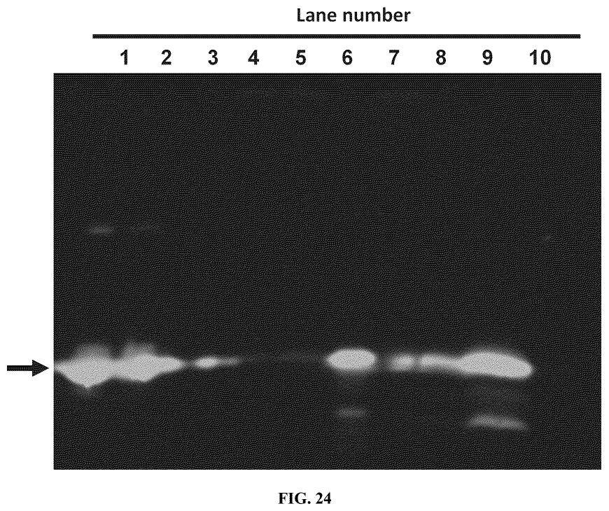

FIG. 23 shows that NBX0018 monomer and NBX0018-NBX0018 concatemer survive the ex vivo gizzard fluids better than NBX0015 or NBX0005. Lane 1 is a molecular weight marker and representative molecular weights are listed to the left of the gel. Lane 2 is the gizzard extract alone which indicates that there are very few background bands in the gizzard extract and that all bands in subsequent lanes are due to the presence of added NBXs. NBX0018 survives the gizzard extract well (lane 4) compared to the untreated control (lane 3). Similarly, the NBX0018-NBX0018 concatemer is also stable in the gizzard extract (lane 6) compared to the untreated control (lane 5). Some NBX0015 is degraded in the gizzard extract (lane 8) compared to the untreated control (lane 7). NBX0005 is completely degraded in the gizzard extract (lane 10) compared to the untreated control (lane 9).

### Example 14—V_{H}Hs can be Detected Intact Throughout the GI Tract of a Chicken After Oral Administration

3 mg of NBX0018 was orally administered to a day-old chick. 30 minutes later, the GI tract was removed from the chicken and segmented by organ. The contents of the organs were collected and 10 μL of organ fluid was separated by size using SDS-PAGE. The material in the gel was transferred to nitrocellulose membranes and visualized by Western Blotting using an anti-His antibody.

FIG. 24 shows that NBX0018 can be detected intact in chicken GI tract. Lanes 1 and 2 are both loaded with purified NBX0018 (marked with an arrow) to show the size of the intact protein. There is intact NBX0018 in the crop (lane 3), proventriculus (lane 4), gizzard (lane 5), duodenum (lane 6), jejunum (lane 7), ileum (lane 8), caecum (lane 9). Degradation products (bands running below the main band) are seen in the duodenum, jejunum, ileum, and caecum. Only the large intestine (lane 10) does not contain intact NBX0018.

### Example 15—V_{H}Hs are Non-Toxic to Chickens After Administration

Detailed Protocol: NBX0018, NBX0018-NBX0018 concatemer or PBS (negative control) was orally administered four times over a 3-day period to 7-day old chicks. Final body weights and spleen weights (as a % of body weights) were determined for each animal.

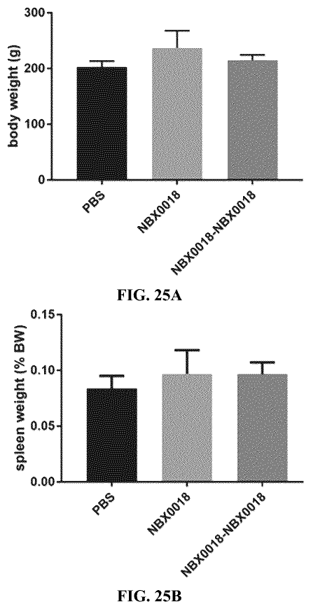

FIG. 25 shows that there were no statistically significant differences in (25A) body weights or (25B) spleen weights as a percentage of body weight (BW) in chickens treated with PBS (negative control), NBX0018, or NBX0018-NBX0018 concatemer. This indicates that there is no significant toxicity induced by NBXs in the chickens. Data represent the means of three (PBS) or five (NBX0018 and NBX0018-NBX0018) chickens per group and error bars represent the standard deviations. In addition, no GI lesions, organ damage, or abnormal behaviours were noted for any of the treatments.

### Example 16—Crude V_{H}H Purification from Periplasmic Extracts and Activity in Bacterial Motility Assay

NBX genes are cloned into the phagemid pRL144 and transformed into E. coli TG-1 cells as part of the phage display portion of antibody discovery. For NBX of interest, crude periplasmic extracts containing the NBX were prepared as follows. Bacteria are grown in 2×TY, 100 μg/ml ampicillin, and 0.1% (wt/vol) glucose for 3 hours at 37° C. 1 mM IPTG is added to induce protein production and the bacteria are incubated overnight at 30° C. Bacteria cells were pelleted by centrifugation and the supernatants were discarded. Cell pellets were frozen at −20° C. overnight. Pellets were thawed at room temperature for 15 minutes, resuspended in 500 μL of PBS, and rotated at 700 RPM for 30 minutes at room temperature to release the periplasmic contents. Remaining bacterial spheroplasts were pelleted and the NBX-containing supernatants were collected for in vitro testing. The live imaging motility assay protocol from example 4 was used to test the activity of crude periplasmic extracts. Results are shown in Table 7.

TABLE 7

| | Minimum Periplasmic Extract Concentration |
|---|---|
| NBX Construct | Required for 50% Motility Inhibition |
| NBX0202 | >66.7% |
| NBX0204 | 6.67% |
| NBX0205 | 6.67% |

Minimum periplasmic extract concentrations containing NBXs required to inhibit Salmonella enterica serovar Enteritidis strain LK5 motility by >50%. The concentration reported for NBX0202 was the highest concentrations tested and had no impact on bacterial motility.

### Example 17—Purification of NBXs from E. coli

TEV protease-cleavable, 6xHis-thioredoxin-NBX fusion proteins are expressed in the cytoplasm of E. coli grown in autoinducing media (Formedium) for 24 hours at 30° C. Bacteria are collected by centrifugation, resuspended in buffer A (10 mM HEPES, pH 7.5, 500 mM NaCl, 20 mM Imidazole) and lysed using homogenization. Insoluble material is removed by centrifugation and the remaining soluble fraction is applied to a HisTrap column (GE Biosciences) pre-equilibrated with buffer A. The protein is eluted from the column using an FPLC with a linear gradient between buffer A and buffer B (10 mM HEPES, pH 7.5, 500 mM NaCl, 500 mM Imidazole). The eluted protein is dialyzed overnight in the presence of TEV protease to buffer C (10 mM HEPES, pH 7.5, 500 mM NaCl). The dialyzed protein is applied to a HisTrap column (GE Biosciences) pre-equilibrated with buffer C. 6xHis-tagged TEV and 6xHis-tagged thioredoxin are bound to the column and highly purified NBX is collected in the flowthrough. NBX proteins are dialyzed overnight to PBS and concentrated to ˜10 mg/ml.

### Example 18—Purification of NBXs from Pichia pastoris

Pichia pastoris strain GS115 with constructs for the expression and secretion of 6xHis-tagged NBX were grown for 5 days at 30° C. with daily induction of 0.5% (vol/vol) methanol. Yeast cells were removed by centrifugation and the NBX-containing supernatant was spiked with 10 mM imidazole. The supernatant was applied to a HisTrap column (GE Biosciences) pre-equilibrated with buffer A (10 mM HEPES, pH 7.5, 500 mM NaCl). The protein was eluted from the column using an FPLC with a linear gradient between buffer A and buffer B (10 mM HEPES, pH 7.5, 500 mM NaCl, 500 mM Imidazole). NBX proteins were dialyzed overnight to PBS and concentrated to ˜1.5 mg/ml.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### Antigen Sequences and Selected V_{H}H Multimer Sequences

Salmonella Typhimurium
SEQ ID NO: 274 FimA:
>sp|P37921|FIMA1_SALTY Type-1 fimbrial protein, A chain OS = Salmonella
typhimurium (strain LT2/SGSC1412/ATCC 700720) GN = fimA PE = 1 SV = 2
MKHKLMTSTIASLMFVAGAAVAADPTPVSVSGGTIHFEGKLVNAACAVSTKSADQTVTLG
QYRTASFTAIGNTTAQVPFSIVLNDCDPKVAANAAVAFSGQADNTNPNLLAVSSADNSTT
ATGVGIEILDNTSSPLKPDGATFSAKQSLVEGTNTLRFTARYKATAAATTPGQANADATF
IMKYE
SEQ ID NO: 275 FliC:
>sp|P06179|FLIC_SALTY Flagellin OS = Salmonella typhimurium (strain LT2/
SGSC1412/ATCC 700720) GN = fliC PE = 1 SV = 4
MAQVINTNSLSLLTQNNLNKSQSALGTAIERLSSGLRINSAKDDAAGQATANRFTANIKG
LTQASRNANDGISIAQTTEGALNEINNNLQRVRELAVQSANSTNSQSDLDSIQAEITQRL
NEIDRVSGQTQFNGVKVLAQDNTLTIQVGANDGETIDIDLKQINSQTLGLDTLNVQQKYK
VSDTAATVIGYADTTIALDNSTFKASATGLGGIDQKIDGDLKFDDITGKYYAKVIVIGGT
GKDGYYEVSVDKTNGEVTLAGGATSPLTGGLPATATEDVKNVQVANADLTEAKAALTAAG
VTGTASVVKMSYTDNNGKTIDGGLAVKVGDDYYSATQNKDGSISINTTKYTADDGTSKTA
LNKLGGADGKIEVVSIGGKTYAASKAEGHNFKAQPDLAEAAATTTENPLQKIDAALAQVD
TLRSDLGAVQNRFNSAITNLGNTVNNLTSARSRIEDSDYATEVSNMSRAQILQQAGTSVL
AQANQVPQNVLSLLR
SEQ ID NO: 276 Prgl:
>sp|P41784|PRGI_SALTY Protein PrgI OS = Salmonella typhimurium (strain
LT2/SGSC1412/ATCC 700720) GN = prgI PE = 1 SV = 1
MATPWSGYLDDVSAKFDTGVDNLQTQVTEALDKLAAKPSDPALLAAYQSKLSEYNLYRNA
QSNTVKVFKDIDAAIIQNFR
Salmonella Enteritidis:
SEQ ID NO: 277 FimA:
>tr|Q53483|Q53483_SALEN FimA OS = Salmonella enteritidis GN = fimA
PE = 4 SV = 1
MKHKLMTSTIASLMFVAGAAVAADPTPVSVSGGTIHFEGKLVNAACAVSTKSADQTVTLG
QYRTASFTAIGNTTAQVPFSIVLNDCDPKVAATAAVAFSGQADNTNPNLLAVSSADNSTT
ATGVGIEILDNTSSPLKPDGATFSAKQALVEGTNTLRFTARYKATATATTPGQANADATF
IMKYE
SEQ ID NO: 278 FliC:
>sp|Q06972|FLIC_SALEN Flagellin OS = Salmonella enteritidis GN = fliC
PE = 3 SV = 2
MAQVINTNSLSLLTQNNLNKSQSSLSSAIERLSSGLRINSAKDDAAGQATANRFTSNIKG
LTQASRNANDGISIAQTTEGALNEINNNLQRVRELSVQATNGTNSDSDLKSIQDEIQQRL
EEIDRVSNQTQFNGVKVLSQDNQMKIQVGANDGETITIDLQKIDVKSLGLDGFNVNGPKE
ATVGDLKSSFKNVTGYDTYAAGADKYRVDINSGAVVTDAAAPDKVYVNAANGQLTTDDAE
NNTAVDLEKTTKSTAGTAEAKAIAGAIKGGKEGDTFDYKGVTFTIDTKTGDDGNGKVSTT
INGEKVTLTVADIATGATDVNAATLQSSKNVYTSVVNGQFTFDDKTKNESAKLSDLEANN
AVKGESKITVNGAEYTANATGDKITLAGKTMFIDKTASGVSTLINEDAAAAKKSTANPLA
SIDSALSKVDAVRSSLGAIQNREDSAITNLGNIVINLNSARSRIEDADYATEVSNMSKAQ
ILQQAGTSVLAQANQVPQNVLSLLR
SEQ ID NO: 279 Prgl:
>tr|A0A0H3T8G7|A0A0H3T8G_SALEN PrgI protein OS = Salmonella enteritidis
GN = AC092_14050 PE = 4 SV = 1
MATPWSGYLDDVSAKFDTGVDNLQTQVTEALDKLAAKPSDPALLAAYQSKLSEYNLYRNA
QSNTVKVFKDIDAAIIQNFR
SEQ ID NO: 460 SipD Amino Acid Sequence (PubMed Locus WP_000932249)
MLNIQNYSASPHPGIVAERPQTPSASEHVETAVVPSTTEHRGTDIISLSQAATKIQQAQQTLQSTPPISEENN
DERTLARQQLTSSLNALAKSGVSLSAEQNENLRSAFSAPTSALFSASPMAQPRTTISDAEIWDMVSQNISAIG
DSYLGVYENVVAVYTDFYQAFSDILSKMGGWLLPGKDGNIVKLDVTSLKNDLNSLVNKYNQINSNTVLFPAQS
GSGVKVATEAEARQWLSELNLPNSCLKSYGSGYVVTVDLTPLQKMVQDIDGLGAPGKDSKLEMDNAKYQAWQS
GFKAQEENMKTTLQTLTQKYSNANSLYDNLVKVLSSTISSSLETAKSFLQG
SEQ ID NO: 461 Prgl-SipD Amino Acid Sequence:
ATPWSGYLDDVSAKFDTGVDNLQTQVTEALDKLAAKPSDPALLAAYQSKLSEYNLYRNAQSNTVKVFKDIDAA
IIQNFRGGSGGTTISDAEIWDMVSQNISAIGDSYLGVYENVVAVYTDFYQAFSDILSKMGGWLLPGKDGNTVK
LDVTSLKNDLNSLVNKYNQINSNTVLFPAQSGSGVKVATEAEARQWLSELNLPNSCLKSYGSGYVVTVDLTPL
QKMVQDIDGLGAPGKDSKLEMDNAKYQAWQSGFKAQEENMKTTLQTLTQKYSNANSLYDNLVKVLSSTISSSL
ETAKSFLQG
SEQ ID NO: 462 NBX0018-NBX0018
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVTVSSGGGGSGGGGSGGGGSQVKLEESGGGLV
QPGGSLEVSCAASGIIFSPNAMGWYRQAPGEQRELVATITSFGIINYADSVKDRFTISRDNAKNTVYLQMTSL
KPEDTAVYYCNAKTFDGTRWHDYWGQGTQVTVSS
SEQ ID NO: 463 NBX0018-NBX0018-NBX0018
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVTVSSGGGGSGGGGSGGGGSQVKLEESGGGLV
QPGGSLEVSCAASGIIFSPNAMGWYRQAPGEQRELVATITSFGIINYADSVKDRFTISRDNAKNTVYLQMTSL
KPEDTAVYYCNAKTFDGTRWHDYWGQGTQVTVSSGGGGSGGGGSGGGGSQVKLEESGGGLVQPGGSLEVSCAA
SGIIFSPNAMGWYRQAPGEQRELVATITSFGIINYADSVKDRFTISRDNAKNTVYLQMTSLKPEDTAVYYCNA
KTFDGTRWHDYWGQGTQVTVSS
SEQ ID NO: 464 NBX0005-NBX0005
QVQLVESGGGLVQPGGSLTLSCIVSGRSVSINPMYWYRQGPGKQRELVVSLLPSGRTHDAHFAKGRFIISRDN
AKNTVYLQMNSLKPEDTAVYYCNTADFWGQGTQVTVSSGGGGSGGGGSGGGGSQVQLVESGGGLVQPGGSLTL
SCIVSGRSVSINPMYWYRQGPGKQRELVVSLLPSGRTHDAHFAKGRFIISRDNAKNTVYLQMNSLKPEDTAVY
YCNTADFWGQGTQVTVSS
SEQ ID NO: 465 NBX0015-NBX0015
QVQLVESGGGLVQAGDSLRLSCTASGRTFSNNAMGWFRQAPGKQRELVAAISRAGNTNYADSMKGRVTISGDN
AKNTVYLQMNSLKPEDTAVYYCKASSGSSVYIGVGSWGQGTQVTVSSGGGGSGGGGSGGGGSQVQLVESGGGL
VQAGDSLRLSCTASGRTFSNNAMGWFRQAPGKQRELVAAISRAGNTNYADSMKGRVTISGDNAKNTVYLQMNS
LKPEDTAVYYCKASSGSSVYIGVGSWGQGTQVTVSS
SEQ ID NO: 466 NBX0018-E9 Immunity Protein
MQVKLEESGGGLVQPGGSLEVSCAASGIIFSPNAMGWYRQAPGEQRELVATITSFGIINYADSVKDRFTISRD
NAKNTVYLQMTSLKPEDTAVYYCNAKTFDGTRWHDYWGQGTQVIVSSELKHSISDYTEAEFLQLVTTICNADT
SSEEELVKLVTHFEEMTEHPSGSDLIYYPKEGDDDSPSGIVNTVKQWRAANGKSGFKQG
SEQ ID NO: 467 NBX0018-Colicin E9
MQVKLEESGGGLVQPGGSLEVSCAASGIIFSPNAMGWYRQAPGEQRELVATITSFGIINYADSVKDRFTISRD
NAKNTVYLQMTSLKPEDTAVYYCNAKTFDGTRWHDYWGQGTQVIVSSESKRNKPGKATGKGKPVGDKWLDDAG
KDSGAPIPDRIADKLRDKEFKSFDDFRKAVWEEVSKDPELSKNLNPCNKSSVSKGYSPFTPKNQQVGGRKVYE
LHADKPISQGGEVYDMDNIRVTTPKRHIDIHRGK
SEQ ID NO: 468 NBX0018-GCN4 PIL
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVIVSSGGGSGGGSRMKQLEDKIEELLSKIYHL
ENEIARLKKLIGER
SEQ ID NO: 469 NBX0018-GCN4 PII
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVTVSSGGGSGGGSRMKQIEDKIEEILSKIYHI
ENEIARIKKLIGER
SEQ ID NO: 470 NBX0018-GCN4 PLI
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVTVSSGGGSGGGSRMKQIEDKLEEILSKLYHI
ENELARIKKLLG
SEQ ID NO: 471 NBX0018-Fos
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVTVSSGGGGGGGGGGLTDTLQAETDQLEDEKS
ALQTEIANLLKEKEKLEFILAA
SEQ ID NO: 472 NBX0018-Jun
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVIVSSGGGGGGGGGGRIARLEEKVKILKAQNS
ELASTANMLREQVAQLKQKVMN
SEQ ID NO: 473 NBX0018-Kv1.2 T1 Domain
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVTVSSGGGGSERVVINISGLRFETQLKTLAQF
PETLLGDPKKRMRYFDPLRNEYFFDRNRPSFDAILYYYQSGGRLRRPVNVPLDIFSEEIRFYELG
SEQ ID NO: 474 NBX0018-CsgC
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVTVSSGGGGSGGGGSGGGGSGGGGSGGGGSAL
SNQITFITTQQGDIYTVIPQVTLNEPCVCQVQILSVRDGVGGQSHTQQKQTLSLPANQPIELSRLSVNISSED
SVKIIVTVSDGQSLHLSQQWPPSAQ
SEQ ID NO: 475 NBX0018
QVKLEESGGGLVQPGGSLEVSCAASGIIFSPNAMGWYRQAPGEQRELVATITSFGIINYADSVKDRFTISRDN
AKNTVYLQMTSLKPEDTAVYYCNAKTFDGTRWHDYWGQGTQVTV
SEQ ID NO: 476 NBX0005
QVQLVESGGGLVQPGGSLTLSCIVSGRSVSINPMYWYRQGPGKQRELVVSLLPSGRTHDAHFAKGRFIISRDN
AKNTVYLQMNSLKPEDTAVYYCNTADFWGQGTQVTV
SEQ ID NO: 477 NBX0015
QVQLVESGGGLVQAGDSLRLSCTASGRTFSNNAMGWFRQAPGKQRELVAAISRAGNTNYADSMKGRVTISGDN
AKNTVYLQMNSLKPEDTAVYYCKASSGSSVYIGVGSWGQGTQVTV

| SEQ ID | Clone | Amino acid sequence |
|---|---|---|
| 478 | >NBX0005 | QVQLVESGGGLVQPGGSLTLSCIVSGRSVSINPMYWYRQGPGKQRE |
| | | LVVSLLPSGRTHDAHFAKGRFIISRDNAKNTVYLQMNSLKPEDTAV |
| | | YYCNTADFWGQGTQVTVSS |
| 479 | >NBX0015 | QVQLVESGGGLVQAGDSLRLSCTASGRTFSNNAMGWFRQAPGKQRE |
| | | LVAAISRAGNTNYADSMKGRVTISGDNAKNTVYLQMNSLKPEDTAV |
| | | YYCKASSGSSVYIGVGSWGQGTQVTVSS |
| 480 | >NBX0018 | QVKLEESGGGLVQPGGSLEVSCAASGIIFSPNAMGWYRQAPGEQRE |
| | | LVATITSFGIINYADSVKDRFTISRDNAKNTVYLQMTSLKPEDTAV |
| | | YYCNAKTFDGTRWHDYWGQGTQVTVSS |
| 481 | >NBX0019 | QVKLEESGGGLVQPGGSLEVSCAASGIIFSPNAMGWYRQAPGEQRE |
| | | LVATITSFGIINYADSVKDRFTISRDNAKNTVYLQMTSLKPEDTAV |
| | | YYCNAKAFDGTRWHDYWGQGTQVIVSS |
| 482 | >NBX0024 | QVKLEESGGGLVQAGGSLRLSCAVSGSIFSTNVMGWFRQAPGKQRG |
| | | FVAHITSGGNIDYADSVNGRFTMSRDNAKNIVYLQMNSLKPEDTAV |
| | | YYCAAQTLGSSYYDAWGQGTQVTVSS |
| 483 | >NBX0027 | QVQLVESGGGLVQAGGSLRLSCAASGRTFSSYAMGWFRQAPGKERE |
| | | FVASINWSGGRIYYADSVKGRFTISRDNAKNTVYLQMNSLKPEDTA |
| | | VYYCNADYDNSGSYYYQKGNYEYDYWGQGTQVTVSS |
| 484 | >NBX0200 | QVQLQESGGGLVQAGGSLRLSCAASGRTSSSAYTAWFRQAPGNERE |
| | | FVASISWSGTTTYYADPVKGRFTISRDNAKNTVYLQMNSLKPDDTA |
| | | VYYCAADRRSTIGSPRQQYAYWGQGTQVTVSS |
| 485 | >NBX0201 | QVQLQESGGGLVQAGGSLRLSCAASTRTSSSSYTAWFRQAPGNERE |
| | | FVASISYSGTTTYYADPVKGRFTISRDSAKNTVYLQMNSLKPDDTA |
| | | VYYCAADRRSTIGSPRQQYAYWGQGTQVTVSS |
| 486 | >NBX0202 | QVQLQESGGGLVQAGGSLRLSCAASGRTSSSAYTAWFRQAPGNERE |
| | | FVASISWSGTTTYHAHPVKGRFTIFRDNAKNIVYLQMNSLKPDDTG |
| | | VYYCAADRRSTIGTPREQYAYWGQGTQVTVSS |
| 487 | >NBX0204 | QVQLQESGGGLVQAGGSLRLSCAASGSTLSNYAVEWYRQAPGNQRE |
| | | YVARISSGGSTNYADSVKGRFTISRDNAKNTVYLQMNSLKPEDTAV |
| | | YYCHTYDFQGWGLRSDYWGQGTQVTVSS |
| 488 | >NBX0205 | QVQLQESGGRLVQAGGSLRLSCAASGRTFSSLAMGWFRQAPGKERE |
| | | FVAAISRSGDYTYFSDSVKGRFAISRDNAKDTVSLQMNNLKPDDTA |
| | | VYTCAATKIVTPWTSTYYYTKAYEWDYWGQGTQVTVSS |
| 489 | >NBX0104 | QVQLVESGGGLVQPGGSLRLSCAASGVAFNSRIMGWYRQAPGKQRE |
| | | LVALITSGGSTNYADSVKGRFTISRNNAKKTVYLQMNSLKPEDTAV |
| | | YYCNIRNYWGQGTQVTVSS |
| 490 | >NBX0105 | QVQLVESGGGLVQAGGSLRLSCAASGRTFNTYYMGWFRQAPGKERE |
| | | FVSAIRWSDGGTWYADSMKGRFTISRDNAKNTGYLQMNSLKPEDTA |
| | | IYYCNANVYDGNRWRTYWGQGTQVTVSS |
| 491 | >NBX0108 | QVKLEESGGGLVRAGGSLTLSCGASRGTFRTYSMGWFRQAPGKERE |
| | | FVAAITWNGKYTYYGDSVQGRFTISKDNAKNTVSLQMNRLNPEDTA |
| | | VYYCAANPIPTAQPPGIMAARSYVHWGQGTQVTVSS |
| 492 | >NBX0203 | QVQLQQSGGGLVQAGGSLRLSCAASGRTSPSSYTAWFRQAPGNERE |
| | | FVASISWSGTTTYYADPVKGRFTISRDNAKNTVYLQMNSLKPDDTA |
| | | VYYCAADRRSTIGSPRQQYAYWGQGTQVTVSS |
| 493 | >NBX0206 | QVQLQESGGRLVQAGGSLRLSCAASGRTFSSLAMGWFRQAPGKERE |
| | | FVAAITRSGDYTYFSDSVKGRFAISRDNAKDTVSLQMNNLKPDDTA |
| | | VYTCAATKIVTPWTSTYYYTKAYEWDYWGQGTQVTVSS |
| 494 | >NBX0207 | QVQLQESGGGLVQAGKSLRLSCAASTAILSIDSMGWNRQAVGNQRE |
| | | LVAVIARGGSTKYADSVKGRFTITRDISKNTIYLQMNSLKPEDTGV |
| | | YYCAADPGGASPLSWGQGTQVTVSL |
| 495 | >NBX0208 | QVQLQESGGGMVQAGGSLRLACTASGDISTIDVMGWNRQAPGKHRE |
| | | LVAIIARGGTIKYADSVKGRFTISRDNTKNTVTVYLQMNNVNAEDT |
| | | AVYYCAVDTGSPRLTWGQGTQVTVSS |
| 496 | >NBX0209 | QVQLQESGGGLVQAGGSLRLSCAASGFTFSSSIMAWYRQAPGKQRE |
| | | AIASIPSFGSAVYADSVKDRFTISRDNNKNMVYLQMNSLKPEDTAV |
| | | YYCNTRLYWGQGTQVTVSS |
| 497 | >NBX0210 | QVQLQESGGGLVQAGGSLRLACTASGDISSISVMGWNRQAPGKQQR |
| | | ELVAAIASGGSVKYADSVKGRFTISRDNIKNIVYLQMNSVNAEDTA |
| | | VYYCAVDTGSPRLTWGQGTQVTVSS |
| 498 | >NBX0211 | QVQLQESGGGLVQAGGSLRLSCAASGFTFSTNILAWYRQAPGKQRE |
| | | AIASITPFGSAVYANSVKDRFTISRDNNKNMVYLQMNSLKPEDTAV |
| | | YSCNTQLYWGQGTQVTVST |
| 499 | >NBX0212 | QVQLQESGGGLVQAGGSLRLSCAASTSILSINAMGWNREAPGNRRE |
| | | MVAIIAPGGTTNYADSVKGRFTITRDISKNTIYLQMNNLKPEDTGV |
| | | YYCAADPGGQSPLSWGQGTQVTVSL |
| 500 | >NBX0213 | QVQLQESGGGSVQAGGSLRLSCAASGSISSITAMGWNRQAPGNQRE |
| | | LVAVIARGGMIKYDDSVKGRFTISRDIAKNTVFLQMDSLKPEDTGV |
| | | YYCAVDNGDPRLHWGQGIQVTVSS |
| 501 | >NBX0214 | QVQLQESGGGLVQAGGSLRLSCAASGSISSITAMGWNRQAPGNQQR |
| | | DLVAVIARGGMTKYADSVQGRFTISRDIAKNTVYLQMNSLKPEDTG |
| | | VYYCALDNGDPRLHWGQGIQVTVSS |
| 502 | >NBX0215 | QVQLQESGGGLVQAGGSLRLSCAASGFTFSSAIMAWYRQAPGKQRE |
| | | AIASIPSFGSAVYADSVKDRFTISRDNNKNMVYLQMDSLKPEDTAV |
| | | YYCNTRLYWGQGTQVTVST |
| 503 | >NBX0216 | QVQLQESGGGLVQAGGSLRLSCAASTSILSIDAMGWNRQAPGNQRR |
| | | DLVAVIARGGSTQYADSVKGRFTITRDISKNTIYLQMNSLKPEYTG |
| | | VYYCAADPGGASGLSWGQGTQVTVSL |
| 504 | >NBX0217 | QVQLQESGGGLVQAGGSLRLSCAASGSISSITAMGWNRQAPGNQQR |
| | | DLVAVIARGGMTKYADSVQGRFTISRDIANNTVYLQMNSLKPEDTG |
| | | VYYCALYNGDPRLHWGQGIQVTVSS |
| 505 | >NBX0218 | QVQLQESGGGLVQAGGSLSLSCTASGSAFSGDAMGWYRRAPGKQRE |
| | | FVAGISSGAITNYMDSVKGRFTISRDNAKNTVYLQMTKLKPEDTAV |
| | | YYCNRIQAVLRGNSGWGQGTQVTVSS |
| 506 | >NBX0219 | QVQLQESGGGLVQPGGSLSLSCTASGSAFSGGDAMGWYRRAPGKQR |
| | | EFVAGISSGGIANYMDSVKGRFTISRDNAKKAVYLQMTSLKPEDTA |
| | | LYYCNSITAVLRGNSGWGQGTQVTVSS |
| 507 | >NBX0220 | QVQLQESGGGLVQAGGSLTLSCTASGSAFSGDAMGWYRRAPGKERE |
| | | FVAGISSGGIPNYMGSVQGRFTISRDNAKNTVYLQMRRLKPEDTAV |
| | | YYCNSISAVLRGNGVWGQGTQVTVSS |
| 508 | >NBX0221 | QVQLQESGGGLVQAGGSLRLSCAASGLTFNNYAMGWFRQAPGKERE |
| | | FVATISRDGTNTRYADSVKGRFTISRDNAKNTVDLQMNSLKPEDTA |
| | | VYYCGVGRGTGYAYTAINEYDYSKWGQGTQVTVSS |
| 509 | >NBX0222 | QVQLQESGGGLVQAGGSLRLSCAASGIDSSFYVMAWYRQAPGKQRE |
| | | LVASLGTPDSATYADFVKGRFIISRDNAKSTVYLQMNSLKPEDTAV |
| | | YYCYGLYRQVYWGQGTQVTVSS |
| 510 | >NBX0223 | QVQLQESGGGLVQAGGSLRLSCVASGIDSSFYVMAWYRQAPGKQRE |
| | | LVASISSADSPRYEDFVKGRFTISRDNGKNTVYLQMNSLKPEDTAV |
| | | YYCYGLYRQVHWGQGTQVTVSS |
| 511 | >NBX0224 | QVQLQQSGGGLVQAGGSLRLSCAASGLTFSSYAMGWFRQAPGKERE |
| | | FIVAIGWSGGSTYYADSVKGRFTISRDNAKNTVYLHMNSLKPEDTA |
| | | VYYCAARRTTAWGKGTDYWGQGTQVTVSS |
| 512 | >NBX0225 | QVQLQESGGGLVQAGGSLRLSCAASESIFSRNAMGWYRQAPGKERD |
| | | LVAPGKERELVAGIGSDGSTNYAESVKGRFTISRDNAKNTVYLQMN |
| | | SLKPEDTAVYYCRVVLATSPYNYWGQGTQVTVSS |
| 513 | >NBX0226 | QVQLQESGGGLVQAGGSLRLSCAASGITSSLYVMAWYRQAPGKQRE |
| | | LVAHINSGDSPRYADFVQGRFTISRDNGKNTVYLQMNSLKPEDTAV |
| | | YYCYGLYRQVHWGQGTQVTVSS |
| 514 | >NBX0227 | QVQLQESGGGLVQAGGSLRLACAASGLTFNNYAMGWFRQAPGKERE |
| | | FVATISRDGTSTRYADSVKGRFTISRDNAKNTVNLQMNRLKPEDTA |
| | | VYYCGVGRGSGYAYSAINEYDYSSWGQGTQVTVSS |
| 515 | >NBX0228 | QVQLQESGGGLVQAGGSLRLSCAASGIDSSFYVMAWYRQAPGQQRE |
| | | LVASISMTSADSPRYADFVKGRFTISRDNAKSTVYLQMNSLKPEDT |
| | | AVYYCYGLYRQVHWGQGTQVTVSS |
| 516 | >NBX0229 | QVQLQESGGGLVQAGGSLRLSCAASGSGILFRISAMGWYRQAPGKE |
| | | RDLVAGISSGGSTNYADSVKGRFTISRDNAKNTVYLQMNSLKPEDT |
| | | AVYYCNIVGRIDSWGQGTQVIVSS |
| 517 | >NBX0230 | QVQLQESGGGSVQAGGSLRLSCAASARTLSNYAMGWFRQAPGKERE |
| | | FVATISRSGGSIHYADSVKGRFTISRDNAKNTVNLQMNSLKVEDTA |
| | | VYYCGRARGTGYAYTALNQYDYDYWGQGTQVTVSS |
| 518 | >NBX0231 | QVQLQESGGGLVQAGGSLTLSCITSGSAFSGDAMGWYRRAPGQERE |
| | | FVAGISSGGITNYMNFVKGRFTISRDNAKNTVYLQMTSLKPEDTAV |
| | | YYCNSIKAVLRGNSGWGQGTQVTVSS |
| 519 | >NBX0232 | QVQLQESGGGLVQAGGSLRLSCAASGLTFHNYAMGWFRQAPGKERE |
| | | FVATISRDGTNTHYADSVKGRFTISRDNAKNTVDLQMNSLKPEDTA |
| | | VYYCGVGRGSGYAYTAINEYDYSKWGQGTQVTVSS |
| 520 | >NBX0233 | QVQLQESGGGLVQAGGSLSLSCTASGSAFSGDAMGWYRRAPGKQRD |
| | | FVAGISSGHITNYMDSVKGRFTISRDNAKNTVYLQMTKLKPEDTAV |
| | | YYCNSITAVLRGNSGWGQGTQVTVSS |
| 521 | >NBX0234 | QVQLQESGGGLVQAGGSLRLSCAASGRTFSTYAMGWFRQAPGKERE |
| | | FVATISRSGDNIYYADSVKGRFTISRDNAKNTVSLQMNSLKVEDTA |
| | | VYYCGRARGTGYAHTALNQYDYDYWGQGTQVTVSS |
| 522 | >NBX0235 | QVQLQESGGGLVQAGGSLSLSCRVSGSAFSGDAMGWYRRAPGKQRE |
| | | FVAGISSGGIENYMDSVKGRFTISRDNAKNTVYLRMSSLKPEDTAV |
| | | YYCNLIKAVLRGNSGWGQGTQVTVSS |
| 523 | >NBX0236 | QVQLQESGGGLVQAGGSLRLSCAASGLTFNNYAMGWFRQAPGKERE |
| | | FVATISRDGTNTRYADSVKGRFTISRDNAKNTVNLQMNSLKPEDTA |
| | | VYYCGVGRGTGYAYTAIREHDYSSWGQGTQVTVSS |
| 524 | >NBX0237 | QVQLQESGGGLVQAGGSLSLSCTASGSAFSGDAMGWYRRAPGKQRE |
| | | FVAGISSGGITNYMNSVKGRFTISRDNAKNTVYLHMTGVKPADTAV |
| | | YYCNSITAVLRGNSGWGPGTQVIVSS |
| 525 | >NBX0238 | QVQLQESGGGLVQPGGSLTLSCTASGSAFSGDAMGWYRRAPGKQRE |
| | | FVAGISSGGIANYMDSTEGRFTISRDDAKNTVYLQMTGVKPADTAV |
| | | YYCNTIKAVLRGNAGWGQGTQVTVSS |
| 526 | >NBX0239 | QVQLQESGGGLVQAGGSLSLSCTASGSAFSGDAMGWYRRAPGKQRE |
| | | FVAGISSGAITNYMDSVKGRFTISRDNAKNMVYLQMTKLKPEDTAV |
| | | YYCNSITAVLRGNSGWGQGTQVTVSS |
| 527 | >NBX0240 | QVQLQESGGGLVQAGGSLSLSCTASGSAFSGDAMGWYRRAPGKQRE |
| | | FVAGISSGGITNYMDSVKGRFTISRDNAKNTVYLQMTSLKPEDTAV |
| | | YYCNIISAVLRGNGGWGQGTQVTVSS |
| 528 | >NBX0241 | QVQLQQSGGGLVQAGGSLSLSCTASISGFSGDAMGWYRRAPGKQRE |
| | | FVAGISSGGITNYMDSVKGRFTISRDNAKNTVYLQMTNLKPEDTAV |
| | | YYCNTITGVLRGNSGWGQGTQVTVSS |
| 529 | >NBX0242 | QVQLQESGGGLVQAGGSLRLSCAGSGIISSAYVMAWYRQRPGKQRE |
| | | LVASITSGDSPRYEDFVKGRFTISRDNAKSTVYLQMNSLKPEDTAV |
| | | YYCYGLYRQVYWGQGTQVTVSS |
| 530 | >NBX0243 | QVQLQQSGGGLVQAGGSLKLSCAASGIAFSTYGMNWFRQTPGKQRE |
| | | YVAYITGNGDDNVAQSMEGRFTISRDNAKNTGYLQMNSLKPEDTGV |
| | | YYCNIGMYWGQGTQVTVSS |
| 531 | >NBX0244 | QVQLQESGGGLVQAGGSLSLSCTASGSAFSGDAMGWYRRAPGKQRE |
| | | FVAGISSGGITNYMGFVKGRFTISRDNAKNTVYLQMTSLKPEDTAV |
| | | YYCNSISAVLRGNSGWGQGTQVTVSA |
| 532 | >NBX0245 | QVQLQESGGGLVQAGGSLSLSCTASGSAFSGDAMGWYRRAPGKQRD |
| | | FVAGISSGGITNYMDSVKGRFTISRDNAKNTVYLQMTSLKPEDTAV |
| | | YYCNSISAVLRGNGGWGQGTQVTVSS |
| 533 | >NBX0246 | QVQLQESGGGLVQPGGSLSLSCTASGSAFSGDAMGWYRRAPGKQRE |
| | | FVAGISSGGITNYMDSVKDRFTISRDNAKNTLYLQMTNLKPEDTAV |
| | | YYCNSITAVLRGNSDWGQGTQVTVSS |
| 534 | >NBX0247 | QVQLQESGGGLVQAGGSLSLSCTTSGSAFSGDAMGWYRRAPGKQRE |
| | | FVAGISSGGIPNYMGFVRGRFTISRDNTKNTVYLQMTSLKPDDTAV |
| | | YYCNIIKTVLRGNAVWGQGTQVTVSS |
| 535 | >NBX0248 | QVQLQESGGGLVQAGGSLSLSCTASGSAFSGGDAMGWYRRAPGKQR |
| | | EFVAGISSGGITNYMDFVKGRFTISRDNAKNTVYLQMTSLKPEDTA |
| | | VYYCNSITAVLRGNSGWGQGTQVTVSS |
| 536 | >NBX0249 | QVQLQESGGGLVQAGGSLRLSCVASGITFSSDAMGWYRQAPGKQRE |
| | | FVAGISSGDITNYPDSVKGRFAISRDNAKNTVYLQMNSLKPEDTAV |
| | | YYCNTITRLLYGMDYWGKGTLVTVSS |
| 537 | >NBX0250 | QVQLQESGGGLVQPGGSLRLSCAASGFTLDGYAIGWFRQAPGKERE |
| | | WVSCIIYRDGSPAYADSVWGRFTISRDNAKNNVYLEMNSLKPEDTA |
| | | VYYCAARPGGACSRYPSNYDTWGQGTQVTASS |
| 538 | >NBX0001 | QVKLEESGGGLVQAGGSLRLSCAASGSIFSINAMGWYRQAPGKQRE |
| | | LVAAITTGGNTANTAYADSVKGRFTISRDKAKNTVYLQMNSLKPED |
| | | TAVYYCAARGLSYEYDYWGQGTQVTVSS |
| 539 | >NBX0002 | QVQLVESGGGLVQAGGSLRLSCAASGSIFSINAMGWYRQAPGKQRE |
| | | LVAAITITSGRGGNTAYADSVKGRFTISRDNAKNTVYLQMNTLKPE |
| | | DTAVYYCAARGAMTYEYDYWGQGTQVTVSS |
| 540 | >NBX0004 | QVQLVESGGGLVQAGGSLRLSCAASGIIFSPNAMGWYRQAPGKQRE |
| | | LVSTITSFGIINYADSVKDTISRDNAKNTVYLQMTSLKPEDTAVYY |
| | | CNAKTFDGTRWRDYWGQGAQVTVSS |
| 541 | >NBX0006 | QVKLEESGGGLVQAGGSLRLSCAASGNIFSINAMGWYRQAPGKQRE |
| | | LVAAITTGGSYGNTNYADSVKGRFTISRDNAKNTVYLQMNSLKPED |
| | | TAVYYCAARGSQTYEYDYWGQGTQVTVSS |
| 542 | >NBX0007 | QVKLEESGGGLVQPGGSLTLSCAASGRIFSIYDMGWFRQAPGKERE |
| | | LVSAITWGNGNTAYGDSVKGRFSISRDFAKNTVYLQIDSLKAEDTA |
| | | VYYCPARIVNGGSWDYWGQGTQVTVSS |
| 543 | >NBX0008 | QVKLEESGGGLVQPGGSLTLSCAASGRIFSIYDMGWFRQAPGKERE |
| | | LVSAITWGNGNTAYGDSVKGRFSISRDFAKNTVYLQIDSLKAEDTA |
| | | VYYCPARIVNGGSWDYWGQGTQVTVSS |
| 544 | >NBX0009 | QVKLEESGGGLVQAGGSLRLSCAASGRMFSSYDMGWFRQAPGKERD |
| | | IVAAITKNGRTTSYANSVKGRFTISRDNTKSTVYLQIHSLKPEDTA |
| | | VYYCAGRRSNADNWDYWGQGTQVTVSS |
| 545 | >NBX0010 | QVKLEESGGGLVQAGGSLRLSCAVSGSIFSINAVGWYRQAPGKQRE |
| | | LVAAIGTGGSSSGNTAYADSVKGRFTISNDAAKNTVYLQMNSLKPD |
| | | DTAVYYCAARGTISYEYDYWGQGTQVTVSS |
| 546 | >NBX0011 | QVQLVESGGGLVQAGGSLTLSCIVSGISVNINPMYWYRPGPGNQRE |
| | | LVVSLLPTGITHDAHFIKGRFIISKDDAKNTVYLLMNSLKPEDTAV |
| | | YYCNTADFWGQGTQVTVSS |
| 547 | >NBX0012 | QVKLEESGGGLVQAGGSLRLSCAASGSTFSINAMGWFRQAPGKQRE |
| | | LVAAISRAGSTNTADSVKGRFTISRDNAKNTVYLQMNSLKPEDTAV |
| | | YYCKASSGSSVYIGFGSWGQGTQVTVSS |
| 548 | >NBX0013 | QVQLVESESGLVLAGGSLTLTRFCSVSSVSINPMYWYRQGPGKQRE |
| | | LVLILLSMARAQNAHFPNGQFLISIYEDDNTMYLQLSIQKPEDADV |
| | | YDCNTTDFWGQGTQVTVSS |
| 549 | >NBX0014 | QVKLEESGGGLVQAGGSLRLSCAASGRTFSRLAMGWFRQAPGKERE |
| | | FVVAISWSGGNTNYADSVKGRFTISRDNAKNTVYLQMNSLNAEDTA |
| | | VYYCAAPERSGSYAYTPSRLNEYAYWGQGTQVTVSS |
| 550 | >NBX0016 | QVQLVESGGGLVQAGGSLRLSCAASGRIFSSYDMGWFRQAPGKERE |
| | | LVAAIRWGNGNTAYGDSVKGRFSISRDFAKNTVYLHIDSLKAEDTA |
| | | VYYCAARGLAYEYEYWGQGTQVTVSS |
| 551 | >NBX0017 | QVQLVESGGGLVQAGGSLRLSCAASGRIFSSYDMGWFRQAPGKERE |
| | | LVAAIRWGNGNTAYGDSVKGRFSISRDFAKNTVYLHIDSLKAEDTA |
| | | VYYCAARIVNGGSWDYWGQGTQVTVSS |
| 552 | >NBX0020 | QVQLVESGGGSVQPAGSLRLSCAVSGIIFSPNALGWYRPAPGKERE |
| | | LVASIISGGRSDYADSVKDRFTIARDNPKNTVTLQMNSLKPEDTAI |
| | | YYCNANVYDGNRWRTYWGQGTQVTVSS |
| 553 | >NBX0021 | QVKLEESGGGLVQAGGSLRLSCAASGRTFRSYTMGWFRQAPGLERE |
| | | IIAAISWSAGSTRYADSMSDRFTISRDNAKNTVYLGMDSLKPEDTA |
| | | VYYCAAGTKYSDTIITWGSWGQGTQVTVSS |
| 554 | >NBX0022 | QVQLVESGGGLVQPGGSLRLSCAVSGSTVTISTVGWYRQAPGNQRV |
| | | LVASISSDSTTNYAHSVKGTISRHNAENPVSRLQMNSLKPEDTAVY |
| | | YCNVVGTYWTGADWRPFDTWGRGTQVIVSS |
| 555 | >NBX0023 | QVKLEESGGGLVQAGGSLRLSCAASGRSFSSYNMGWFRQAPGKERE |
| | | FVAAITWSGNTYYADSVKGTISRDNAKNTVYLQMNSLKPEDTAVYF |
| | | CKVRAEDTDYWGRGTQVTVSS |
| 556 | >NBX0025 | QVKLEESGGGLVQPGGSLRLSCAASGFTFSMYGMTWVRQAPGKGLE |
| | | WVSAINSGGARTSYADSVKGRFTISRDNAKNTLYLQMNSLKPEDTA |
| | | VYYCAKASLPWFDGSSPDYWGQGTQVTVSS |
| 557 | >NBX0026 | QVKLEESGGGLVQAGGSLRLSCAASGLTFSSYGMGWFRQGPGKERE |
| | | SVAAIKMSGDTYYTDSVKGRFTISRDNAKNTVYLQMDSLKPEDTAV |
| | | YFCAAARVRTPGWGPQKSYDYWGQGTQVTVSS |
| 558 | >NBX0028 | QVKLEESGGGLVQAGGSLRLSCAASGRTFGSLHMGWFRQAPGKERE |
| | | FVSAISAAGGVTDYADSAKGRFTISRDNAKNTVYLQMNSLKPEDTA |
| | | VYYCAAVKYWGRRQRADEYDYWGQGTQVTVSS |
| 559 | >NBX0029 | QVRLEKSGGGLVQPGGSLTLSCTASGSISSIKAMGWYRQAPGKQRE |
| | | LAALWRMYSGTAYGDSVKGRSNLSVNHTNNTAYLQMNSLRPEDTAV |
| | | YWCYLEIPESRGAFWGHGTQVTVSS |
| 560 | >NBX0030 | QVQLVESGGGLVQAGGSLRLSCAASGRTFSRDAMGWFRQAPGKERE |
| | | FVATINWNGRSTYYTESVKGRFTISRDNAKNTVYLQMNSLKPEDTA |
| | | VYYCAAGEWGIRPYNYDYWGQGTQVTVSS |
| 561 | >NBX0031 | QVQLVESGGGLVQAGGSLRLSCAASGRTFSSYAMGWFRQAPGKERE |
| | | FVASINWSGGRIYYADSVKGRFTISRDNAKNTVYLQMNSLKPEDTA |
| | | VYYCNTDYDNSGSYYYQKGNYEYDYWGQGTQVTVSS |
| 562 | >NBX0032 | QVQLVESGGGLVQPGGSLKVSCAASGIIFSPNAMGWYRQAPGKQRD |
| | | LVATITSSGIINYADSVKGTISRDNAKNTVYLQMTSLKPEDTAVYY |
| | | CNAKAFDGTRWYDYWGQGTQVTVSS |
| 563 | >NBX0033 | QVKLEESGGGLVQAGGSLRLSCAASGRTFSSYVMGWFREAPGKERR |
| | | FVASISWSGGSSSYADSVKGTISRDYAENMVYLQMNSLKPEDTATY |
| | | YCAARTALGGTYDYWGQGTQVTVSS |
| 564 | >NBX0034 | QVQLAESGVGLVEPAGSLKFSCAASGIIFNPNAMGWHRQAPENQRE |
| | | LVATITSFGIINYADSVKDSISRDHDTNAVYLQMTNLRPDDPAVYY |
| | | CNAITFYGTRWLDYWGQGTQVTVSS |
| 565 | >NBX0035 | QVQLVESGGGLVQPGGSLRLSCAVSGIIFSPNALGWYRQAPGKERE |
| | | LVASIISGGRSDYADSVKDTIARDNPKNTVTLQMNSLKPEDTAIYY |
| | | CNADVYDGNRWRTYWGQGTQVTVSS |
| 566 | >NBX0036 | QVKLEESGGGLVQPGGSLKVSCAASGIIFSPNAMGWYRQAPGKQRE |
| | | LVATITSFGIINYADSVKDTISRDNAKNTVYLQMTSLKPEDTAVFY |
| | | CFAKTFDGTRWCDYWGQGTQVTVSS |
| 567 | >NBX0037 | QVQLVESGAGLVQPGGSLRLSCAVSGIIFSPNALGWYRQAPGKERE |
| | | LVASIISGGRSDYADSVKDTIARDNPKNAVTLQMNSLKPEDTAIYY |
| | | CNAIVYDGNRWRTYWGQGTQVTVSS |
| 568 | >NBX0038 | QVKLEESGEGLVQAGGSLRLSCAASGRTFSSYAMAWFRQAPGKERE |
| | | FVARIRWTRSSTVYADSVKGTISGDNAKNTMYLQMNSLKPEDTAVY |
| | | YCAADRYYRTDIYRASSYEYWGQGTQVTVSS |
| 569 | >NBX0039 | QVKLEESGGGLVQAGGSLRLSCVVSGRPFINYNMGWFRQAPGKEHE |
| | | FVAAISWSGDSTYYEDSVKGTVSRDNAKNTVYLQMNNLKPEDTAVY |
| | | YCAADNQHDIPLRPGGWQGTQVTVSS |
| 570 | >NBX0040 | QVQLVESGGGLVEAGGSLTLSCAASGLAFNTKTMAWFRQAPDKERA |
| | | VVATITWGTINTSYADSVKGRFTISRDNAKNMVYLRMDSLKPEDTD |
| | | VYYCESEALLETTPSRRPYEYNYWGPGTQVTVSS |
| 571 | >NBX0041 | QVKLEESGGGLVQAGGSLRLSCAASGRIFGSLHMGWFRQAPGKERE |
| | | FVSPITAAGGVTDYDSSNEGIHSVLHKQRQEHVSSPMNSLKPDTHG |
| | | RLLLCRTLGCSYYERADEYNYWGQGTQVTVSS |
| 572 | >NBX0100 | QVKLEESGGGLVQAGGSLRLSCAVSGSIFSTNLMGWYRQAPGKQRG |
| | | FVAHITSGGNTDYLDSVKGRFTISRDNAKNTVYLQMNSLKPEDTAV |
| | | YYCAAQTLGSSYYDAWGQGTQVTVSS |

Exemplary Framework regions for use with the CDRs of tables 1 or 2

| Seq ID | FR1 | Seq ID | FR2 | Seq ID | FR3 | Seq ID | FR4 |
|---|---|---|---|---|---|---|---|
| 573. | QVQLVES | 614. | YWYRQGPGKQ | 670. | HDAHFAKGRFIISR | 753. | WGQGTQ |
| | GGGLVQP | | RELVVS | | DNAKNTVYLQMNS | | VTVSS |
| | GGSLTLSCI | | | | LKPEDTAVYYC | | |
| | VS | | | | | | |
| 574. | QVQLVES | 615. | GWFRQAPGKQ | 671. | NYADSMKGRVTIS | 754. | WGQGTQ |
| | GGGLVQA | | RELVAA | | GDNAKNTVYLQM | | VTVSL |
| | GDSLRLSC | | | | NSLKPEDTAVYYC | | |
| | TAS | | | | | | |
| 575. | QVKLEESG | 616. | GWYRQAPGEQ | 672. | NYADSVKDRFTISR | 755. | WGQGTQ |
| | GGLVQPG | | RELVAT | | DNAKNTVYLQMTS | | VTVST |
| | GSLEVSCA | | | | LKPEDTAVYYC | | |
| | AS | | | | | | |
| 576. | QVKLEESG | 617. | GWFRQAPGKQ | 673. | DYADSVNGRFTMS | 756. | WGQGIQ |
| | GGLVQAG | | RGFVAH | | RDNAKNTVYLQM | | VTVSS |
| | GSLRLSCA | | | | NSLKPEDTAVYYC | | |
| | VS | | | | | | |
| 577. | QVQLVES | 618. | GWFRQAPGKER | 674. | YYADSVKGRFTISR | 757. | WGPGTQ |
| | GGGLVQA | | EFVAS | | DNAKNTVYLQMNS | | VTVSS |
| | GGSLRLSC | | | | LKPEDTAVYYC | | |
| | AAS | | | | | | |
| 578. | QVQLVES | 619. | AWFRQAPGNER | 675. | YYADPVKGRFTISR | 758. | WGQGTQ |
| | GGGLVQP | | EFVAS | | DNAKNTVYLQMNS | | VTVSA |
| | GGSLRLSC | | | | LKPDDTAVYYC | | |
| | AAS | | | | | | |
| 579. | QVKLEESG | 620. | EWYRQAPGNQ | 676. | YYADPVKGRFTISR | 759. | WGKGTL |
| | GGLVRAG | | REYVAR | | DSAKNTVYLQMNS | | VTVSS |
| | GSLTLSCG | | | | LKPDDTAVYYC | | |
| | AS | | | | | | |
| 580. | QVQLQQS | 621. | GWFRQAPGKER | 677. | YHAHPVKGRFTIFR | 760. | WGQGTQ |
| | GGGLVQA | | EFVAA | | DNAKNTVYLQMNS | | VTASS |
| | GGSLRLSC | | | | LKPDDTGVYYC | | |
| | AAS | | | | | | |
| 581. | QVQLQES | 622. | GWYRQAPGKQ | 678. | NYADSVKGRFTISR | 761. | WGQGAQ |
| | GGRLVQA | | RELVAL | | DNAKNTVYLQMNS | | VTVSS |
| | GGSLRLSC | | | | LKPEDTAVYYC | | |
| | AAS | | | | | | |
| 582. | QVQLQES | 623. | GWFRQAPGKER | 679. | YFSDSVKGRFAISR | 762. | WGRGTQ |
| | GGGLVQA | | EFVSA | | DNAKDTVSLQMN | | VTVSS |
| | GKSLRLSC | | | | NLKPDDTAVYTC | | |
| | AAS | | | | | | |
| 583. | QVQLQES | 624. | GWNRQAVGNQ | 680. | NYADSVKGRFTISR | 763. | WGHGTQ |
| | GGGMVQ | | RELVAV | | NNAKKTVYLQMNS | | VTVSS |
| | AGGSLRLA | | | | LKPEDTAVYYC | | |
| | CTAS | | | | | | |
| 584. | QVQLQES | 625. | GWNRQAPGKH | 681. | WYADSMKGRFTIS | 764. | WQGTQV |
| | GGGLVQA | | RELVAI | | RDNAKNTGYLQM | | TVSS |
| | GGSLRLSC | | | | NSLKPEDTAIYYC | | |
| | AAS | | | | | | |
| 585. | QVQLQES | 626. | AWYRQAPGKQ | 682. | YYGDSVQGRFTISK | | |
| | GGGLVQA | | REAIAS | | DNAKNTVSLQMN | | |
| | GGSLRLAC | | | | RLNPEDTAVYYC | | |
| | TAS | | | | | | |
| 586. | QVQLQES | 627. | GWNRQAPGKQ | 683. | KYADSVKGRFTITR | | |
| | GGGSVQA | | QRELVAA | | DISKNTIYLQMNSL | | |
| | GGSLRLSC | | | | KPEDTGVYYC | | |
| | AAS | | | | | | |
| 587. | QVQLQES | 628. | GWNREAPGNR | 684. | KYADSVKGRFTISR | | |
| | GGGLVQA | | REMVAI | | DNTKNTVTVYLQM | | |
| | GGSLSLSC | | | | NNVNAEDTAVYYC | | |
| | TAS | | | | | | |
| 588. | QVQLQES | 629. | GWNRQAPGNQ | 685. | KYADSVKGRFTISR | | |
| | GGGLVQP | | RELVAV | | DNTKNTVYLQMNS | | |
| | GGSLSLSC | | | | VNAEDTAVYYC | | |
| | TAS | | | | | | |
| 589. | QVQLQES | 630. | GWNRQAPGNQ | 686. | VYANSVKDRFTISR | | |
| | GGGLVQA | | QRDLVAV | | DNNKNMVYLQMN | | |
| | GGSLTLSC | | | | SLKPEDTAVYSC | | |
| | TAS | | | | | | |
| 590. | QVQLQES | 631. | GWNRQAPGNQ | 687. | NYADSVKGRFTITR | | |
| | GGGLVQA | | RRDLVAV | | DISKNTIYLQMNNL | | |
| | GGSLTLSC | | | | KPEDTGVYYC | | |
| | ITS | | | | | | |
| 591. | QVQLQES | 632. | GWYRRAPGKQR | 688. | KYDDSVKGRFTISR | | |
| | GGGLVQA | | EFVAG | | DIAKNTVFLQMDSL | | |
| | GGSLSLSC | | | | KPEDTGVYYC | | |
| | RVS | | | | | | |
| 592. | QVQLQES | 633. | GWYRRAPGKER | 689. | KYADSVQGRFTISR | | |
| | GGGLVQP | | EFVAG | | DIAKNTVYLQMNS | | |
| | GGSLTLSC | | | | LKPEDTGVYYC | | |
| | TAS | | | | | | |
| 593. | QVQLQQS | 634. | GWFRQAPGKER | 690. | VYADSVKDRFTISR | | |
| | GGGLVQA | | EFVAT | | DNNKNMVYLQMD | | |
| | GGSLSLSC | | | | SLKPEDTAVYYC | | |
| | TAS | | | | | | |
| 594. | QVQLQES | 635. | AWYRQAPGKQ | 691. | QYADSVKGRFTITR | | |
| | GGGLVQA | | RELVAS | | DISKNTIYLQMNSL | | |
| | GGSLRLSC | | | | KPEYTGVYYC | | |
| | AGS | | | | | | |
| 595. | QVQLQQS | 636. | GWFRQAPGKER | 692. | KYADSVQGRFTISR | | |
| | GGGLVQA | | EFIVAI | | DIANNTVYLQMNS | | |
| | GGSLKLSC | | | | LKPEDTGVYYC | | |
| | AAS | | | | | | |
| 596. | QVQLQES | 637. | GWYRQAPGKER | 693. | NYMDSVKGRFTISR | | |
| | GGGLVQA | | DLVAP | | DNAKNTVYLQMTK | | |
| | GGSLSLSC | | | | LKPEDTAVYYC | | |
| | ITS | | | | | | |
| 597. | QVQLQES | 638. | AWYRQAPGKQ | 694. | NYMDSVKGRFTISR | | |
| | GGGLVQA | | RELVAH | | DNAKKAVYLQMTS | | |
| | GGSLRLSC | | | | LKPEDTALYYC | | |
| | VAS | | | | | | |
| 598. | QVQLQES | 639. | AWYRQAPGQQ | 695. | NYMGSVQGRFTIS | | |
| | GGGLVQP | | RELVAS | | RDNAKNTVYLQMR | | |
| | GGSLRLSC | | | | RLKPEDTAVYYC | | |
| | AAS | | | | | | |
| 599. | QVKLEESG | 640. | GWYRQAPGKER | 696. | RYADSVKGRFTISR | | |
| | GGLVQAG | | DLVAG | | DNAKNTVDLQMN | | |
| | GSLRLSCA | | | | SLKPEDTAVYYC | | |
| | AS | | | | | | |
| 600. | QVKLEESG | 641. | GWYRRAPGQER | 697. | TYADFVKGRFIISRD | | |
| | GGLVQPG | | EFVAG | | NAKSTVYLQMNSL | | |
| | GSLTLSCA | | | | KPEDTAVYYC | | |
| | AS | | | | | | |
| 601. | QVKLEESG | 642. | GWYRRAPGKQR | 698. | RYEDFVKGRFTISR | | |
| | EGLVQAG | | DFVAG | | DNGKNTVYLQMN | | |
| | GSLRLSCA | | | | SLKPEDTAVYYC | | |
| | AS | | | | | | |
| 602. | QVQLVES | 643. | AWYRQRPGKQ | 699. | YYADSVKGRFTISR | | |
| | GGGLVQA | | RELVAS | | DNAKNTVYLHMNS | | |
| | GGSLTLSCI | | | | LKPEDTAVYYC | | |
| | VS | | | | | | |
| 603. | QVQLVES | 644. | NWFRQTPGKQ | 700. | NYAESVKGRFTISR | | |
| | ESGLVLAG | | REYVAY | | DNAKNTVYLQMNS | | |
| | GSLTLTRF | | | | LKPEDTAVYYC | | |
| | CS | | | | | | |
| 604. | QVQLVES | 645. | GWFRQAPGKER | 701. | RYADFVQGRFTISR | | |
| | GGGSVQP | | EWVSC | | DNGKNTVYLQMN | | |
| | AGSLRLSC | | | | SLKPEDTAVYYC | | |
| | AVS | | | | | | |
| 605. | QVQLVES | 646. | GWYRQAPGKQ | 702. | RYADSVKGRFTISR | | |
| | GGGLVQP | | RELVAA | | DNAKNTVNLQMN | | |
| | GGSLRLSC | | | | RLKPEDTAVYYC | | |
| | AVS | | | | | | |
| 606. | QVKLEESG | 647. | GWYRQAPGKQ | 703. | RYADFVKGRFTISR | | |
| | GGLVQPG | | RELVST | | DNAKSTVYLQMNS | | |
| | GSLRLSCA | | | | LKPEDTAVYYC | | |
| | AS | | | | | | |
| 607. | QVRLEKSG | 648. | GWFRQAPGKER | 704. | HYADSVKGRFTISR | | |
| | GGLVQPG | | ELVSA | | DNAKNTVNLQMN | | |
| | GSLTLSCT | | | | SLKVEDTAVYYC | | |
| | AS | | | | | | |
| 608. | QVQLVES | 649. | GWFRQAPGKER | 705. | NYMNFVKGRFTISR | | |
| | GGGLVQP | | ELVAA | | DNAKNTVYLQMTS | | |
| | GGSLKVSC | | | | LKPEDTAVYYC | | |
| | AAS | | | | | | |
| 609. | QVQLAES | 650. | GWFRQAPGKER | 706. | HYADSVKGRFTISR | | |
| | GVGLVEP | | DIVAA | | DNAKNTVDLQMN | | |
| | AGSLKFSC | | | | SLKPEDTAVYYC | | |
| | AAS | | | | | | |
| 610. | QVKLEESG | 651. | YWYRPGPGNQR | 707. | YYADSVKGRFTISR | | |
| | GGLVQPG | | ELVVS | | DNAKNTVSLQMNS | | |
| | GSLKVSCA | | | | LKVEDTAVYYC | | |
| | AS | | | | | | |
| 611. | QVQLVES | 652. | YWYRQGPGKQ | 708. | NYMDSVKGRFTISR | | |
| | GAGLVQP | | RELVLI | | DNAKNTVYLRMSS | | |
| | GGSLRLSC | | | | LKPEDTAVYYC | | |
| | AVS | | | | | | |
| 612. | QVKLEESG | 653. | GWFRQAPGKER | 709. | RYADSVKGRFTISR | | |
| | GGLVQAG | | EFVVA | | DNAKNTVNLQMN | | |
| | GSLRLSCV | | | | SLKPEDTAVYYC | | |
| | VS | | | | | | |
| 613. | QVQLVES | 654. | GWYRPAPGKER | 710. | NYMNSVKGRFTISR | | |
| | GGGLVEA | | ELVAS | | DNAKNTVYLHMTG | | |
| | GGSLTLSC | | | | VKPADTAVYYC | | |
| | AAS | | | | | | |
| | | 655. | GWFRQAPGLER | 711. | NYMDSTEGRFTISR | | |
| | | | EIIAA | | DDAKNTVYLQMTG | | |
| | | | | | VKPADTAVYYC | | |
| | | 656. | GWYRQAPGNQ | 712. | NYMDSVKGRFTISR | | |
| | | | RVLVAS | | DNAKNMVYLQMT | | |
| | | | | | KLKPEDTAVYYC | | |
| | | 657. | TWVRQAPGKGL | 713. | NYMDSVKGRFTISR | | |
| | | | EWVSA | | DNAKNTVYLQMTS | | |
| | | | | | LKPEDTAVYYC | | |
| | | 658. | GWFRQGPGKER | 714. | NYMDSVKGRFTISR | | |
| | | | ESVAA | | DNAKNTVYLQMT | | |
| | | | | | NLKPEDTAVYYC | | |
| | | 659. | GWYRQAPGKQ | 715. | RYEDFVKGRFTISR | | |
| | | | RELAAL | | DNAKSTVYLQMNS | | |
| | | | | | LKPEDTAVYYC | | |
| | | 660. | GWYRQAPGKQ | 716. | NVAQSMEGRFTIS | | |
| | | | RDLVAT | | RDNAKNTGYLQM | | |
| | | | | | NSLKPEDTGVYYC | | |
| | | 661. | GWFREAPGKER | 717. | NYMGFVKGRFTISR | | |
| | | | RFVAS | | DNAKNTVYLQMTS | | |
| | | | | | LKPEDTAVYYC | | |
| | | 662. | GWHRQAPENQ | 718. | NYMDSVKDRFTISR | | |
| | | | RELVAT | | DNAKNTLYLQMTN | | |
| | | | | | LKPEDTAVYYC | | |
| | | 663. | GWYRQAPGKER | 719. | NYMGFVRGRFTISR | | |
| | | | ELVAS | | DNTKNTVYLQMTS | | |
| | | | | | LKPDDTAVYYC | | |
| | | 664. | GWYRQAPGKQ | 720. | NYMDFVKGRFTISR | | |
| | | | RELVAT | | DNAKNTVYLQMTS | | |
| | | | | | LKPEDTAVYYC | | |
| | | 665. | AWFRQAPGKER | 721. | NYPDSVKGRFAISR | | |
| | | | EFVAR | | DNAKNTVYLQMNS | | |
| | | | | | LKPEDTAVYYC | | |
| | | 666. | GWFRQAPGKEH | 722. | AYADSVWGRFTISR | | |
| | | | EFVAA | | DNAKNNVYLEMNS | | |
| | | | | | LKPEDTAVYYC | | |
| | | 667. | AWFRQAPDKER | 723. | AYADSVKGRFTISR | | |
| | | | AVVAT | | DKAKNTVYLQMNS | | |
| | | | | | LKPEDTAVYYC | | |
| | | 668. | GWFRQAPGKER | 724. | AYADSVKGRFTISR | | |
| | | | EFVSP | | DNAKNTVYLQMN | | |
| | | | | | TLKPEDTAVYYC | | |
| | | 669. | GWYRQAPGKQ | 725. | NYADSVKDTISRDN | | |
| | | | RGFVAH | | AKNTVYLQMTSLK | | |
| | | | | | PEDTAVYYC | | |
| | | | | 726. | AYGDSVKGRFSISR | | |
| | | | | | DFAKNTVYLQIDSL | | |
| | | | | | KAEDTAVYYC | | |
| | | | | 727. | SYANSVKGRFTISR | | |
| | | | | | DNTKSTVYLQIHSL | | |
| | | | | | KPEDTAVYYC | | |
| | | | | 728. | AYADSVKGRFTISN | | |
| | | | | | DAAKNTVYLQMNS | | |
| | | | | | LKPDDTAVYYC | | |
| | | | | 729. | HDAHFTKGRFIISK | | |
| | | | | | DDAKNTVYLLMNS | | |
| | | | | | LKPEDTAVYYC | | |
| | | | | 730. | NTADSVKGRFTISR | | |
| | | | | | DNAKNTVYLQMNS | | |
| | | | | | LKPEDTAVYYC | | |
| | | | | 731. | QNAHFPNGQFLISI | | |
| | | | | | YEDDNTMYLQLSIQ | | |
| | | | | | KPEDADVYDC | | |
| | | | | 732. | NYADSVKGRFTISR | | |
| | | | | | DNAKNTVYLQMNS | | |
| | | | | | LNAEDTAVYYC | | |
| | | | | 733. | AYGDSVKGRFSISR | | |
| | | | | | DFAKNTVYLHIDSL | | |
| | | | | | KAEDTAVYYC | | |
| | | | | 734. | DYADSVKDRFTIAR | | |
| | | | | | DNPKNTVTLQMNS | | |
| | | | | | LKPEDTAIYYC | | |
| | | | | 735. | RYADSMSDRFTISR | | |
| | | | | | DNAKNTVYLGMDS | | |
| | | | | | LKPEDTAVYYC | | |
| | | | | 736. | NYAHSVKGTISRHN | | |
| | | | | | AENPVSRLQMNSL | | |
| | | | | | KPEDTAVYYC | | |
| | | | | 737. | YYADSVKGTISRDN | | |
| | | | | | AKNTVYLQMNSLK | | |
| | | | | | PEDTAVYFC | | |
| | | | | 738. | SYADSVKGRFTISR | | |
| | | | | | DNAKNTLYLQMNS | | |
| | | | | | LKPEDTAVYYC | | |
| | | | | 739. | YYTDSVKGRFTISR | | |
| | | | | | DNAKNTVYLQMDS | | |
| | | | | | LKPEDTAVYFC | | |
| | | | | 740. | DYADSAKGRFTISR | | |
| | | | | | DNAKNTVYLQMNS | | |
| | | | | | LKPEDTAVYYC | | |
| | | | | 741. | AYGDSVKGRSNLSV | | |
| | | | | | NHTNNTAYLQMN | | |
| | | | | | SLRPEDTAVYWC | | |
| | | | | 742. | YYTESVKGRFTISRD | | |
| | | | | | NAKNTVYLQMNSL | | |
| | | | | | KPEDTAVYYC | | |
| | | | | 743. | NYADSVKGTISRDN | | |
| | | | | | AKNTVYLQMTSLK | | |
| | | | | | PEDTAVYYC | | |
| | | | | 744. | SYADSVKGTISRDY | | |
| | | | | | AENMVYLQMNSL | | |
| | | | | | KPEDTATYYC | | |
| | | | | 745. | NYADSVKDSISRDH | | |
| | | | | | DTNAVYLQMTNLR | | |
| | | | | | PDDPAVYYC | | |
| | | | | 746. | DYADSVKDTIARDN | | |
| | | | | | PKNTVTLQMNSLK | | |
| | | | | | PEDTAIYYC | | |
| | | | | 747. | NYADSVKDTISRDN | | |
| | | | | | AKNTVYLQMTSLK | | |
| | | | | | PEDTAVFYC | | |
| | | | | 748. | DYADSVKDTIARDN | | |
| | | | | | PKNAVTLQMNSLK | | |
| | | | | | PEDTAIYYC | | |
| | | | | 749. | VYADSVKGTISGDN | | |
| | | | | | AKNTMYLQMNSLK | | |
| | | | | | PEDTAVYYC | | |
| | | | | 750. | YYEDSVKGTVSRDN | | |
| | | | | | AKNTVYLQMNNLK | | |
| | | | | | PEDTAVYYC | | |
| | | | | 751. | SYADSVKGRFTISR | | |
| | | | | | DNAKNMVYLRMD | | |
| | | | | | SLKPEDTDVYYC | | |
| | | | | 752. | DYLDSVKGRFTISR | | |
| | | | | | DNAKNTVYLQMNS | | |
| | | | | | LKPEDTAVYYC | | |

1.-40. (canceled)

41. A polypeptide comprising a a plurality of variable region fragments of a heavy chain antibody (V_{H}H), wherein the VIM specifically binds Salmonella enterica, wherein the plurality of V_{H}Hs comprise at least three V_{H}Hs and wherein one or more of the plurality of V_{H}Hs comprise an amino acid sequence with at least 80% identity to the amino acid sequence set forth in SEQ ID NO: 463.

42. The polypeptide of claim 41, wherein a minimum concentration of the polypeptide required for 50% motility inhibition of Salmonella enterica is reduced by at least 20-fold when compared to a minimum concentration of a control polypeptide required for 50% motility inhibition of Salmonella enterica, the control polypeptide comprising a single V_{H}H that is identical to any one of the plurality of V_{H}Hs of the polypeptide.

43. The polypeptide of claim 41, wherein the V_{H}H specifically binds a Salmonella enterica virulence factor, wherein the virulence factor is involved in bacterial motility, adhesion, invasion, or biofilm formation.

44. The polypeptide of claim 43, wherein the virulence factor comprises a flagellum, FliC, PrgI, FimA, or SipD.

45. The polypeptide of claim 43, wherein the virulence factor comprises a flagellum FliC, PrgI, FimA.

46. The polypeptide of claim 41, wherein the amino acid sequence of the V_{H}H comprises:
a) a CDR1 sequence set forth in SEQ ID NO: 1, a CDR2 sequence set forth in SEQ ID NO: 92, and a CDR3 sequence set forth in SEQ ID NO: 183;
b) a CDR1 sequence set forth in SEQ ID NO: 4, a CDR2 sequence set forth in SEQ ID NO: 95, and a CDR3 sequence set forth in SEQ ID NO: 186;
c) a CDR1 sequence set forth in SEQ ID NO: 12, a CDR2 sequence set forth in SEQ ID NO: 103, and a CDR3 sequence set forth in SEQ ID NO: 194;
d) a CDR1 sequence set forth in SEQ ID NO: 20, a CDR2 sequence set forth in SEQ ID NO: 111, and a CDR3 sequence set forth in SEQ ID NO: 202
e) a CDR1 sequence set forth in SEQ ID NO: 21, a CDR2 sequence set forth in SEQ ID NO: 112, and a CDR3 sequence set forth in SEQ ID NO: 203; or
f) a CDR1 sequence set forth in SEQ ID NO: 60, a CDR2 sequence set forth in SEQ ID NO: 151, and a CDR3 sequence set forth in SEQ ID NO: 242.

47. The polypeptide of claim 41, wherein the amino acid sequence of the V_{H}H comprises:
a) a CDR1 sequence set forth in SEQ ID NO: 5, a CDR2 sequence set forth in SEQ ID NO: 96, and a CDR3 sequence set forth in SEQ ID NO: 187;
b) a CDR1 sequence set forth in SEQ ID NO: 285, a CDR2 sequence set forth in SEQ ID NO: 335, and a CDR3 sequence set forth in SEQ ID NO: 385; or
c) a CDR1 sequence set forth in SEQ ID NO: 284, a CDR2 sequence set forth in SEQ ID NO: 334, and a CDR3 sequence set forth in SEQ ID NO: 384.

48. The polypeptide of claim 41, wherein the amino acid sequence of the V_{H}H comprises an amino acid sequence at least 80% identical to that set forth in any one SEQ ID NOs: 478 to 488.

49. The polypeptide of claim 41, wherein the V_{H}H reduces Salmonella enterica bacterial motility compared to a negative control antibody by at least 40%.

50. The polypeptide of claim 41, wherein the V_{H}H reduces biofilm formation by at least 10%.
